SEQUENCE LISTING (56 sequences)

SEQ ID NO: 1 - DNA, 508 bp, Artificial Sequence
  Human cytomegalovirus
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 2 - DNA, 20 bp, Artificial Sequence
  T7 priming site
taatacgactcactataggg

SEQ ID NO: 3 - DNA, 239 bp, Artificial Sequence
  BGH polyA
agcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctagtgggctct

SEQ ID NO: 4 - DNA, 679 bp, Artificial Sequence
  pUC origin of replication
ccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 5 - DNA, 800 bp, Artificial Sequence
  Kanamycin resistance marker
ttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 6 - DNA, 3779 bp, Artificial Sequence
  Wild type human Maxi-K alpha subunit (Slo)
atggcaaacggtggcggcggcggcggcggcagcagcggcggcggcggcggcggcggcggaggcagcggtcttagaatgagcagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctactttggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagctgggttcatccatttggtggagaattcaggggacccatgggaaaatttccaaaacaaccaggctctcacctactgggaatgtgtcatttactcatggtcacaatgtccaccgttggttatggggatgtttatgcaaaaaccacacttcggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatccgagctggaattggaaagaaggtgatgacgcaatctgcctcgcagagttgaagttgggcttcatagcccagagctgcctggctcaaggcctctccaccatgcttgccaaccttctccatgaggtcattcataaagattgaggaagacacatggcagaaatactacttggaaggagtctcaaatcaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccattttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttgatatgtgtatccaccgccactgtgtgaaactgtatctgccactcatttccccagttggtgtttccaacaaagtaactttccctgttttcccctgtagtccccccctttttttttacacatatttgcatatgtatgatagtgtgcatgtggttgtcatttttatttcaccaccataaaacccttgagcacaacagcaaataagcagacgggctccggaattcctgcagcccgggggatccactag

SEQ ID NO: 7 - DNA, 3533 bp, Artificial Sequence
  hSlo ORF, NA; wild type human Maxi-K alpha subunit (Slo)
atggcaaatggtggcggcggcggcggcggcagcagcggcggcggcggcggcggcggaggcagcagtcttagaatgagtagcaatatccacgcgaaccatctcagcctagacgtgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagagagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctt

SEQ ID NO: 8 - protein, 1178 aa, Artificial Sequence
  hSlo T352S mutant
MANGGGGGGGSSGGGGGGGGSSLRMSSNIHANHLSLDVSSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 9 - DNA, 1419 bp, Artificial Sequence
  SM22alpha promoter sequence
gaattcaggacgtaatcagtggctggaaagcaagagctctagaggagctctgacccttccttcagatgccacaaggaggtgctggagttctatgcaccaacagccaggctggctgtagtggattgagcgtctgaggctgcacctctctgggttctgggtgagactgaccctgcctgagggttctctccttccctctctctccctctccctctccctctctctgtttcctgaggtttccaggattggggatgacaccactaaagccttaccttttaagaagttgcattcagtgagtgtgtgcagataggggcagaggagagctggttctgtctccactgtgtttggtcttgtcagaccatcaggtgtgatagcagttgtctttaaccctaaccctgagccttcccttcccaagaccactgaagctaggtgcaagataagtggggaccctttaggatctttcacgataaggactattttgaagggagggagggtgacactgtttaccctagtgtctccagccttgccaggccttaaacatccgcccattgtcaaggggccagggttgacttgctgctaaacaaggcactccctagagaagcagcataccatacctgtgggcaggatgacccatgttctgccacgcacttggtaggccactttgaacctcaattttctcaactgttaaatggggtggtaactgataaaggggaacgtgaaaggaaggcgtttgcatagtgcctggttgtgcaggtcaagactagttcccaccaactcgattttaaagccttgcaagaaggtggccttgcaggttcctttgtcgggccaaactctagaatgcctccccctttctagagcagacccaagtccgggtaacaaggaagggtttcagggtcctgcccattcccggccgccctcagcaccgccccgccccgacccccgcagcatctccacagcttattatagcttaaaccctgcagccaactcctttctgggactcagaagacatagcaggtactgaacgtctcacctgctgaggtggtcctagtcctcacccgctctagcccgctagaagccttggaactatctcataccaggctgcacttgtttgtcttctcattgataaaaggtttaagcatgcagagaatgtctccggctgcccccgacagactgctccaacttggtgtctttccccaaatatggagcctgtgtggagtgagtggggcggcccggggtggtgagccaagcagacttccatgggcagggaggggcgccagcggacggcagaggggtgacatcactgcctaggcggcctttaaacccctcacccagccggcgccccagcccgtctgccccagcccagacaccgaagctactctccttccagtccacaaacgaccaagccttgtaagtgcaagtcat

SEQ ID NO: 10 - DNA, 2999 bp, Artificial Sequence
  pVAX vector
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 11 - DNA, 33 bp, Artificial Sequence
  Mutated Slo subsequence
atggtcacaatgtcctccgttggttatggggat

SEQ ID NO: 12 - DNA, 33 bp, Artificial Sequence; identical to SEQ ID NO: 11
  Primer to generate mutant

SEQ ID NO: 13 - DNA, 33 bp, Artificial Sequence
  Primer to generate mutant
atccccataaccaacggaggacattgtgaccat

SEQ ID NO: 14 - DNA, 3534 bp, Artificial Sequence
  Wild type SLO, NA
atggcaaatggtggcggcggcggcggcggcagcagcggcggcggcggcggcggcggaggcagcagtcttagaatgagtagcaatatccacgcgaaccatctcagcctagacgtgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctt

SEQ ID NO: 15 - protein, 1178 aa, Artificial Sequence; identical to SEQ ID NO: 8
  Wild type SLO, Protein

SEQ ID NO: 16 - DNA, 6880 bp, Artificial Sequence
  pVAX/hSlo1 WT
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagcttcttgttctttttgcagaagctcagaataaacgctcaactttggcagaatcgataagcttgatcgagcccctgcgcctgccgcccattgctagctatggcaaacggtggcggcggcggcggcggcagcagcggcggcggcggcggcggcggcggaggcagcggtcttagaatgagcagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctactttggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagctgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttcggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttgatatgtgtatccaccgccactgtgtgaaactgtatctgccactcatttccccagttggtgtttccaacaaagtaactttccctgttttcccctgtagtcccccccctttttttttacacatatttgcatatgtatgatagtgtgcatgtggttgtcatttttatttcaccaccataaaacccttgagcacaacagcaaataagcagacgggctccggaattctgcagcccgggggatccactagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 17 - protein, 1236 aa, Artificial Sequence
  Maxi-K alpha subunit (Slo), isoform 1
MANGGGGGGGSSGGGGGGGGSSLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRPKMSIYKRMRRACCFDCGRSERDCSCMSGRVRGNVDTLERAFPLSSVSVNDCSTSFRAFEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 18 - protein, 1219 aa, Artificial Sequence
  Maxi-K alpha subunit (Slo), isoform 2
MANGGGGGGGSSGGGGGGGGSSLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILVTGWMPYLGPRVLMTCLDIGVVCMPTDIQSTSPASIKKFKETRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 19 - protein, 1240 aa, Artificial Sequence
  Maxi-K alpha subunit (Slo), isoform 3
MANGGGGGGGSSGGGGGGGGSSLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRSRKRILINPGNHLKIQEGTLGFFIASDAKEVKRAFFYCKACHDDITDPKRIKKCGCKRPKMSIYKRMRRACCFDCGRSERDCSCMSGRVRGNVDTLERAFPLSSVSVNDCSTSFRAFEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 20 - protein, 1207 aa, Artificial Sequence
  Maxi-K alpha subunit (Slo), isoform 4
MANGGGGGGGSSGGGGGGGGSSLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLKVAARSRYSKDPFEFKKETPNSRLVTEPVEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 21 - protein, 1178 aa, Artificial Sequence
  Maxi-K alpha subunit (Slo), isoform 5
MANGGGGGGGSSGGGGGGGGSSLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 22 - protein, 168 aa, Artificial Sequence
  Maxi-K alpha subunit (Slo), isoform 6
MANGGGGGGGSSGGGGGGGGSSLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKATHFGSPEMPPAARSWSGSPPEAAVLRGASSLALEVARCRRL

SEQ ID NO: 23 - protein, 1239 aa, Artificial Sequence
  Maxi-K alpha subunit (Slo), isoform 7
MANGGGGGGGSSGGGGGGGGSSLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRRWEEHCSLWRLESKGNVRRLNYCRGQQTFSVKVKVAARSRYSKDPFEFKKETPNSRLVTEPVEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 24 - protein, 191 aa, Artificial Sequence
  Maxi-K beta 1 subunit (Slo), isoform 1
MVKKLVMAQKRGETRALCLGVTMVVCAVITYYILVTTVLPLYQKSVWTQESKCHLIETNIRDQEELKGKKVPQYPCLWVNVSAAGRWAVLYHTEDTRDQNQQCSYIPGSVDNYQTARADVEKVRAKFQEQQVFYCFSAPRGNETSVLFQRLYGPQALLFSLFWPTFLLTGGLLIIAMVKSNQYLSILAAQK

SEQ ID NO: 25 - protein, 130 aa, Artificial Sequence
  Maxi-K beta 1 subunit (Slo), isoform 2
MVKKLVMAQKRGETRALCLGVTMVVCAVITYYILVTTVLPLYQKSVWTQESKCHLIETNIRDQEELKGKKVPQYPCLWVNVSAAGRWAVLYHTEDTRDQNQQVLNWRDGDTSLYPCQVCEPVPNCPCPRG

SEQ ID NO: 26 - protein, 235 aa, Artificial Sequence
  Maxi-K beta 2 subunit (Slo)
MFIWTSGRTSSSYRHDEKRNIYQKIRDHDLLDKRKTVTALKAGEDRAILLGLAMMVCSIMMYFLLGITLLRSYMQSVWTEESQCTLLNASITETFNCSFSCGPDCWKLSQYPCLQVYVNLTSSGEKLLLYHTEETIKINQKCSYIPKCGKNFEESMSLVNVVMENFRKYQHFSCYSDPEGNQKSVILTKLYSSNVLFHSLFWPTCMMAGGVAIVAMVKLTQYLSLLCERIQRINR

SEQ ID NO: 27 - protein, 279 aa, Artificial Sequence
  Maxi-K beta 3 subunit (Slo), isoform 1
MDFSPSSELGFHFVAFILLTRHRTAFPASGKKRETDYSDGDPLDVHKRLPSSAGEDRAVMLGFAMMGFSVLMFFLLGTTILKPFMLSIQREESTCTAIHTDIMDDWLDCAFTCGVHCHGQGKYPCLQVFVNLSHPGQKALLHYNEEAVQINPKCFYTPKCHQDRNDLLNSALDIKEFFDHKNGTPFSCFYSPASQSEDVILIKKYDQMAIFHCLFWPSLTLLGGALIVGMVRLTQHLSLLCEKYSTVVRDEVGGKVPYIEQHQFKLCIMRRSKGRAEKS

SEQ ID NO: 28 - protein, 277 aa, Artificial Sequence
  Maxi-K beta 3 subunit (Slo), isoform 2
MQPFSIPVQITLQGSRRRQGRTAFPASGKKRETDYSDGDPLDVHKRLPSSAGEDRAVMLGFAMMGFSVLMFFLLGTTILKPFMLSIQREESTCTAIHTDIMDDWLDCAFTCGVHCHGQGKYPCLQVFVNLSHPGQKALLHYNEEAVQINPKCFYTPKCHQDRNDLLNSALDIKEFFDHKNGTPFSCFYSPASQSEDVILIKKYDQMAIFHCLFWPSLTLLGGALIVGMVRLTQHLSLLCEKYSTVVRDEVGGKVPYIEQHQFKLCIMRRSKGRAEKS

SEQ ID NO: 29 - protein, 275 aa, Artificial Sequence
  Maxi-K beta 3 subunit (Slo), isoform 3
MFPLLYELTAVSPSPFPQRTAFPASGKKRETDYSDGDPLDVHKRLPSSAGEDRAVMLGFAMMGFSVLMFFLLGTTILKPFMLSIQREESTCTAIHTDIMDDWLDCAFTCGVHCHGQGKYPCLQVFVNLSHPGQKALLHYNEEAVQINPKCFYTPKCHQDRNDLLNSALDIKEFFDHKNGTPFSCFYSPASQSEDVILIKKYDQMAIFHCLFWPSLTLLGGALIVGMVRLTQHLSLLCEKYSTVVRDEVGGKVPYIEQHQFKLCIMRRSKGRAEKS

SEQ ID NO: 30 - protein, 257 aa, Artificial Sequence
  Maxi-K beta 3 subunit (Slo), isoform 4
MTAFPASGKKRETDYSDGDPLDVHKRLPSSAGEDRAVMLGFAMMGFSVLMFFLLGTTILKPFMLSIQREESTCTAIHTDIMDDWLDCAFTCGVHCHGQGKYPCLQVFVNLSHPGQKALLHYNEEAVQINPKCFYTPKCHQDRNDLLNSALDIKEFFDHKNGTPFSCFYSPASQSEDVILIKKYDQMAIFHCLFWPSLTLLGGALIVGMVRLTQHLSLLCEKYSTVVRDEVGGKVPYIEQHQFKLCIMRRSKGRAEKS

SEQ ID NO: 31 - protein, 173 aa, Artificial Sequence
  Maxi-K beta 3 subunit (Slo), isoform 5
MQPFSIPVQITLQGSRRRQGRTAFPASGKKRETDYSDGDPLDVHKRLPSSAGEDRAVMLGFAMMGFSVLMFFLLGTTILKPFMLSIQREESTCTAIHTDIMDDWLDCAFTCGVHCHGQGKYPCLQVFVNLSHPGQKALLHYNEEAVQINPKRDVTDCRVKEKQTLTVSDEHKQ

SEQ ID NO: 32 - protein, 210 aa, Artificial Sequence
  Maxi-K beta 4 subunit (Slo)
MAKLRVAYEYTEAEDKSIRLGLFLIISGVVSLFIFGFCWLSPALQDLQATEANCTVLSVQQIGEVFECTFTCGADCRGTSQYPCVQVYVNNSESNSRALLHSDEHQLLTNPKCSYIPPCKRENQKNLESVMNWQQYWKDEIGSQPFTCYFNQHQRPDDVLLHRTHDEIVLLHCFLWPLVTFVVGVLIVVLTICAKSLAVKAEAMKKRKFS

SEQ ID NO: 33 - DNA, 3711 bp, Homo sapiens
atggcaaatggtggcggcggcggcggcggcagcagcggcggcggcggcggcggcggaggcagcagtcttagaatgagtagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcccaagatgtccatctacaagagaatgagacgggcatgttgttttgattgcggacgttctgagcgtgactgctcatgcatgtcaggccgtgtgcgtggtaacgtggacacccttgagagagccttcccactttcttctgtctctgttaatgattgctccaccagtttccgtgcctttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttga

SEQ ID NO: 34 - DNA, 3660 bp, Homo sapiens
atggcaaatggtggcggcggcggcggcggcagcagcggcggcggcggcggcggcggaggcagcagtcttagaatgagtagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctggtcacaggctggatgccgtatctgggacctagggttttaatgacttgcctggatattggtgtagtatgcatgccaactgatattcaatcaacatctcctgccagcataaaaaagtttaaggagactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttga

SEQ ID NO: 35 - DNA, 3537 bp, Homo sapiens
atggcaaatggtggcggcggcggcggcggcagcagcggcggcggcggcggcggcggaggcagcagtcttagaatgagtagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttga

SEQ ID NO: 36 - DNA, 507 bp, Homo sapiens
atggcaaatggtggcggcggcggcggcggcagcagcggcggcggcggcggcggcggaggcagcagtcttagaatgagtagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggccacccactttgggtccccggaaatgccaccagcagcgcggagctggagcgggagtccgcctgaggccgcggttttacgcggagcgtcttccctggcgctcgaggtggctagatgtcgtcggctttag

SEQ ID NO: 37 - DNA, 576 bp, Homo sapiens
atggtgaagaagctggtgatggcccagaagcggggagagacacgagccctttgcctgggtgtaaccatggtggtgtgtgccgtcatcacctactacatcctggtcacgactgtgctgcccctctaccagaaaagcgtgtggacccaggaatccaagtgccacctgattgagaccaacatcagggaccaggaggagctgaagggcaagaaggtgccccagtacccatgcctgtgggtcaacgtgtcagctgccggcaggtgggctgtgctgtaccacacggaggacactcgggaccagaaccagcagtgctcctacatcccaggcagcgtggacaattaccagacggcccgggccgacgtggagaaggtcagagccaaattccaagagcagcaggtcttctactgcttctccgcacctcgggggaacgaaaccagcgtcctattccagcgcctctacgggccccaggccctcctcttctccctcttctggcccaccttcctgctgaccggtggcctcctcattatcgccatggtgaagagcaaccagtacctgtccatcctggcggcccagaagtag

SEQ ID NO: 38 - DNA, 708 bp, Homo sapiens
atgtttatatggaccagtggccggacctcttcatcttatagacatgatgaaaaaagaaatatttaccagaaaatcagggaccatgacctcctggacaaaaggaaaacagtcacagcactgaaggcaggagaggaccgagctattctcctgggactggctatgatggtgtgctccatcatgatgtattttctgctgggaatcacactcctgcgctcatacatgcagagcgtgtggaccgaagagtctcaatgcaccttgctgaatgcgtccatcacggaaacatttaattgctccttcagctgtggtccagactgctggaaactttctcagtacccctgcctccaggtgtacgttaacctgacttcttccggggaaaagctcctcctctaccacacagaagagacaataaaaatcaatcagaagtgctcctatatacctaaatgtggaaaaaattttgaagaatccatgtccctggtgaatgttgtcatggaaaacttcaggaagtatcaacacttctcctgctattctgacccagaaggaaaccagaagagtgttatcctaacaaaactctacagttccaacgtgctgttccattcactcttctggccaacctgtatgatggctgggggtgtggcaattgttgccatggtgaaacttacacagtacctctccctactatgtgagaggatccaacggatcaatagataa

SEQ ID NO: 39 - DNA, 840 bp, Homo sapiens
atggacttttcaccaagctctgaactgggatttcattttgttgcattcatcctgctcacgagacacaggacagcctttcctgcctcagggaagaagagagagacagactacagtgatggagacccactagatgtgcacaagaggctgccatccagtgctggagaggaccgagccgtgatgctggggtttgccatgatgggcttctcagtcctaatgttcttcttgctcggaacaaccattctaaagccttttatgctcagcattcagagagaagaatcgacctgcactgccatccacacagatatcatggacgactggctggactgtgccttcacctgtggtgtgcactgccacggtcaggggaagtacccgtgtcttcaggtgtttgtgaacctcagccatccaggtcagaaagctctcctacattataatgaagaggctgtccagataaatcccaagtgcttttacacacctaagtgccaccaagatagaaatgatttgctcaacagtgctctggacataaaagaattcttcgatcacaaaaatggaacccccttttcatgcttctacagtccagccagccaatctgaagatgtcattcttataaaaaagtatgaccaaatggctatcttccactgtttattttggccttcactgactctgctaggtggtgccctgattgttggcatggtgagattaacacaacacctgtccttactgtgtgaaaaatatagcactgtagtcagagatgaggtaggtggaaaagtaccttatatagaacagcatcagttcaaactgtgcattatgaggaggagcaaaggaagagcagagaaatcttaa

SEQ ID NO: 40 - DNA, 834 bp, Homo sapiens
atgcagcccttcagcatccccgtgcaaatcacacttcagggcagccggaggcgccaggggaggacagcctttcctgcctcagggaagaagagagagacagactacagtgatggagacccactagatgtgcacaagaggctgccatccagtgctggagaggaccgagccgtgatgctggggtttgccatgatgggcttctcagtcctaatgttcttcttgctcggaacaaccattctaaagccttttatgctcagcattcagagagaagaatcgacctgcactgccatccacacagatatcatggacgactggctggactgtgccttcacctgtggtgtgcactgccacggtcaggggaagtacccgtgtcttcaggtgtttgtgaacctcagccatccaggtcagaaagctctcctacattataatgaagaggctgtccagataaatcccaagtgcttttacacacctaagtgccaccaagatagaaatgatttgctcaacagtgctctggacataaaagaattcttcgatcacaaaaatggaacccccttttcatgcttctacagtccagccagccaatctgaagatgtcattcttataaaaaagtatgaccaaatggctatcttccactgtttattttggccttcactgactctgctaggtggtgccctgattgttggcatggtgagattaacacaacacctgtccttactgtgtgaaaaatatagcactgtagtcagagatgaggtaggtggaaaagtaccttatatagaacagcatcagttcaaactgtgcattatgaggaggagcaaaggaagagcagagaaatcttaa

SEQ ID NO: 41 - DNA, 828 bp, Homo sapiens
atgttcccccttctttatgagctcactgcagtatctccttctccctttccccaaaggacagcctttcctgcctcagggaagaagagagagacagactacagtgatggagacccactagatgtgcacaagaggctgccatccagtgctggagaggaccgagccgtgatgctggggtttgccatgatgggcttctcagtcctaatgttcttcttgctcggaacaaccattctaaagccttttatgctcagcattcagagagaagaatcgacctgcactgccatccacacagatatcatggacgactggctggactgtgccttcacctgtggtgtgcactgccacggtcaggggaagtacccgtgtcttcaggtgtttgtgaacctcagccatccaggtcagaaagctctcctacattataatgaagaggctgtccagataaatcccaagtgcttttacacacctaagtgccaccaagatagaaatgatttgctcaacagtgctctggacataaaagaattcttcgatcacaaaaatggaacccccttttcatgcttctacagtccagccagccaatctgaagatgtcattcttataaaaaagtatgaccaaatggctatcttccactgtttattttggccttcactgactctgctaggtggtgccctgattgttggcatggtgagattaacacaacacctgtccttactgtgtgaaaaatatagcactgtagtcagagatgaggtaggtggaaaagtaccttatatagaacagcatcagttcaaactgtgcattatgaggaggagcaaaggaagagcagagaaatcttaa

SEQ ID NO: 42 - DNA, 774 bp, Homo sapiens
atgacagcctttcctgcctcagggaagaagagagagacagactacagtgatggagacccactagatgtgcacaagaggctgccatccagtgctggagaggaccgagccgtgatgctggggtttgccatgatgggcttctcagtcctaatgttcttcttgctcggaacaaccattctaaagccttttatgctcagcattcagagagaagaatcgacctgcactgccatccacacagatatcatggacgactggctggactgtgccttcacctgtggtgtgcactgccacggtcaggggaagtacccgtgtcttcaggtgtttgtgaacctcagccatccaggtcagaaagctctcctacattataatgaagaggctgtccagataaatcccaagtgcttttacacacctaagtgccaccaagatagaaatgatttgctcaacagtgctctggacataaaagaattcttcgatcacaaaaatggaacccccttttcatgcttctacagtccagccagccaatctgaagatgtcattcttataaaaaagtatgaccaaatggctatcttccactgtttattttggccttcactgactctgctaggtggtgccctgattgttggcatggtgagattaacacaacacctgtccttactgtgtgaaaaatatagcactgtagtcagagatgaggtaggtggaaaagtaccttatatagaacagcatcagttcaaactgtgcattatgaggaggagcaaaggaagagcagagaaatcttaa

SEQ ID NO: 43 - DNA, 522 bp, Homo sapiens
atgcagcccttcagcatccccgtgcaaatcacacttcagggcagccggaggcgccaggggaggacagcctttcctgcctcagggaagaagagagagacagactacagtgatggagacccactagatgtgcacaagaggctgccatccagtgctggagaggaccgagccgtgatgctggggtttgccatgatgggcttctcagtcctaatgttcttcttgctcggaacaaccattctaaagccttttatgctcagcattcagagagaagaatcgacctgcactgccatccacacagatatcatggacgactggctggactgtgccttcacctgtggtgtgcactgccacggtcaggggaagtacccgtgtcttcaggtgtttgtgaacctcagccatccaggtcagaaagctctcctacattataatgaagaggctgtccagataaatcccaagcgtgatgttacagactgcagagttaaagaaaagcagacattgacagtttctgatgagcataaacagtaa

SEQ ID NO: 44 - DNA, 633 bp, Homo sapiens
atggcgaagctccgggtggcttacgagtacacggaagccgaggacaagagcatccggctcggcttgtttctcatcatctccggcgtcgtgtcgctcttcatcttcggcttctgctggctgagtcccgcgctgcaggatctgcaagccacggaggccaattgcacggtgctgtcggtgcagcagatcggcgaggtgttcgagtgcaccttcacctgtggcgccgactgcaggggcacctcgcagtacccctgcgtccaggtctacgtgaacaactctgagtccaactctagggcgctgctgcacagcgacgagcaccagctcctgaccaaccccaagtgctcctatatccctccctgtaagagagaaaatcagaagaatttggaaagtgtcatgaattggcaacagtactggaaagatgagattggttcccagccatttacttgctattttaatcaacatcaaagaccagatgatgtgcttctgcatcgcactcatgatgagattgtcctcctgcattgcttcctctggcccctggtgacatttgtggtgggcgttctcattgtggtcctgaccatctgtgccaagagcttggcggtcaaggcggaagccatgaagaagcgcaagttctcttaa

SEQ ID NO: 45 - DNA, 6880 bp, Artificial Sequence
  pVAX-hSlo1-C911A
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagcttcttgttctttttgcagaagctcagaataaacgctcaactttggcagaatcgataagcttgatcgagcccctgcgcctgccgcccattgctagctatggcaaacggtggcggcggcggcggcggcagcagcggcggcggcggcggcggcggcggaggcagcggtcttagaatgagcagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctactttggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagctgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttcggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgccgctgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttgatatgtgtatccaccgccactgtgtgaaactgtatctgccactcatttccccagttggtgtttccaacaaagtaactttccctgttttcccctgtagtcccccccctttttttttacacatatttgcatatgtatgatagtgtgcatgtggttgtcatttttatttcaccaccataaaacccttgagcacaacagcaaataagcagacgggctccggaattctgcagcccgggggatccactagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 46 - DNA, 6591 bp, Artificial Sequence
  pVAX-hSlo1-deltaNX
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagaccaccatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctactttggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagctgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttcggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttgatatgtgtatccaccgccactgtgtgaaactgtatctgccactcatttccccagttggtgtttccaacaaagtaactttccctgttttcccctgtagtcccccccctttttttttacacatatttgcatatgtatgatagtgtgcatgtggttgtcatttttatttcaccaccataaaacccttgagcacaacagcaaataagcagacgggctccggaattctgcagcccgggggatccactagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 47 - protein, 1113 aa, Artificial Sequence
  hSlo encoded by deltaNX
MDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLRRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 48 - DNA, 11730 bp, Artificial Sequence
  pSMAA-hSlo
acatgtgcacagttcagaagcactcccagaatccatccaaaatatctctatcgtgaatggaatcagaaccttggcttgcaggaggaaagtacagaaatgtaaagtcactgactgtccatcaaagccaacgatctgatgcctttgaagaatgatagggtcacttgaggtcacttgatctctgtttctgtccagtgggctcatagtcatggaggagagtgagcaggcttcatttcaacatttcaaatttcttttacaaagttttttttttttatttttttatgacagggtgactggtgatctctgtgggcaaaggatggtccttaatcatgctgttaagggtcagtaaaaagccagcaacatgcggaatgttaagggttaaagcagttacagtgattctgacttctaagttactctttgggcaacacaggctggttaatcctcactacatacttcagttcctggtttcattactacaacacaaagacacaatgtataagtacaatgtagcttccataaaaacatgactcctctgcatatttatgggtgactcgaagcatcttttgatctaggctaccttttgcaacagtgttgcttaaaaatcgcagctagtcagagacaggcccttccttatccaagtcctcagctaatggcccaaaagactagccagacaggggctggcatcttctgaggaatgtgcaaaccgtgcctgcgtctgtcccatgacactagcccagtgtctgggcatttgagcagttgttctgagggcttaggatgtttatccccataagcagctgagctgcctcctgtttcgggagcagaacagaggaatgcagtggaagagacccaggcctctggccacccagattagagagttttgtgctgaggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagggtaagtagccccagcccagggatatgacttcgagttttcccaggctcttttatcatccaatgtagccagacattgtctgtgggaatctgaatgactcacgtgttttgaatttttgaataaagatttatactgttaaaatgattgtagctttttagcttgcatgattttacatccgaatagggctgatttactggaaacaacgcttgatttactggaaaaggaaatggatagaaaattaaagtttgttcatgtgtgtcatctgcaaaacctgtttacactaaaccaactgctctgatcccgcagcgtactgtaggggtggagtctagctgtatgtggtaaattatacgtttgtttctacttaggcaaaagttggaaacttttggatgtatcatgatgtagcatgaggtatttagtgcagctgaggtaactggaagtgaatatcaggaatgaactgaggtagttgcctgctctctgatgttggctgagtggacgcattgcttctgggtttccggggctctaagagctggtgtcctatgctggaaatgtgtatcttgtgagtgtgttggtgcccttacaagtcagacctatgccattggtcatttgcagcatagcatagcttttctactttctgcaaagaaaggaggaagtgtctcatccaggggagatctgatttgcatttctctgcctcacgtgtccctcagccgctaagtatctgtggaaccagccttgccaccccacattgtaactcagggctcggtagcttcatcagggaatggagttttctcgataagattttcctcctgttttgtgattcatgactaaatatggtttgcatttgagactcataagctgggaagggtactgtcctttcctcccctcccccctccccccaacaattcatttttggcaccagatgagctccactgggctgcaccaaactccccgccccggtgcagttccaaaagcagaggctggagcccagtgtgttttacctaattaggaaatgctccccgcttcaaaccgagctgctcattcaggttagataagagttgcaaaccacagcggctgcgtcctctggaaacacacagacttcttctccagtgacaacgctcctttcagagcttaataagacaattttttcctggatatttttgatgaaatagaaatacatctttacggaatttgacagtattttttcctgcatttttttaaaaaccaggtagcttatttttctgaatatactaaggcacaaccttaagccatcctgcccaacaaaaagttaatatgagggttatccttccccattttcagagggtatcctagttccaagtggcttatcccattgtcggggggggtactaagtatggaaaacaggcttagtggacacacagactctgtgttggtcctttggggtttcggcatataccagtcacctggattctgtgcctcctggtggtgtgaacctttcttctgagtccttatcatgcactggaaaggaagtaagcataaatcagaggcatagtggaaagaggaaagatcaagtgctgaagaaacccattttcccattcttgcaaggggaacacattgaagatttcactatgattttggggacagaggttgaaagaaaaccaagatcgcaaacagaatctttgggtaggaataatagttacttgatgatatccacgcacaatgcttgttcaacactatggatgtccatgggagctctcaaaaatccaagcttaaatgtcaattccttaaattgttgttaaaaacaaccctaaggggtatatactcagttaatcaagcttagaagaagataccagagctcagggaagaaaaaaagtctacaaaagctgatgcttgccacttcaaaagaatcctagtaaacattttggacagaataagtaagctttgggggtagaggaacaactcacattttattaaggtcatattctgtctctttctgtaacttatcagtcttaaacaagaatagctctcagcaacctgttgggttttcagcttaacagtgactttaataaatgaagaaatgttataactcgtaaaatttcaaacaccatatttggaaatttctatccaagtttccatattagaccagctccttaacttgtgatcctcctgcctcagcctcccaagtgctaggatcataggtgtacatcatcacacccagccttgattcatatttaatacctcaccggctcacaagtctttagagccaaaagttttctcttttaaacatttaatatgagtaaacattttaacattttcaaattctcacatgctgcccattccttgaaaatctacctttggtgggggggggggggggactatatatatatatgtccctatagaactctgctctctacactgcatctctcatctgtgctctatgatctattcacacactaatgctctgaccagcttgagagtgttaatagagcgagtgacactcccgctattggtgctgaggacttgtggtgttaacctggaagtcagggtttcggatcatcaaagtctttacagcatagtgaaagcatttcaagataaagggtgttagttgagaactgtggagagcctccagctaaaataacccaacaggtccaagaaccctgtctgtgggtggagtgaataggctatagccaaaagctatgcgttacagtagcatttcgctcgaagtctctgcagaaccctgaggcgctggatcctctagtccagtggggtggaattctgcagatatccagcacagtggcggccgctcgagcttcttgttctttttgcagaagctcagaataaacgctcaactttggcagaatcgataagcttgatcgagcccctgcgcctgccgcccattgctagctatggcaaacggtggcggcggcggcggcggcagcagcggcggcggcggcggcggcggcggaggcagcggtcttagaatgagcagcaatatccacgcgaaccatctcagcctagacgcgtcctcctcctcctcctcctcctcttcctcttcttcttcttcctcctcctcttcctcctcgtcccggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctactttggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagctgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgcgcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttgatatgtgtatccaccgccactgtgtgaaactgtatctgccactcatttccccagttggtgtttccaacaaagtaactttccctgttttcccctgtagtcccccccctttttttttacacatatttgcatatgtatgatagtgtgcatgtggttgtcatttttatttcaccaccataaaacccttgagcacaacagcaaataagcagacgggctccggaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 49 - DNA, 6880 bp, Artificial Sequence
  pVax-hSlo Variant 1
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagcttcttgttctttttgcagaagctcagaataaacgctcaactttggcagaatcgataagcttgatcgagcccctgcgcctgccgcccattgctagctatggcaaacggtggcggcggcggcggcggcagcagcggcggcggcggcggcggcggcggaggcagcagtcttagaatgagtagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttgatatgtgtatccaccgccactgtgtgaaactgtatctgccactcatttccccagttggtgtttccaacaaagtaactttccccgttttcccctgtagtcccccccctttttttttacacatatttgcatatgtatgatagtgtgcatgtggttgtcatttttatttcaccaccataaaacccttgagcacaacagcaaataagcagacgggctccggaattctgcagcccgggggatccactagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 50 - DNA, 6879 bp, Artificial Sequence
  pVax-hSlo Variant 2
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagcttcttgttctttttgcagaagctcagaataaacgctcaactttggcagaatcgataagcttgatcgagcccctgcgcctgccgcccattgctagctatggcaaacggtggcggcggcggcggcggcagcagcggcggcggcggcggcggcggcggaggcagcggtcttagaatgagcagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagctgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttgatatgtgtatccaccgccactgtgtgaaactgtatctgccactcatttccccagttggtgtttccaacaaagtaactttccctgttttcccctgtagtccccccctttttttttacacatatttgcatatgtatgatagtgtgcatgtggttgtcatttttatttcaccaccataaaacccttgagcacaacagcaaataagcagacgggctccggaattcctgcagcccgggggatccactagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 51 - DNA, 3538 bp, Artificial Sequence
  pVax-hSlo ORF
atggcaaacggtggcggcggcggcggcggcagcagcggcggcggcggcggcggcggcggaggcagcggtcttagaatgagcagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctactttggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagctgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttcggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggcttt

SEQ ID NO: 52 - DNA, 3538 bp, Artificial Sequence
  pVax-hSlo ORF
atggcaaacggtggcggcggcggcggcggcagcagcggcggcggcggcggcggcggcggaggcagcagtcttagaatgagtagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggcttt

SEQ ID NO: 53 - DNA, 3538 bp, Artificial Sequence
  pVax-hSlo ORF
atggcaaacggtggcggcggcggcggcggcagcagcggcggcggcggcggcggcggcggaggcagcggtcttagaatgagcagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagctgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggcttt

SEQ ID NO: 54 - protein, 1179 aa, Artificial Sequence
  hSlo - Translated ORF
MANGGGGGGGSSGGGGGGGGGSGLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLRRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 55 - protein, 1179 aa, Artificial Sequence
  hSlo - Translated ORF
MANGGGGGGGSSGGGGGGGGGSSLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 56 - protein, 1179 aa, Artificial Sequence
  hSlo - Translated ORF
MANGGGGGGGSSGGGGGGGGGSGLRMSSNIHANHLSLDASSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

(19) United States
(12) Patent Application Publication
(10) Pub. No.: US 2022/0133849 A1
(43) Pub. Date: May 5, 2022

(54) COMPOSITIONS AND METHODS FOR THE TREATMENT OF SMOOTH MUSCLE DYSFUNCTION

(71) Applicant: UROVANT SCIENCES GMBH, Basel (CH)

(72) Inventor: Arnold MELMAN, Ardsley, NY (US)

(73) Assignee: UROVANT SCIENCES GMBH, Basel (CH)

(21) Appl. No.: 17/294,296

(22) PCT Filed: Nov. 14, 2019
(86) PCT No.: PCT/IB2019/059805

§ 371 Date: May 14, 2021

Related U.S. Application Data
(60) Provisional application No. 62/767,457, filed on Nov. 14, 2018.
(60) Provisional application No. 62/873,064, filed on Jul. 11, 2019.

(51) Int. Cl. A61K 38/17; A61K 9/00; C12N 15/63

(52) U.S. Cl. CPC A61K 38/177; C12N 15/63; A61K 9/0034

(57) ABSTRACT

The present disclosure provides compositions and methods to treat diseases and conditions related to smooth muscle dysfunction, or to ameliorate symptoms thereof comprising gene therapy, wherein one or more polynucleotides encoding one or more subunits of the Maxi-K channel, or mutants, variants, functional fragments, or derivatives thereof (e.g., fusions and chimaeras) are administered to a subject in need thereof, and wherein the administration of the polypeptides result in the expression of functional Maxi-K channels in the targeted smooth muscle. In some aspects, the composition of the disclosure comprise plasmid vectors containing at least one nucleic acid encoding a Maxi-K channel peptide. Also disclosed are pharmaceutical compositions, articles or manufacture, and kits.

### INCORPORATION OF SEQUENCE LISTING

The content of the electronically submitted sequence listing (Name: 3987.0260002_SequenceListing_ST25.txt, Size: 267,414 bytes; and Date of Creation: May 13, 2021) submitted in this application is incorporated herein by reference in its entirety.

### TECHNICAL FIELD

The present invention relates generally to the field of gene therapy to improve one or more symptoms related to smooth muscle dysfunction.

### BACKGROUND

Smooth muscle is found, for example, in blood vessels, the airways of the lungs, the gastro-intestinal tract, the uterus and the urinary tract. There are many physiological dysfunctions or disorders which are caused by the deregulation of smooth muscle tone, including uncontrolled contraction of smooth muscle. Included among these are asthma; benign hyperplasia of the prostate gland (BPH); coronary artery disease; erectile dysfunction; genitourinary dysfunctions of the bladder, endopelvic fascia, prostate gland, ureter, urethra, urinary tract, and vas deferens; irritable bowel syndrome; migraine headaches; premature labor; Raynaud's syndrome; varicose veins; and thromboangiitis obliterans.

The uncontrolled contraction of smooth muscle is also involved in states such as hypertension (a known risk factor for heart disease) or menstrual cramps. Hypertension or high blood pressure, is the most common disease affecting the heart and blood vessels. Statistics indicate that hypertension afflicts one out of every five American adults. Asthma is a chronic disease characterized by airway hyperactivity, it occurs in 5-8% of the U.S. population, and is an extraordinarily common cause of pulmonary impairment. Irritable bowel syndrome is a common syndrome characterized by frequently alternating constipation and diarrhea, usually with abdominal pain. Often stress induced, it is also caused by such physical factors as spicy foods, lack of dietary fiber, and excessive caffeine consumption. Menstrual cramping is a painful spasmodic contraction of the uterine muscles.

Urinary incontinence is the lack of voluntary control over micturition. In infants it is normal because neurons to the external sphincter muscle are not completely developed and the brain has not developed inhibitory function to prevent micturition. In the adult it may occur as a result of unconsciousness, injury to the spinal nerves controlling the urinary bladder, irritation due to abnormal constituents in urine, disease of the urinary bladder, and inability of the detrusor muscle to relax due to emotional stress.

Erectile dysfunction is a common illness that is estimated to affect 10 to 30 million men in the United States. Among the primary disease-related causes of erectile dysfunction are aging, atherosclerosis, chronic renal disease, diabetes, hypertension and antihypertensive medication, pelvic surgery and radiation therapy, and psychological anxiety.

Abnormal bladder function is another common problem which significantly affects the quality of life of millions of men and women in the United States. Many common diseases (e.g., BPH, diabetes mellitus, multiple sclerosis, and stroke) alter normal bladder function. Significant untoward changes in bladder function are also a normal result of advancing age.

Despite multiple attempts to develop a cure or treatment for diseases caused by altered smooth muscle tone, current therapies have limitations because they provide limited efficacy and/or significant side effects. Thus, there is a long-felt need in the art for a pharmaceutical and/or medical intervention to address the underlying cause of altered smooth muscle tone by increasing efficacy with minimal side effects, and to provide long term treatment solutions.

### BRIEF SUMMARY

The present disclosure provides methods to treat a smooth muscle dysfunction, e.g., a urinary bladder dysfunction such as overactive bladder (OAB), in a subject in need thereof comprising administering at least one dose of a composition comprising an isolated nucleic acid encoding a Maxi-K potassium channel polypeptide to the subject (e.g., a pVAX-hSlo vector of SEQ ID NO: 16, 49, or 50), wherein the expression of the Maxi-K potassium channel polypeptide in smooth muscle cells of the subject modulates smooth muscle contractility.

In some aspects, the Maxi-K potassium channel polypeptide comprises (i) a polypeptide encoding a Maxi-K alpha subunit (Slo) or a fragment, variant, mutant, or derivative thereof; (ii) a polypeptide encoding a Maxi-K beta subunit or a fragment, variant, mutant, or derivative thereof, wherein the Maxi-K beta subunit is a beta1 subunit, a beta2 subunit, a beta3 subunit, a beta4 subunit, or a combination thereof; or, (iii) a combination thereof.

In some aspects, the fragment is a functional fragment. In some aspects, the variant is a splice variant. In some aspects, the variant is an allelic (polymorphic) variant. In some aspects, the mutant is a point mutant. In some aspects, the mutant is a deletion and/or an insertion mutant. In some aspects, the mutant is a gain-of-function mutant. In some aspects, the mutant is a loss-of-function mutant.

In some aspects, the isolated nucleic acid encoding the Maxi-K potassium channel polypeptide or the Maxi-K potassium channel polypeptide comprises a sequence disclosed in TABLE 1 or a variant thereof. In some aspects, the Maxi-K potassium channel polypeptide comprises a mutation disclosed in TABLE 2.

In some aspects, the derivative is a fusion protein. In some aspects, the derivative is a chimaera. In some aspects, the modulation of smooth muscle contractility comprises an increase in contractility. In other aspects, the modulation of smooth muscle contractility comprises a decrease in contractility. In some aspects, the smooth muscle dysfunction is, e.g., selected from the group consisting of overactive bladder (OAB); erectile dysfunction (ED); asthma; benign prostatic hyperplasia (BPH); coronary artery disease; genitourinary dysfunctions of the bladder, endopelvic fascia, prostate gland, ureter, urethra, urinary tract, and vas deferens; irritable bowel syndrome; migraine headaches; premature labor; Raynaud's syndrome; detrusor overactivity; glaucoma; ocular hypertension; and thromboanginitis obliterans or a symptom or sequelae thereof.

In some aspects, the smooth muscle dysfunction is idiopathic. In some aspects, the smooth muscle dysfunction is neurogenic. In some aspects, the smooth muscle dysfunction is non-neurogenic.

In some aspects, the isolated nucleic acid is a DNA. In some aspects, the DNA is a naked DNA. In some aspects, the isolated nucleic acid is an RNA. In some aspects, the RNA is an mRNA. In some aspects, the isolated nucleic acid comprises at least one chemically modified nucleobase, sugar, backbone, or any combination thereof. In some aspects, the at least one chemically modified nucleobase is selected from the group consisting of pseudouracil (ψ), N1-methylpseudouracil (m1ψ), 2-thiouracil (s2U), 4′-thiouracil, 5-methylcytosine, 5-methyluracil, and any combinations thereof. In some aspects, the isolated nucleic acid has been modified by substituting at least one nucleobase, wherein the substitution is synonymous.

In some aspects, the isolated nucleic acid sequence is codon optimized. In some aspects, the isolated nucleic acid is a vector. In some aspects, the vector is a viral vector. In some aspects, the viral vector in an adenoviral vector. In some aspects, the adenoviral vector is a third generation adenoviral vector. In some aspects, the viral vector is a retroviral vector. In some aspects, the retroviral vector is a lentiviral vector. In some aspects, the lentiviral vector is a third or fourth generation lentiviral vector. In some aspects, the isolated nucleic acid or vector is administered with a delivery agent. In some aspects, the delivery agent comprises, e.g., a lipidoid, a liposome, a lipoplex, a lipid nanoparticle, a polymeric compound, a peptide, a protein, a cell, a nanoparticle mimic, a nanotube, or a conjugate.

In some aspects, the isolated nucleic acid or vector is incorporated into a cell in vivo, in vitro, or ex vivo. In some aspects, the cell is a stem cell, a muscle cell, or a fibroblast. In some aspects, the composition is administered topically or parenterally. In some aspects, the parenteral administration is by injection. In some aspects, the injection is intramuscular injection, e.g., injection into bladder muscular tissue. In some aspects, the isolated nucleic acid or vector is administered via instillation (e.g., instillation in the bladder of a subject in need thereof in an appropriate vehicle, e.g., a gel).

In some aspects, the injections of Maxi-K compositions of the present disclosure are administered at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more injection sites. In some aspects, the injections are administered to the bladder of the subject. In some aspects, the injections are administered to the bladder wall. In some aspects, the injections are administered to the detrusor. In some aspects, the injections are administered to the trigone. In some aspects, the volume of each injection is about 0.5 ml, about 1 ml, about 1.5 ml, or about 2 ml. In some aspects, the injection sites are about 0.5 cm, about 1 cm, about 1.5 cm, or about 2 cm apart. In some aspects, the injections are administered at a depth of injection of about 2 mm, about 2.5 mm, about 3 mm, about 3.5 mm, or about 4 mm.

In some aspects, the composition is administered by instillation into the lumen of an organ, e.g., urinary bladder or uterus. In some aspects, the dose is a single unit dose. In some aspects, the dose is between 5,000 mcg and 50,000 mcg. In some aspects, the dose is between 5,000 mcg and 100,000 mcg. In some aspects, the dose is at least 10,000 mcg. In some aspects, the dose is between 50,000 mcg and 100,000 mcg. In some aspects, the dose is 16,000, 24,000 mcg, or 48,000 mcg. In some aspects, the administration of the composition results in the amelioration of at least one symptom of a smooth muscle dysfunction.

### BRIEF DESCRIPTION OF THE DRAWINGS/FIGURES

FIGS. 1A, 1B, 1C and 1D show the impact of 2 weeks of obstruction on the relevant micturition parameters in the two treatment groups, relative to the Sham-operated, age-matched control rats. The data corresponds to data summarized in TABLE 3.

FIGS. 2A, 2B, and 2C show representative examples of approximately 1 hour of cystometric recordings following 2 weeks of obstruction from distinct rats in each treatment group: a control group (FIG. 2A), a vector only (pVAX) group (FIG. 2B), and a group treated with Maxi-K alpha subunit (hSlo) (FIG. 2C).

FIG. 3 shows three graphs of cystometric recordings in a rat given vector only (pVAX), and 300 and 1000 ug of pVAX-hSlo. Note the regular, periodic emptying and the virtual absence of intermicturition pressure fluctuations in the treated animals.

FIG. 4 is a bar graph of biodistribution, i.e., average number of copies of plasmid/ug total DNA in tissues of female animals after injection of 1,000 ug of pVAX-hSlo vector at 24 hours and 1 week (N=4 animals per time point; measured in duplicate. The background value for control tissue (animals that were not injected with pVAX-hSlo, average of 39 tissues) was 8.9×10⁻³ ng plasmid/ug total DNA, with an upper value of 8×10⁻² ng plasmid/ug total DNA. Therefore, only values greater than 9.6×10⁵ copies/ug total DNA were considered to be above control animal values (indicated by thick horizontal line).

FIG. 5 is a diagram which shows injection sites of the pVAX-hSlo vector in human subjects.

FIG. 6 is a bar graph showing the change in mean number of voids per day over time by treatment in human subjects (population efficacy). Error bars represent standard error of the means (SEM).

FIG. 7 is a bar graph showing the change in mean urgency episodes over time by treatment in human subjects (population efficacy). Error bars represent standard error of the means (SEM).

FIG. 8 is a schematic diagram depicting the plasmid pVAX-hSlo (total plasmid size: 6880 bp). hSlo is under control of the CMV promoter positioned upstream of the transgene. The construct also contains the Bovine Growth Hormone poly A site, kanamycin resistance gene and pUC origin of replication. In another embodiment, hSlo can be placed under the control of a promoter that specifically expresses the gene in the smooth muscle of a targeted organ. The positions of the different elements along the vector sequence and original are as follows. Cytomegalovirus (CMV) promoter (positions 137 to 724; viral); hSlo cDNA (positions 888 to 4428 bp; human); bovine growth hormone (BGH) polyadenylation signal (positions 4710 to 4940; bovine); kanamycin gene (positions 5106 to 5901; bacterial); and pUC origin (positions 6200 to 6874; bacterial).

FIG. 9 is a schematic depiction of the role of the Maxi-K channel in modulating transmembrane calcium flux and free intracellular calcium concentration in a bladder smooth muscle cell.

FIG. 10 is a graph depicting the effect of a point-mutation, T352S, in the pore of the hSlo channel on the channel's electrical properties. The T352S mutant hSlo channel displays significantly higher current compared to a wild type hSlo channel. 293 cells transfected with a sequence containing the T352S point mutation were used for this patch-clamp experiment.

FIG. 11 is a graph depicting the results of the patch clamp experiment described in EXAMPLE 4. Each of the constructs depicted were transfected into HEK cells. The current was measured after 24-48 hours in a high glucose (22.5 mM) environment. The T352S single point mutation confers resistance to oxidative stress. The double point mutations (C1, C2, C3, Ml, M2, and/or M3) can compromise the resistance of the T352S single point mutation to oxidative stress. Cl represents T352S plus C496A mutant; C2 represents T352S plus C681A mutant; C3 represents T352S plus C977A mutant; M1 represents T352S plus M602L mutant; M2 represents T352S plus M788L mutant; M3 represents T352S plus M805L mutant.

FIG. 12 is a chart showing the effect of different promoters on bladder function in the PUO model of OAB. pVAX=vector only, pUro-hSlo (hSlo expressed from the with uroplakin UPKII promoter), pVAX-hSlo (hSlo expressed from the CMV promoter), pSMAA-hSlo (hSlo expressed from the smooth muscle alpha actin promoter.) *=p<0.05.

FIG. 13A presents results from cystometry experiment showing cumulative volume of excreted urine from control (non-diabetic) rat.

FIG. 13B presents results from cystometry experiment showing cumulative volume of excreted urine from diabetic rat (2 month STZ-diabetic rat).

FIG. 13C presents results from organ bath experiment showing intravesical pressure from control (non-diabetic) rat.

FIG. 13D presents results from organ bath experiment showing intravesical pressure from diabetic rat (2 month STZ-diabetic rat).

FIG. 13E presents results from organ bath experiment showing isometric recordings of bladder strip from control (non-diabetic) bladder.

FIG. 13F presents results from organ bath experiment showing isometric recordings of bladder strip from diabetic (2 month STZ-diabetic rat) bladder illustrating marked spontaneous phasic contractions in the diabetic strip, characteristic of detrusor overactivity.

FIG. 13G presents results from organ bath experiment showing relative increase in amplitude of spontaneous contractions induced by treatment with increasing concentration of iberiotoxin (IBTX), a Maxi-K channel blocker. Data represent an average from 5 animals.

FIG. 13H shows results from single-cell patch clamping studies with stepwise increases in voltage performed in detrusor SM cells isolated from control and 2 month STZ-rats with bladder hyperactivity before and after incubation of cells with 300 nM IBTX. Stepwise application of voltage across the cell membrane results in opening of channels and outward current flow. The mean ratio of the maximum current at a particular voltage (Imax) to Imax after incubation with 300 nM IBTX is shown.

FIG. 14 shows spontaneous activity (SA) of PUO rat bladder. PUO rats were treated intravesically with empty pVAX (control) and pVAX for expression of wild type hSlo and mutant hSlo T352S genes. Our initial cystometry studies with PUO rats treated with 30 μg of pVAX-hSlo T352S indicate that when compared to our previously obtained data this hSlo mutant can be more efficient in reducing DO than the wild type gene (FIG. 11). Note the significantly higher effect of mutant hSlo T352S in reducing the bladder SA of PUO rats. Data correspond to mean±SEM; pVAX=14; pVAX-hSlo=17; pVAX-hSlo T352S=6; ANOVA followed by Dunnett's multiple comparison: *p<0.05, **p<0.01 vs. control; Student's t-test, pVAX-hSlo vs. pVAX-hSlo T352S, $ p<0.05.

FIG. 15A shows nanoparticles viewed by electron microscopy.

FIG. 15B shows FITC-labeled nanoparticles in solution, viewed by epifluorescence microscopy (20× magnification).

FIG. 15C shows FITC-labeled nanoparticles after application to the rat penis surface. One hour after application the animals were sacrificed and the penis cross-sectioned. Tissue sections were examined with an epifluorescence microscope at 4× and 20× (shown in inset) magnification. Fluorescent nanoparticles appear as small red spots and can be seen penetrating the penis periphery (dermis), as well as the cavernous vein lining and corpus spongiosum.

FIG. 16A shows in vitro monitoring of Maxi-K alpha subunit gene expression. Nanoparticles were generated by encapsulating the mCherry plasmid, which expresses a red fluorescent protein, and were added to a culture of HeLa cells. After 7 hours, the cells were visualized using phase contrast (left panel) and epifluorescence (middle panel) microscopy. Overlay of the two images (right panel) demonstrated that nearly all cells (approximately 95%) were expressing the mCherry fluorophore.

FIG. 16B shows in vitro monitoring of Maxi-K alpha subunit gene expression. Nanoparticles were generated encapsulating the human Maxi-K (hSlo) plasmid and added at different concentrations to a culture of HEK293 cells. After 20 hrs expression of human Maxi-K gene was determined by qRT-PCR. Bars represent the average fold change in Maxi-K expression over background from experiments repeated in triplicate.

FIG. 16C shows in vivo monitoring of Maxi-K alpha subunit gene expression. Whole animal fluorescence imaging 3 days after saline injection (left) or pmCherry-N1 (right) into the detrusor.

FIG. 16D shows ex vivo monitoring of Maxi-K alpha subunit gene expression. Bladders from animals in FIG. 16C were removed and imaged for mCherry fluorescence. On the heat map the red color indicates higher fluorescence.

FIG. 17 includes a schematic representation of the Maxi-K channel, showing a pore forming Maxi-K alpha subunit and a Maxi-K beta regulatory subunit. Two alternative schematic representations of the Maxi-K alpha subunit are shown (top and bottom left representations). Also presented (bottom right) is a representation of a top down view of the arrangement of the Maxi-K alpha subunit transmembrane helices showing in particular the location of the voltage sensing bundle and the pore and selective filter. Also shown are the two transmembrane helices of a beta subunit, nested between the voltage sensing bundle and the pore and selectivity filter. Maxi-K channels can be formed by alpha subunits only or by the association of alpha and beta subunits.

FIG. 18 shows a multiple sequence alignment between the nucleotide sequences of canonical pVAX-hSlo1 (SEQ ID NO: 16) and two variants, designated “Variant 1” (SEQ ID NO: 49) and “Variant 2” (SEQ ID NO: 50). The locations of differences between the sequences are indicated as boxed bases, which are numbered N1 to N16. The starting and ending points of the human Maxi-K alpha subunit (hSlo) ORF are also indicated.

FIG. 19 shows a multiple sequence alignment between the protein sequences encoded by the human Maxi-K alpha subunit (hSlo) ORFs in canonical pVAX-hSlo1 (SEQ ID NO: 16) and its two variants “Variant 1” (SEQ ID NO: 49) and “Variant 2” (SEQ ID NO: 50). The locations of differences between the sequences are indicated as boxed bases, which are numbered P1 and P2.

FIG. 20 is a CONSORT diagram corresponding to the ION-02 intravesical instillation study.

FIG. 21 is a CONSORT diagram corresponding to the ION-03 direct injection study.

FIG. 22 shows the change from baseline in mean number of urgency episodes per 24 hours in the ION-03 study.

FIG. 23 shows the change from baseline in mean number of void per 24 hours in the ION-03 study.

FIG. 24 shows a schematic of the design of the 2-cohort, dose-escalation study presented in Example 13.

FIGS. 25 and 26 show the bioactivity of URO-902 versus PBS-20% sucrose in retired breeder Sprague-Dawley rats. FIG. 25 shows ICB/BP ratio in response to neurostimulation. FIG. 26 shows visual penile erection (%) in response to neurostimulation.

FIGS. 27 and 28 show Maxi-K currents elicited at different voltages and internal calcium ion concentrations. FIG. 27 shows the currents elicited when the internal buffer contains 1 mM CaCl₂. FIG. 28 shows currents elicited when the internal buffer contains 5 mM CaCl₂.

FIG. 29 shows the concentration-response relationship of TEACl on Maxi-K current.

FIG. 30 shows stability of URO-902 in urine.

### DETAILED DESCRIPTION

The present disclosure provides compositions and methods of gene therapy for the treatment of smooth muscle dysfunctions and symptoms thereof. A primary goal of the compositions and methods disclosed herein is to restore normal smooth muscle function. In one aspect, the present disclosure provides compositions (“Maxi-K compositions of the present disclosure”) comprising at least one polynucleotide that contains at least one open reading frame encoding a polypeptide comprising a subunit of the Maxi-K channel (Maxi-K), e.g., a Maxi-K alpha-subunit, a beta-subunit, or any combination thereof, suitable for administration to smooth muscle, to a subject in need thereof having a smooth muscle dysfunction (e.g., a subject with a dysfunction of the bladder such as overactive bladder or urinary incontinence). After administration (e.g., topically, parenterally, or via instillation) of the Maxi-K composition using any gene therapy method known in the art, e.g., naked DNA or mRNA, encapsulated DNA or mRNA (e.g., in lipid nanoparticles), plasmids, viral vectors, gene editing methods (e.g., CRISPR), or transfected autologous or heterologous cells (e.g., stem cells), the Maxi-K channel polypeptide(s) are expressed in smooth muscle cells of the target tissue. The resulting Maxi-K activity in the target tissue significantly alleviates, treats, or prevents the symptoms of the smooth muscle dysfunction.

An important characteristic of the disclosed compositions and methods is that, advantageously with respect to conventional therapeutic intervention, they can be used for chronic diseases, i.e., diseases that otherwise would require the continued administration of a drug. Additionally, the disclosed gene therapy methods comprising the administration of a Maxi-K compositions would require a single administration, e.g., one every six months, or a series of administrations at long time intervals (several months). As a result, adherence to treatment issues which are prevalent in chronic diseases can be obviated.

Furthermore, the disclosed compositions and methods are suitable not only for the treatment of nerve induced smooth muscle dysfunctions (neurogenic dysfunction), as is the case with botulinum neurotoxins, but also for the treatment of non-nerve induced smooth muscle dysfunction (non-neurogenic dysfunction).

### I. TERMS

In order that the present disclosure can be more readily understood, certain terms are first defined. As used in this application, except as otherwise expressly provided herein, each of the following terms shall have the meaning set forth below. Additional definitions are set forth throughout the application.

The disclosure includes aspects in which exactly one member of the group is present in, employed in, or otherwise relevant to a given product or process. The disclosure includes aspects in which more than one, or all of the group members are present in, employed in, or otherwise relevant to a given product or process.

The compositions and methods of this disclosure as described herein can employ, unless otherwise indicated, techniques and descriptions of molecular biology (including recombinant techniques), cell biology, biochemistry, immunochemistry and ophthalmic techniques, which are within the skill of those who practice in the art. Such techniques include, e.g., methods for observing and analyzing smooth muscle function in a subject, cloning and propagation of recombinant virus, formulation of a pharmaceutical compositions, and biochemical purification and immunochemistry. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual (2007), Dieffenback, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, N.Y. (1995); Gait, “Oligonucleotide Synthesis: A Practical Approach” IRL Press, London (1984); Nelson and Cox, Lehninger, Principles of Biochemistry, 3rd Ed., W.H. Freeman Pub., New York (2000); and Berg et al., Biochemistry, 5th Ed., W.H. Freeman Pub., New York (2002), all of which are herein incorporated by reference in their entirety for all purposes.

The Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention.

Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

About: The term “about” as used herein refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 20%. Furthermore, particularly with respect to biological systems or processes, the terms can mean up to an order of magnitude or up to 5-fold of a value.

When particular values or compositions are provided in the application and claims, unless otherwise stated, the meaning of “about” should be assumed to be within an acceptable error range for that particular value or composition. When the term “about” is used in conjunction with a numerical range, it modifies that range by extending the boundaries above and below the numerical values set forth. Thus, “about 10-20” means “about 10 to about 20.” In general, the term “about” can modify a numerical value above and below the stated value by a variance of, e.g., 10 percent, up or down (higher or lower).

Administered in combination: As used herein, the term “administered in combination,” “combined administration,” or “combination therapy” means that two or more therapeutic agents, e.g., a Maxi-K composition of the present disclosure, and a second agent, are administered to a subject at the same time or within an interval such that there can be an overlap of an effect of each agent on the patient. In some aspects, the administrations of the agents are spaced sufficiently closely together such that a combinatorial (e.g., a synergistic) effect is achieved. Simultaneous administration is not necessary for a therapy to be considered a combination therapy. For example, for the treatment of erectile dysfunction (ED), ED treatments (e.g., cGMP-specific phosphodiesterase type 5 inhibitors) can be administered weeks or months after gene therapy comprising a Maxi-K composition of the present disclosure (e.g., a pVAX-hSlo vector of SEQ ID NO: 16, 49, or 50) was administered. In other words, in the context of gene therapy, a combination therapy does not require simultaneous administration of two or more therapeutic agents. Instead, any additional treatment while the transgene is effectively being expressed in the target tissue is considered a combination therapy.

And/or: “And/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Amino acid substitution: The term “amino acid substitution” refers to replacing an amino acid residue present in a parent or reference sequence (e.g., a wild type Maxi-K sequence) with another amino acid residue. An amino acid can be substituted in a parent or reference sequence (e.g., a wild type Maxi-K polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

Approximately: As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein with respect to a smooth muscle dysfunction, the term “associated with” means that the symptom, measurement, characteristic, or status in question is linked to the diagnosis, development, presence, or progression of that dysfunction. An association can, but need not, be causatively linked to the disease. For example loss of vision is a condition associated with glaucoma, a smooth muscle dysfunction. In other aspects, a smooth muscle dysfunction (e.g., poor bladder control) can be associated with, for example, a lesion (e.g., spinal cord injury), a neurodegenerative (e.g., multiple sclerosis), or aging.

Benign prostatic hyperplasia: As used herein, the term “benign prostatic hyperplasia” (abbreviated as “BPH”) denotes a histologic diagnosis that refers to the proliferation of smooth muscle and epithelial cells within the prostatic transition zone. In some aspects, the compositions and methods disclosed herein can be used to treat BPH.

Conservative amino acid substitution: A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. In another aspect, a string of amino acids can be conservatively replaced with a structurally similar string that differs in order and/or composition of side chain family members.

Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can be readily identified by persons of ordinary skill in the art. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of an polynucleotide or polypeptide or can apply to a portion, region or feature thereof.

Comprising: It is understood that wherever aspects are described herein with the language “comprising,” otherwise analogous aspects described in terms of “consisting of” and/or “consisting essentially of” are also provided.

Detrusor: As used herein, the term “detrusor” or “detrusor muscle” refers to the muscle of the bladder. By “intradetrusorally” is meant into the detrusor muscle. In some aspects, the compositions disclosed herein are injected intradetrusorally (i.e., in the detrusor muscle).

Detrusor overactivity: As used herein, the term “detrusor overactivity” refers to the occurrence of involuntary detrusor muscle contractions, e.g., during filling cystometry. These contractions, which can be spontaneous or provoked, are unable to be suppressed by the patient. They can take a wave (phasic) form, of variable duration and amplitude, on the cystometrogram. Urgency is generally associated in women with normal bladder sensation though contractions can be asymptomatic or can be interpreted as a normal desire to void. Urinary incontinence may or may not occur. A gradual increase in detrusor pressure without subsequent decrease is best regarded as a change in compliance. The term “detrusor overactivity” is defined by the International Continence Society (ICS) as follows: Detrusor overactivity is a urodynamic observation characterized by involuntary detrusor contractions during the filling phase that can be spontaneous or provoked (Abrams P et al., Urology 2003, 62(Supplement 5B): 28-37 and 40-42).

Effective Amount: As used herein, the term “effective amount” of a Maxi-K composition of the present disclosure in any dosage form, pharmaceutical composition, or formulation, is that amount sufficient to effect beneficial or desired results. In some aspects, the beneficial or desired results are, for example, clinical results, and, as such, an “effective amount” depends upon the context in which it is being applied. The term “effective amount” can be used interchangeably with “effective dose,” “therapeutically effective amount,” or “therapeutically effective dose.”

Expression vector: An “expression vector” is a polynucleotide which, when introduced into an appropriate host cell, can be transcribed and translated into a Maxi-K polypeptide of the present disclosure. Polynucleotides encoding a Maxi-K polypeptide can be transfected into target cells (e.g., a smooth muscle cell in a target tissue, or a stem cell for subsequent administration to the target tissue) by any means known in the art, and be transcribed and translated into a Maxi-K polypeptide of the present disclosure in the target tissue. Such transfection methods are widely known in the state of the art.

Homology: As used herein, the term “homology” refers to the overall relatedness between polymeric molecules, e.g., between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (polynucleotide or polypeptide sequences).

hSlo: The terms “Maxi-K alpha subunit,” “hSlo,” and “hSlo1” are used interchangeably throughout the present specification.

Identity: As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared.

When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. When comparing DNA and RNA, thymine (T) and uracil (U) can be considered equivalent.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

Irritable bowel syndrome: As used herein, the term “irritable bowel syndrome” (abbreviated as “IBS”) refers to a disorder, often recurrent, characterized by abnormally increased motility of the small and large intestines, producing abdominal pain, constipation, or diarrhea. One method of characterizing IBS is the Rome criteria for functional bowel disorders, including the Rome III or IV criteria. The term encompasses all classifications of irritable bowel syndrome including but not limited to each of diarrhea-predominant (IB S-D), constipation-predominant (IBS-C), mixed (IBS-M), alternating (IBS-A), and IBS with unknown subtype (IBS-U). Rome IV is the most recent criteria developed for diagnosis of IBS, and it increases sensitivity and specificity of the criteria with respect to abdominal pain, as compared to Rome III. See Lacy et al. “Rome Criteria and a Diagnostic Approach to Irritable Bowel Syndrome,” J. Clin. Med. 6, 99 (2017). Under Rome IV, IBS is diagnosed as: recurrent abdominal pain on average at least 1 day/week in the last 3 months, associated with two or more of the following criteria: (1) related to defecation; (2) associated with a change in the frequency of stool; and (3) associated with a change in the form (appearance) of stool. Under previously used Rome III, IBS is diagnosed as: recurrent abdominal pain or discomfort (defined as an uncomfortable sensation not described as pain) for at least 3 days/month in the last 3 months, associated with two or more of the following: (1) improvement with defecation; (2) onset associated with a change in the frequency of stool; and (3) onset associated with a change in the form (appearance) of stool. For both Rome III and IV, the criteria should be fulfilled for the last 3 months with symptoms onset at least 6 months before diagnosis.

In some aspects, the compositions and methods disclosed herein can be used to treat IBS, and/or prevent or ameliorate symptoms associated with IBS.

Isolated: As used herein, the term “isolated” refers to a substance or entity (e.g., polypeptide, polynucleotide, vector, cell, or composition which is in a form not found in nature) that has been separated from at least some of the components with which it was associated (whether in nature or in an experimental setting). Isolated substances (e.g., nucleotide sequence or protein sequence) can have varying levels of purity in reference to the substances from which they have been associated.

Isolated substances and/or entities can be separated from at least about 10%, at least about 15%, at least about 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least 95%, or more of the other components with which they were initially associated.

In some aspects, isolated substances are more than about 80%, about 85%, about 90%, about 91%, about 92%, about 93%, about 94%, about 95%, about 96%, about 97%, about 98%, about 99%, or more than about 99% pure.

As used herein, a substance is “pure” if it is substantially free of other components. The term “substantially isolated” means that the compound is substantially separated from the environment in which it was formed or detected. Partial separation can include, for example, a composition enriched in the compound of the present disclosure. Substantial separation can include compositions containing at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% by weight of the compound of the present disclosure, or salt thereof.

In some aspects, a polynucleotide, vector, polypeptide, cell, or any composition disclosed herein which is “isolated” is a polynucleotide (e.g., a nucleic acid encoding a Maxi-K polypeptide), vector, polypeptide, cell, or composition which is in a form not found in nature. Isolated polynucleotides, vectors, polypeptides, or compositions include those which have been purified to a degree that they are no longer in a form in which they are found in nature. In some aspects, a polynucleotide, vector, polypeptide, or composition which is isolated is substantially pure.

Isolated nucleic acid: As intended herein, the expression “isolated nucleic acid” refers to any type of isolated nucleic acid, it can notably be natural or synthetic, DNA or RNA, single or double stranded. In particular, where the nucleic acid is synthetic, it can comprise non-natural modifications of the bases or bonds, in particular for increasing the resistance to degradation of the nucleic acid. Where the nucleic acid is RNA, the modifications notably encompass capping its ends or modifying the 2′ position of the ribose backbone so as to decrease the reactivity of the hydroxyl moiety, for instances by suppressing the hydroxyl moiety (to yield a 2′-deoxyribose or a 2′-deoxyribose-2′-fluororibose), or substituting the hydroxyl moiety with an alkyl group, such as methyl group (to yield a 2′-O-methyl-ribose.)

Modulate smooth muscle contraction: As used herein, the language “modulating smooth muscle contraction” is intended to include the capacity to inhibit or stimulate smooth muscle contraction to various levels, e.g., which allows for the treatment of targeted states. The language is also intended to include the inducement of relaxation of smooth muscle, e.g., total relaxation, and the contraction of smooth muscle which is in relaxed state and it is desired to have the muscle in a more contracted state, e.g., the sphincter in esophageal reflux.

Mutation: In the content of the present disclosure, the terms “mutation” and “amino acid substitution” as defined above (sometimes referred simply as a “substitution”) are considered interchangeable. In some aspects, the term mutation refers to the deletion, insertion, or substitution of any nucleotide, by chemical, enzymatic, or any other means, in a nucleic acid encoding a Maxi-K polypeptide (e.g., a Maxi-K alpha subunit) such that the amino acid sequence of the resulting polypeptide is altered at one or more amino acid residues. In some aspects, a mutation in a nucleic acid sequence disclosed herein results in an amino acid substitution. In other aspects, the mutation of a codon in a nucleic acid sequence disclosed herein wherein the resulting codon is a synonymous codon does not result in an amino acid substitution. Accordingly, in some aspects, the nucleic acid sequences disclosed herein can be codon optimized by introducing one or more synonymous codon changes. Such codon optimization can, for example, (i) improve protein yield in recombinant protein expression, or (ii) improve the stability, half life, or other desirable property of an mRNA or a DNA encoding a binding molecule disclosed herein, wherein such mRNA or DNA is administered to a subject in need thereof.

Nocturia: As used herein, the term “nocturia” refers to a complaint of interruption of sleep one or more times because of the need to micturate. Each void is preceded and followed by sleep. In some aspects, the compositions and methods disclosed herein can be used to treat, prevent, or ameliorate nocturia.

Overactive bladder: As used herein, the term “overactive bladder” refers to urinary urgency, usually accompanied by frequency and nocturia, with or without urgency urinary incontinence, in the absence of urinary tract infection or other obvious pathology. The term “overactive bladder” is defined by the International Continence Society (ICS) as follows: Overactive bladder (OAB) is a symptom complex consisting of urgency with or without urge incontinence, usually with frequency and nocturia, in the absence of local pathologic or hormonal factors (Abrams P et al., Urology 2003, 61(1): 37-49; Abrams P et al., Urology 2003, 62(Supplement 5B): 28-37 and 40-42). Synonyms of overactive bladder (OAB) include “urge syndrome” and “urge frequency syndrome”. In some aspects, the compositions and methods disclosed herein can be used to treat, prevent, or ameliorate overactive bladder.

Patient: As used herein, “patient” refers to a subject who may seek or be in need of treatment, requires treatment, is receiving treatment, will receive treatment, or a subject who is under care by a trained professional for a particular disease or condition. The term also encompasses any a human or non-human mammal affected or likely to be affected with a smooth muscle dysfunction.

Pharmaceutical composition: The term “pharmaceutical composition” refers to a preparation which is in such form as to permit the biological activity of the active ingredient (e.g., a Maxi-K composition of the present disclosure) to be effective, and which contains no additional components which are unacceptably toxic to a subject to which the composition would be administered. Such composition can be sterile.

Pharmaceutically acceptable: The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio. In general, approval by a regulatory agency of the Federal or state governments (or listed in the U.S. Pharmacopeia or other generally recognized pharmacopeia) for use in animals, and more particularly in humans implies that those compounds, materials, compositions, and/or dosage forms are pharmaceutically acceptable. Compounds, materials, compositions, and/or dosage forms that are generally acceptable as safe for therapeutically purposes are “therapeutically acceptable.” Compounds, materials, compositions, and/or dosage forms that are generally acceptable as safe for diagnostic purposes are “diagnostically acceptable.”

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration.

Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Excipients that are generally accepted as safe for therapeutic purposes are “therapeutically acceptable excipients.”

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like.

Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like.

Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates can be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In particular aspects, the polynucleotide comprises a DNA or an RNA, e.g., an mRNA. In other aspect, the DNA or RNA, e.g., an mRNA, is a synthetic DNA or RNA, e.g., an mRNA. In some aspects, the synthetic DNA or an RNA, e.g., an mRNA, comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A,C, T and U in the case of a synthetic DNA, or A, C, T, and U in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding RNA sequences (comprising U) are considered codon-optimized nucleotide sequence of the present disclosure. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ΨΨC codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH₂, of cytidine and the C2-NH₂, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al. (1993) J. Am. Chem. Soc. 115:4461-4467, and references cited therein; and isoguanine nucleotides can be prepared using the method described by Switzer et al., 1993, supra, and Mantsch et al. (1993) Biochem. 14:5593-5601, or by the method described in U.S. Pat. No. 5,780,610 to Collins et al.

Other nonnatural base pairs can be synthesized by the method described in Piccirilli et al. (1990) Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term polypeptide, as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

Preventing: As used herein, the term “preventing” refers to partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

Prophylactic: As used herein, “prophylactic” refers to a therapeutic or course of action used to prevent the onset of a disease or condition, or to prevent or delay a symptom associated with disease related to smooth muscle dysfunction. In some aspects, the compositions and methods disclosed herein can be applied prophylactically.

Prophylaxis: As used herein, the term “prophylaxis” refers to a measure taken to maintain health and prevent or delay the onset of a disease or condition related to smooth muscle dysfunction or to mitigate its extent and/or severity of the symptoms. Thus, a prophylactic use of a therapeutic agent disclosed herein corresponds to that amount sufficient to effect beneficial or desired results.

Ranges: As described herein, any concentration range, percentage range, ratio range or integer range is to be understood to include the value of any integer within the recited range and, when appropriate, fractions thereof (such as one tenth and one hundredth of an integer), unless otherwise indicated.

Renal impairment: The term “renal impairment” as used herein is inclusive of renal or kidney failure, renal or kidney insufficiency, renal or kidney malfunction, acute kidney injury, and chronic kidney disease, and related conditions, as well as the clinical symptoms, laboratory and other diagnostic measurements, and complications associated with each of these conditions.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Smooth muscle: The language “smooth muscle” is intended to include smooth muscle sensitive to the Maxi-K compositions of the present disclosure. Smooth muscle is sensitive to a Maxi-K composition of the present disclosure if the transgenically expressed Maxi-K polypeptide modulates the contraction of the smooth muscle. Examples of smooth muscle include smooth muscle of a blood vessel, the airways of the lungs, the gastro-intestinal tract, the uterus, and the urinary tract.

Smooth muscle dysfunction: As used herein the term smooth muscle dysfunction related to any disease, condition, symptom, or sequelae that can be treated, prevented, or ameliorated by the transgenic expression of the Maxi-K compositions of the present disclosure (e.g., a pVAX-hSlo vector of SEQ ID NO: 16, 49, or 50).

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain aspects, the mammal is a human subject. In some aspects, the subject is a human. In some aspects, the subject is a human patient. In a particular aspect, a subject is a human patient with a smooth muscle dysfunction.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some aspects, an individual who is susceptible to a disease, disorder, and/or condition (for example, cancer) can be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

Therapeutic agent: As used herein, the term “therapeutic agent” is used in a broad sense to include a Maxi-K composition of the present disclosure (e.g., a pVAX-hSlo vector of SEQ ID NO: 16, 49, or 50) that can provide a significant therapeutic benefit to a subject in need thereof, in particular, a subject suffering from a smooth muscle dysfunction.

The term therapeutic agent also encompasses prophylactic agents comprising a composition disclosed herein, wherein the therapeutic agent is administered, e.g., parenterally, topically, or via instillation. In some aspects, the therapeutic agent is administered via injection into the bladder wall. In other aspects, the therapeutic agent is administered via instillation into the subject's bladder. Therapeutic agents of the present disclosure include not only agents that smooth muscle dysfunctions, but also agents that can ameliorate and/or prevent any symptom associated with the presence of such dysfunction. Thus, as defined herein, the term therapeutic agent would include, for example, agents that can reduce or suppress a particular symptom caused by the smooth muscle dysfunction, e.g., inflammation or pain.

Target tissue: As used herein “target tissue” refers to any one or more tissue types of interest in which the delivery of a therapeutic and/or prophylactic agent of the present disclosure would result in a desired biological and/or pharmacological effect. Examples of target tissues of interest include specific tissues, organs, and systems or groups thereof. In particular applications, the target tissue can be any tissue comprising smooth muscle, e.g., bladder wall tissue, bowel tissue, vascular tissue, etc.

Topical administration: As used herein, the term “topical administration” refers to any administration of a Maxi-K composition of the present disclosure (e.g., a pVAX-hSlo vector of SEQ ID NO: 16, 49, or 50) by the local route, for example over the skin, an orifice, or a mucous membrane. Topical administration as used herein, includes the cutaneous, aural, nasal, vaginal, urethral, and rectal routes of administration.

Treating, treatment, therapy: As used herein, the terms “treating” or “treatment” or “therapy” refer to partially or completely alleviating, ameliorating, improving, relieving, delaying onset of, inhibiting progression of, reducing severity of, reducing incidence of one or more symptoms or features of disease, or any combination thereof.

A treatment comprising a Maxi-K composition of the present disclosure (e.g., a pVAX-hSlo vector of SEQ ID NO: 16, 49, or 50) can be administered to a subject who does not exhibit signs of a disease, disorder, and/or condition, and/or to a subject who exhibits only early signs of a disease, disorder, and/or condition for the purpose of, e.g., (i) decreasing the risk of developing a pathology associated with the disease, disorder, and/or condition, (ii) delaying the onset of the disease, disorder, and/or condition, or a pathology associated with said disease, disorder, and/or condition, or (iii) mitigating the symptoms and/or sequels of the disease, disorder, and/or condition or a pathology associated with said disease, disorder, and/or condition.

Thus, in general, the term “treatment” refers to countering the effects caused as a result of the disease or pathological condition of interest in a subject including (i) inhibiting the progress of the disease or pathological condition, in other words, slowing or stopping the development or progression thereof, or one or more symptoms of such disorder or condition; (ii) relieving the disease or pathological condition, in other words, causing said disease or pathological condition, or the symptoms thereof, to regress; (iii) stabilizing the disease or pathological condition or one or more symptoms of such disorder or condition, (iv) reversing the disease or pathological condition or one or more symptoms of such disorder or condition to a normal state, (v) preventing the disease or pathological condition or one or more symptoms of such disorder or condition, and (vi) any combination thereof.

ug, uM, uL: As used herein, the terms “ug,” “uM,” and “uL” are used interchangeably with “μg,” “μM,” and “μL” respectively.

Urge incontinence: As used herein, the term “urge incontinence” refers to a complaint of involuntary loss of urine.

Urgency urinary incontinence: As used herein, the term “urgency urinary incontinence” refers to a complaint of involuntary loss of urine associated with urgency.

Urinary urgency: As used herein, the term “urinary urgency” refers to a complaint of a sudden, compelling desire to void which is difficult to defer.

Urinary frequency: As used herein, the term “urinary frequency” refers to a complaint by the patient who considers that he/she voids too often by day.

Vector: A “vector” is a nucleic acid molecule, in particular self-replicating, which transfers an inserted nucleic acid molecule into and/or between host cells. The term includes vectors that function primarily for insertion of DNA or RNA into a cell (e.g., chromosomal integration), replication of vectors that function primarily for the replication of DNA or RNA, and expression vectors that function for transcription and/or translation of the DNA or RNA. In some aspects, the administration and/or expression of a nucleic acid (DNA or RNA, such as an mRNA) encoding a binding molecule disclosed herein can take place in vitro (e.g., during recombinant protein production), whereas in other cases it can take place in vivo (e.g., administration of an mRNA to a subject), or ex vivo (e.g., DNA or RNA introduced into an autologous or heterologous cells for administration to a subject in need thereof). Also included are vectors that provide more than one of the functions as described.

As used herein, the term “vector” also refers in general to any nucleic acid molecule capable of transporting another nucleic acid to which it has been linked. One type of vector is “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are replicated along with the host genome. Moreover, certain vectors, expression vectors, are capable of directing the expression of genes to which they are operably linked.

Additional definitions related to urological conditions can be found, e.g., in Chapple et al. (2018) “Terminology report from the International Continence Society (ICS) Working Group on Underactive Bladder (UAB)” Neurology and Urodynamics 37:2928-2931. Additional definitions related to benign prostatic hyperplasia can be found, e.g., at the “Guidelines for Management of Benign Prostatic Hyperplasia,” available at www.auanet.org/benign-prostatic-hyperplasia-(2010-reviewed-and-validity-confirmed-2014). Additional definitions related to irritable bowel syndrome and chronic idiopathic constipation can be found, for example, in Ford et al. (2014) “American College of Gastroenterology Monograph on the Management of Irritable Bowel Syndrome and Chronic Idiopathic Constipation” Am J Gastroenterol 109:S2-S26. All these documents are herein incorporated by reference in their entireties.

### II. METHODS OF TREATMENT OF SMOOTH MUSCLE DYSFUNCTION

The present disclosure provides methods of gene therapy for treating smooth muscle dysfunction. In particular, the methods disclosed herein relate to gene therapy comprising the administration of Maxi-K compositions of the present disclosure (e.g., a pVAX-hSlo vector of SEQ ID NO: 16, 49, or 50) to treat or prevent a smooth muscle dysfunction in a subject in need thereof. As used herein, the terms “Maxi-K compositions of the present disclosure,” “compositions of the present disclosure,” and grammatical variants thereof comprise, e.g.,

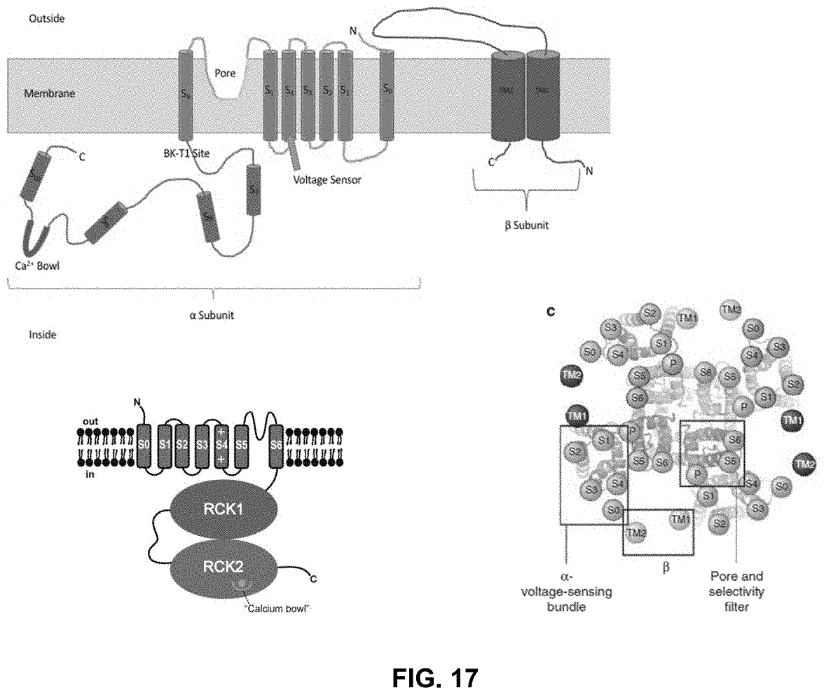

(a) one or more polynucleotides encoding one or more Maxi-K polypeptides schematically presented in FIG. 17, and domains or combination of domains thereof (according to the domain boundaries known in the art);

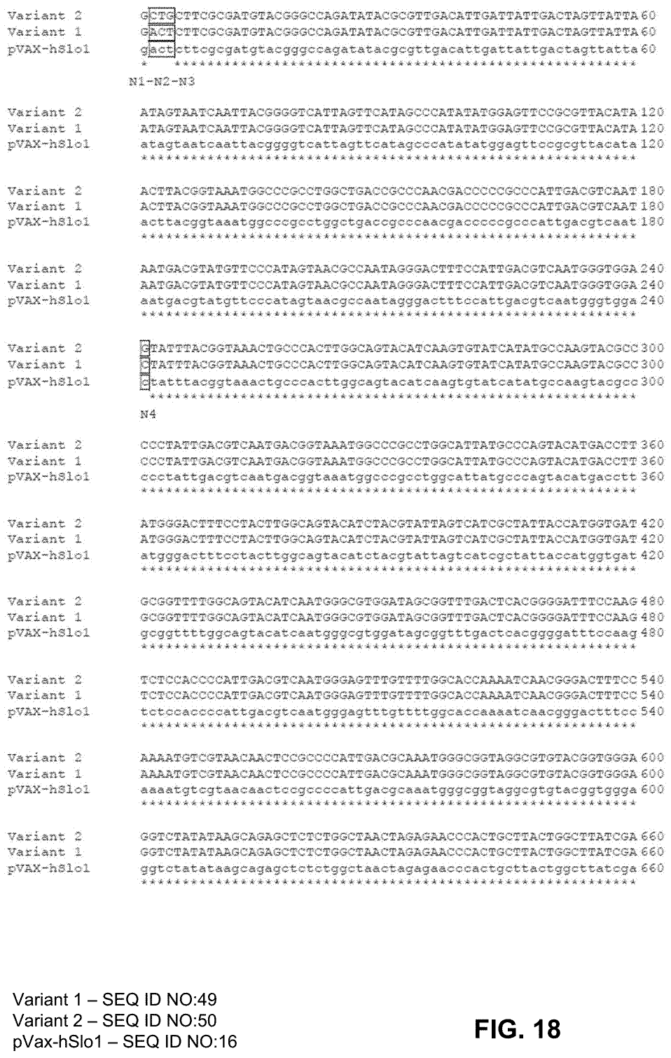

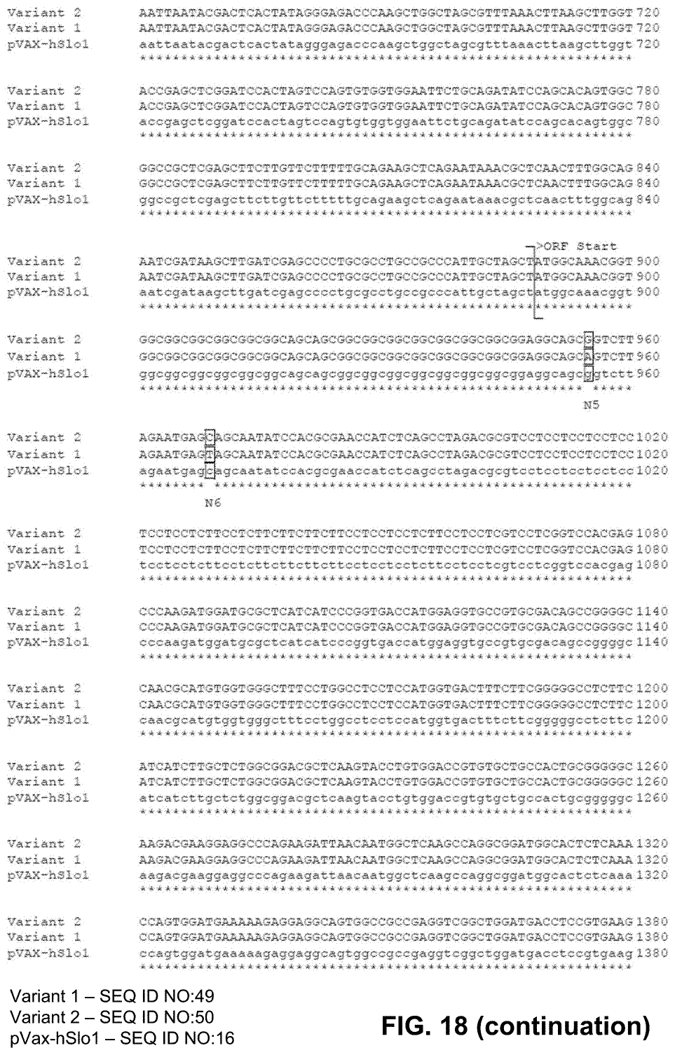

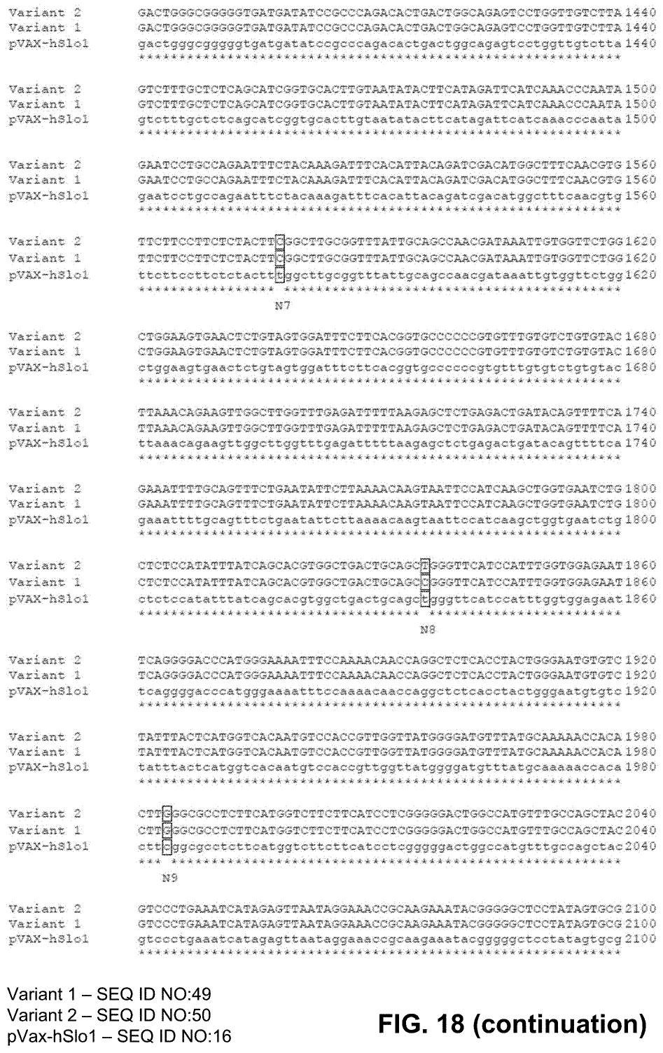

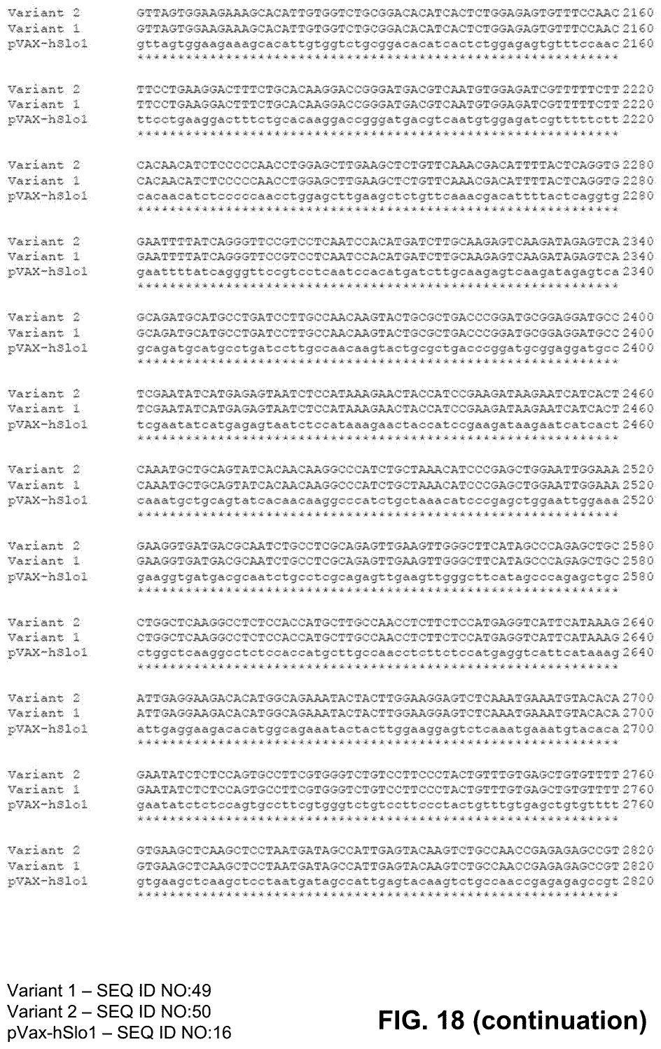

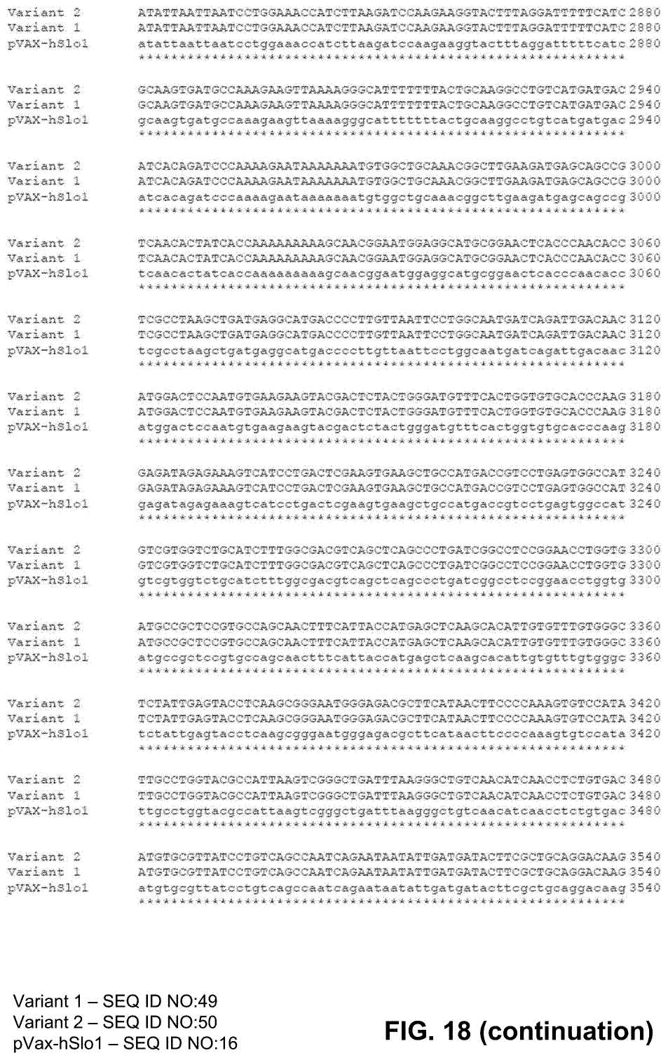

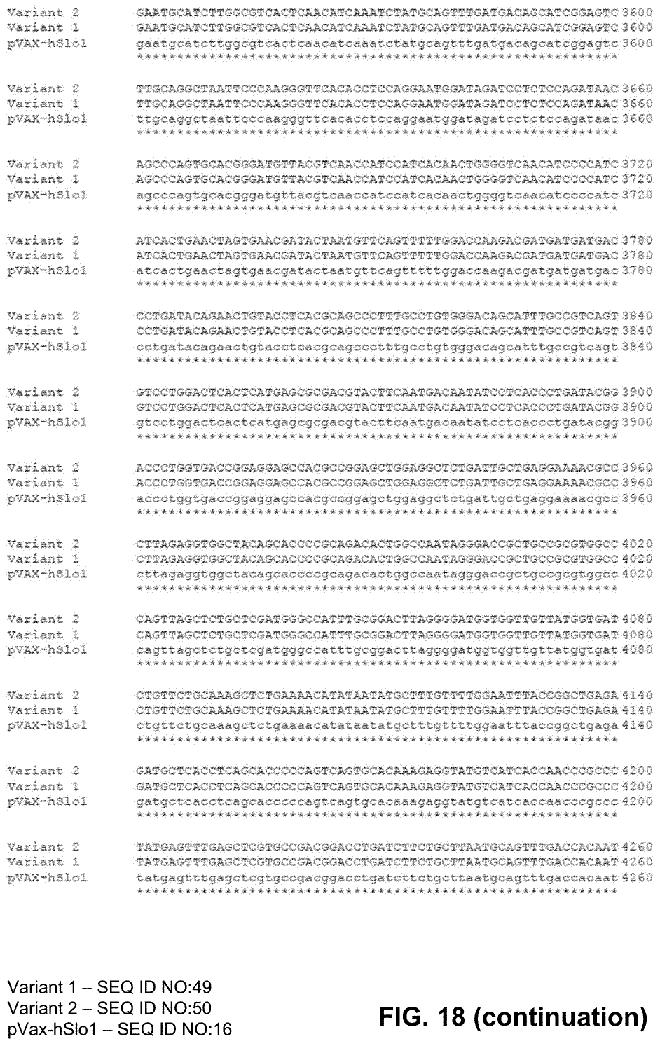

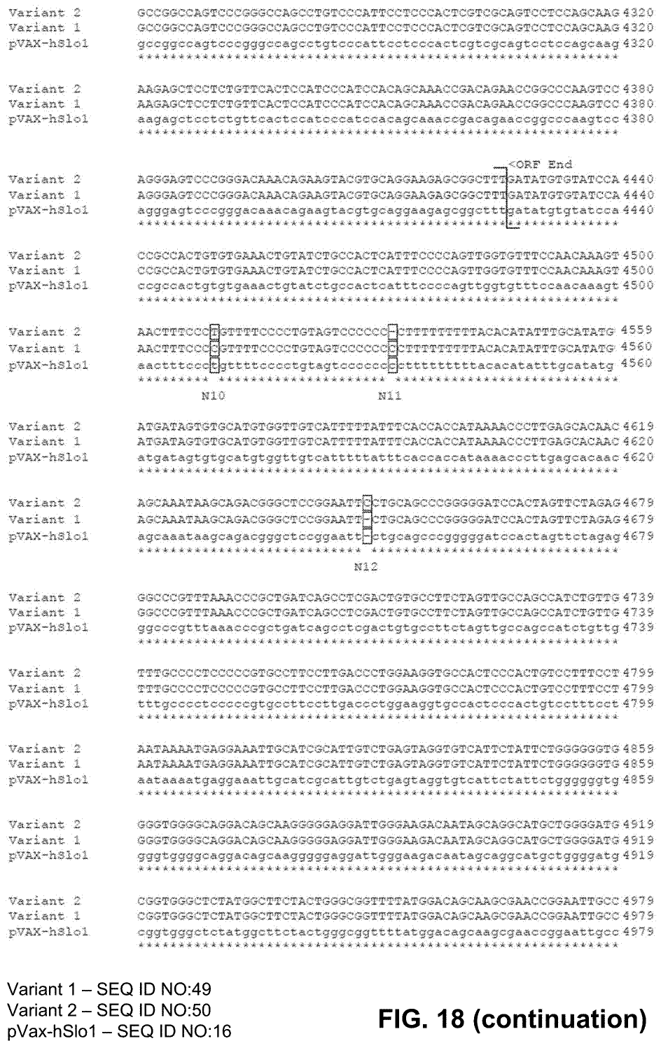

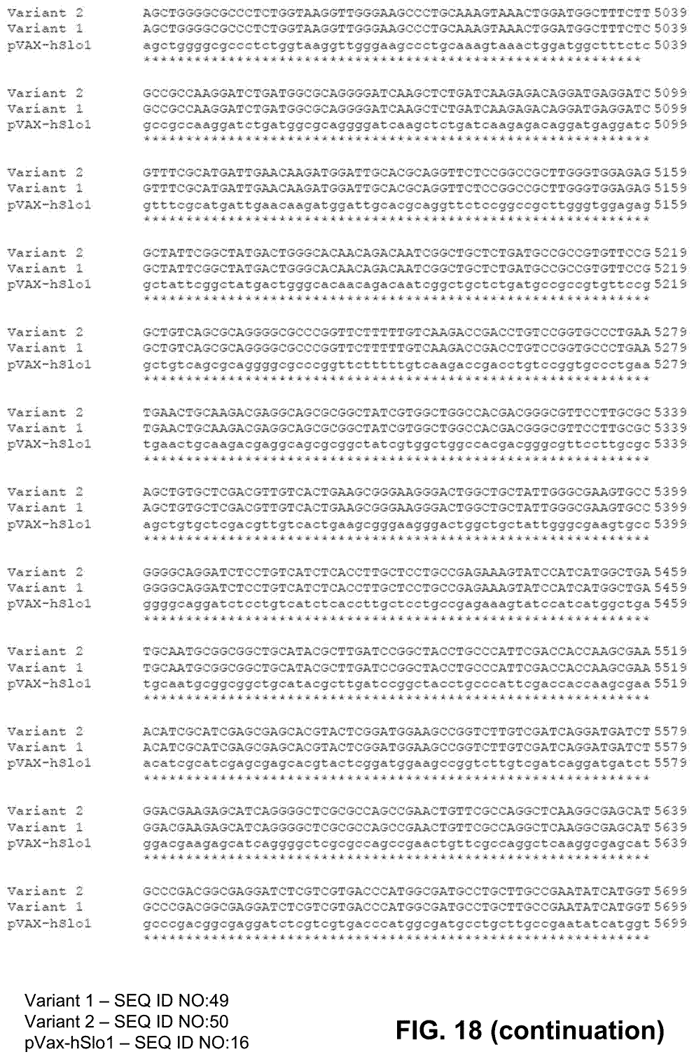

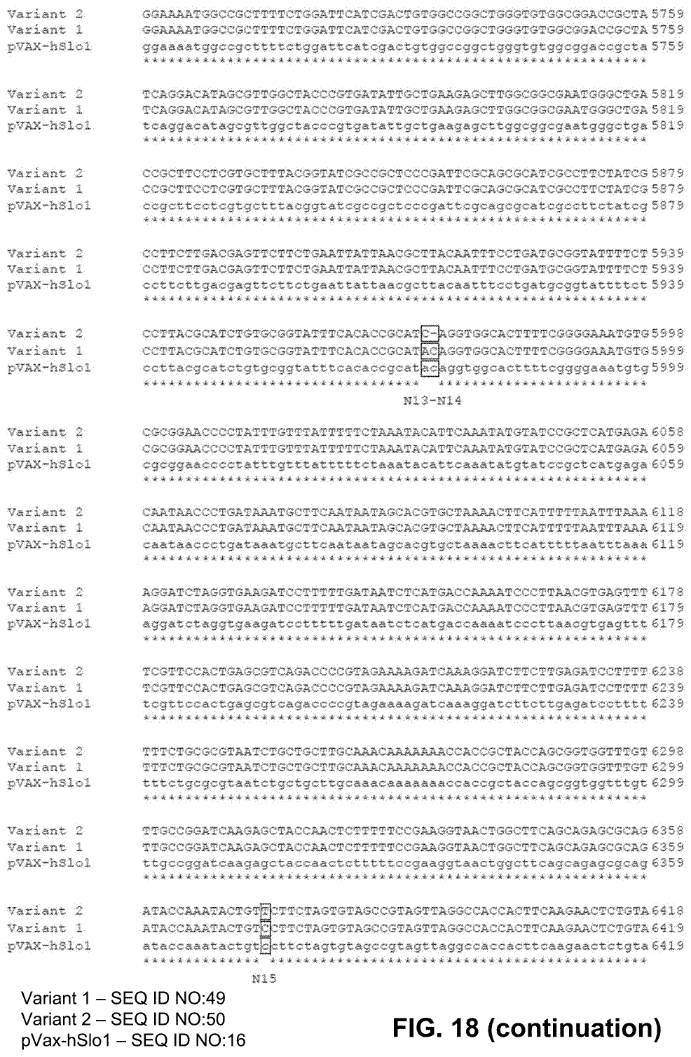

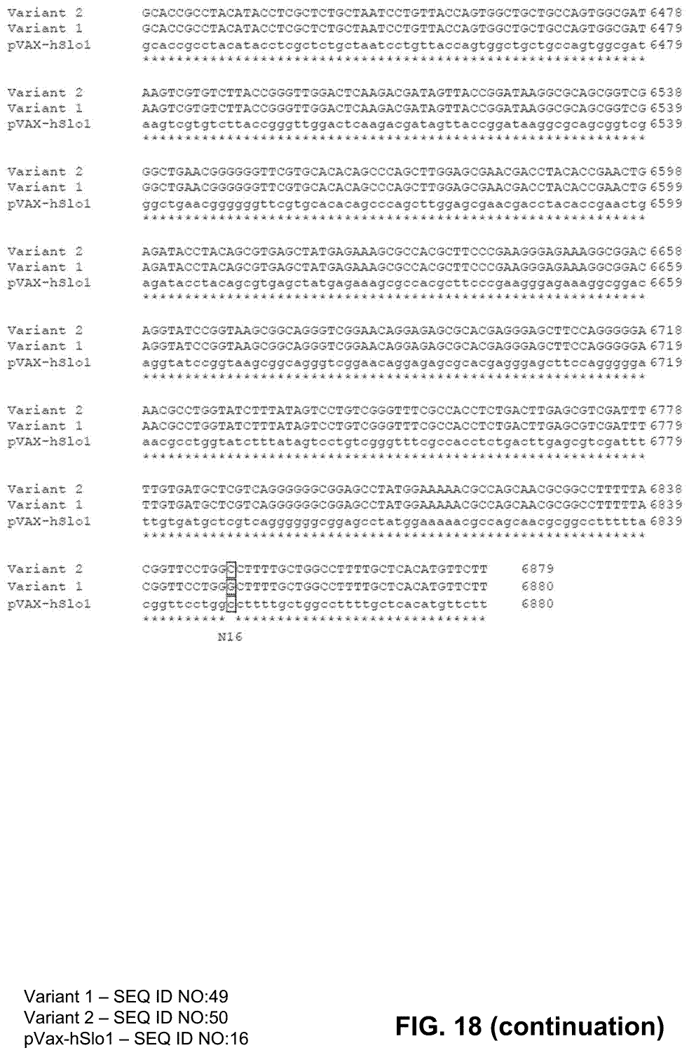

(b) one or more polynucleotides encoding one or more Maxi-K polypeptide sequences presented in TABLE 1 (e.g., Maxi-K alpha subunits, Maxi-K beta subunits, or combinations thereof), or fragments (e.g., an alpha subunit lacking one of more of the domains depicted in the FIG. 17 representation), isoforms, mutants, variants, or derivatives thereof, including, e.g., the polynucleotides presented in FIG. 18 and variants thereof comprising at least one of the variations N1 to N16 shown in FIG. 18 or any combination thereof;

(c) one or more polynucleotides encoding fusions or chimeric proteins comprising Maxi-K polypeptides disclosed herein, e.g., a Maxi-K alpha subunit genetically fused to a non-Maxi-K polypeptide conferring a desirable property, or a fusion between two or more Maxi-K polypeptides, e.g., a Maxi-K alpha subunit and a Maxi-K beta subunit;

(d) plasmids or vectors comprising the polynucleotides of (a), (b), (c) or any combination thereof;

(e) cells comprising the polynucleotides of (a), (b), or (c), the plasmids or vectors of (d), or any combination thereof;

(f) pharmaceutical compositions comprising the polynucleotides of (a), (b), or (c), the plasmids or vectors of (d), the cells of (e); or,

(g) any combination thereof.

In some aspects, the present disclosure provides a method to treat OAB comprising administering a Maxi-K composition of the present disclosure (e.g., a pVAX-hSlo vector of SEQ ID NO: 16, 49, or 50) to a subject in need thereof, e.g., by injection, implantation, or instillation into the subject's urinary bladder (e.g., by direct injection into the detrusor muscle).

In some aspects, the present disclosure provides a method to prevent OAB comprising administering a Maxi-K composition of the present disclosure (e.g., a pVAX-hSlo vector of SEQ ID NO: 16, 49, or 50) to a subject in need thereof, e.g., by injection, implantation, or instillation into the subject's urinary bladder (e.g., by direct injection into the detrusor muscle).

In some aspects, the present disclosure provides a method to treat or ameliorate at least one symptom of OAB comprising administering a Maxi-K composition of the present disclosure (e.g., a pVAX-hSlo vector of SEQ ID NO: 16, 49, or 50) to a subject in need thereof, e.g., by injection, implantation, or instillation into the subject's urinary bladder (e.g., by direct injection into the detrusor muscle).

Also provided is a method to reduce urgency and/or frequency of urination, e.g., associated with OAB, comprising administering a Maxi-K composition of the present disclosure (e.g., a pVAX-hSlo vector of SEQ ID NO: 16, 49, or 50) to a subject in need thereof, e.g., by injection, implantation, or instillation into the subject's urinary bladder (e.g., by direct injection into the detrusor muscle).

The present disclosure also provides a method to reduce UUI (urge urinary incontinence), e.g., associated with OAB, comprising administering a Maxi-K composition of the present disclosure (e.g., a pVAX-hSlo vector of SEQ ID NO: 16, 49, or 50) to a subject in need thereof, e.g., by injection, implantation, or instillation into the subject's urinary bladder (e.g., by direct injection into the detrusor muscle).

The present disclosure also provides a method to restore bladder function in a subject in need thereof comprising administering a Maxi-K composition of the present disclosure (e.g., a pVAX-hSlo vector of SEQ ID NO: 16, 49, or 50) to the subject, e.g., by injection, implantation, or instillation into the subject's urinary bladder (e.g., by direct injection into the detrusor muscle).

Also provided is a method to decrease bladder spasms, e.g., associated with OAB, in a subject in need thereof comprising administering a Maxi-K composition of the present disclosure (e.g., a pVAX-hSlo vector of SEQ ID NO: 16, 49, or 50) to the subject, e.g., by injection, implantation, or instillation into the subject's urinary bladder (e.g., by direct injection into the detrusor muscle).

Also provided is a method to prevent or treat or reduce loss of smooth muscle control in bladder, e.g., associated with OAB, in a subject in need thereof comprising administering a Maxi-K composition of the present disclosure (e.g., a pVAX-hSlo vector of SEQ ID NO: 16, 49, or 50) to the subject, e.g., by injection, implantation, or instillation into the subject's urinary bladder (e.g., by direct injection into the detrusor muscle).

The present disclosure also provides a method to increase the number and/or activity of Maxi-K channels in the detrusor smooth muscle cell membrane in a subject in need thereof comprising administering a Maxi-K composition of the present disclosure (e.g., a pVAX-hSlo vector of SEQ ID NO: 16, 49, or 50) to the subject, e.g., by injection, implantation, or instillation into the subject's urinary bladder (e.g., by direct injection into the detrusor muscle).

Also provided is a method to maintain or increase urinary bladder smooth muscle cell tone in a subject in need thereof comprising administering a Maxi-K composition of the present disclosure (e.g., a pVAX-hSlo vector of SEQ ID NO: 16, 49, or 50) to the subject, e.g., by injection, implantation, or instillation into the subject's urinary bladder (e.g., by direct injection into the detrusor muscle).

In some aspects, the Maxi-K composition of the present disclosure is a canonical pVAX-hSlo1 construct of SEQ ID NO: 16. In other aspects, the Maxi-K composition of the present disclosure is a pVAX-hSlo1 Variant 1 construct of SEQ ID NO: 49. In other aspects, the Maxi-K composition of the present disclosure is a pVAX-hSlo1 Variant 1 construct of SEQ ID NO: 50. In some aspects, the Maxi-K composition of the present disclosure comprises a combination thereof.

In some aspects, the Maxi-K composition of the present disclosure comprises a polynucleotide sequence comprising a nucleic acid sequence of SEQ ID NO: 51, 52 or 53, wherein the nucleic acid sequence encodes a Maxi-K alpha subunit (hSlo1).

In some aspects, the Maxi-K composition of the present disclosure comprises a polynucleotide sequence comprising a nucleic acid sequence encoding a Maxi-K alpha subunit (hSlo1) of SEQ ID NO: 54, 55, or 56.

In some aspects, the Maxi-K composition of the present disclosure encodes a Maxi-K alpha subunit (hSlo1) comprising a Glycine amino acid at position 23. In some aspects, the Maxi-K composition of the present disclosure encodes a Maxi-K alpha subunit (hSlo1) comprising a Serine amino acid at position 23. In some aspects, the Maxi-K composition of the present disclosure encodes a Maxi-K alpha subunit (hSlo1) comprising an Arginine amino acid at position 366. In some aspects, the Maxi-K composition of the present disclosure encodes a Maxi-K alpha subunit (hSlo1) comprising a Glycine amino acid at position 366.

In some aspects, the Maxi-K composition of the present disclosure encodes a Maxi-K alpha subunit (hSlo1) comprising a Glycine amino acid at position 23 and an Arginine amino acid at position 366, e.g., a Maxi-K alpha subunit of SEQ ID NO: 54. In some aspects, the Maxi-K composition of the present disclosure encodes a Maxi-K alpha subunit (hSlo1) comprising a Glycine amino acid at position 23 and a Glycine amino acid at position 366, e.g., a Maxi-K alpha subunit of SEQ ID NO: 55. In some aspects, the Maxi-K composition of the present disclosure encodes a Maxi-K alpha subunit (hSlo1) comprising a Serine amino acid at position 23 and an Glycine amino acid at position 366, e.g., a Maxi-K alpha subunit of SEQ ID NO: 56.

In some aspects, the Maxi-K composition of the present disclosure is a pVAX-hSlo1 construct of SEQ ID NO:16 comprising 1, 2, 3, 4, 5, 6, 7, 8, 9, 10, 11, 12, 13, 14, 15, or 16 of the N1-N16 variations identified in FIG. 18, or any combination thereof.

In some aspects, the Maxi-K composition of the present disclosure is a pVAX-hSlo construct derived from a pVAX-hSlo disclosed herein comprising at least a silent mutation which results in the expression of an Maxi-K alpha subunit polypeptide disclosed herein. Due to the degeneracy of the genetic code, a codon can be replaced in a pVAX-hSlo construct disclosed therein to yield the same protein product. In some cases, codons encoding the same amino acid differ only in their third position; thus, the two codons would have 66% sequence identity. In some case codons encoding the same amino acid can differ in two positions (e.g., CGC and AGA both of which encode Arginine), in which case two codons would have 33% sequence identity. Also, it is possible to have two codons encoding the same amino acid but having 0% sequence identity, for example, AGU and UCA, both of which encode serine. As a result, polynucleotides with very low percentages of sequence identity can nonetheless be functionally equivalent and encode the same polypeptide. Accordingly, in some aspects, the Maxi-K composition of the present disclosure comprises a polynucleotide (e.g., a vector or an ORF) having at least about 25%, at least about 30%, at least about 35%, at least about 40%, at least about 50%, at least about 55%, at least about 60%, at least about 65%, at least about 70%, at least about 75%, at least 80%, at least about 85%, at least about 90%, at least about 95%, or at least about 99% sequence identity to a Maxi-K-encoding polynucleotide sequence disclosed herein.

The Maxi-K compositions of the present disclosure (e.g., a pVAX-hSlo vector of SEQ ID NO: 16, 49, or 50) can be administered using gene transfer techniques known in the art (e.g., naked DNA or mRNA, plasmids, viral vectors, or gene editing technologies such as CRISPR), resulting in the expression of a Maxi-K polypeptide (e.g., a Maxi-K alpha subunit) or a combination of Maxi-K polypeptides (e.g., a Maxi-K alpha subunit and a Maxi-K beta subunit) in the target tissue. In some aspects, delivery of a Maxi-K composition of the present disclosure to a subject in need thereof can be referred to as gene therapy.

The Maxi-K channel (also known as the BK channel) provides an efflux pathway for potassium ions from the cell, allowing relaxation of smooth muscle by inhibition of the voltage sensitive Ca²⁺ channel, and thereby effecting the normalization of organ function by reducing pathological heightened smooth muscle tone. The terms “Maxi-K channel” and “BK channel” are used interchangeably herein. Structurally, Maxi-K channels are composed of alpha and beta subunits. Four alpha subunits form the pore of the channel, and these alpha subunits are encoded by a single Slo1 gene (also called Slo, hSlo, potassium calcium-activated channel subfamily M alpha 1, or KCNMA1).

There are four Maxi-K beta subunits which can modulate Maxi-K channel function. Each Maxi-K beta subunit has distinct tissue specific expression and modulatory functions, with the Maxi-K beta 1 subunit (potassium calcium-activated channel subfamily M regulator beta subunit 1, or KCNMB1) primarily expressed in smooth muscle cells.

Strategic clusters of Maxi-K channels in close proximity to the ryanodine-sensitive calcium stores of the underlying sarcoplasmic reticulum provide an important mechanism for the local modulation of calcium signals and membrane potential in diverse smooth muscle, e.g., urinary bladder smooth muscle.

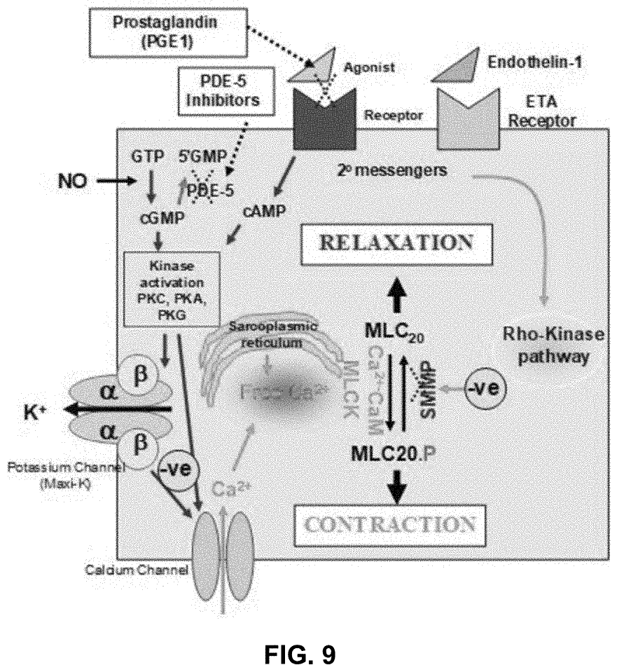

As shown in FIG. 9, the signal that activates a muscarinic M3 receptor causes an increase in intracellular calcium levels. The increase in the intracellular calcium level increases the open probability of the Maxi-K channel, thus increasing the outward movement of K⁺ through the calcium sensitive Maxi-K channel. The efflux of K⁺ causes a net movement of positive charge out of the cell, making the cell interior more negatively charged with respect to the outside. This has two major effects. First, the increased membrane potential ensures that the calcium channel spends more time closed than open. Second, because the calcium channel is more likely to be closed, there is a decreased net flux of Ca²⁺ into the cell and a corresponding reduction in the free intracellular calcium levels.

The reduced intracellular calcium promotes smooth muscle relaxation. The major implication of having more or less Maxi-K channels in the cell membrane or modulating their activity, e.g., via mutations in the Maxi-K alpha subunit or by upregulating or downregulating the function of the Maxi-K alpha subunit via interactions with wild type or mutant Maxi-K beta subunits, is that smooth muscle cell contractility can be modulated. Accordingly, transgenic expression of different combinations of Maxi-K alpha and/or beta subunits can be used to modify smooth muscle tone as appropriate to treat smooth muscle dysfunctions.

The present disclosure provides methods to treat a smooth muscle dysfunction (e.g., overactive bladder) in a subject in need thereof comprising administering a Maxi-K composition of the present disclosure, i.e., at least one dose of a composition comprising an isolated nucleic acid encoding a Maxi-K potassium channel polypeptide (e.g., a pVAX-hSlo vector of SEQ ID NO: 16, 49, or 50), to the subject, wherein the expression of the Maxi-K potassium channel polypeptide in smooth muscle cells of the subject modulates smooth muscle contractility. As used herein, the terms “Maxi-K potassium channel polypeptide” or “Maxi-K polypeptide” are used interchangeably and refer, e.g., to

(i) a polypeptide encoding a Maxi-K alpha subunit (Slo) or a fragment, variant, mutant, or derivative thereof;

(ii) a polypeptide encoding a Maxi-K beta subunit or a fragment, variant, mutant, or derivative thereof, wherein the Maxi-K beta subunit is a Maxi-K beta1 subunit, a Maxi-K beta2 subunit, a Maxi-K beta3 subunit, a Maxi-K beta4 subunit, or a combination thereof; or,

(iii) a combination thereof.

It is to be understood that in some aspects the Maxi-K polypeptide expressed as a result of gene therapy with a Maxi-K composition of the present disclosure is a single polypeptide (e.g., a Maxi-K alpha subunit or a Maxi-K beta subunit) whereas in other aspects the Maxi-K polypeptide comprises more than one polypeptide (e.g., a Maxi-K alpha subunit and a Maxi-K beta subunit, e.g., a Maxi-K beta1 subunit).

As used herein the term “administered,” as applied to a Maxi-K polypeptide of the present disclosure (e.g., hSlo) does not refer to the administration of a recombinant polypeptide. Instead, it refers to the administration of a Maxi-K composition comprising a nucleic acid comprising a polynucleotide encoding a Maxi-K polypeptides (e.g., a Maxi-K alpha subunits, a Maxi-K beta subunit, or both).

Maxi-K polypeptides (e.g., hSlo) can be administered, for example, using multiple vectors, each one comprising a nucleic acid encoding a single Maxi-K polypeptide (e.g., a first plasmid comprising a first nucleic acid encoding a Maxi-K alpha subunit and a second plasmid comprising a second nucleic acid encoding a Maxi-K beta subunit), or using a single vector comprising multiple open reading frames encoding different Maxi-K polypeptides (e.g., a plasmid comprising a first nucleic acid encoding a Maxi-K alpha subunit, and a second nucleic acid encoding a Maxi-K beta subunit).

A person of ordinary skill in the art would understand that alternative arrangements are also possible, e.g., a first plasmid for the expression of a Maxi-K alpha subunit and a second plasmid for the expression of two Maxi-K beta subunits. These same arrangement of nucleic acids encoding Maxi-K polypeptides are also applicable to viral vectors (e.g., adenoviral or lentiviral vectors). Similarly, the Maxi-K polypeptides of the present disclosure can be administered, for example, as monocistronic, bicistronic, or polycistronic mRNAs.

In some aspects, the Maxi-K polypeptide is a fragment, e.g., a Maxi-K functional fragment (e.g., a hSlo fragment). As used herein, the terminal “functional fragment” refers to a polypeptide that can function as a Maxi-K channel in the case of a Maxi-K alpha subunit, or as a regulatory subunit in the case of a Maxi-K beta subunit. In some aspects, the Maxi-K polypeptide functional fragment retains at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the activity of the corresponding full sequence Maxi-K polypeptide.

In some aspects, the Maxi-K polypeptide functional fragment exhibits an increase in activity with respect to the activity of the full sequence Maxi-K polypeptide. Accordingly, in some aspects, the Maxi-K polypeptide functional fragment exhibits an increase in activity of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% with respect to the activity of the corresponding full sequence Maxi-K polypeptide.

The term “variant” as used herein refers to a Maxi-K polypeptide sequence that possesses some modification of a structural property of the native protein. For example, the variant can be truncated at either the amino or carboxy termini, or both termini, or can have amino acids deleted or substituted. As used herein, the terms “amino terminus” and “N terminus” of a polypeptide can be used interchangeably. Similarly, the terms “carboxy terminus” and “C terminus” can be used interchangeably. Specific variants of Maxi-K are, for example, SEQ ID NOS: 54, 55 or 56.

In some aspects, the variant is the result of naturally occurring alternative splicing. Thus, in some aspects, the Maxi-K polypeptide (e.g., hSlo) is a splice variant. Exemplary splice variant forms of the Maxi-K alpha and beta subunits are included in TABLE 1.

In some aspects, a variant can be generated through recombinant DNA or RNA technologies, well known to those skilled in the art. For example, recombinant DNA or RNA technologies or methods to induce mutagenesis known in the art can be used to generate mutant Maxi-K polypeptides. In some aspects, the mutant is a point mutant, i.e., a Maxi-K polypeptide in which an amino acid at a certain position has been substituted with an alternative amino acid. This substitution can be conservative or non-conservative. In some aspects, a Maxi-K polypeptide of the present disclosure can comprise 1, 2, 3, 4, 5, 6, 7, 8, 9, 10 or more than 10 mutations with respect to the corresponding wild type Maxi-K polypeptide.

In some aspects, a Maxi-K polypeptide of the present disclosure (e.g., hSlo) can be an insertion and/or a deletion mutant, i.e., a mutant in which a subsequence of amino acids (e.g., 2, 3, 4, 5, 6, 7, 8, 9, 10 or more consecutive amino acids), is either inserted into or deleted from the sequence of the corresponding wild type Maxi-K polypeptide. In some aspects, a Maxi-K polypeptide of the present disclosure can comprise one or more than one deletions and/or one or more than one insertions. In some aspects, a subsequence can be deleted from a Maxi-K polypeptide and replaced with an alternative sequence inserted at the site of the deletion.

In some aspects, the Maxi-K polypeptide (e.g., hSlo) can comprise one or more mutations that are naturally occurring, or contain allelic variations (i.e., the Maxi-K polypeptide can be an allelic variant or a polymorphic variant). Exemplary polymorphisms and mutations in Maxi-K polypeptides are disclosed, for example, in TABLE 2.

In some aspects, the Maxi-K polypeptide (e.g., hSlo) is a gain-of-function mutant. The term “gain-of-function mutant” or “gain-of-function mutation” as used herein, refers to any mutation in a Maxi-K gene in which the Maxi-K polypeptide encoded by said gene (i.e., the mutant protein) acquires a function not normally associated with the wild type protein, or an existing function is increased or enhanced.

For example, for a channel such as Maxi-K a gain-of-function can refer, for example, to a change in channel conductivity, a change in ion selectivity, a change in sensitivity to modulators, or any combination thereof. The gain-of-function mutation can be a deletion, addition, or substitution of a nucleotide or nucleotides in the gene which gives rise to the change in the function of the encoded protein. In one aspect, the gain-of-function mutation can change the function of the mutant protein or cause or modulate its interactions with other proteins.

In some aspects, a gain-of-function mutation can cause a decrease in or removal of the normal wild-type protein from the target tissue, for example, by interaction of the altered, mutant protein with a normal, wild-type protein. In some aspect, transfecting a target smooth muscle cell with an altered Maxi-K beta subunit capable of increasing the activity of the Maxi-K alpha subunit can bind to endogenous wild type Maxi-K alpha subunits and displace the binding of endogenous wild type forms of the Maxi-K beta subunit.

In other aspects, the Maxi-K polypeptide (e.g., hSlo) is a loss-of-function mutant. The term “loss-of-function mutant” or “loss-of-function mutation” as used herein, refers to any mutation in a gene in which the protein encoded by said gene (i.e., the mutant protein) loses a function normally associated with the protein (i.e., the wild type protein), or an existing function is decreased. For example, for a channel such as Maxi-K a loss-of-function can refer, e.g., to a decrease or loss of channel conductivity, a decrease or loss of selectivity, a decrease or loss of sensitivity to modulators, or any combination thereof.

The loss-of-function mutation can be a deletion, addition, or substitution of a nucleotide or nucleotides in the Maxi-K gene, which gives rise to the change in the function of the encoded protein. In one aspect, the loss-of-function mutation can, e.g., change the function of the mutant protein or cause or modulate its interactions with other proteins. In some aspects, a loss-of-function mutation can cause a decrease in or removal of normal wild-type protein, for example, by interaction of the altered, mutant protein with said normal, wild-type protein. In some aspects, an altered Maxi-K beta subunit capable of decreasing the activity of the Maxi-K alpha subunit can bind to Maxi-K alpha subunit and displace the binding of wild type forms of the Maxi-K beta subunit).

In some aspects, an isolated nucleic acid encoding a Maxi-K potassium channel polypeptide of the present disclosure comprises a nucleic acid sequence disclosed in TABLE 1 or a fragment thereof capable of expressing a functional Maxi-K polypeptide. In some aspects, an isolated nucleic acid encoding the Maxi-K potassium channel polypeptide or the Maxi-K potassium channel polypeptide of the present disclosure comprises a nucleic acid sequence disclosed in TABLE 1 (or a fragment thereof capable of expressing a functional Maxi-K polypeptide) comprising one or more mutations disclosed in TABLE 2 and elsewhere in the present application.

Due to the presence of, e.g., mutations, insertion, deletions, or post-translational fragmentation, a Maxi-K polypeptide of the present disclosure can be at least about 50%, 51%, 52%. 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 96%, 97%, 98%, 99% or 100% identical to the wild sequence of a human Maxi-K polypeptide, e.g., a wild type Maxi-K polypeptide sequence disclosed in TABLE 1.

In some aspects, the Maxi-K polypeptide is a derivative. As used herein, the term “derivative” refers to a Maxi-K polypeptide which comprises one or more heterologous moieties which confer an additional functionality to the Maxi-K polypeptide. The Maxi-K polypeptide can comprise, e.g., a heterologous moiety that can increase or decrease the proteolytic rate of the expressed polypeptide, or a heterologous moiety capable of modulating the activity of the Maxi-K channel, for example, additional RCK (regulator of potassium conductance) domains in addition to RCK1 and RCK2—see, e.g., FIG. 17).

In some aspects, the derivative is a fusion protein. As used herein, the term “fusion protein” refers to a polypeptide resulting from the genetic fusion of at least two polypeptides, wherein at least one of the polypeptides is a Maxi-K polypeptide. An exemplary fusion protein is a Maxi-K polypeptide resulting from the genetic fusion of a Maxi-K alpha subunit and a Maxi-K beta subunit, wherein the Maxi-K beta subunit is covalently attached to the Maxi-K alpha subunit either directly or via a linker, e.g., a (Gly₄Ser)ₙ liker or any suitable linker known in the art. A person of ordinary skill in the art would understand that multiple copies of the Maxi-K alpha subunit (the same or different isoforms) and/or the Maxi-K beta subunit (the same or different isoforms) can be fused in any order and topological arrangement.

In other aspects, the derivative is chimaera. As used herein, the term “chimaera” refers to a polypeptide resulting from the substitution of a domain of a first polypeptide with an analogous domain from a second polypeptide. An exemplary chimaera is a Maxi-K polypeptide resulting from the substitution of a domain in the Maxi-K alpha subunit, e.g., an RCK domain of Maxi-K alpha subunit, with an analogous RCK domain from another protein (i.e., an RCK from any protein comprising in its architecture an Interpro “IPR003148 regulator of K+ conductance, N-terminal” domain). See, e.g., Meera et al. (2000) Proc. Natl. Acad. USA 97: 5562-5567, describing a Maxi-K beta subunit chimaera in which the extracellular loop of the smooth muscle beta 1 subunit and neuronal beta 4 subunits were exchanged.

In some aspects, the modulation of smooth muscle contractility by Maxi-K polypeptides following gene therapy with a Maxi-K composition of the present disclosure comprises an increase in contractility. In some aspects, the increase in contractility can be at least about 10%, at least about 15%, at least about 20%, at least about 25%, at least about 30%, at least about 35%, at least 40%, at least 45%, at least about 50%, at least about 55%, at least about 60%, at least about 65%, at least about 70%, at least about 75%, at least about 80%, at least about 85%, at least about 90% or at least about 100% with respect to the contractility prior to the administration of Maxi-K gene therapy according to the present disclosure.

In some aspects, the modulation of smooth muscle contractility by Maxi-K polypeptides following gene therapy with a Maxi-K composition of the present disclosure comprises a decrease in contractility. In some aspects, the decrease in contractility can be of at least about 10%, at least about 15%, at least about 20%, at least about 25%, at least about 30%, at least about 35%, at least 40%, at least 45%, at least about 50%, at least about 55%, at least about 60%, at least about 65%, at least about 70%, at least about 75%, at least about 80%, at least about 85%, at least about 90% or at least about 100% with respect to the contractility prior to the administration of Maxi-K gene therapy according to the present disclosure.

In some aspects, the Maxi-K compositions of the present disclosure (e.g., a pVAX-hSlo vector of SEQ ID NO: 16, 49, or 50) can be administered to treat or prevent a smooth muscle dysfunction selected, e.g., from the group consisting of overactive bladder (OAB); erectile dysfunction (ED); asthma; benign prostatic hyperplasia (BPH); coronary artery disease; genitourinary dysfunctions of the bladder, endopelvic fascia, prostate gland, ureter, urethra, urinary tract, and vas deferens; irritable bowel syndrome; migraine headaches; premature labor or menstrual cramps; Raynaud's syndrome; detrusor overactivity; glaucoma; ocular hypertension; and thromboanginitis obliterans or a symptom or sequel thereof. A more comprehensive list of diseases and conditions as well as their symptoms and sequelae that can be treated or prevented by the administration of gene therapy according to the present disclosure is provide in Section IV, below.

In some aspects, the smooth muscle dysfunction treated with a Maxi-K composition of the present disclosure is idiopathic. As used herein, the term idiopathic refers to a medical disease or condition having no known associated disease or cause, wherein the disease or condition is characterized by altered smooth muscle contractility. In some aspects, the smooth muscle dysfunction is neurogenic, i.e., the smooth muscle dysfunction is due to a disease or injury of the central nervous system or peripheral nerves not involved in bladder smooth muscle control, for example, neurogenic bladder, spinal cord injury, or neurodegenerative diseases.

Any condition that impairs bladder and bladder outlet afferent and efferent signaling can cause neurogenic bladder. It is often associated with spinal cord diseases (such as syringomyelia/hydromyelia), injuries (like herniated disks or spinal cord injury), and neural tube defects including spina bifida. It can also be caused by brain tumors and other diseases of the brain, pregnancy and by peripheral nerve diseases such as diabetes, peripheral neuropathy caused by prolonged exposure to Agent Orange, alcoholism, and vitamin B12 deficiency, and it is also a common complication of major surgery in the pelvis, such as for removal of sacrococcygeal teratoma, cancerous bladder, prostate tumors, rectal tumors, and other tumors. In some aspects, the neurogenic smooth muscle dysfunction is cause by a neurodegenerative disease, e.g., Parkinson's disease or multiple sclerosis.

In some aspects, the smooth muscle dysfunction is non-neurogenic, i.e., it is not caused by pathological changes in smooth muscle innervation.

In some aspects, the isolated nucleic acid sequence encoding a Maxi-K polypeptide of the present disclosure (e.g., a Maxi-K alpha subunit) is a DNA, e.g., a naked DNA. In other aspects, the isolated nucleic acid sequence encoding a Maxi-K polypeptide of the present disclosure (e.g., a Maxi-K alpha subunit) is an RNA, for example, an mRNA (e.g., a naked RNA). A “naked nucleic acid,” e.g., a “naked DNA” or a “naked RNA” is defined herein as a nucleic acid, e.g., a DNA or an RNA, not contained in a non-viral vector.

In some aspects, RNA nucleic acids (e.g., mRNAs) can include but are not limited to a transcript of a gene of interest (e.g., a Maxi-K alpha subunit), introns, untranslated regions, termination sequences and the like. In other cases, DNA nucleic acids can include but are not limited to sequences such as hybrid promoter gene sequences, strong constitutive promoter sequences, the gene of interest (e.g., a Maxi-K alpha subunit), untranslated regions, termination sequences and the like. In some cases, a combination of DNA and RNA can be used.

In some aspects, the isolated nucleic acid sequence encoding a Maxi-K polypeptide of the present disclosure (e.g., a Maxi-K alpha subunit) comprises at least one chemically modified nucleobase, sugar, backbone, or any combination thereof. In some aspects, the at least one chemically modified nucleobase is selected from the group consisting of pseudouracil (ψ), N1-methylpseudouracil (m1ψ), 2-thiouracil (s2U), 4′-thiouracil, 5-methylcytosine, 5-methyluracil, and any combinations thereof.

In some aspects, the isolated nucleic acid sequence encoding a Maxi-K polypeptide of the present disclosure (e.g., a Maxi-K alpha subunit) has been modified by substituting at least one nucleobase, wherein the substitution is synonymous. Due to the degeneracy of the genetic code it is possible to design polynucleotides with very low sequence identity which nonetheless result in the expression of the same polypeptide. Accordingly, in some aspects the nucleic acid encoding a Maxi-K polypeptide of the present disclosure can be at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 61%, at least about 62%, at least about 63%, at least about 64%, at least about 65%, at least about 66%, at least about 67%, at least about 68%, at least about 69%. at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical, e.g., to:

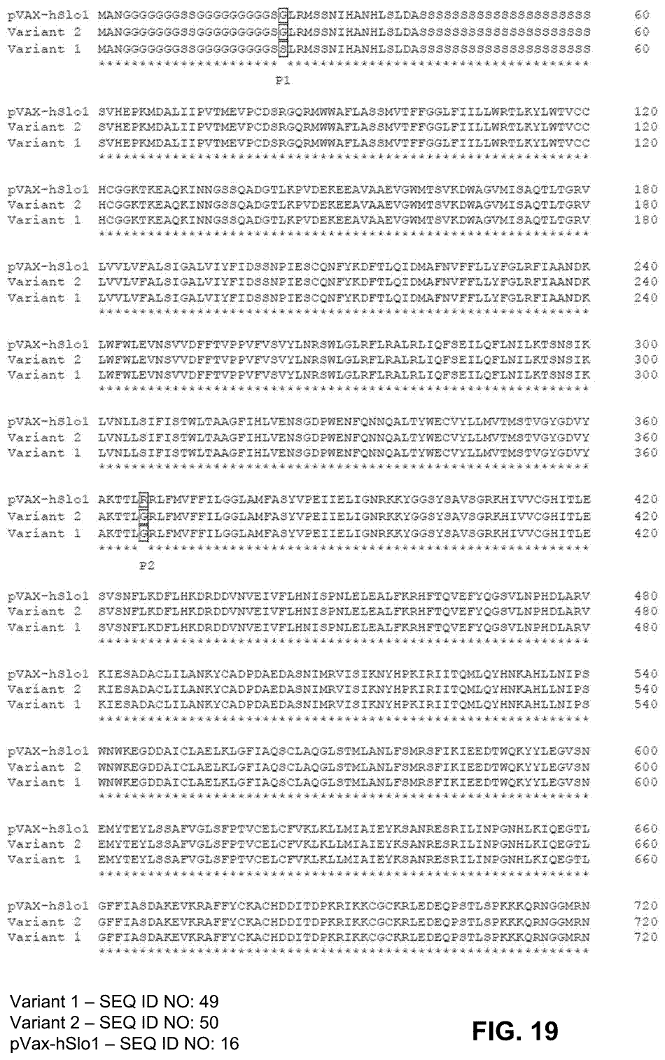

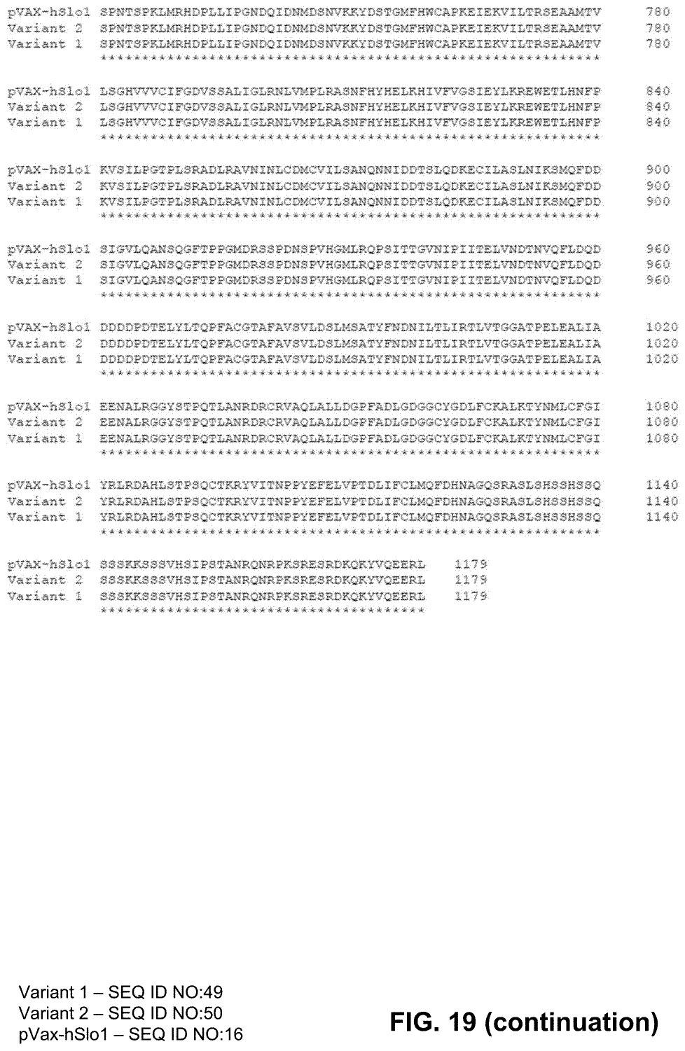

(a) a wild type polynucleotide sequence encoding a Maxi-K polypeptide disclosed in TABLE 1 or elsewhere in the present application, including, e.g., the polynucleotides presented in FIG. 18 and variants thereof comprising at least one of the variations N1 to N16 shown in FIG. 18 or any combination thereof, or a polynucleotide encoding any of the polypeptides presented in FIG. 19 and variants thereof comprising at least one of the P1 or P2 variations shown in FIG. 19

(b) a codon optimized polynucleotide sequence encoding a Maxi-K polypeptide disclosed, e.g., in U.S. Patent Appl. Publ. Nos. US2018/311381 or US2018/0126003, which are herein incorporated by reference in their entireties;

(c) any other natural or non-natural (e.g., codon optimized sequences, mutants, fusion, or chimaeras) Maxi-K polynucleotide sequences known in the art at the time the present application was filed; or

(d) a polynucleotide sequence encoding a Maxi-K ortholog;

(e) a polynucleotide sequence encoding a Maxi-K paralog, wherein the paralog is functionally equivalent or partially equivalent to Maxi-K with regard to modulation of smooth muscle contractility.

In some aspects, the isolated nucleic acid sequence encoding a Maxi-K polypeptide of the present disclosure (e.g., a Maxi-K alpha subunit) is codon optimized. As used herein, the terms “codon optimization,” “codon optimized,” and grammatical variants thereof refer to the modification of the primary sequence of a nucleic acid by replacing synonymous codons in order to increase its translational efficiency. Accordingly, codon optimization comprises switching the codons used in a transgene (e.g., a polynucleotide sequence encoding a Maxi-K polypeptide of the present disclosure) without changing the amino acid sequence that it encodes for, which typically dramatically increases the abundance of the protein the codon optimized gene encodes because it generally removes “rare” codons and replaces them with abundant codons, or removes codon with a low tRNA recharge rate with codon with high tRNA recharge rates.

Maxi-K polynucleotide sequences of the present disclosure (e.g., a pVAX-hSlo vector of SEQ ID NO: 16, 49, or 50) can be codon optimized using any methods known in the art at the time the present application was filed.

In some aspects, the isolated nucleic acid sequence encoding a Maxi-K polypeptide of the present disclosure (e.g., a Maxi-K alpha subunit) has been sequence optimized. As used herein, the term “sequence optimized” refers to the modification of the sequence of a nucleic acid by to introduce features that increase its translational efficiency, remove features that reduce its translational efficiency, or in general improve properties related to expression efficacy after administration in vivo. Such properties include, but are not limited to, improving nucleic acid stability (e.g., mRNA stability), increasing translation efficacy in the target tissue, reducing the number of truncated proteins expressed, improving the folding or prevent misfolding of the expressed proteins, reducing toxicity of the expressed products, reducing cell death caused by the expressed products, or increasing and/or decreasing protein aggregation.

In some aspects, the sequence optimized nucleotide sequence encoding a Maxi-K polypeptide of the present disclosure is codon optimized for expression in human subjects, having structural and/or chemical features that avoid one or more of the problems in the art, for example, features which are useful for optimizing formulation and delivery of nucleic acid-based therapeutics while retaining structural and functional integrity; overcoming a threshold of expression; improving expression rates; half-life and/or protein concentrations; optimizing protein localization; or avoiding deleterious bio-responses such as the immune response and/or degradation pathways.

In some aspects, the sequence optimized nucleotide sequence encoding a Maxi-K polypeptide of the present disclosure has been sequence optimized according to a method comprising, e.g.:

(i) substituting at least one codon in a reference nucleotide sequence (e.g., an ORF encoding a wild type Maxi-K polypeptide) with an alternative codon to increase or decrease uridine content to generate a uridine-modified sequence;

(ii) substituting at least one codon in a reference nucleotide sequence (e.g., an ORF encoding a wild type Maxi-K polypeptide) with an alternative codon having a higher codon frequency in the synonymous codon set;

(iii) substituting at least one codon in a reference nucleotide sequence (e.g., an ORF encoding a wild type Maxi-K polypeptide) with an alternative codon to increase G/C content; or

(iv) a combination thereof.

The presence of local high concentrations of uridine in a nucleic acid sequence can have detrimental effects on translation, e.g., slow or prematurely terminated translation, especially when modified uridine analogs are used in the production of synthetic mRNAs. Furthermore, high uridine content can also reduce the in vivo half-life of synthetic mRNAs due to TLR activation. Accordingly, a Maxi-K nucleic acid sequence (e.g., a pVAX-hSlo vector of SEQ ID NO: 16, 49, or 50 or Maxi-K encoding sequence therein) can be sequence optimized using a method comprising at least one uridine content optimization step. Such a step comprises, e.g., substituting at least one codon in the reference nucleic acid with an alternative codon to generate a uridine-modified sequence, wherein the uridine-modified sequence has at least one of the following properties:

(i) increase or decrease in global uridine content;

(ii) increase or decrease in local uridine content (i.e., changes in uridine content are limited to specific subsequences);

(iii) changes in uridine distribution without altering the global uridine content;

(iv) changes in uridine clustering (e.g., number of clusters, location of clusters, or distance between clusters); or

(v) combinations thereof.

A Maxi-K nucleic acid sequence can also be sequence optimized using methods comprising altering the Guanine/Cytosine (G/C) content (absolute or relative) of the reference nucleic acid sequence. Such optimization can comprise altering (e.g., increasing or decreasing) the global G/C content (absolute or relative) of the reference nucleic acid sequence; introducing local changes in G/C content in the reference nucleic acid sequence (e.g., increase or decrease G/C in selected regions or subsequences in the reference nucleic acid sequence); altering the frequency, size, and distribution of G/C clusters in the reference nucleic acid sequence, or combinations thereof.

Numerous codon optimization methods known in the art are based on the substitution of codons in a reference nucleic acid sequence with codons having higher frequencies. Thus, in some embodiments, a nucleic acid sequence encoding a Maxi-K polypeptide disclosed herein can be sequence optimized using methods comprising the use of modifications in the frequency of use of one or more codons relative to other synonymous codons in the sequence optimized nucleic acid with respect to the frequency of use in the non-codon optimized sequence.

As used herein, the term “codon frequency” refers to codon usage bias, i.e., the differences in the frequency of occurrence of synonymous codons in coding DNA/RNA. It is generally acknowledged that codon preferences reflect a balance between mutational biases and natural selection for translational optimization. Optimal codons help to achieve faster translation rates and high accuracy. As a result of these factors, translational selection is expected to be stronger in highly expressed genes.

In the field of bioinformatics and computational biology, many statistical methods have been proposed and used to analyze codon usage bias. See, e.g., Comeron & Aguadé (1998) J. Mol. Evol. 47: 268-74. Methods such as the ‘frequency of optimal codons’ (Fop) (Ikemura (1981) J. Mol. Biol. 151 (3): 389-409), the Relative Codon Adaptation (RCA) (Fox & Eril (2010) DNA Res. 17 (3): 185-96) or the ‘Codon Adaptation Index’ (CAI) (Sharp & Li (1987) Nucleic Acids Res. 15 (3): 1281-95) are used to predict gene expression levels, while methods such as the ‘effective number of codons’ (Nc) and Shannon entropy from information theory are used to measure codon usage evenness. Multivariate statistical methods, such as correspondence analysis and principal component analysis, are widely used to analyze variations in codon usage among genes (Suzuki et al. (2008) DNA Res. 15 (6): 357-65; Sandhu et al., In Silico Biol. 2008; 8(2): 187-92).

There is a variety of motifs that can affect sequence optimization, which fall into various non-exclusive categories, for example:

(i) Primary sequence based motifs: Motifs defined by a simple arrangement of nucleotides.

(ii) Structural motifs: Motifs encoded by an arrangement of nucleotides that tends to form a certain secondary structure.

(iii) Local motifs: Motifs encoded in one contiguous subsequence.

(iv) Distributed motifs: Motifs encoded in two or more disjoint subsequences.

(v) Advantageous motifs: Motifs which improve nucleotide structure or function.

(vi) Disadvantageous motifs: Motifs with detrimental effects on nucleotide structure or function.

There are many motifs that fit into the category of disadvantageous motifs. Some examples include, for example, restriction enzyme motifs, which tend to be relatively short, exact sequences such as the restriction site motifs for Xba1 (TCTAGA), EcoRI (GAATTC), EcoRII (CCWGG, wherein W means A or T, per the IUPAC ambiguity codes), or HindIII (AAGCTT); enzyme sites, which tend to be longer and based on consensus not exact sequence, such in the T7 RNA polymerase (GnnnnWnCRnCTCnCnnWnD, wherein n means any nucleotide, R means A or G, W means A or T, D means A or G or T but not C); structural motifs, such as GGGG repeats (Kim et al. (1991) Nature 351(6324):331-2); or other motifs such as CUG-triplet repeats (Querido et al. (2014) J. Cell Sci. 124:1703-1714).

Accordingly, the nucleic acid sequence encoding a Maxi-K polypeptide disclosed herein can be sequence optimized using methods comprising substituting at least one destabilizing motif in a reference nucleic acid sequence, and removing such disadvantageous motif or replacing it with an advantageous motif.

In some aspects, sequence optimization of a nucleic acid sequence encoding a Maxi-K polypeptide disclosed herein can be conducted using a limited codon set, e.g., a codon set wherein less than the native number of codons is used to encode the 20 natural amino acids, a subset of the 20 natural amino acids, or an expanded set of amino acids including, for example, non-natural amino acids.

In some aspects, the property improved via sequence optimization is an intrinsic property of the nucleic acid sequence. For example, the nucleotide sequence can be sequence optimized for in vivo or in vitro stability. In some aspects, the nucleotide sequence can be sequence optimized for expression in a particular target tissue or cell. In some aspects, the nucleic acid sequence can be sequence optimized to increase its plasma half by preventing its degradation by endo and exonucleases.

In other aspects, the nucleic acid sequence can be sequence optimized to increase its resistance to hydrolysis in solution, for example, to lengthen the time that the sequence optimized nucleic acid or a pharmaceutical composition comprising the sequence optimized nucleic acid can be stored under aqueous conditions with minimal degradation.

In other aspects, the sequence optimized nucleic acid can be optimized to increase its resistance to hydrolysis in dry storage conditions, for example, to lengthen the time that the sequence optimized nucleic acid can be stored after lyophilization with minimal degradation.

In some aspects, the expression of heterologous therapeutic proteins encoded by a nucleic acid sequence can have deleterious effects in the target tissue or cell, reducing protein yield, or reducing the quality of the expressed product (e.g., due to the presence of protein fragments or precipitation of the expressed protein in inclusion bodies), or causing toxicity. Accordingly, in some aspects, the sequence optimization of a nucleic acid sequence disclosed herein can be used to increase the viability of target cells expressing the Maxi-K polypeptide encoded by the sequence optimized nucleic acid.

Heterologous protein expression can also be deleterious to cells transfected with a nucleic acid sequence for autologous or heterologous transplantation. Accordingly, in some aspects of the present disclosure the sequence optimization of a nucleic acid sequence disclosed herein can be used to increase the viability of target cells expressing the Maxi-K polypeptide encoded by the sequence optimized nucleic acid sequence. Changes in cell or tissue viability, toxicity, and other physiological reaction can be measured according to methods known in the art.

Maxi-K polynucleotides comprising a sequence optimized nucleic acid can be tested to determine whether at least one nucleic acid sequence property (e.g., stability when exposed to nucleases) or expression property has been improved with respect to the non-sequence optimized nucleic acid using methods known in the art.

Maxi-K compositions of the present disclosure (e.g., a pVAX-hSlo vector of SEQ ID NO: 16, 49, or 50), in particular polynucleotides can be introduced into a smooth muscle cell by a number of procedures known to one skilled in the art, such as electroporation, DEAE Dextran, monocationic liposome fusion, polycationic liposome fusion, protoplast fusion, polynucleotide (e.g., DNA)-coated microprojectile bombardment, creation of an in vivo electrical field, injection with recombinant replication-defective viruses, homologous recombination, nanoparticles, and naked polynucleotide (e.g., DNA) transfer by, for example, intravesical instillation. It is to be appreciated by one skilled in the art that any of the above methods of polynucleotide (e.g., DNA) transfer can be combined.

In some aspects, the isolated nucleic acid encoding a Maxi-K polypeptide disclosed herein is a vector, e.g., a viral vector. In some aspects, the viral vector is an adenoviral vector (e.g., a third generation adenoviral vector). ADEASY™ is by far the most popular method for creating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™. PADEASY™ is ˜33 Kb adenoviral plasmid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY™ and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with Pad to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In other aspects, the viral vector is a retroviral vector, e.g., a lentiviral vector (e.g., a third or fourth generation lentiviral vector). Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelop gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

In some aspects, a nucleic acid sequence comprising a polynucleotide encoding a Maxi-K polypeptide of the present disclosure can be inserted into the genome of a target cell (e.g., a muscle cell in the target tissue) or a host cell (e.g., a stem cell for transplantation to the target tissue) by using CRISPR/Cas systems and genome edition alternatives such as zinc-finger nucleases (ZFNs), transcription activator-like effector nucleases (TALENs), and meganucleases (MNs).

In some aspects, the Maxi-K composition of the present disclosure (e.g., a pVAX-hSlo vector of SEQ ID NO: 16, 49, or 50) is administered with a delivery agent, e.g., a lipidoid, a liposome, a lipoplex, a lipid nanoparticle, a polymeric compound, a peptide, a protein, a cell, a nanoparticle mimic, a nanotube, or a conjugate. In some particular aspects, the delivery agent is a thermoreversible hydrogel, e.g., RTGel™. See, e.g., U.S. Appl. Publ. Nos. US2014/0142191, US2013/0046275, and US2006/0057208, all of which are herein incorporated by reference in their entireties.

In some aspects, the isolated nucleic acid or vector is incorporated into a cell in vivo, in vitro, or ex vivo. For example, the cell can be a stem cell, a muscle cell, or a fibroblast transfected with a Maxi-K composition of the present disclosure (e.g., a pVAX-hSlo vector of SEQ ID NO: 16, 49, or 50), wherein the cell expresses a Maxi-K polypeptide (e.g., a Maxi-K alpha, a Maxi-K beta subunit, or both). In some aspects, the cells, e.g., stem cells, can undergo one or more treatments with, e.g., a MAPK inhibitor, an inhibitor of stem cell proliferation, a stimulatory cytokine, or a combination thereof, to increase the efficacy of the transplantation process and/or one or more cycles of expansion (e.g., cell culture).

In some aspects, the Maxi-K compositions of the present disclosure (e.g., a pVAX-hSlo vector of SEQ ID NO: 16, 49, or 50) are administered or targeted to a target tissue. In particular, the Maxi-K compositions of the present disclosure (e.g., a pVAX-hSlo vector of SEQ ID NO: 16, 49, or 50) can be administered or targeted to smooth muscle cells in a particular target organ or tissue (e.g., smooth muscle cells in a detrusor urinary muscle).

In some aspects, a Maxi-K composition of the present disclosure (e.g., a pVAX-hSlo vector of SEQ ID NO: 16, 49, or 50) can be administered directly to a target cell or target tissue (e.g., via direct injection into smooth muscle in the urinary bladder wall, or inhalation for administration to smooth muscle cells in the respiratory tract) or administered at a distal location using a delivery system that specifically targets a particular organ or tissue. For example, a Maxi-K composition of the present disclosure (e.g., a pVAX-hSlo vector of SEQ ID NO: 16, 49, or 50) can be encapsulated in a liposome or nanoparticle comprising at least one antigen-binding moiety, e.g. an antibody or fragment thereof, to target the liposome or nanoparticle to an antigen in a specific tissue or target organ.

The methods of the present disclosure provide for any suitable method for delivery of a Maxi-K composition of the present disclosure (e.g., a pVAX-hSlo vector of SEQ ID NO: 16, 49, or 50) to a target tissue and in particular to smooth muscle cells in such target tissue in a subject in need thereof. In some aspects, a Maxi-K composition of the present disclosure (e.g., a pVAX-hSlo vector of SEQ ID NO: 16, 49, or 50) is administered topically or parenterally. In some aspects, the parenteral administration is by injection (e.g., by direct injection into the detrusor muscle), implantation, or instillation.

Routes of injection include, but are not limited to, subcutaneous, intravenous, intramuscular, or intrapelvic injections. In some aspects, the injection is intramuscular injection, in particular, injection into the smooth muscle of a target tissue or organ, e.g., into the bladder or uterine wall, or the penis of a subject. In some aspects, injections are administered at 1, 2, 3, 4, 5, 10, 15, 20, 30, 40, 50, or more injection sites.

Locations for implantation include, but are not limited to, subcutaneous, intravenous, intramuscular, or intrapelvic areas of the body. For example, a Maxi-K composition of the present disclosure (e.g., a pVAX-hSlo vector of SEQ ID NO: 16, 49, or 50) can be implanted within the pelvis, bladder, colon, uterus, or penis of a subject. In some aspects, implantation can take place at 1, 2, 3, 4, 5, 10, 15, 20, 30, 40, 50, or more implantation sites.

In some aspects, the Maxi-K composition of the present disclosure (e.g., a pVAX-hSlo vector of SEQ ID NO: 16, 49, or 50) is administered by instillation into the lumen of an organ. In a particular aspect, a Maxi-K composition of the present disclosure is introduced by instillation into the lumen of the bladder or the lumen of the uterus.

A person of ordinary skill in the art would understand that the route of administration is generally contingent on the specific target tissue. For example, smooth muscle dysfunction of the bladder (e.g., OAB) can be treated, e.g., by injection, instillation, catheter infusion, or high pressure application to the bladder wall; smooth muscle dysfunction of the prostate (e.g., BPH) can also be treated, e.g., by injection or infusion; smooth muscle dysfunction of the lungs (e.g., asthma) can be treated, e.g., by inhalation; smooth muscle dysfunction of the penis (e.g., ED) can be treated, e.g., by injection or topical application; intestinal smooth muscle dysfunction (e.g., IBS) can be treated, e.g., by enema; uterine smooth muscle dysfunction (e.g., menstrual cramps or uterine contractions during premature labor) can be treated, e.g., by injection, instillation, or catheter infusion; ocular smooth muscle dysfunction (e.g., high intraocular pressure or glaucoma) can be treated, e.g., by injection.

In some aspects, the dose of a Maxi-K composition of the present disclosure (e.g., a pVAX-hSlo vector of SEQ ID NO: 16, 49, or 50) is a single unit dose. In some aspects, the dose of a Maxi-K composition of the present disclosure (e.g., a pVAX-hSlo vector of SEQ ID NO: 16, 49, or 50) comprises at least about 5,000 mcg, at least about 6,000 mcg, at least about 7,000 mcg, at least about 8,000 mcg, at least about 9,000 mcg, at least about 10,000 mcg, at least about 11,000 mcg, at least about 12,000 mcg, at least about 13,000 mcg, at least about 14,000 mcg, at least about 15,000 mcg, at least about 16,000 mcg, at least about 17,000 mcg, at least about 18,000 mcg, at least about 19,000 mcg, at least about 20,000 mcg, at least about 21,000 mcg, at least about 22,000 mcg, at least about 23,000 mcg, at least about 24,000 mcg, at least about 25,000 mcg, at least about 26,000 mcg, at least about 27,000 mcg, at least about 28,000 mcg, at least about 29,000 mcg, at least about 30,000 mcg, at least about 31,000 mcg, at least about 32,000 mcg, at least about 33,000 mcg, at least about 34,000 mcg, at least about 35,000 mcg, at least about 36,000 mcg, at least about 37,000 mcg, at least about 38,000 mcg, at least about 39,000 mcg, at least about 40,000 mcg, at least about 41,000 mcg, at least about 42,000 mcg, at least about 43,000 mcg, at least about 44,000 mcg, at least about 45,000 mcg, at least about 46,000 mcg, at least about 47,000 mcg, at least about 48,000 mcg, at least about 49,000 mcg, at least about 50,000 mcg, at least about 51,000 mcg, at least about 52,000 mcg, at least about 53,000 mcg, at least about 54,000 mcg, at least about 55,000 mcg, at least about 56,000 mcg, at least about 57,000 mcg, at least about 58,000 mcg, at least about 59,000 mcg, at least about 60,000 mcg, at least about 61,000 mcg, at least about 62,000 mcg, at least about 63,000 mcg, at least about 64,000 mcg, at least about 65,000 mcg, at least about 66,000 mcg, at least about 67,000 mcg, at least about 68,000 mcg, at least about 69,000 mcg, at least about 70,000 mcg, at least about 71,000 mcg, at least about 72,000 mcg, at least about 73,000 mcg, at least about 74,000 mcg, at least about 75,000 mcg, at least about 76,000 mcg, at least about 77,000 mcg, at least about 78,000 mcg, at least about 79,000 mcg, at least about 80,000 mcg, at least about 81,000 mcg, at least about 82,000 mcg, at least about 83,000 mcg, at least about 84,000 mcg, at least about 85,000 mcg, at least about 86,000 mcg, at least about 87,000 mcg, at least about 88,000 mcg, at least about 89,000 mcg, at least about 90,000 mcg, at least about 91,000 mcg, at least about 92,000 mcg, at least about 93,000 mcg, at least about 94,000 mcg, at least about 95,000 mcg, at least about 96,000 mcg, at least about 97,000 mcg, at least about 98,000 mcg, at least about 99,000 mcg, or at least about 100,000 mcg of the composition (e.g., a naked nucleic acid, a plasmid, or a vector). As used herein mcg and μg are used interchangeably. In some aspects, the dose of a Maxi-K composition of the present disclosure (e.g., a pVAX-hSlo vector of SEQ ID NO: 16, 49, or 50) is about 6,000 mcg of the composition (e.g., a naked nucleic acid, a plasmid, or a vector). In some aspects, the dose of a Maxi-K composition of the present disclosure (e.g., a pVAX-hSlo vector of SEQ ID NO: 16, 49, or 50) is about 12,000 mcg of the composition (e.g., a naked nucleic acid, a plasmid, or a vector). In some aspects, the dose of a Maxi-K composition of the present disclosure (e.g., a pVAX-hSlo vector of SEQ ID NO: 16, 49, or 50) is about 24,000 mcg of the composition (e.g., a naked nucleic acid, a plasmid, or a vector). In some aspects, the dose of a Maxi-K composition of the present disclosure (e.g., a pVAX-hSlo vector of SEQ ID NO: 16, 49, or 50) is about 48,000 mcg of the composition (e.g., a naked nucleic acid, a plasmid, or a vector).

In some aspects, the dose of a Maxi-K composition of the present disclosure (e.g., a pVAX-hSlo vector of SEQ ID NO: 16, 49, or 50) is between about 5,000 mcg and about 10,000, or between about 10,000 and about 15,000 mcg, or between about 15,000 mcg and about 20,000 mcg, or between about 20,000 mcg and about 25,000 mcg, or between about 25,000 mcg and about 30,000 mcg, or between about 30,000 mcg and about 35,000 mcg, or between about 35,000 mcg and about 40,000 mcg, or between about 40,000 mcg and about 45,000 mcg, or between about 45,000 mcg and about 50,000 mcg, or between about 50,000 mcg and about 55,000 mcg, or between about 55,000 mcg and about 60,000 mcg, or between about 60,000 mcg and about 65,000 mcg, or between about 65,000 mcg and about 70,000 mcg, or between about 70,000 mcg and about 75,000 mcg, or between about 75,000 mcg and about 80,000 mcg, or between about 80,000 mcg and about 85,000 mcg, or between about 85,000 mcg and about 90,000 mcg, or between about 90,000 mcg and about 95,000 mcg, or between about 95,000 mcg and about 100,000 mcg.

During experimental administration of the Maxi-K compositions of the present disclosure (e.g., a pVAX-hSlo vector of SEQ ID NO: 16, 49, or 50) no toxicity has ever been identified, even at the highest concentrations tested. The limiting factor in the administration of the Maxi-K composition of the present disclosure (e.g., a pVAX-hSlo vector of SEQ ID NO: 16, 49, or 50) has been the solubility of the compositions.

Accordingly, in some aspects of the present disclosure, the dose of Maxi-K composition of the present disclosure (e.g., a pVAX-hSlo vector of SEQ ID NO: 16, 49, or 50) can be above 50,000 mcg. Accordingly, in some aspects of the present disclosure, the dose of Maxi-K composition of the present disclosure (e.g., a pVAX-hSlo vector of SEQ ID NO: 16, 49, or 50) can be above 100,000 mcg. Given that solubility can be a limiting factor in the administration of the Maxi-K compositions of the present disclosure, in some aspects, the Maxi-K compositions can be optimized to improve their solubility and/or to reduce precipitation and/or precipitation using methods known in the art, for example by incorporating (e.g., conjugating) hydrophilic polymers such as polyethylene glycols or polyglycerols in the delivery system.

In some aspects, the total dose of Maxi-K composition of the present disclosure (e.g., a pVAX-hSlo vector of SEQ ID NO: 16, 49, or 50) can be administered in a single administration (e.g., a single injection) or in multiple administrations (e.g., multiple injections). In some aspects, the multiple injection are administered simultaneously (for example, within a short period of time, e.g., within 30 minutes, an hour, two hours, or the same day), wherein in other aspects a substantial period of time elapses between injection (e.g., one or more days between injections).

In some aspects, multiple doses are administered, for example, every month, every two months, every three months, every four months, every five months or every six months.

In a specific aspect, a subject with a urinary bladder smooth muscle dysfunction (e.g., OAB) can receive a total dose of, e.g., 16,000 mcg, or 24,000 mcg, or 48,000 mcg of a Maxi-K composition of the present disclosure (e.g., a plasmid such as a pVAX plasmid comprising a polynucleotide sequence encoding a Maxi-K alpha subunit), administered as, e.g., 20-30 intramuscular injections into the bladder wall (e.g., a target site below or inferior to the bladder midline). In a specific aspect, a subject with a urinary bladder smooth muscle dysfunction (e.g., OAB) can receive a total dose of, e.g., 16,000 mcg, or 24,000 mcg, or 48,000 mcg of a Maxi-K composition of the present disclosure (e.g., a plasmid such as a pVAX plasmid comprising a polynucleotide sequence encoding a Maxi-K alpha subunit), administered as, e.g., 20-30 intramuscular injections into the detrusor muscle. In a specific aspect, a subject with a urinary bladder smooth muscle dysfunction (e.g., OAB) can receive a total dose of, e.g., 16,000 mcg, or 24,000 mcg, or 48,000 mcg of a Maxi-K composition of the present disclosure (e.g., a plasmid such as a pVAX plasmid comprising a polynucleotide sequence encoding a Maxi-K alpha subunit), administered as, e.g., 20-30 intramuscular injections into the trigone.

In some aspects, a Maxi-K composition of the present disclosure (e.g., a plasmid such as a pVAX plasmid comprising a polynucleotide sequence encoding a Maxi-K alpha subunit) is administered at 10 to 50 injection sites (e.g., at least about 10, at least about 15, at least about 20, at least about 25, at least about 30, at least about 35, at least about 40, at least about 45, or at least about 50 injections) in the bladder wall (e.g., detrusor muscle).

In some aspects, the injection target site comprises the bladder base, the posterior and lateral bladder wall, or both. In some aspects, the target site below (or inferior to) the bladder midline is selected from the regions consisting of the bladder base, the posterior and lateral bladder wall, the bladder base exclusive of the trigone, the bladder base exclusive of the trigone and the bladder neck, the trigone only, and the bladder neck only. In one aspect, the bladder midline corresponds to approximately 2-3 cm above an imaginary line intersecting the trigone above the ureteral orifices.

In some aspects, a Maxi-K composition of the present disclosure (e.g., a pVAX-hSlo vector of SEQ ID NO: 16, 49, or 50) is injected at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more sites in the bladder wall (e.g., in the bladder wall only).

In some aspects, a Maxi-K composition of the present disclosure (e.g., a pVAX-hSlo vector of SEQ ID NO: 16, 49, or 50) is injected at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more sites in the detrusor muscle (e.g., in the detrusor muscle only).

In some aspects, a Maxi-K composition of the present disclosure (e.g., a pVAX-hSlo vector of SEQ ID NO: 16, 49, or 50) is injected at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more sites in the trigone (e.g., in the trigone only).

In some aspects, a Maxi-K composition of the present disclosure (e.g., a pVAX-hSlo vector of SEQ ID NO: 16, 49, or 50) is injected at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more sites in the bladder base (e.g., in the bladder base only).

In some aspects, a Maxi-K composition of the present disclosure (e.g., a pVAX-hSlo vector of SEQ ID NO: 16, 49, or 50) is injected at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more sites in the posterior bladder wall (e.g., in the posterior bladder wall only).

In some aspects, a Maxi-K composition of the present disclosure (e.g., a pVAX-hSlo vector of SEQ ID NO: 16, 49, or 50) is injected at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more sites in the lateral bladder wall (e.g., in the lateral bladder wall only).

In some aspects, a Maxi-K composition of the present disclosure (e.g., a pVAX-hSlo vector of SEQ ID NO: 16, 49, or 50) is injected at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more sites below the bladder midline (e.g., below the bladder midline only).

In some aspects, a Maxi-K composition of the present disclosure (e.g., a pVAX-hSlo vector of SEQ ID NO: 16, 49, or 50) is injected at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more sites in the bladder base exclusive of the trigone.

In some aspects, a Maxi-K composition of the present disclosure (e.g., a pVAX-hSlo vector of SEQ ID NO: 16, 49, or 50) is injected at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more sites in the bladder base exclusive of the trigone and the bladder neck.

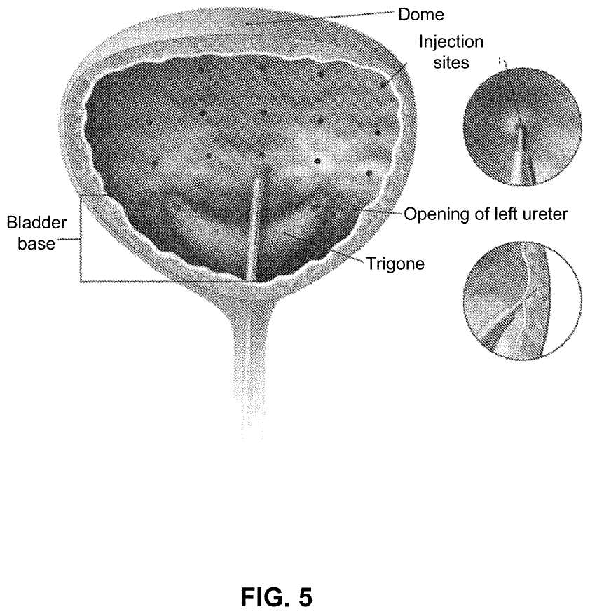

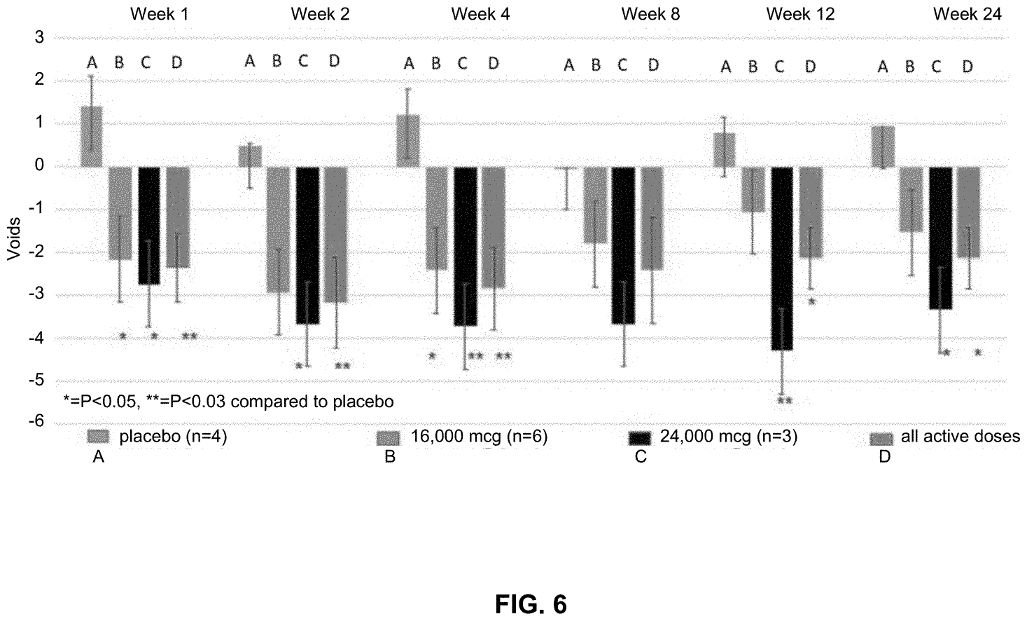

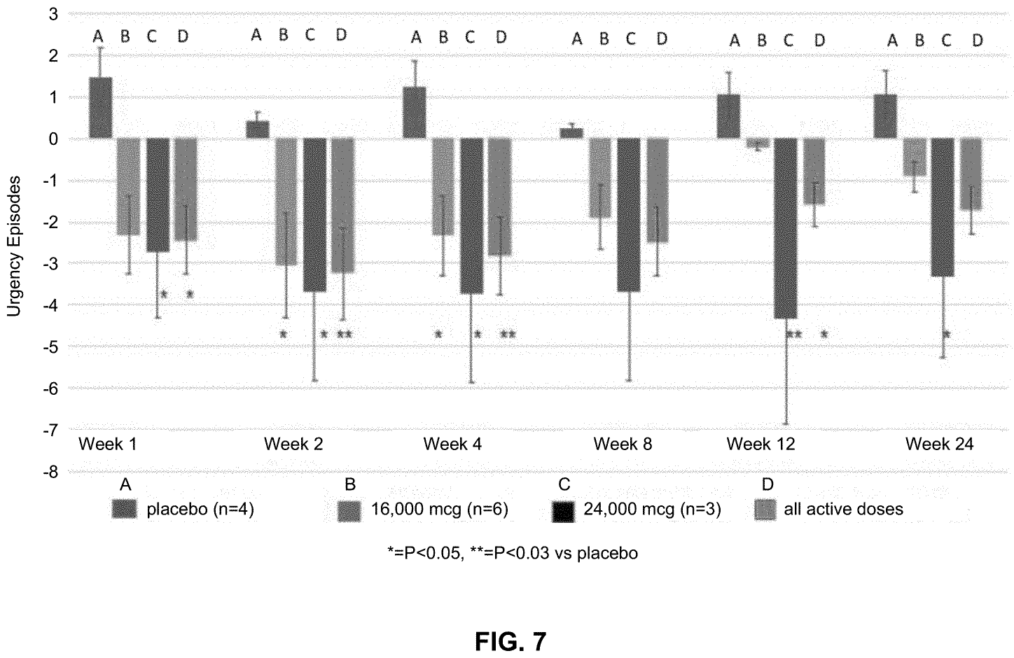

A frontal cross-sectional view of a human bladder is shown in FIG. 5. The hollow organ has a vertex or apex, a superior surface (also referred to as the dome), and an inferior surface or base. The base comprises the posteriorly and inferiorly facing surfaces of the organ. The trigone lies at (and within) the base of the bladder and borders the posterior side of the bladder neck. The bladder neck is within the bladder base and corresponds to a region where the walls of the bladder converge and connect with the urethra. At lateral points of the trigone the ureters empty into the bladder cavity through the ureteral orifices. The detrusor muscle is a layer in the bladder wall of smooth muscle fibers.

In some aspects, some of the injection sites are in the bladder wall (e.g., the lower part of the bladder wall, for example, the lower part of the back of the bladder wall below the bladder midline). In some aspects, some of the injection sites are in the trigone. In some aspects, some of the injection sites are in the detrusor.

In some aspects, all the injection sites are in the bladder wall (e.g., the lower part of the bladder wall, for example, the lower part of the back of the bladder wall below the bladder midline). In some aspects, all of the injection sites are in the trigone. In some aspects, all the injection sites are in the detrusor.

In some aspects, no injection sites are in the detrusor. In some aspects, no injection sites are in the trigone.

In some aspects, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the injection sites are in the trigone.

In some aspects, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the injection sites are in the detrusor.

In some aspects, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the injection sites are in the lower part of the bladder wall.

In some aspects, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the injection sites are in the base of the bladder.

In some aspects, the injections are located equidistantly in a grid pattern. In some aspects, the distance between injection sites is at least about 0.5 cm, at least about 0.75 cm, at least about 1 cm, at least about 1.25 cm, at least about 1.5 cm, at least about 1.75 cm, or at least about 2 cm.

In some aspects, the depth of injection is about 1.5 mm, about 2 mm, about 2.5 mm, about 3.0 mm, about 3.5 mm, or about 4.0 mm into the detrusor, i.e., the needle is inserted approximately 1.5 mm, 2 mm, 2.5 mm, 3 mm, 3.5 mm, or 4 mm into the detrusor. In some aspects, the depth of injection is about 1.5 mm, about 2 mm, about 2.5 mm, about 3.0, about 3.5 mm, or about 4.0 mm into the trigone, i.e., the needle is inserted approximately 1.5 mm, 2 mm, 2.5 mm, 3 mm, 3.5 mm, or 4 mm into the trigone. In some aspects, the depth of injection is about 1.5 mm, about 2 mm, about 2.5 mm, about 3.0 mm, about 3.5 mm, or about 4.0 mm into the bladder wall, i.e., the needle is inserted approximately 1.5 mm, 2 mm, 2.5 mm, 3.0 mm, 3.5 mm, or 4 mm into the bladder wall.

In some aspects, the injection volume is about 0.5 ml, about 0.6 ml, about 0.7 ml, about 0.8 ml, about 0.9 ml, about 1 ml. about 1.1 ml, about 1.2 ml, about 1.3 ml, about 1.4 ml, or about 1.5 ml of a solution comprising a Maxi-K composition of the present disclosure (e.g., a plasmid such as a pVAX plasmid comprising a polynucleotide sequence encoding a Maxi-K alpha subunit).

In a particular aspect, a subject with a urinary bladder smooth muscle dysfunction (e.g., OAB) can receive a total dose of, e.g., 16,000 mcg, 24,000 mcg or 48,000 mcg of a Maxi-K composition of the present disclosure (e.g., a plasmid such as a pVAX plasmid comprising a polynucleotide sequence encoding a Maxi-K alpha subunit) administered as, e.g., approximately 20 intramuscular injections into the lower part of the bladder wall. See, e.g., U.S. Prov. Appl. 62/505,382, International Application PCT/US2018/032574 (published as Int. Publ. WO2018209351A1) and U.S. Appl. Publ. Nos. 2017/0258878, and 2017/0136106, all of which are herein incorporated by reference in their entireties.

In some aspects, administration of a Maxi-K composition of the present disclosure (e.g., a pVAX-hSlo vector of SEQ ID NO: 16, 49, or 50) is by instillation into the bladder of the subject. As used herein the term “instillation” refers to a procedure during which a tube (e.g., a catheter) is first inserted into the bladder, and a medication is infused through so that it can coat the inside of the bladder for a short time. In some aspects, the administration by instillation is conducted in an empty bladder. In some aspects, the patient is mildly dehydrated to increase absorption of the instilled composition by the bladder.

In some aspects, the volume of solution instilled inside the bladder is at least about 50 ml, at least about 60 ml, at least about 70 ml, at least about 80 ml, at least about 90 ml, at least about 100 ml, at least about 110 ml, at least about 120 ml, at least about 130 ml, at least about 140 ml, at least about 150 ml, at least about 160 ml, at least about 170 ml, at least about 180 ml, at least about 190 ml, at least about 200 ml, at least about 210 ml, at least about 220 ml, at least about 230 ml, at least about 240 ml, at least about 250 ml, at least about 260 ml, at least about 270 ml, at least about 280 ml, at least about 290, or at least about 300 ml.

In some aspects, the solution instilled inside the bladder is held for at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, or at least about 60 minutes, before being emptied. In some aspects, the administration of a Maxi-K composition of the present disclosure (e.g., a plasmid such as a pVAX plasmid comprising a polynucleotide sequence encoding a Maxi-K alpha subunit) comprises 1, 2, 3, 4, 5, 6, 7, 8, 9, or 10 instillations.

This disclosure also provides methods of treating a patient having or being at risk of having a disease or disorder related to smooth muscle tone, comprising administering a Maxi-K composition of the present disclosure (e.g., a pVAX-hSlo vector of SEQ ID NO: 16, 49, or 50) to the patient if a determination of the potential clinical effect of the administration of the Maxi-K composition according to the methods disclosed herein indicates that the patient can benefit from treatment with the Maxi-K composition.

Also provided are methods of treating a patient having or at risk of having a disease or disorder related to smooth muscle tone, comprising administering a therapeutic agent comprising a Maxi-K composition of the present disclosure (e.g., a pVAX-hSlo vector of SEQ ID NO: 16, 49, or 50) to the patient if analysis of a sample obtained from the patient indicates that the patient would benefit from such treatment (e.g., because of upregulation or downregulation in the expression of Maxi-K in the sample). In some aspects, a sample is obtained from the patient and is submitted for functional or genetic testing, for example, to a clinical laboratory.

Also provided are methods of treating a patient having or at risk of having a disease or disorder related to smooth muscle tone comprising (a) submitting a sample taken from the patient for testing (e.g., genetic testing); and, (b) administering a therapeutic agent comprising a Maxi-K composition of the present disclosure (e.g., a pVAX-hSlo vector of SEQ ID NO: 16, 49, or 50) to the patient if analysis of the sample indicates that the patient would benefit from such treatment (e.g., because of upregulation or downregulation in the expression of Maxi-K in the sample).

The disclosure also provides methods of treating a patient having or at risk of having a disease or disorder related to smooth muscle tone comprising (a) measuring muscle tone and/or Maxi-K expression in a sample obtained from a patient having or at risk of having a disease or disorder; (b) determining whether the patient can benefit from the treatment with a therapeutic agent comprising a Maxi-K composition of the present disclosure (e.g., a pVAX-hSlo vector of SEQ ID NO: 16, 49, or 50) based on the presence/absence of normal muscle tone and/or Maxi-K expression levels; and, (c) advising a healthcare provider to administer the therapeutic agent to the patient if the muscle tone and/or Maxi-K expression levels are abnormal. In some aspects, muscle tone is evaluated via surrogate measurements that are indicative of an altered muscle tone (e.g., frequency of micturition is urinary bladder smooth muscle dysfunctions such a OAB).

In certain aspects, a clinical laboratory (e.g., a genetic testing laboratory) or clinician determining smooth muscle function according to methods known in the art will advise the healthcare provider or health care benefits provider as to whether the patient can benefit from treatment with a particular Maxi-K composition of the present disclosure.

In some aspects, the clinical laboratory can advise the healthcare provider (e.g., a medical doctor or hospital) or healthcare benefits provider (e.g., a benefits administrator or a health care insurance company) as to whether the patient can benefit from the initiation, cessation, or modification of treatment with a particular Maxi-K composition of the present disclosure (e.g., a pVAX-hSlo vector of SEQ ID NO: 16, 49, or 50).

In some aspects, results of a test procedure determining the presence or absence of a smooth muscle dysfunction, risk of occurrence of a smooth muscle dysfunction, or presence or absence of a symptom related to a smooth muscle dysfunction conducted according to methods known in the art can be submitted to a healthcare provider or a healthcare benefits provider for determination of whether the patient's insurance will cover treatment with a certain Maxi-K composition of the present disclosure (e.g., a pVAX-hSlo vector of SEQ ID NO: 16, 49, or 50).

For example, for urinary bladder smooth muscle dysfunctions, urodynamic studies can be used to assess the severity of the dysfunction, the response or lack of response to treatment with a Maxi-K composition of the present disclose (e.g., a pVAX-hSlo vector of SEQ ID NO: 16, 49, or 50), or to stratify a population of patients.

In certain aspects, the disclosure provides a method of treating a patient having a smooth muscle dysfunction or at risk of having a smooth muscle dysfunction, wherein the method comprises (i) diagnosing, e.g., in a genetic testing laboratory or by a clinician, the presence or absence of a smooth muscle dysfunction or presence or absence of a symptom associated with such smooth muscle dysfunction; and (ii) advising a healthcare provider to administer or a health benefits provider to authorize the administration of a particular Maxi-K composition of the present disclosure (e.g., a pVAX-hSlo vector of SEQ ID NO: 16, 49, or 50) to the patient if the diagnosis indicates that the patient can benefit from the treatment with the Maxi-K composition.

In certain aspects, the treatment method can comprise: (i) diagnosing, e.g., in a genetic testing laboratory or by a clinician, the presence or absence of a smooth muscle dysfunction or presence or absence of a symptom associated with such smooth muscle dysfunction; (ii) determining whether the diagnosis indicates that the patient can benefit from the treatment with a Maxi-K composition of the present disclosure (e.g., a pVAX-hSlo vector of SEQ ID NO: 16, 49, or 50); and (iii) advising a healthcare provider the adjust the dosage or a health benefits provider to authorize the adjustment of the dosage of the Maxi-K composition of the present disclosure if indicated, e.g., to

(a) to increase or maintain the amount or frequency of the administration of the Maxi-K composition of the present disclosure (e.g., a pVAX-hSlo vector of SEQ ID NO: 16, 49, or 50) to the patient,

(b) to discontinue the administration of the Maxi-K composition of the present disclosure (e.g., a pVAX-hSlo vector of SEQ ID NO: 16, 49, or 50), or

(c) to maintain or reduce the amount of Maxi-K composition of the present disclosure administered (e.g., a pVAX-hSlo vector of SEQ ID NO: 16, 49, or 50)or the frequency of the administration of the Maxi-K composition of the present disclosure.

The determination of (i) the presence or absence of a smooth muscle dysfunction, (ii) the risk of appearance of a smooth muscle dysfunction, (iii) the presence or absence of symptoms or sequelae resulting from the smooth muscle dysfunction, (iv) the risk of appearance of symptoms or sequelae resulting from the muscle dysfunction, (v) the severity of the smooth muscle dysfunction or symptoms or sequelae associated with the smooth muscle dysfunction, (vi) the patient's response or lack thereof to standard treatments of the smooth muscle dysfunction or symptoms or sequelae associated with the smooth muscle dysfunction, (vii) the patient's response or lack thereof to the administration of Maxi-K compositions of the present disclosure (e.g., a pVAX-hSlo vector of SEQ ID NO: 16, 49, or 50) to treat the smooth muscle dysfunction or symptoms or sequelae associated with the smooth muscle dysfunction, or (viii) any combinations thereof can be used, as discussed above, as part of the treatment of a smooth muscle dysfunction or symptoms or sequelae associated with the smooth muscle dysfunction. Furthermore, these determinations can be used, e.g.,

(a) to select a patient for treatment with a Maxi-K composition of the present disclosure (e.g., a pVAX-hSlo vector of SEQ ID NO: 16, 49, or 50);

(b) to exclude a patient from treatment with a Maxi-K compositions of the present disclosure (e.g., a pVAX-hSlo vector of SEQ ID NO: 16, 49, or 50);

(c) to add a Maxi-K composition of the present disclosure (e.g., a pVAX-hSlo vector of SEQ ID NO: 16, 49, or 50) to a standard treatment (combination treatment);

(d) to increase the dose of Maxi-K composition of the present disclosure (e.g., a pVAX-hSlo vector of SEQ ID NO: 16, 49, or 50);

(e) to decrease the dose of Maxi-K composition of the present disclosure (e.g., a pVAX-hSlo vector of SEQ ID NO: 16, 49, or 50);

(f) to increase the frequency of administration of the Maxi-K composition of the present disclosure (e.g., a pVAX-hSlo vector of SEQ ID NO: 16, 49, or 50);

(g) to decrease the frequency of administration of the Maxi-K composition of the present disclosure (e.g., a pVAX-hSlo vector of SEQ ID NO: 16, 49, or 50);

(h) to select and alternative route of administration for a Maxi-K composition of the present disclosure (e.g., a pVAX-hSlo vector of SEQ ID NO: 16, 49, or 50);

(i) to select a specific Maxi-K composition of the present disclosure (e.g., a pVAX-hSlo vector of SEQ ID NO: 16, 49, or 50) among several potential Maxi-K composition of the present disclosure as options for treatment;

(j) to select a patient for a clinical trial with a Maxi-K composition of the present disclosure (e.g., a pVAX-hSlo vector of SEQ ID NO: 16, 49, or 50);

(k) to exclude a patient for a clinical trial with a Maxi-K composition of the present disclosure (e.g., a pVAX-hSlo vector of SEQ ID NO: 16, 49, or 50);

(l) to determine the prognosis of the patient; or

(m) any combination thereof.

In response to the potential phenotypic impact of the administration of a Maxi-K composition disclosed herein (e.g., a pVAX-hSlo vector of SEQ ID NO: 16, 49, or 50), a healthcare provider, healthcare benefits provider, or counselor can provide treatment advice and/or lifestyle advice as part of a treatment. Thus, in response to the identification of a smooth muscle dysfunction treatable with a Maxi-K composition of the present disclosure (e.g., a pVAX-hSlo vector of SEQ ID NO: 16, 49, or 50), a subject can be advised, e.g., to adjust his or her diet, to cease smoking, or to cease or reduce the ingestion of alcohol, in addition to being administered a Maxi-K composition of the present disclosure (e.g., a pVAX-hSlo vector of SEQ ID NO: 16, 49, or 50).

In a particular aspect, the present disclosure specifically provides methods of gene therapy wherein the administration of a Maxi-K composition of the present disclosure (e.g., a pVAX-hSlo vector of SEQ ID NO: 16, 49, or 50) modulates relaxation of smooth muscle in the urinary bladder. These Maxi-K polypeptides expressed in muscle of the urinary bladder wall as a result of gene therapy with the Maxi-K composition of the present disclosure (e.g., a pVAX-hSlo vector of SEQ ID NO: 16, 49, or 50) promotes or enhances relaxation of smooth muscle, and thus decreases smooth muscle tone. In particular, where smooth muscle tone is decreased in the bladder, bladder capability is increased. In this particular aspect, the method of the present disclosure can be used to alleviate a hyperreflexic bladder. A hyperreflexic bladder can result from a variety of disorders, including neurogenic and arteriogenic dysfunctions, as well as other conditions which cause incomplete relaxation or heightened contractility of the smooth muscle of the bladder.

In a particular aspect, the methods of the present disclosure are used to treat or alleviate a symptom of overactive bladder (OAB) syndrome or detrusor overactivity by introducing into bladder smooth muscle cells of the subject a Maxi-K composition of the present disclosure (e.g., a pVAX-hSlo vector of SEQ ID NO: 16, 49, or 50), e.g., via injection into the bladder wall (e.g., detrusor muscle) and in particular, specific locations in the bladder wall (e.g., the trigone). The nucleic acid is expressed in the bladder smooth cells such that bladder smooth muscle tone is regulated; thus, the regulation of bladder smooth muscle tone results in less heightened contractility of smooth muscle in the subject.

In some aspects of the present disclosure, the methods and compositions disclosed herein are applied to a patient suffering from refractory overactive bladder. In particular aspects of the methods of the present disclosure, the subject is a female patient or a population of female patients suffering from overactive bladder and urge urinary incontinence. In another aspect, the subject is a male patient or a population of male patients suffering from overactive bladder and urge urinary incontinence. In yet another aspect, the subject is a population of male and female patients suffering from overactive bladder and urge urinary incontinence. In a particular aspect, such patients are administered a Maxi-K composition of the present disclosure, e.g., a vector such as pVAX comprising a polynucleotide sequence encoding a Maxi-K alpha subunit.

In some aspects, the Maxi-K compositions of the present disclosure (e.g., a pVAX-hSlo vector of SEQ ID NO: 16, 49, or 50) are administered to such patients via injection into the urinary bladder, e.g., at 20 to 30 sites in the urinary bladder detrusor muscle, at a depth of approximately 2 mm into the muscle, with a spacing of approximately 1 cm between injection sites, wherein each injection comprises 16000 ug, 24000 ug, or 48000 ug of a Maxi-K composition of the present disclosure (e.g., pVAX-hSlo1).

Other diseases and conditions that can treated by using the compositions and methods disclosed herein are presented in Section IV of the present application.

### III. MAXI-K COMPOSITIONS FOR THE TREATMENT OF SMOOTH MUSCLE DYSFUNCTION

The present disclosure provides Maxi-K compositions (e.g., a pVAX-hSlo vector of SEQ ID NO: 16, 49, or 50) that can be administered, for example, according to the methods disclosed above. As discussed above, the Maxi-K compositions of the present disclosure comprise, e.g.,

(a) one or more polynucleotides encoding one or more Maxi-K polypeptides schematically presented in FIG. 17, and domains or combination of domains thereof (according to the domain boundaries known in the art);

(b) one or more polynucleotides encoding one or more Maxi-K polypeptide sequences presented in TABLE 1 (e.g., Maxi-K alpha subunits, Maxi-K beta subunits, or combinations thereof), or fragments (e.g., an alpha subunit lacking one of more of the domains depicted in the FIG. 17 representation), isoforms, mutants, variants, or derivatives thereof;

(c) one or more polynucleotides encoding fusions or chimeric proteins comprising Maxi-K polypeptides disclosed herein, e.g., a Maxi-K alpha subunit genetically fused to a non-Maxi-K polypeptide conferring a desirable property, or a fusion between two or more Maxi-K polypeptides, e.g., an alpha subunit and a beta subunit;

(d) plasmids or vectors comprising the polynucleotides of (a), (b), (c) or any combination thereof;

(e) cells comprising the polynucleotides of (a), (b), or (c), the plasmids or vectors of (d), or any combination thereof;

(f) pharmaceutical compositions comprising the polynucleotides of (a), (b), or (c), the plasmids or vectors of (d), the cells of (e); or, (g) any combination thereof.

In some aspects, the Maxi-K composition comprises a vector (e.g., a pVAX-hSlo vector of SEQ ID NO: 16, 49, or 50). Suitable vectors include, e.g., viral vectors such as adenoviruses, adeno-associated viruses (AAV), and retroviruses (e.g., lentiviruses), liposomes, other lipid-containing complexes, nanoparticles, and any other molecules or other macromolecular complexes capable of mediating delivery of a polynucleotide to a target cell. The recombinant vectors and plasmids of the present disclosure can also contain a nucleotide sequence encoding suitable regulatory elements, so as to effect expression of the vector construct in a suitable host cell. As used herein, the term “expression” refers to the ability of the vector to transcribe the inserted DNA sequence into mRNA so that synthesis of the protein encoded by the inserted nucleic acid can occur.

Those skilled in the art will appreciate that a variety of enhancers and promoters are suitable for use in the constructs in the Maxi-K compositions of the present disclosure (e.g., a pVAX-hSlo vector of SEQ ID NO: 16, 49, or 50); and that the constructs will contain the necessary start, termination, and control sequences or proper transcription and processing of the DNA sequence encoding a protein involved in the regulation of smooth muscle tone, upon introduction of the recombinant vector construct into a host tell.

Non-viral vectors provided by the present disclosure, for the expression in a smooth muscle cell of the nucleic sequence encoding a Maxi-K polypeptide (e.g., a Maxi-K alpha subunit, a Maxi-K beta subunit, or a combination thereof) can comprise all or a portion of any of the following vectors known to one skilled in the art: pVax (Thermo Fisher Scientific), pCMVβ (Invitrogen), pcDNA3 (Invitrogen), pET-3d (Novagen), pProEx-1 (Life Technologies), pFastBac 1 (Life Technologies), pSFV (Life Technologies), pcDNA2 (Invitrogen), pSL301 (Invitrogen), pSE280 (Invitrogen), pSE380 (Invitrogen), pSE420 (Invitrogen), pTrcHis A, B, C (Invitrogen), pRSET A, B, C (Invitrogen), pYES2 (Invitrogen), pAC360 (Invitrogen), pVL1392 and pVI1392 (Invitrogen), pCDM8 (Invitrogen), pCDNA I (Invitrogen), pREP4 (Invitrogen), pREP8 (Invitrogen), pREP9 (Invitrogen), pREP10 (Invitrogen), pCEP4 (Invitrogen), pEBVHis (Invitrogen), and λPop6. Other vectors can be used as well. In a particular aspect, the vector is pVax, and the Maxi-K open reading in pVax encodes a Maxi-K alpha subunit (e.g., a wild type Maxi-K alpha subunit or Maxi-K mutant subunit disclosed herein).

In some aspects, the pVax vector sequence comprises a sequence of SEQ ID NO: 10. In some aspects, the pVAX vector sequence comprises a sequence with at least about 60%, at least about 65%, at least about 70%, at least about 75%, at least about 80%, at least about 85%, at least about 90%, at least about 95%, at least about 96%, at least about 97%, at least about 98% or at least about 99% identity to SEQ ID NO: 10. In some aspects, the pVAX sequence comprises a substitution of G for A at position 2 of SEQ ID NO: 10, an additional G at position 5 of SEQ ID NO: 10, a substitution of T for C at position 1158 of SEQ ID NO: 10, a missing A at position 2092 of SEQ ID NO: 10, a substitution of T for C at position 2493 of SEQ ID NO: 10, or a combination thereof.

Additional variations in the pVax vector are shown in FIG. 18 (variations N1-N4, and N10-16). In some aspects, the pVax vector comprises a sequence of SEQ ID NO: 16, 49, or 50, except the Maxi-K-encoding portion (i.e., SEQ ID NO: 51, 52, or 53). In some aspects, the pVax vector comprises a sequence of SEQ ID NO: 16 minus the Maxi-K-encoding portion (i.e., SEQ ID NO: 51) and at least one of the N1, N2, N3, N4, N10, N11, N12, N13, N14, N15, or N16 variations of FIG. 18, or a combination thereof. In some aspects, the pVax vector comprises a sequence of SEQ ID NO: 49 minus the Maxi-K-encoding portion (i.e., SEQ ID NO: 52) and at least one of the N1, N2, N3, N4, N10, N11, N12, N13, N14, N15, or N16 variations of FIG. 18, or a combination thereof. In some aspects, the pVax vector comprises a sequence of SEQ ID NO: 50 minus the Maxi-K-encoding portion (i.e., SEQ ID NO: 53) and at least one of the N1, N2, N3, N4, N10, N11, N12, N13, N14, N15, or N16 variations of FIG. 18, or a combination thereof.

In some aspects, the nucleic acid molecule is operably-linked to a promoter. In some aspects, the promoter is not an urothelium specific expression promoter. For example, the promoter is a CMV promoter (VAX) or a smooth muscle specific expression promoter (SMAA).

Promoters suitable for the practice of the methods of the present disclosure include, but are not limited to, constitutive promoters, tissue-specific promoters, and inducible promoters. In some aspects, the promoter is a smooth muscle promoter. In other aspects, the promoter is a muscle cell promoter. In some aspects, the promoter is not an urothelium specific expression promoter.

In one aspect, expression of the Maxi-K polynucleotide sequence encoding a Maxi-K polypeptide disclosed herein (e.g., a Maxi-K alpha subunit, a Maxi-K beta subunit, or a combination thereof) is controlled and affected by the particular vector into which the Maxi-K polynucleotide sequence has been introduced. Some eukaryotic vectors have been engineered so that they are capable of expressing inserted nucleic acids to high levels within the host cell. Such vectors utilize one of a number of powerful promoters to direct the high level of expression. Eukaryotic vectors use promoter-enhancer sequences of viral genes, especially those of tumor viruses.

In some aspects, expression of the Maxi-K polynucleotide sequence encoding the Maxi-K polypeptide protein is regulated through the use of inducible promoters. Non-limiting examples of inducible promoters include, e.g., metallothionein promoters and mouse mammary tumor virus promoters. Depending on the vector, expression of the Maxi-K polypeptide sequence in the smooth muscle cell can be induced by the addition of a specific compound at a certain point in the growth cycle of the cell. Other examples of promoters and enhancers effective for use in the recombinant vectors of the present disclosure include, but are not limited to, CMV (cytomegalovirus), SV40 (simian virus 40), HSV (herpes simplex virus), EBV (Epstein-Barr virus), retrovirus, adenoviral promoters and enhancers, and smooth-muscle-specific promoters and enhancers.

An example of a smooth-muscle-specific promoter is SM22a. Exemplary smooth muscle promoters are described in U.S. Pat. No. 7,169,764, the contents of which are herein incorporated by reference in its entirety. In some particular aspects of the present disclosure, the vector comprises a SM22a promoter sequence, which can include but is not limited to sequences such as SEQ ID NO: 9.

In some aspects, the vector comprises a promoter is a human cytomegalovirus intermediate-early promoter (CMEV) sequence, which can include but is not limited to sequences such as SEQ ID NO: 1. In some aspects, the vector comprises a T7 priming site, which can include but is not limited to sequences such as SEQ ID NO: 2.

In some aspects, the recombinant virus and/or plasmid used to express a Maxi-K polypeptide of the disclosure comprises a polyA (polyadenylation) sequence, such as those provided herein, (e.g., a BGH polyA sequence). Generally, any suitable polyA sequence can be used for the desired expression of the transgene. For example, in some cases, the present disclosure provides for a sequence comprising BGH polyA sequence, or portion of a BGH polyA sequence. In some cases, the present disclosure provides for polyA sequences comprising a combination of one or more polyA sequences or sequence elements. In some cases, no polyA sequence is used. In some cases, one or more polyA sequences may be referred to as untranslated regions (UTRs), 3′UTRs, or termination sequences.

A polyA sequence can comprise a length of about 1-10 bp, about 10-20 bp, about 20-50 bp, about 50-100 bp, about 100-500 bp, about 500 bp-1 Kb, about 1 Kb-2 Kb, about 2 Kb-3 Kb, about 3 Kb-4 Kb, about 4 Kb-5 Kb, about 5 Kb-6 Kb, about 6 Kb-7 Kb, about 7 Kb-8 Kb, about 8 Kb-9 Kb, or about 9 Kb-10 Kb in length. A polyA sequence can comprise a length of at least 1 bp, at least 2 bp, at least 3 bp, at least 4 bp, at least 5 bp, at least 6 bp, at least 7 bp, at least 8 bp, at least 9 bp, at least about 10 bp, at least about 20 bp, at least about 30 bp, at least about 40 bp, at least about 50 bp, at least about 60 bp, at least about 70 bp, at least about 80 bp, at least about 90 bp, at least about 100 bp, at least about 200 bp, at least about 300 bp, at least about 400 bp, at least about 500 bp, at least about 600 bp, at least about 700 bp, at least about 800 bp, at least about 900 bp, at least about 1 Kb, at least about 1.5 Kb, at least about 2 Kb, at least about 2.5 Kb, at least about 3 Kb, at least about 3.5 Kb, at least about 4 Kb, at least about 4.5 Kb, at least about 5 Kb, at least about 5.5 Kb, at least about 6 Kb, at least about 6.5 Kb, at least about 7 Kb, at least about 7.5 Kb, at least about 8 Kb, at least about 8.5 Kb, at least about 9 Kb, at least about 9.5 Kb, or at least about 10 Kb in length.

In some aspects, a BGH polyA can include but is not limited to sequences such as SEQ ID NO: 3. In some aspects, polyA sequences can be optimized for various parameters affecting protein expression, including but not limited to mRNA half-life of the transgene in the cell, stability of the mRNA of the transgene or transcriptional regulation. For example, polyA sequences can be altered to increase mRNA transcription of the transgene, which can result in increased protein expression. In some aspects, the polyA sequences can be altered to decrease the half-life of the mRNA transcript of the transgene, which can result in decreased protein expression.

In some aspects, the vector, comprises a sequence encoding a replication origin sequence, such as those provided herein. Origin of replication sequences, generally provide sequence useful for propagating a plasmid/vector. In some aspects, the origin of replication is a pUC origin of replication. In some cases, a pUC origin of replication sequence can include, but is not limited to sequences such as SEQ ID NO: 4.

In some aspects, the vector can also comprise a selectable marker. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Examples of negative selectable markers may include the inclusion of resistance genes to antibiotics, such as ampicillin or kanamycin. Such marker genes can provide an added measure of control that can be advantageous in gene therapy contexts. A large variety of such vectors are known in the art and are generally available.

In some cases, the vector can comprises a nucleic acid encoding resistance to kanamycin. In some aspects, the nucleic acid encoding resistance to kanamycin can include, but is not limited to the sequence of SEQ ID NO: 5.

In some aspects, the vector comprise a polynucleotide encoding a Maxi-K polypeptide (e.g., a Maxi-K alpha subunit, a Maxi-K beta subunit, or a combination), a mutant Maxi-K polypeptide, a Maxi-K polypeptide fragment (e.g., a functional fragment), a variant, a derivative, a fusion or a chimaera as disclosed in the previous section in the present application. An exemplary nucleic acid encoding a Maxi-K polypeptide includes the nucleic acid sequence of SEQ ID NO: 6 (wild type human Maxi-K alpha subunit), or SEQ ID NOs: 51, 52, or 53.

Modifications of the Maxi-K gene (e.g., in Maxi-K alpha subunit and/or Maxi-K beta subunits) can be used to effectively treat human disease that is caused, for example, alterations of the Maxi-K channel expression, activity, upstream signaling events, and/or downstream signaling events. Modifications to a wild type Maxi-K polynucleotide or polypeptide include, but are not limited to, deletions, insertions, frameshifts, substitutions, and inversions.

Contemplated modifications to the wild type Maxi-K alpha subunit polynucleotide sequence include substitutions of at least one nucleotide (e.g., a single nucleotide) in a DNA, cDNA, or RNA (e.g., mRNA) sequence encoding Maxi-K and/or substitutions of at least one amino acid in (e.g., a single amino acid) the Maxi-K polypeptide sequence.

A single point mutation in the alpha, or pore-forming, subunit of the human Maxi-K channel is more efficient in reducing smooth muscle dysfunction, e.g., detrusor overactivity (DO) in urinary bladder smooth muscle, than the wild type Maxi-K alpha subunit gene. Specifically, a single point mutation at nucleotide position 1054 of the Maxi-K alpha subunit gene which results in a substitution of a Threonine (T) for a Serine (S) at position 352 of the amino acid sequence (T352S) causes increased current of the Maxi-K channel at lower intracellular calcium ion concentrations when compared to the channels expressed by the non-mutated gene.

The single mutation improves conductivity in high glucose of high oxidative stress environments compared to genes having multiple mutations. The Maxi-K alpha subunit encoded the T352S mutant (e.g., incorporation into a pVAX to yield a pVAX-hSlo-T352S construct) is more physiologically effective than a Maxi-K channel encoded by a wild type sequence or a construct comprising a Maxi-K polynucleotide encoding the wild type sequence to treat age- and disease-induced alternations in wild-type Maxi-K channel function.

In some aspects, the Maxi-K polynucleotide encoding Maxi-K alpha subunit comprises a point mutation at nucleic acid position 1054 when numbered in accordance with SEQ ID NO: 7. This point mutation results in an amino acid substitution at position 352 of the Maxi-K alpha subunit when numbered in accordance with SEQ ID NO: 7. For example, the point mutation is a substitution of a Serine (S) for a Threonine (T) (e.g., T352S).

Optionally, additional modifications in the Maxi-K alpha subunit wild type sequence include point mutations that result in one or more amino acid substitutions at amino acid positions 496, 602, 681, 778, 805, 977, or any combination thereof when numbered in accordance with SEQ ID NO: 8. In particular aspects, the mutations at such positions are C496A (“C2 mutation”), M602L (“M1 mutation”), C681A (“C3 mutation”), M778L (“M2 mutation”), M805L (“M3 mutation”) or C977A (“C1 mutation”), which are highlighted by white lettering on a black background and accompanied by the name of the mutation in SEQ ID NO:8, below:

| (SEQ ID NO: 11) | | |
|---|---|---|
| 1 | ATGGCAAATGGTGGCGGCGGCGGCGGCGGCAGCAGCGGCGGCGGCGGCGGCGGCGGAGGC | 60 |
| 1 | M A N G G G G G G G S S G G G G G G G G | |
| 61 | AGCAGTCTTAGAATGAGTAGCAATATCCACGCGAACCATCTCAGCCTAGACGTGTCCTCC | 120 |
| 21 | S S L R M S S N I H A N H L S L D V S S | |
| 121 | TCCTCCTCCTCCTCCTCTTCCTCTTCTTCTTCTTCCTCCTCCTCTTCCTCCTCGTCCTCG | 180 |
| 41 | S S S S S S S S S S S S S S S S S S S S | |
| 181 | GTCCACGAGCCCAAGATGGATGCGCTCATCATCCCGGTGACCATGGAGGTGCCGTGCGAC | 240 |
| 61 | V H E P K M D A L I I P V T M E V P C D | |
| 241 | AGCCGGGGCCAACGCATGTGGTGGGCTTTCCTGGCCTCCTCCATGGTGACTTTCTTCGGG | 300 |
| 81 | S R G Q R M W W A F L A S S M V T F F G | |
| 301 | GGCCTCTTCATCATCTTGCTCTGGCGGACGCTCAAGTACCTGTGGACCGTGTGCTGCCAC | 360 |
| 101 | G L F I I L L W R T L K Y L W T V C C H | |
| 361 | TGCGGGGGCAAGACGAAGGAGGCCCAGAAGATTAACAATGGCTCAAGCCAGGCGGATGGC | 420 |
| 121 | C G G K T K E A Q K I N N G S S Q A D G | |
| 421 | ACTCTCAAACCAGTGGATGAAAAAGAGGAGGCAGTGGCCGCCGAGGTCGGCTGGATGACC | 480 |
| 141 | T L K P V D E K E E A V A A E V G W M T | |
| 481 | TCCGTGAAGGACTGGGCGGGGGTGATGATATCCGCCCAGACACTGACTGGCAGAGTCCTG | 540 |
| 161 | S V K D W A G V M I S A Q T L T G R V L | |
| 541 | GTTGTCTTAGTCTTTGCTCTCAGCATCGGTGCACTTGTAATATACTTCATAGATTCATCA | 600 |
| 181 | V V L V F A L S I G A L V I Y F I D S S | |
| 601 | AACCCAATAGAATCCTGCCAGAATTTCTACAAAGATTTCACATTACAGATCGACATGGCT | 660 |
| 201 | N P I E S C Q N F Y K D F T L Q I D M A | |
| 661 | TTCAACGTGTTCTTCCTTCTCTACTTCGGCTTGCGGTTTATTGCAGCCAACGATAAATTG | 720 |
| 221 | F N V F F L L Y F G L R F I A A N D K L | |
| 721 | TGGTTCTGGCTGGAAGTGAACTCTGTAGTGGATTTCTTCACGGTGCCCCCCGTGTTTGTG | 780 |
| 241 | W F W L E V N S V V D F F T V P P V F V | |
| 781 | TCTGTGTACTTAAACAGAAGTTGGCTTGGTTTGAGATTTTTAAGAGCTCTGAGACTGATA | 840 |
| 261 | S V Y L N R S W L G L R F L R A L R L I | |
| 841 | CAGTTTTCAGAAATTTTGCAGTTTCTGAATATTCTTAAAACAAGTAATTCCATCAAGCTG | 900 |
| 281 | Q F S E I L Q F L N I L K T S N S I K L | |
| 901 | GTGAATCTGCTCTCCATATTTATCAGCACGTGGCTGACTGCAGCCGGGTTCATCCATTTG | 960 |
| 301 | V N L L S I F I S T W L T A A G F I H L | |
| 961 | GTGGAGAATTCAGGGGACCCATGGGAAAATTTCCAAAACAACCAGGCTCTCACCTACTGG | 1020 |
| 321 | V E N S G D P W E N F Q N N Q A L T Y W | |
| 1021 | GAATGTGTCTATTTACTCATGGTCACAATGTCCACCGTTGGTTATGGGGATGTTTATGCA | 1080 |
| | ATGGTCACAATGTCCTCCGTTGGTTATGGGGAT | |
| 341 | E C V Y L L M V T M S T V G Y G D V Y A | |
| (SEQ ID NO: 7) | | |
| 1081 | AAAACCACACTTGGGCGCCTCTTCATGGTCTTCTTCATCCTCGGGGGACTGGCCATGTTT | 1140 |
| 361 | K T T L G R L F M V F F I L G G L A M F | |
| 1141 | GCCAGCTACGTCCCTGAAATCATAGAGTTAATAGGAAACCGCAAGAAATACGGGGGCTCC | 1200 |
| 381 | A S Y V P E I T E L I G N R K K Y G G S | |
| 1201 | TATAGTGCGGTTAGTGGAAGAAAGCACATTGTGGTCTGCGGACACATCACTCTGGAGAGT | 1260 |
| 401 | Y S A V S G R K H I V V C G H I T L E S | |
| 1261 | GTTTCCAACTTCCTGAAGGACTTTCTGCACAAGGACCGGGATGACGTCAATGTGGAGATC | 1320 |
| 421 | V S N F L K D F L H K D R D D V N V E I | |
| 1321 | GTTTTTCTTCACAACATCTCCCCCAACCTGGAGCTTGAAGCTCTGTTCAAACGACATTTT | 1380 |
| 441 | V E L H N I S P N L E L E A L F K R H F | |
| 1381 | ACTCAGGTGGAATTTTATCAGGGTTCCGTCCTCAATCCACATGATCTTGCAAGAGTCAAG | 1440 |
| 461 | T Q V E F Y Q G S V L N P H D L A R V K | |
| 1441 | ATAGAGTCAGCAGATGCATGCCTGATCCTTGCCAACAAGTACTGCGCTGACCCGGATGCG | 1500 |
| 481 | I E S A D A C L I L A N K Y C A D P D A | |
| | C496/A | |
| 1501 | GAGGATGCCTCGAATATCATGAGAGTAATCTCCATAAAGAACTACCATCCGAAGATAAGA | 1560 |
| 501 | E D A S N I M R V I S I K N Y H P K I R | |
| 1561 | ATCATCACTCAAATGCTGCAGTATCACAACAAGGCCCATCTGCTAAACATCCCGAGCTGG | 1620 |
| 521 | I I T Q M L Q Y H N K A H L L N I P S W | |
| 1621 | AATTGGAAAGAAGGTGATGACGCAATCTGCCTCGCAGAGTTGAAGTTGGGCTTCATAGCC | 1680 |
| 541 | N W K E G D D A I C L A E L K L G F I A | |
| 1681 | CAGAGCTGCCTGGCTCAAGGCCTCTCCACCATGCTTGCCAACCTCTTCTCCATGAGGTCA | 1740 |
| 561 | Q S C L A Q G L S T M L A N L F S M R S | |
| 1741 | TTCATAAAGATTGAGGAAGACACATGGCAGAAATACTACTTGGAAGGAGTCTCAAATGAA | 1800 |
| 581 | F I K I E E D T W Q K Y Y L E G V S N E | |
| 1801 | ATGTACACAGAATATCTCTCCAGTGCCTTCGTGGGTCTGTCCTTCCCTACTGTTTGTGAG | 1860 |
| 601 | M Y T E Y L S S A F V G L S E P T V C E | |
| M602/L | | |
| 1861 | CTGTGTTTTGTGAAGCTCAAGCTCCTAATGATAGCCATTGAGTACAAGTCTGCCAACCGA | 1920 |
| 621 | L C F V K L K L L M I A I E Y K S A N R | |
| 1921 | GAGAGCCGTATATTAATTAATCCTGGAAACCATCTTAAGATCCAAGAAGGTACTTTAGGA | 1980 |
| 641 | E S R I L I N P G N H L K I Q E G T L G | |
| 1981 | TTTTTCATCGCAAGTGATGCCAAAGAAGTTAAAAGGGCATTTTTTTACTGCAAGGCCTGT | 2040 |
| 661 | F F I A S D A K E V K R A F F Y C K A C | |
| | C681/A | |
| 2041 | CATGATGACATCACAGATCCCAAAAGAATAAAAAAATGTGGCTGCAAACGGCTTGAAGAT | 2100 |
| 681 | H D D I T D P K R I K K C G C K R L E D | |
| 2101 | GAGCAGCCGTCAACACTATCACCAAAAAAAAAGCAACGGAATGGAGGCATGCGGAACTCA | 2160 |
| 701 | E Q P S T L S P K K K Q R N G G M R N S | |
| 2161 | CCCAACACCTCGCCTAAGCTGATGAGGCATGACCCCTTGTTAATTCCTGGCAATGATCAG | 2220 |
| 721 | P N T S P K L M R H D P L L I P G N D Q | |
| 2221 | ATTGACAACATGGACTCCAATGTGAAGAAGTACGACTCTACTGGGATGTTTCACTGGTGT | 2280 |
| 741 | I D N M D S N V K K Y D S T G M F H W C | |
| 2281 | GCACCCAAGGAGATAGAGAAAGTCATCCTGACTCGAAGTGAAGCTGCCATGACCGTCCTG | 2340 |
| 761 | A P K E I E K V I L T R S E A A M T V L | |
| | M778/L | |
| 2341 | AGTGGCCATGTCGTGGTCTGCATCTTTGGCGACGTCAGCTCAGCCCTGATCGGCCTCCGG | 2400 |
| 781 | S G H V V V C I F G D V S S A L I G L R | |
| 2401 | AACCTGGTGATGCCGCTCCGTGCCAGCAACTTTCATTACCATGAGCTCAAGCACATTGTG | 2460 |
| 801 | N L V M P L R A S N E H Y H E L K H I V | |
| | M805/L | |
| 2461 | TTTGTGGGCTCTATTGAGTACCTCAAGCGGGAATGGGAGACGCTTCATAACTTCCCCAAA | 2520 |
| 821 | F V G S I E Y L K R E W E T L H N F P K | |
| 2S21 | GTGTCCATATTGCCTGGTACGCCATTAAGTCGGGCTGATTTAAGGGCTGTCAACATCAAC | 2580 |
| 841 | V S I L P G T P L S R A D L R A V N I N | |
| 2581 | CTCTGTGACATGTGCGTTATCCTGTCAGCCAATCAGAATAATATTGATGATACTTCGCTG | 2640 |
| 861 | L C D M C V I L S A N Q N N I D D T S L | |
| 2641 | CAGGACAAGGAATGCATCTTGGCGTCACTCAACATCAAATCTATGCAGTTTGATGACAGC | 2700 |
| 881 | Q D K E C I L A S L N I K S M Q F D D S | |
| 2701 | ATCGGAGTCTTGCAGGCTAATTCCCAAGGGTTCACACCTCCAGGAATGGATAGATCCTCT | 2760 |
| 901 | I G V L Q A N S Q G F T P P G M D R S S | |
| 2761 | CCAGATAACAGCCCAGTGCACGGGATGTTACGTCAACCATCCATCACAACTGGGGTCAAC | 2820 |
| 921 | P D N S P V H G M L R Q P S I T T G V N | |
| 2821 | ATCCCCATCATCACTGAACTAGTGAACGATACTAATGTTCAGTTTTTGGACCAAGACGAT | 2880 |
| 941 | I P I I T E L V N D T N V Q F L D Q D D | |
| 2881 | GATGATGACCCTGATACAGAACTGTACCTCACGCAGCCCTTTGCCTGTGGGACAGCATTT | 2940 |
| 961 | D D D P D T E L Y L T Q P F A C G T A F | |
| | C977/A | |
| 2941 | GCCGTCAGTGTCCTGGACTCACTCATGAGCGCGACGTACTTCAATGACAATATCCTCACC | 3000 |
| 981 | A V S V L D S L M S A T Y E N D N I L T | |
| 3001 | CTGATACGGACCCTGGTGACCGGAGGAGCCACGCCGGAGCTGGAGGCTCTGATTGCTGAG | 3060 |
| 1001 | L I R T L V T G G A T P E L E A L I A E | |
| 3061 | GAAAACGCCCTTAGAGGTGGCTACAGCACCCCGCAGACACTGGCCAATAGGGACCGCTGC | 3120 |
| 1021 | E N A L R G G Y S T P Q T L A N R D R C | |
| 3121 | CGCGTGGCCCAGTTAGCTCTGCTCGATGGGCCATTTGCGGACTTAGGGGATGGTGGTTGT | 3180 |
| 1041 | R V A Q L A L L D G P F A D L G D G G C | |
| 3181 | TATGGTGATCTGTTCTGCAAAGCTCTGAAAACATATAATATGCTTTGTTTTGGAATTTAC | 3240 |
| 1061 | Y G D L F C K A L K T Y N M L C F G I Y | |
| 3241 | CGGCTGAGAGATGCTCACCTCAGCACCCCCAGTCAGTGCACAAAGAGGTATGTCATCACC | 3300 |
| 1081 | R L R D A H L S T P S Q C T K R Y V I T | |
| 3301 | AACCCGCCCTATGAGTTTGAGCTCGTGCCGACGGACCTGATCTTCTGCTTAATGCAGTTT | 3360 |
| 1101 | N P P Y E F E L V P T D L I F C L M Q F | |
| 3361 | GACCACAATGCCGGCCAGTCCCGGGCCAGCCTGTCCCATTCCTCCCACTCGTCGCAGTCC | 3420 |
| 1121 | D H N A G Q S R A S L S H S S H S S Q S | |
| 3421 | TCCAGCAAGAAGAGCTCCTCTGTTCACTCCATCCCATCCACAGCAAACCGACAGAACCGG | 3480 |
| 1141 | S S K K S S S V H S I P S T A N R Q N R | |
| 3481 | CCCAAGTCCAGGGAGTCCCGGGACAAACAGAAGTACGTGCAGGAAGAGCGGCTT | 3538 |
| (SEQ ID NO: 8) | | |
| 1161 | P K S R E S R D K Q K Y V Q E E R L | |

The present disclosure further provides compositions comprising a cell, e.g., a smooth muscle cell or a stem cell, which expresses an exogenous DNA or RNA (e.g., mRNA) sequence encoding a protein involved in the regulation of smooth muscle tone, e.g., a Maxi-K polypeptide such as a Maxi-K alpha subunit, a Maxi-K beta subunit, or a combination thereof. As used herein, “exogenous” means any DNA or RNA (e.g., an mRNA) that is introduced into an organism or cell.

Exemplary nucleic acid molecules that can be used to practice the methods of the present disclosure include the vectors pVAX-hSlo-T352S; pVAX-hSlo-T352S-C997; pVAX-hSlo-T352S-C496A; pVAX-hSlo-T352S-C681; pVAX-hSlo-T352S-M602L; pVAX-hSlo-T352S-M778L; pVAX-hSlo-T352S-M805L; pSMAA-hSlo-T352S; pSMAA-hSlo-T352S-C997; pSMAA-hSlo-T352S-C496A; pSMAAhSlo-T352S-C681A; pSMAA-hSlo-T352S-M602L; pSMAA-hSlo-T352S-M778L; and pSMAA-hSlo-T352S-M805L.

The present application incorporates the following documents by reference in their entireties:
U.S. Patent Appl. Publ. 2008/0269159,
International Application Publication WO2013151665A2 and U.S. Patent Appl. Publ. No. US2018311381 (and in particular SEQ ID NOs: 23235, 23242, 23240, and 23238 disclosed therein and related codon optimized sequences disclosed therein), and
U.S. Patent Appl. Publ. 2018/0126003 (and in particular SEQ ID NOS: 126837, 282951, 282944, and 282928 disclosed therein and related codon optimized sequences disclosed therein).

The Maxi-K sequences disclosed in the patents and application publications above can also be used as Maxi-K compositions of the disclosure, for the manufacture of such compositions, and for the treatment of smooth muscle dysfunctions as disclosed herein. For example, the Maxi-K sequences disclosed in the incorporated patents and application publications can be used in plasmids/vectors, e.g., for naked administration, in viral vectors, or in any system known in the art that can effectively introduce a nucleic acid into a host cell for expression in such host cell (e.g., a smooth muscle cell).

Maxi-K polynucleotide sequences and corresponding polypeptides that can be used according to the present disclosure, are presented in TABLE 1.

TABLE 1
Maxi-K polypeptide and polynucleotide sequences.

| SEQ ID | |
|---|---|
| NO | Description |
| 1 | Human cytomegalovirus (see WO2018209351A1, sequence 1) |
| 2 | T7 priming site (see WO2018209351A1, sequence 2) |
| 3 | BGH polyA (see WO2018209351A1, sequence 3) |
| 4 | pUC origin of replication (see WO2018209351A1, sequence 4) |
| 5 | Kanamycin resistance marker (see WO2018209351A1, sequence 5) |
| 6 | Wild type human Maxi-K alpha subunit (Slo) (see WO2018209351A1, |
| | sequence 6) |
| 7 | hSlo ORF, NA; wild type human Maxi-K alpha subunit (Slo) (see |
| | WO2018209351A1, sequence 7) |
| 8 | hSlo T352S mutant (see WO2018209351A1, sequence 8) |
| 9 | SM22alpha promoter sequence (see WO2018209351A1, sequence 9) |
| 10 | pVAX vector (see WO2018209351A1, sequence 10) |
| 11 | Mutated Slo subsequence (see WO2018209351A1, sequence 11) |
| 12 | Primer to generate mutant (see US2016/0184455, sequence 1) |
| 13 | Primer to generate mutant (see US2016/0184455, sequence 2) |
| 14 | Wild type Slo, NA (see US2016/0184455, sequence 3) |
| 15 | Wild type Slo, Protein (see US2016/0184455, sequence 4) |
| 16 | pVAX-hSlo1 WT |
| 17 | Maxi-K alpha subunit (Slo), isoform 1 - Gene name: KCNMA1 - Uniprot: |
| | Q12791-1 - Isoform 1 of calcium-activated potassium channel subunit |
| | alpha-1 |
| 18 | Maxi-K alpha subunit (Slo), isoform 2 - Gene name: KCNMA1 - |
| | Uniprot: Q12791-2 - Isoform 2 of calcium-activated potassium channel |
| | subunit alpha-1 |
| 19 | Maxi-K alpha subunit (Slo), isoform 3 - Gene name: KCNMA1 - Uniprot: |
| | Q12791-3 - Isoform 3 of calcium-activated potassium channel subunit |
| | alpha-1 |
| 20 | Maxi-K alpha subunit (Slo), isoform 4 - Gene name: KCNMA1 - Uniprot: |
| | Q12791-4 - Isoform 4 of calcium-activated potassium channel subunit |
| | alpha-1 |
| 21 | Maxi-K alpha subunit (Slo), isoform 5 - Gene name: KCNMA1 - Uniprot: |
| | Q12791-5 - Isoform 5 of calcium-activated potassium channel subunit |
| | alpha-1 |
| 22 | Maxi-K alpha subunit (Slo), isoform 6 - Gene name: KCNMA1 - Uniprot: |
| | Q12791-6 - Isoform 6 of calcium-activated potassium channel subunit |
| | alpha-1 |
| 23 | Maxi-K alpha subunit (Slo), isoform 7 - Gene name: KCNMA1 - Uniprot: |
| | Q12791-7 - Isoform 7 of calcium-activated potassium channel subunit |
| | alpha-1 |
| 24 | Maxi-K beta 1 subunit (Slo), isoform 1 - Gene name: KCNMB1 - Uniprot: |
| | Q16558-1 - Isoform 1 of calcium-activated potassium channel subunit |
| | beta-1 |
| 25 | Maxi-K beta 1 subunit (Slo), isoform 2 - Gene name: KCNMB1 - Uniprot: |
| | Q16558-2 - Isoform 2 of calcium-activated potassium channel subunit |
| | beta-1 |
| 26 | Maxi-K beta 2 subunit (Slo) - Gene name: KCNMB2 - Uniprot: Q9Y691 - |
| | Calcium-activated potassium channel subunit beta-2 |
| 27 | Maxi-K beta 3 subunit (Slo), isoform 1 - Gene name: KCNMB3 - Uniprot: |
| | Q9NPA1-1 - Isoform 1 of calcium-activated potassium channel subunit |
| | beta-3 |
| 28 | Maxi-K beta 3 subunit (Slo), isoform 2 - Gene name: KCNMB3 - Uniprot: |
| | Q9NPA1-2 - Isoform 2 of calcium-activated potassium channel subunit |
| | beta-3 |
| 29 | Maxi-K beta 3 subunit (Slo), isoform 3 - Gene name: KCNMB3 - Uniprot: |
| | Q9NPA1-3 - Isoform 3 of calcium-activated potassium channel subunit |
| | beta-3 |
| 30 | Maxi-K beta 3 subunit (Slo), isoform 4 - Gene name: KCNMB3 - Uniprot: |
| | Q9NPA1-4 - Isoform 4 of calcium-activated potassium channel subunit |
| | beta-3 |
| 31 | Maxi-K beta 3 subunit (Slo), isoform 5 - Gene name: KCNMB3 - Uniprot: |
| | Q9NPA1-5 - Isoform 5 of calcium-activated potassium channel subunit |
| | beta-3 |
| 32 | Maxi-K beta 4 subunit (Slo) - Gene name: KCNMB4 - Uniprot: Q86W47 - |
| | Calcium-activated potassium channel subunit beta-4 |
| 33 | Maxi K alpha subunit, isoform 1, mRNA - NM_001161352.1:178-3888 |
| | Homo sapiens potassium calcium-activated channel subfamily M alpha 1 |
| | (KCNMA1), transcript variant 3, mRNA |
| 34 | Maxi-K alpha subunit, isoform 2, mRNA - NM_001161353.1:178-3837 |
| | Homo sapiens potassium calcium-activated channel subfamily M alpha 1 |
| | (KCNMA1), transcript variant 4, mRNA |
| 35 | Maxi-K alpha subunit, isoform 5, mRNA - NM_002247.3:178-3714 Homo |
| | sapiens potassium calcium-activated channel subfamily M alpha 1 |
| | (KCNMA1), transcript variant 2, mRNA |
| 36 | Maxi-K alpha subunit, isoform 6, mRNA - NM_001271522.1:178-684 |
| | Homo sapiens potassium calcium-activated channel subfamily M alpha 1 |
| | (KCNMA1), transcript variant 9, mRNA |
| 37 | Maxi-K beta 1 subunit, mRNA - NM_004137.3:444-1019 Homo sapiens |
| | potassium calcium-activated channel subfamily M regulatory beta subunit 1 |
| | (KCNMB1), mRNA |
| 38 | Maxi-K beta 2 subunit, mRNA - NM_001278911.1:353-1060 Homo |
| | sapiens potassium calcium-activated channel subfamily M regulatory beta |
| | subunit 2 (KCNMB2), transcript variant 3, mRNA |
| 39 | Maxi-K beta 3 subunit, isoform 1, mRNA - NM_014407.3:513-1352 Homo |
| | sapiens potassium calcium-activated channel subfamily M regulatory beta |
| | subunit 3 (KCNMB3), transcript variant 4, mRNA |
| 40 | Maxi-K beta 3 subunit, isoform 2, mRNA - NM_171828.2:341-1174 Homo |
| | sapiens potassium calcium-activated channel subfamily M regulatory beta |
| | subunit 3 (KCNMB3), transcript variant 1, mRNA |
| 41 | Maxi-K beta 3 subunit, isoform 3, mRNA - NM_171830.1:868-1695 |
| | Homo sapiens potassium calcium-activated channel subfamily M regulatory |
| | beta subunit 3 (KCNMB3), transcript variant 3, mRNA |
| 42 | Maxi-K beta 3 subunit, isoform 4, mRNA - NM_171829.2:943-1716 Homo |
| | sapiens potassium calcium-activated channel subfamily M regulatory beta |
| | subunit 3 (KCNMB3), transcript variant 2, mRNA |
| 43 | Maxi-K beta 3 subunit isoform 5, mRNA - NM_001163677.1:341-862 |
| | Homo sapiens potassium calcium-activated channel subfamily M regulatory |
| | beta subunit 3 (KCNMB3), transcript variant 5, mRNA |
| 44 | Maxi-K beta 4 subunit, mRNA - NM_014505.5:454-1086 Homo sapiens |
| | potassium calcium-activated channel subfamily M regulatory beta subunit 4 |
| | (KCNMB4), mRNA |
| 45 | pVAX-hSlo1-C911A |
| 46 | pVAX-hSlo1-deltaNX |
| 47 | hSlo encoded by deltaNX |
| 48 | pSMAA-hSlo |
| 49 | pVax-hSlo Variant 1 |
| 50 | pVax-hSlo Variant 2 |
| 51 | pVax-hSlo ORF (Maxi-K ORF in SEQ ID NO: 16) |
| 52 | pVax-hSlo ORF (Maxi-K ORF from pVax-hSlo Variant 1, SEQ ID NO: 49) |
| 53 | pVax-hSlo ORF (Maxi-K ORF from pVax-hSlo Variant 2, SEQ ID NO: 50) |
| 54 | hSlo - Translated ORF (SEQ ID NO: 51) from canonical pVax-hSlo (SEQ |
| | ID NO: 16) |
| 55 | hSlo - Translated ORF (SEQ ID NO: 52) from pVax-hSlo Variant 1 (SEQ |
| | ID NO: 49) |
| 56 | hSlo - Translated ORF (SEQ ID NO: 53) from pVax-hSlo Variant 2 (SEQ |
| | ID NO: 50) |

The table below (TABLE 2) presents additional mutations in Maxi-K polypeptides (alpha and beta subunits) that can be used according to the methods of the present disclosure.

TABLE 2
Mutations in Maxi-K polypeptides.

| Maxi-K subunit | Mutation | Description |
|---|---|---|
| α subunit (Slo) | G23S | Observed in pVAX-hSlo1 Variant 1 |
| α subunit (Slo) | C118A | Decreased or abolished location to plasma membrane. |
| α subunit (Slo) | C119A | Decreased or abolished location to plasma membrane. |
| α subunit (Slo) | C121A | Decreased or abolished location to plasma membrane. |
| α subunit (Slo) | L269R/H | No effect in coupling between calcium and channel |
| | | opening. |
| α subunit (Slo) | E272E | Reduction in coupling between calcium and channel |
| | | opening. |
| α subunit (Slo) | R275N | Reduction in coupling between calcium and channel |
| | | opening. |
| α subunit (Slo) | R278Q | Reduction in coupling between calcium and channel |
| | | opening. |
| α subunit (Slo) | Q281R | No effect in coupling between calcium and channel |
| | | opening. |
| α subunit (Slo) | E284K | No effect in coupling between calcium and channel |
| | | opening. |
| α subunit (Slo) | T352S | Activated at more negative voltages. Slower rate of |
| | | inactivation. Impaired inhibition by HMIMP. No effect |
| | | on channel inhibition by Iberiotoxin. |
| α subunit (Slo) | 356-356 | Loss of function. |
| | GYG > AAA | |
| α subunit (Slo) | R366G | Observed in pVAX-hSlo1 Variants 1 and 2 |
| α subunit (Slo) | F380A | Loss of function. |
| α subunit (Slo) | A381S | Activated at more negative voltages. No effect on |
| | | inhibition by HMIMP. |
| α subunit (Slo) | V384I | No effect on activation voltage. No effect on inhibition |
| | | by HMIMP. |
| α subunit (Slo) | C680S | Loss of heme-induced channel inhibition. |
| α subunit (Slo) | H681R | Loss of heme-induced channel inhibition. |
| α subunit (Slo) | D434G | Natural polymorphic variant. |
| α subunit (Slo) | E884K | Natural polymorphic variant. |
| α subunit (Slo) | N1053S | Natural polymorphic variant. |
| β1 subunit | E65K | Natural polymorphic variant. Has a protective effect |
| | | against diastolic hypertension. |
| β1 subunit | V110L | Natural polymorphic variant. |
| β1 subunit | R140W | Natural polymorphic variant. |
| β3 subunit | D44G | Natural polymorphic variant. |
| β3 subunit | A53T | Natural polymorphic variant. |
| β3 subunit | L75V | Natural polymorphic variant. |
| β3 subunit | N165S | Natural polymorphic variant. |
| β3 subunit | M230T | Natural polymorphic variant. |
| β4 subunit | T11A | Suppresses the effect of okadaic acid and increases |
| | | activation time constant; when associated with A-17 and |
| | | A-210. |
| β4 subunit | T11D | Suppresses its effect on KCNMA1 channel activation |
| | | and on deactivation kinetics; when associated with E-17 |
| | | and E-210. |
| β4 subunit | S17A | Suppresses the effect of okadaic acid and increases |
| | | activation time constant; when associated with A-11 and |
| | | A-210. |
| β4 subunit | S17E | Suppresses its effect on KCNMA1 channel activation |
| | | and on deactivation kinetics; when associated with D-11 |
| | | and E-210. |
| β4 subunit | N53A | Loss of N-glycosylation and reduced protection against |
| | | charybdotoxin; when associated with A-90. |
| β4 subunit | N90A | Loss of N-glycosylation and reduced protection against |
| | | charybdotoxin; when associated with A-53. |
| β4 subunit | S210A | Suppresses the effect of okadaic acid and increases |
| | | activation time constant; when associated with A-11 and |
| | | A-17. |
| β4 subunit | S210E | Suppresses its effect on KCNMA1 channel activation |
| | | and on deactivation kinetics; when associated with D-11 |
| | | and E-17. |
| β4 subunit | V199I | Natural polymorphic variant. |

The alpha subunit of Maxi-K contains the Voltage Sensor Domain (VSD) and two RCK (regulator of potassium conductance) domains, RCK1 and RCK2. There is a calcium binding site in RCK2. These domains contain two high affinity Ca²⁺ binding sites: one in the RCK1 domain and the other in a region termed the Ca²⁺ bowl that consists of a series of Aspartic acid (Asp) residues that are located in the RCK2 domain. The Mg²⁺ binding site is located between the VSD and the cytosolic domain, which is formed by: Asp residues within the S0-S1 loop, Asparagine residues in the cytosolic end of S2, and Glutamine residues in RCK1. In forming the Mg²⁺ binding site, two residues come from the RCK1 of one Slo1 subunit and the other two residues come from the VSD of the neighboring subunit. Specific mutations of those sites may alter the sensitivity of the channel to divalent cation modulation. The present disclosure also comprises Maxi-K alpha subunits in which mutations have been effected in these specific locations, sites, and domains.

Inhibition of Maxi-K channel activity by phosphorylation of Ser695 by protein kinase C (PKC) is dependent on the phosphorylation of Ser1151 in C terminus of the Maxi-K alpha-subunit. Only one of these phosphorylations in the tetrameric structure needs to occur for inhibition to be successful. Thus, the activity of Maxi-K can be modulated via mutation of Ser695 and/or Ser1151 of the Maxi-K alpha subunit.

The Maxi-K beta 4 subunit can be phosphorylated, and that phosphorylation dramatically alters its interaction with the Maxi-K alpha subunit. Accordingly, mutations in amino acids that are phosphorylated in the Maxi-K beta 4 subunit can modulate the activity of the Maxi-K alpha subunit.

The Maxi-K polypeptides of the present disclosure also include variants in which amino acid positions susceptible of phosphorylation (e.g., Serines 765, 778, 782, 978, 982, 1221, or 1224, or threonines 763 or 970 in Maxi-K alpha subunit), lipidation locations (e.g., positions 118, 119, or 121 in Maxi-K alpha subunit), glycosylation locations, or combination thereof are mutated. See, e.g., Jin et al. (2002) J. Biol. Chen. 277:43724-43729, disclosing that the Maxi-K beta 4 subunit comprises two consensus N-linked glycosylation sites in its extracellular domains. The extracellular loop of Maxi-K beta 4 can be glycosylated, as it also been shown to occur in the Maxi-K beta 1 subunit. However, the Maxi-K alpha subunit promotes additional Maxi-K beta 4 glycosylation in the Golgi compartment. In turn, Maxi-K beta 4 influences its modulation of the toxin sensitivity of the Maxi-K alpha subunit. Thus, reciprocal modulation exists between the pore-forming Maxi-K alpha subunit of the Maxi-K channel and its auxiliary Maxi-K beta subunit.

The Maxi-K polypeptides of the present disclosure also include Maxi-K alpha subunit variants in which any of the amino acids at positions 352-355 (region responsible for potassium selectivity); 1003-1025 (calcium bowl); 1012, 1015, 1018 or 1020 (specific calcium binding amino acids); 671-681 (heme-binding motif); 439, 462, and 464 (magnesium binding amino acids) are mutated; or any combination thereof, optionally including or more mutations disclosed in TABLE 2, or any mutations known in the art at the time the present application was filed.

The Maxi-K polypeptides of the present disclosure also include Maxi-K alpha subunit variants comprising one or more mutations at amino acid positions lining the channel pore, or variants comprising one or more mutations at amino acid positions at the interface between Maxi-K alpha and any of its auxiliary beta subunits.

The Maxi-K polypeptides of the present disclosure also include Maxi-K alpha subunit variants comprising one or more mutations that increase or decrease the phosphorylation of the Maxi-K alpha subunit by kinases such as PKA and/or PKG.

The Maxi-K polypeptides of the present disclosure also include Maxi-K alpha subunit variants comprising one or more mutations that modulate the palmitoylation of the Maxi-K alpha subunit by ZDHHC22 (Zinc Finger DHHC Domain-Containing Protein 22) and ZDHHC23 (Zinc Finger DHHC Domain-Containing Protein 23) within the intracellular linker between the SO and Si transmembrane domains, which regulate location to the plasma membrane; and/or depalmitoylation by LYPLA1 (Acyl-protein thioesterase 1) and/or LYPLAL1 (Lysophospholipase-like 1), which lead to delayed exit from the trans-Golgi network.

### IV. CONDITIONS RELATED TO SMOOTH MUSCLE DYSFUNCTION

The present disclosure provides Maxi-K compositions (e.g., a pVAX-hSlo vector of SEQ ID NO: 16, 49, or 50) and methods for the treatment of smooth muscle dysfunction in general. For example, the present Maxi-K compositions (e.g., a pVAX-hSlo vector of SEQ ID NO: 16, 49, or 50) and methods can be used to treat diseases and conditions primarily caused by a smooth muscle dysfunction, and symptoms associated with such dysfunction. In some aspects, the smooth muscle dysfunction is idiopathic. In other aspects, the present Maxi-K compositions (e.g., a pVAX-hSlo vector of SEQ ID NO: 16, 49, or 50) and methods can be used to treat smooth muscle dysfunction which are the result of an underlying disease, condition, or lesion (e.g., neurogenic smooth muscle dysfunctions). In some particular aspects, the subject has over active bladder (OAB) syndrome, erectile dysfunction (ED), asthma; benign hyperplasia of the prostate gland (BPH); coronary artery disease (infused during angiography); genitourinary dysfunctions of the bladder, endopelvic fascia, prostate gland, ureter, urethra, urinary tract, and vas deferens; irritable bowel syndrome; migraine headaches; premature labor; Raynaud's syndrome; or thromboangitis obliterans.

Abnormal bladder function, a common problem which significantly affects the quality of life of millions of men and women in the United States, can be the result of many common diseases, e.g., BPH, diabetes mellitus, multiple sclerosis, and stroke. In one aspect, the present disclosure provides methods to treat abnormal bladder function comprising administering a Maxi-K composition of the present disclosure.

Significant untoward changes in bladder function are also a normal result of advancing age. There are two principal clinical manifestations of altered bladder physiology: the atonic bladder and the hyperreflexic bladder. The present disclosure, by providing Maxi-K compositions (e.g., a pVAX-hSlo vector of SEQ ID NO: 16, 49, or 50) that can upregulate or downregulate Maxi-K function, provides methods to treat conditions related to atonic bladder and conditions related to hyperreflexic bladder comprising administering Maxi-K compositions of the present disclosure.

The atonic bladder or detrusor underactivity has diminished capacity to empty its urine contents because of ineffective contractility of the detrusor smooth muscle (the outer smooth muscle of the bladder wall). In the atonic or underactive state, diminished smooth muscle contractility is implicated in the etiology of bladder dysfunction. Thus, it is not surprising that pharmacological modulation of smooth muscle tone is insufficient to correct the underlying problem. In fact, the prevailing method for treating this condition uses clean intermittent catheterization; this is a successful means of preventing chronic urinary tract infection, pyelonephritis, and eventual renal failure. As such, treatment of the atonic bladder ameliorates the symptoms of disease but does not correct the underlying cause.

Conversely, the hyperreflexic, uninhibited, or bladder that exhibits detrusor overactivity contract spontaneously during the filling of the bladder. This may result in urinary frequency, urinary urgency, and urge incontinence, where the individual is unable to control the passage of urine. The hyperreflexic bladder is a more difficult problem to treat. Medications that have been used to treat this condition are usually only partially effective, and have severe side effects that limit the patient's use and enthusiasm. The currently-accepted treatment options (e.g., oxybutynin and tolteradine) are largely nonspecific, and most frequently involve blockade of the muscarinic-receptor pathways and/or the calcium channels on the bladder myocytes. Given the central importance of these two pathways in the cellular functioning of may organ systems in the body, such therapeutic strategies are not only crude methods for modulating bladder smooth muscle tome. Rather, because of their very mechanism(s) of action, they are also virtually guaranteed to have significant and undesirable systemic effects.

Aging and disease can result in changes in the expression of Maxi-K alpha (hSlo) subunit of the Maxi-K channel. Those changes can result in reduced organ-specific physiological modification of the tone of the smooth muscle that comprises the organ. The effect is heightened tone of the smooth muscle cells in the organs that cause human diseases such as erectile dysfunction (ED) in the penis, urinary urgency, frequency, nocturia, and incontinence in the bladder (e.g., over active bladder (OAB) syndrome), asthma in the lungs, irritable bowel in the colon, glaucoma in the eyes, and bladder outlet obstruction in the prostate. Accordingly, the present disclosure provides methods to treat such diseases by administering a Maxi-K composition of the present disclosure.

Aging results in Maxi-K alpha subunit transcript downregulation in smooth muscle. Furthermore, there is also an age-related decrease in expression of Maxi-K beta 1 subunits. See Nishimaru et al. (2004) J. Physiol. 559:849-862. The decrease expression of Maxi-K alpha and beta 1 subunits have a major functional impact on basal tone and stimulated contraction. In the elderly, coronary arteries are hyperreactive and this hyperreactivity can cause sudden and intense coronary spasm. Accordingly, smooth muscle dysfunctions related to an age-dependent decrease in Maxi-K expression (e.g., altered tone in coronary arteries, hypertension, erectile dysfunction, poor bladder control, etc.) can be treated with Maxi-K compositions of the present disclosure comprising nucleic acid encoding Maxi-K alpha subunit, Maxi-K beta subunits (e.g., beta 1 subunits), or both.

Detrusor overactivity is defined as a urodynamic observation characterized by involuntary detrusor contractions during the filling phase that may be spontaneous or provoked. Detrusor overactivity is subdivided into idiopathic detrusor overactivity and neurogenic detrusor overactivity. The present disclosure provides methods to treat either idiopathic detrusor overactivity or neurogenic detrusor activity comprising administering a Maxi-K composition of the present disclosure to a subject in need thereof.

Increased intercellular communication among detrusor myocytes occurs in both animal models of partial urethral obstruction (PUO) and humans with detrusor overactivity (DO). With respect to increased intercellular communication, the impact of increased calcium signaling may be augmented when compared to a normal bladder with potentially lower levels of intercellular coupling. This increased calcium signaling contributes, at least in part, to the “non-voiding contractions” observed in the PUO rat model. However, if there were a parallel increase in Maxi-K channel expression (for example, as a result of over-expression of a Maxi-K channel encoding transgene of a composition or method of the disclosure), then presumably the resultant recombinant and/or transgenic Maxi-K channels expressed by these transfected cells may “short circuit” abnormally increased calcium signals. This prevent further spread through gap junctions, and thus, prevents sufficient augmentation of abnormal and increased calcium signaling (by, for example, non-transfected myocyte recruitment) to mitigate abnormal contractile responses. The reduction of abnormal contractile responses in individual cells or groups of cells, by over-expression of a Maxi-K channel encoding transgene of a composition or method of the disclosure eliminates or ameliorates the non-voiding contractions characteristic of DO, the clinical correlate or urgency.

Conversely, because the involvement of spinal reflexes in the micturition response produces coordinated detrusor contractions well in excess of the abnormally increase calcium signaling associated with DO, Maxi-K transgene over-expression may effectively reduce or inhibit the weaker abnormally increase calcium signal that contributes to the DO (as measured in an animal model as a decrease in IMP (intermicturition pressure) or SA (spontaneous activity compared to control levels), without significantly or detectably affecting the more robust micturition contraction response.

Erectile dysfunction is a common illness that is estimated to affect 10 to 30 million men in the United States. Existing therapies have deleterious side effects. The use of phosphodiesterase type 5 (PDE5) inhibitors has a success rate of only 60%. Surgical implants to treat ED cost in excess of $20,000 for the device and surgical procedures. Furthermore, existing therapies require ED patients to plan for sexual intercourse.

Among the primary disease-related causes of erectile dysfunction are aging, atherosclerosis, chronic renal disease, diabetes, hypertension and antihypertensive medication, pelvic surgery and radiation therapy, and psychological anxiety. The erectile dysfunction may result from a variety of disorders, including neurogenic, arteriogenic, and veno-occlusive dysfunctions, as well as other conditions which cause incomplete relaxation of the smooth muscle. Thus, the methods of the present disclosure can treat, prevent, or ameliorate a symptom of a disease or condition selected, for example from the group consisting, e.g., of aging, atherosclerosis, chronic renal disease, diabetes, hypertension, side effects from medication (e.g., antihypertensive medication), pelvic surgery, radiation therapy, and psychological anxiety, wherein said symptom is erectile dysfunction.

The present disclosure also provides methods of regulating penile smooth muscle tone in a subject, comprising the introduction, into penile smooth muscle cells of the subject, of a Maxi-K polynucleotide sequence encoding a Maxi-K alpha subunit, a Maxi-K beta subunit, or a combination thereof, when expression Maxi-K alpha subunit, Maxi-K beta subunit, or a combination thereof in a sufficient number of penile smooth muscle cells of the subject induces penile erection in the subject. In this aspect, the method of the present disclosure is used to alleviate erectile dysfunction.

Penile flaccidity can be caused by heightened contractility of penile smooth muscle in a subject. This condition can be treated by introducing into penile smooth muscle cells of the subject a Maxi-K composition of the present disclosure. The nucleic acid encoding a Maxi-K polypeptide is expressed in the penile smooth muscle cells such that penile smooth muscle tone is regulated. Thus, the regulation of penile smooth muscle tone results in less heightened contractility of penile smooth muscle.

In general, smooth muscle cells for which the present method of gene therapy can be used include, but are not limited to, visceral smooth muscle cells of the bladder, bowel, bronchi of the lungs, penis (corpus cavernosum), prostate gland, ureter, urethra (corpus spongiosum), urinary tract, and vas deferens, as well as the smooth and/or skeletal muscle cells of the endopelvic fascia. Specifically, the claimed methods of gene therapy can be used in bladder smooth muscle cells, colonic smooth muscle cells, corporal smooth muscle cells, gastrointestinal smooth muscle cells, prostatic smooth muscle, and urethral smooth muscle. Given the many gross histological and physiological similarities in the factors that regulate the tone of smooth muscle tissue and of other vascular tissue, it follows naturally that similar principles would permit the application of the present method of gene therapy to the arterial smooth muscle cells of, e.g., the bladder, bowel, bronchi of the lungs, penis (corpus cavernosum), prostate gland, ureter, urethra (corpus spongiosum), urinary tract, and vas deferens.

The Maxi-K compositions of the present disclosure (e.g., a pVAX-hSlo vector of SEQ ID NO: 16, 49, or 50) can also be used to treat diseases and conditions related to smooth muscle dysfunction as disclosed, e.g., in International Application PCT/US2018/032574, U.S. Pat. Nos. 6,150,338, 6,239,117, 6,271,211, and 7,030,096, and U.S. Patent Appl. Publ. Nos. 2014/0088176 and 2016/0184455, all of which are herein incorporated by reference in their entireties.

The Maxi-K compositions disclosed herein (e.g., a pVAX-hSlo vector of SEQ ID NO: 16, 49, or 50) can also be used to treat, e.g., ischemia or stroke (see Herman et al. Biomolecules. 5 (3): 1870-911 (2015), The Neuroscientist. 7 (2): 166-77 (2001)), reduced coronary blood flow, high blood pressure or fluid retention (Grimm et al. (2010) Kidney International 78:956-962), or chronic pain (Review of Neurobiology. 128: 281-342 (2016)).

### V. PHARMACEUTICAL COMPOSITIONS AND DELIVERY SYSTEMS

The present disclosure also provides pharmaceutical compositions comprising a Maxi-K composition of the present disclosure (e.g., a pVAX-hSlo vector of SEQ ID NO: 16, 49, or 50). For example, the Maxi-K compositions of the present disclosure (e.g., a pVAX-hSlo vector of SEQ ID NO: 16, 49, or 50) can be administered with a delivery agent, e.g., a lipidoid, a liposome, a lipoplex, a lipid nanoparticle, a polymeric compound, a peptide, a protein, a cell, a nanoparticle mimic, a nanotube, or a conjugate. In some particular aspects, the delivery agent is a thermoreversible hydrogen, e.g., RTGEL™. See, e.g., U.S. Appl. Nos. US20140142191, US20130046275, and US20060057208, all of which are herein incorporated by reference in their entireties.

A pharmaceutical composition is a formulation containing one or more active ingredients, e.g., one or more Maxi-K compositions of the present disclosure (e.g., a pVAX-hSlo vector of SEQ ID NO: 16, 49, or 50), as well as one or more excipients, carriers, stabilizers or bulking agents, which is suitable for administration to a human patient to achieve a desired diagnostic result or therapeutic or prophylactic effect (e.g., increase or decrease smooth muscle contractility).

For storage stability and convenience of handling, a pharmaceutical composition comprising a Maxi-K composition of the present disclosure can be formulated as a lyophilized (i.e. freeze dried) or vacuum dried power which can be reconstituted with saline or water prior to administration to a patient. Alternately, the pharmaceutical composition comprising a Maxi-K composition of the present disclosure (e.g., a pVAX-hSlo vector of SEQ ID NO: 16, 49, or 50) can be formulated as an aqueous solution.

A pharmaceutical composition comprising a Maxi-K composition of the present disclosure (e.g., a pVAX-hSlo vector of SEQ ID NO: 16, 49, or 50) can contain a proteinaceous ingredient. Various excipients, such as albumin and gelatin have been used with differing degrees of success to try and stabilize a pharmaceutical composition. Additionally, cryoprotectants such as alcohols have been sued to reduce denaturation under the freezing conditions of lyophilization.

Pharmaceutical compositions comprising a Maxi-K composition of the present disclosure (e.g., a pVAX-hSlo vector of SEQ ID NO: 16, 49, or 50) suitable for internal use include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS).

In all cases, the composition comprising a Maxi-K composition of the present disclosure (e.g., a pVAX-hSlo vector of SEQ ID NO: 16, 49, or 50) must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactant such as polysorbates (Tween™), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100™), N,N-dimethyldodecylamine-N-oxide, hexadecyltrimethylammonium bromide (HTAB), polyoxyl 10 lauryl ether, BRIJ 721™, bile salts (sodium deoxycholate, sodium cholate), pluronic acids (F-68, F-127), polyoxyl castor oil (CREMOPHOR™) nonylphenol ethoxylate (TERGITOL™), cyclodextrins and, ethylbenzethonium chloride (HYMAINE™)

Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions comprising a Maxi-K composition of the present disclosure (e.g., a pVAX-hSlo vector of SEQ ID NO: 16, 49, or 50) can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The pharmaceutical compositions comprising a Maxi-K composition of the present disclosure (e.g., a pVAX-hSlo vector of SEQ ID NO: 16, 49, or 50) can be included in a container, pack or dispenser together with instructions for administration.

Certain Maxi-K compositions of the present disclosure also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, generally with an excess in the latter substance, can result in a substantial reduction of the amount of nucleic acid recovered in the liver, kidney or other extra circulatory reservoirs, presumably due to competition between the carrier compound and the nucleic acid for a common receptor. For example, the recovery of a partially phosphorothioate oligonucleotide in hepatic tissue can be reduced when it is co-administered with polyinosinic acid, dextran sulphate, polycytidic acid or 4-acetamido-4″isothiocyano-stilbene-2,2′disulfonic acid (Miyao et al., Antisenses Res. Dev., 1995, 5, 115-121; Takakura et al., Antisense & Nucl. Acid Drug Dev., 1996, 6, 177-183).

For Maxi-K compositions of the present disclosure comprising vectors (e.g., a pVAX-hSlo vector of SEQ ID NO: 16, 49, or 50), the vectors can be incorporated into pharmaceutical compositions for administration to mammalian patients, particularly humans. The vectors or virions can be formulated in nontoxic, inert, pharmaceutically acceptable aqueous carriers, preferably at a pH ranging from 3 to 8, more preferably ranging from 6 to 8, most preferably ranging from 6.8 to 7.2. Such sterile compositions will comprise the vector containing the nucleic acid encoding the Maxi-K therapeutic molecule dissolved in an aqueous buffer having an acceptable pH upon reconstitution.

In some aspects, the pharmaceutical compositions provided herein comprise a therapeutically effective amount of a Maxi-K composition of the present disclosure (e.g., a pVAX-hSlo vector of SEQ ID NO: 16, 49, or 50), e.g., a vector, in admixture with a pharmaceutically acceptable carrier and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol.

In some aspects, the pharmaceutical composition provided herein comprises a Maxi-K composition of the present disclosure (e.g., a pVAX-hSlo vector of SEQ ID NO: 16, 49, or 50) and a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. In some aspects, the pharmaceutical composition contains sodium phosphate, sodium chloride, sucrose, or a combination thereof.

In some aspects, the pharmaceutical composition comprising a Maxi-K composition of the present disclosure (e.g., a pVAX-hSlo vector of SEQ ID NO: 16, 49, or 50) comprises substances which increase the viscosity of the suspension, such as sodium carboxymethyl cellulose, sorbitol, sucrose or dextran, in the amount about 1-30 percent, such as 1, 2, 3, 4, 5, 6, 7, 8, 9, 10, 11, 12, 13, 14, 15, 16, 17, 18, 19 or 20% (v/v). Preferably the sucrose is about 10-30% (v/v), most preferably the sucrose is about 20% (v/v).

Prior to administration the pharmaceutical composition comprising a Maxi-K composition of the present disclosure (e.g., a pVAX-hSlo vector of SEQ ID NO: 16, 49, or 50) is free of components used during the production, e.g., culture components, host cell protein, host cell DNA, plasmid DNA and substantially free of mycoplasma, endotoxin, and microbial contamination. In some aspects, the pharmaceutical composition comprising a Maxi-K composition of the present disclosure (e.g., a pVAX-hSlo vector of SEQ ID NO: 16, 49, or 50) has less than 10, 5, 3, 2 or 1 CFU/swab. In some aspects, the pharmaceutical composition has 0 CFU/swab. The endotoxin level in the pharmaceutical composition can be less than 20 EU/ml, less than 10 EU/ml or less than 5 EU/ml.

In some aspects, a Maxi-K composition of the present disclosure can be encapsulated in nanoparticles, suitable for systemic (e.g., oral or parenteral) or topical administration to a subject in need thereof. In some aspects, the nanoparticle is a biocompatible nanoparticle platform having intrinsic plasticity to enable the user to chemically tune both the internal (e.g. hydrophobicity, charge) and external (e.g. surface charge, PEGylation) properties. The material of the biocompatible nanoparticle platform may be converted into powders composed of nanoparticles with average diameters of about 10 to about 99 nanometers (nm). In some aspects, the Maxi-K compositions of the present disclosure (e.g., a pVAX-hSlo vector of SEQ ID NO: 16, 49, or 50) are merely associated to the components of the nanoparticle or encapsulated within the nanoparticle. In other aspects, the Maxi-K compositions of the present disclosure (e.g., a pVAX-hSlo vector of SEQ ID NO: 16, 49, or 50) are conjugated to a component of the nanoparticle, for example, a lipid molecule.

Powders composed of nanoparticles can deliver specific concentrations of encapsulated Maxi-K compositions of the present disclosure over extended time periods. This platform can deliver bioactive molecules both systemically and topically. No indications of induced inflammation or toxicity have been observed. Appreciable cell uptake of the nanoparticles occurs without cytotoxicity. Following uptake, nanoparticles release the Maxi-K composition of the present disclosure (e.g., a pVAX-hSlo vector of SEQ ID NO: 16, 49, or 50).

Nanoparticles may be tuned to accommodate a wide range of biomolecules by manipulating the internal charge and hydrophobicity through the use of dopant trimethoxysilanes with the fourth site having the desired chemical moiety (e.g. alkyl or amine groups), in lieu of the fourth methoxy group that is present in the basic building block for the nano platform-tetramethoxysilane (TMOS). TMOS particles contacted with silanes having positive charge (amines) are contemplated for plasmid encapsulation.

Topical delivery offers several other advantages over other routes of administration (oral or injection) with regards to target specific impact, decreased systemic toxicity, avoidance of first pass metabolism, variable dosing schedules, and broadened utility to diverse patient populations. Chemical penetration enhancers can be used in order to perturb the epidermal barrier (e.g. membrane keratin and lipid bilayer).

The urothelium of the bladder has evolved mechanisms to impede exogenous molecules from passage. Consequently, topical bladder therapy has a unique and advantageous set of physiologic attributes that circumvent the challenge of traversing the urothelium. The nanoparticles disclosed herein display increased efficiency compared to naked DNA in crossing the urothelium barrier, a characteristic that is particularly advantageous when the nanoparticles are used to treat bladder condition such as over active bladder (OAB) syndrome.

### V. KITS AND ARTICLES OF MANUFACTURE

The present disclosure also provides kits and articles of manufacture comprising Maxi-K compositions of the present disclosure (e.g., a pVAX-hSlo vector of SEQ ID NO: 16, 49, or 50). Packaged Maxi-K compositions of the present disclosure (e.g., a pVAX-hSlo vector of SEQ ID NO: 16, 49, or 50) in kits can facilitate the application of the Maxi-K compositions to a subject in need thereof.

In some aspects, the kit comprises a Maxi-K polynucleotide of the disclosure, e.g., a DNA, an RNA (e.g., an mRNA) or a plasmid (e.g., a pVAX-hSlo vector of SEQ ID NO: 16, 49, or 50). In some aspects, the kit comprises a viral expression vector, e.g., an adenoviral vector or a lentiviral vector. In other aspects, the kit comprises cells transfected with a Maxi-K composition of the present disclosure.

In certain aspects, the kit comprises (i) a Maxi-K composition of the present disclosure (e.g., a pVAX-hSlo vector of SEQ ID NO: 16, 49, or 50), or a combination thereof, and (ii) instructions for use. The instructions can be in any desired form, including but not limited to, printed on a kit insert, printed on one or more containers, as well as electronically stored instructions provided on an electronic storage medium, such as a computer readable storage medium that permits the user to integrate the information and calculate a control dose.

Instructions included in the kits and articles of manufacture can be affixed to packaging material or can be included as a package insert. While the instructions are typically written or printed materials they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some aspects, the kit comprises a Maxi-K composition of the present disclosure (e.g., a pVAX-hSlo vector of SEQ ID NO: 16, 49, or 50), in one or more containers. In some aspects, the kit contains all the components necessary and/or sufficient to administer a Maxi-K composition of the present disclosure (e.g., a pVAX-hSlo vector of SEQ ID NO: 16, 49, or 50), including vials or other container with the Maxi-K composition of the present disclosure, syringes, needles, controls, directions for performing assays, or any combination thereof.

One skilled in the art will readily recognize that the Maxi-K compositions of the present disclosure (e.g., a pVAX-hSlo vector of SEQ ID NO: 16, 49, or 50) can be readily incorporated into one of the established kit formats which are well known in the art.

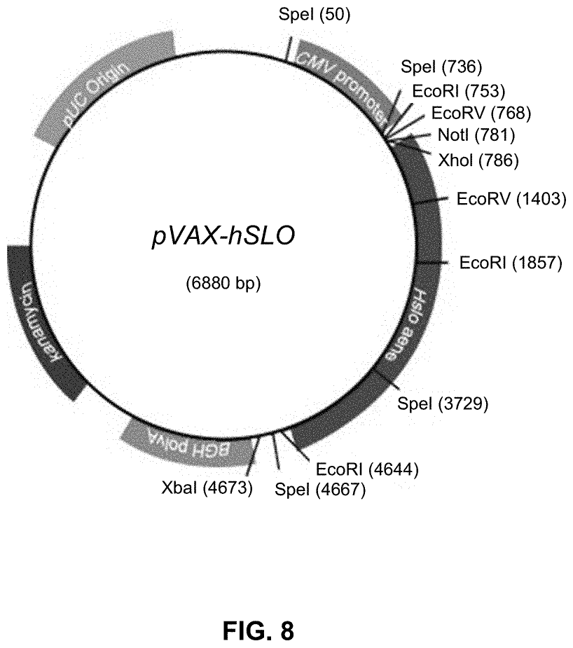

In one particular aspect, a kit comprises: (a) a recombinant plasmid provided herein, e.g., pVAX-hSlo (see FIG. 8) and (b) instructions to administer to cells or an individual a therapeutically effective amount of the recombinant plasmid. In some aspects, the kit comprises pharmaceutically acceptable salts or solutions for administering a Maxi-K composition of the present disclosure (e.g., a pVAX-hSlo vector of SEQ ID NO: 16, 49, or 50).

Optionally, the kit can further comprise instructions for suitable operational parameters in the form of a label or a separate insert. For example, the kit may have standard instructions informing a physician or laboratory technician to prepare a dose of a Maxi-K composition of the present disclosure (e.g., a pVAX-hSlo vector of SEQ ID NO: 16, 49, or 50).

Optionally, the kit can further comprise a standard or control information so that a patient sample can be compared with the control information standard to determine if the test amount of a Maxi-K composition of the present disclosure (e.g., a pVAX-hSlo vector of SEQ ID NO: 16, 49, or 50) is a therapeutic amount.

Optionally, the kit could further comprise devices for administration, such as a syringe, filter needle, extension tubing, cannula, or any combination thereof.

In some aspects, kit or article of manufacture can comprise multiple vials, each one of them containing a single dose of a Maxi-K composition of the present disclosure (e.g., a pVAX-hSlo vector of SEQ ID NO: 16, 49, or 50). In other aspects, the kit or article of manufacture can comprise one or more vials, each one of them comprising more than one dose of a Maxi-K composition of the present disclosure (e.g., a pVAX-hSlo vector of SEQ ID NO: 16, 49, or 50).

In some aspects, the article of manufacture is a bag containing a solution of a Maxi-K composition of the present disclosure (e.g., a pVAX-hSlo vector of SEQ ID NO: 16, 49, or 50). In other aspects, the article of manufacture is a bottle (e.g., a glass bottle or a plastic bottle) containing a Maxi-K composition of the present disclosure (e.g., a pVAX-hSlo vector of SEQ ID NO: 16, 49, or 50). In some aspects, the article of manufacture is a bag containing a Maxi-K composition of the present disclosure (e.g., a pVAX-hSlo vector of SEQ ID NO: 16, 49, or 50) in powder form for reconstitution in an appropriate solvent. In other aspects, the article of manufacture is a bottle (e.g., a glass bottle or a plastic bottle) containing a Maxi-K composition of the present disclosure (e.g., a pVAX-hSlo vector of SEQ ID NO: 16, 49, or 50) in powder form for reconstitution in an appropriate solvent.

### EXAMPLES

### Example 1

### Non-Clinical Studies With of hMaxi-K Gene Transfer

### Rat Model Study

The pathophysiology of partial urinary outlet obstruction in the rat model recapitulates many relevant aspects of the corresponding lower urinary tract symptoms observed in humans. The noted physiological and pathophysiological similarities made it reasonable to assume that studies on the rat bladder could provide insight into at least some aspects of human bladder physiology and dysfunction.

Because the physiology of the rat bladder parallels many aspects of the human bladder, studies examined the potential utility of bladder instilled K channel gene therapy with hSlo cDNA (i.e., the maxi-K channel alpha subunit) to ameliorate bladder overactivity in a rat model of partial urinary outlet obstruction.

In one study, twenty-two female Sprague-Dawley rats were subjected to partial urethral (i.e., outlet, PUO) obstruction, with 17 sham-operated control rats run in parallel. After 6 weeks of obstruction, suprapubic catheters were surgically placed in the dome of the bladder in all rats. Twelve obstructed rats received bladder instillation of 100 ug of hSlo/pcDNA in 1 ml PBS-20% sucrose during catheterization and another 10 obstructed rats received 1 ml PBS-20% sucrose (7 rats) or 1 ml PBS-20% sucrose containing pcDNA only (3 rats). Two days after surgery cystometry was performed on all animals to examine the characteristics of the micturition reflex in conscious and unrestrained rats. Obstruction was associated with a three- to four-fold increase in bladder weight and alterations in virtually every micturition parameter estimate (see TABLE 3).

Obstructed rats injected with PBS-20% sucrose routinely displayed spontaneous bladder contractions between micturitions. In contrast, hSlo injection eliminated the obstruction-associated bladder hyperactivity, without detectably affecting any other cystometric parameter. Presumably, expression of hSlo in rat bladder functionally antagonizes the increased contractility normally observed in obstructed animals and thereby ameliorates bladder overactivity.

Another study examined the ability of hSlo gene transfer to alter and/or ameliorate the intermicturition pressure fluctuations observed in an obstructed male rat model. For these studies rats were obstructed for 2 weeks using a perineal approach. Following 2 weeks of obstruction, the rats were catheterized for cystometric investigations and placed into 1 of 2 treatment groups. Age-Matched Control rats were subjected to a sham obstruction and run in parallel.

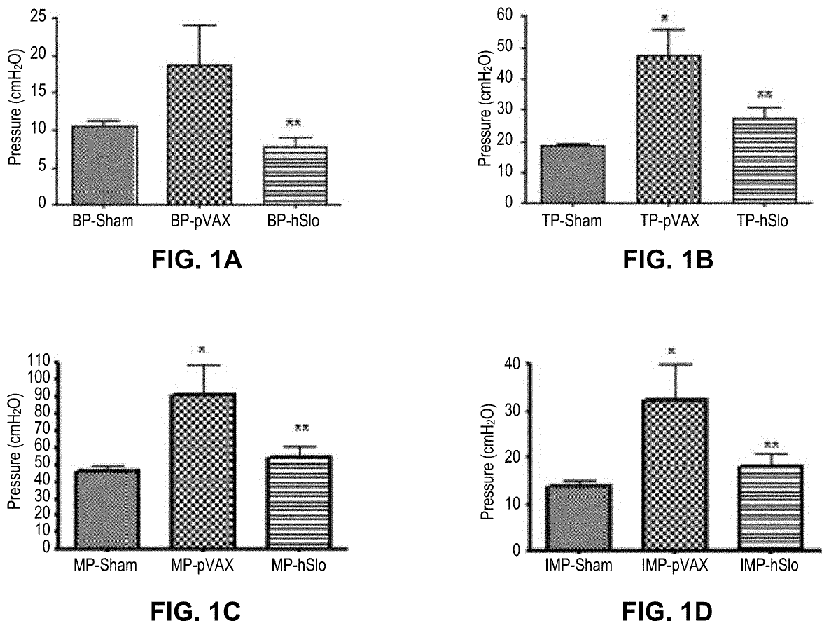

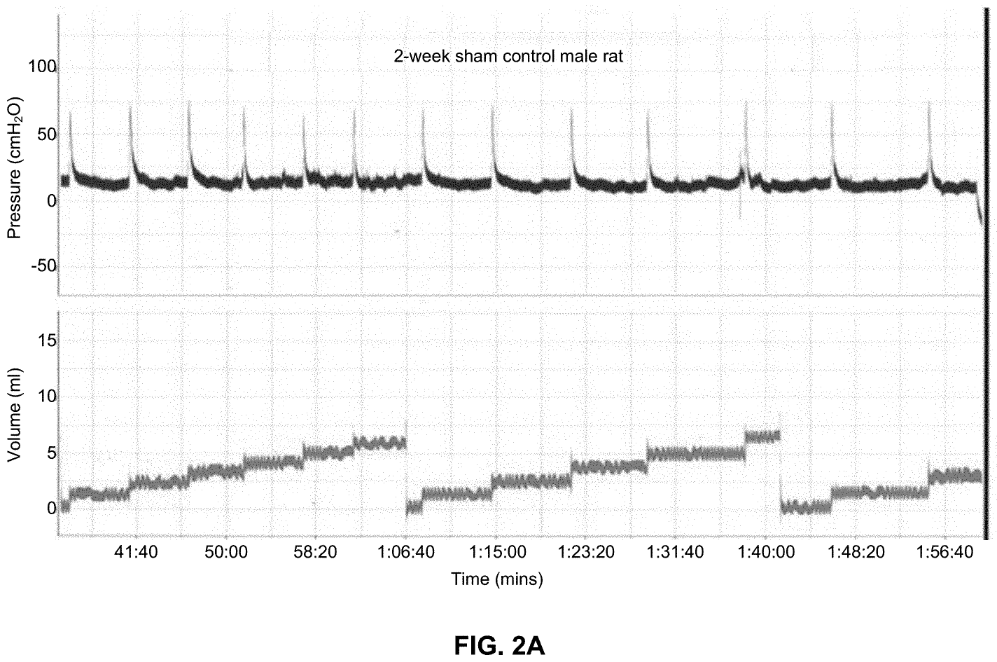

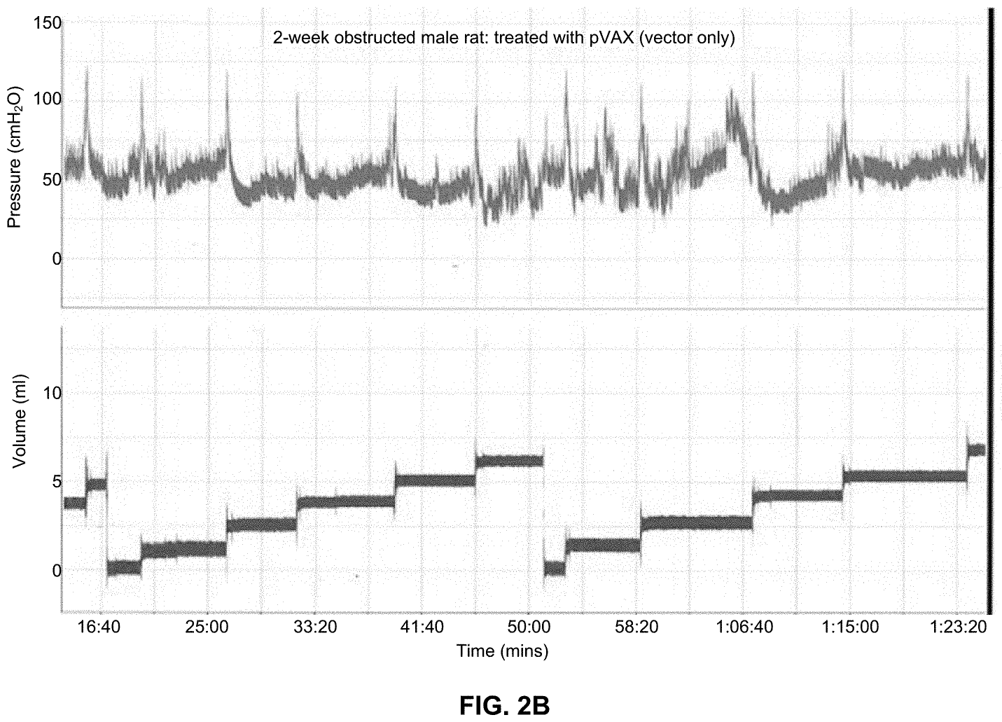

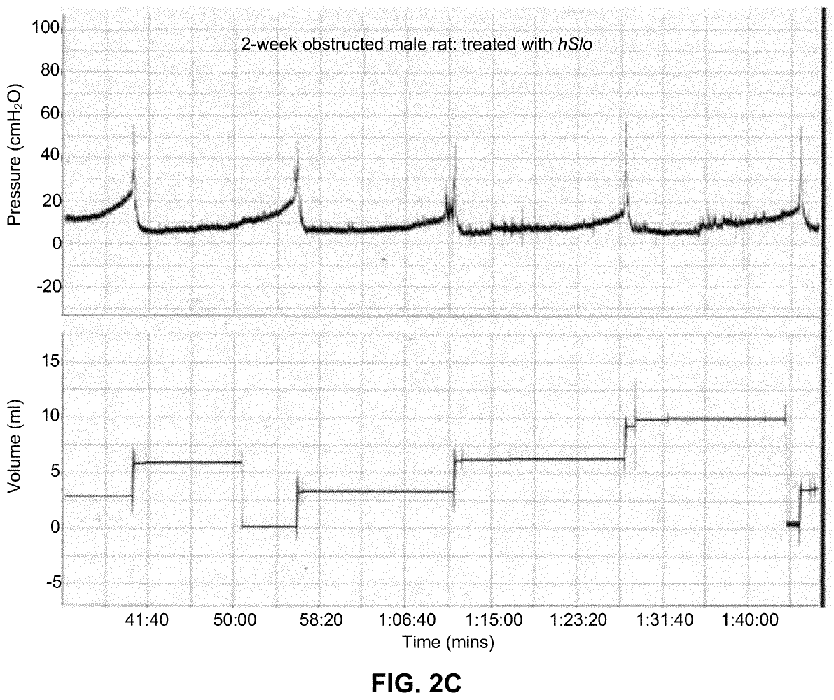

The mean values for the micturition parameters in all experimental animals are summarized in TABLE 4, and the salient features of these findings are graphically depicted in FIGS. 1A, 1B, 1C, and 1D and FIGS. 2A, 2B, and 2C. Importantly, as with the study in the 6-week obstructed female rat a single intravesical instillation of 100 ug hSlo/pVAX was associated with statistically significant changes in several micturition parameters of major physiological relevance.

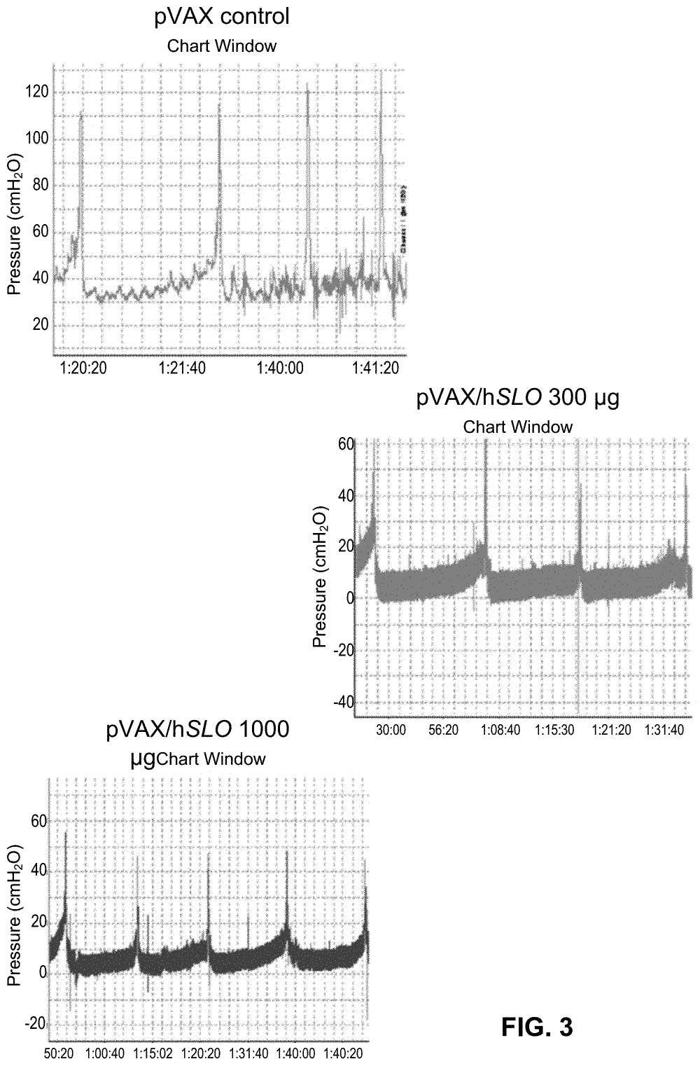

A third study evaluated the effects of hSlo gene transfer following 2 weeks of partial urethral outlet obstruction in female rats. In order to create a partial urethral outlet obstruction (PUO), a ligature was placed on the urethra of female Sprague-Dawley rats weighing 200-250 g (Christ et al., 2001) as described above. Two weeks after placement of the ligature, the rats were subjected to surgery for placement of a suprapubic catheter. Two days later, bladder function studies (i.e., cystometry) were performed on conscious, unrestrained rats in metabolic cages. As illustrated in TABLE 5 and FIG. 3, following the 2 weeks of partial urethral outlet obstruction female rats exhibit significant changes in bladder function, as evidenced by the more than 2-fold increase in bladder capacity and the appearance of significant spontaneous bladder contractions. The increased spontaneous bladder contractions were observed as pressure fluctuations between micturitions (see FIG. 3), and quantified as shown in TABLE 5 by the corresponding increases observed in the SA and IMP values. A single intraluminal bladder injection of 300 ug and 1000 ug of pVAX-hSlo (in 1 ml PBS-20% sucrose) resulted in a nearly complete ablation of detrusor overactivity. This effect is reflected by the significant decrease in IMP and SA in the hSlo-treated, obstructed rats when compared with the rats treated with pVAX vector only (see TABLE 5). Although, a true DO effect relationship for hSlo gene transfer was not shown in this model, this study did demonstrate that over a 1-log unit variation in DO (from 100 to 1000 ug), there is a statistically significant, and moreover, physiologically relevant, diminution in DO, in the absence of any detectable effect on the ability of the bladder to empty. That is, in this animal model, pVAX-hSlo was able to ameliorate the pathophysiological effects of outflow obstruction-related DO, without having any detrimental effect on bladder function. Similar effects were observed after instillation of 100 ug pVAX-hSlo in the 6-week obstructed female Sprague-Dawley rats, which are shown below.

TABLE 3
Summary of treatment effects on mean micturition parameters
in 6 week obstructed female rats and sham-operated controls

| | WT | | | | | | | MIP |
|---|---|---|---|---|---|---|---|---|
| | (mg) | MP | THP | BP | BC | MV | RV | (IP-BP) |
| Control: | 171 ± | 73.9 ± | 22.3 ± | 12.6 ± | 1.2 ± | 1.13 ± | 0.13 ± | 3.49 ± |
| unobstructed | 15.0 | 4.99 | 2.1 | 1.09 | 0.1 | 0.10 | 0.04 | 0.79 |
| (n = 17) | | | | | | | | |
| ^{a}Obstructed: | *547.6 ± | *128.9 ± | *36.3 ± | *22.1 ± | *3.44 ± | *3.22 ± | *^{,} **0.3 ± | **5.59 ± |
| pVAX-hSlo | 55.4 | 16.1 | 4.30 | 43.9 | 0.41 | 0.39 | 0.10 | 1.05 |
| injected | | | | | | | | |
| (n = 12) | | | | | | | | |
| ^{b}Obstructed: | *473.1 ± | *132.7 ± | *39.3 ± | *18.8 ± | *2.91 ± | *2.94 ± | 0.09 ± | 9.37 ± |
| untreated | 56.6 | 17.9 | 3.6 | 1.9 | 0.62 | 0.65 | 0.05 | 1.79 |
| (n = 10) | | | | | | | | |

^{a}100 μg pVAX-hSlo in 200 μl PBS-20% sucrose
^{b}3 of these rats received 1000 μg pcDNA in PBS-20% sucrose. Control: Sham operated, unobstructed age-matched control animals, WT: bladder weight (mg), MP: micturition pressure (cm H₂O), THP: threshold pressure (cm H₂O), BP: basal pressure (cm H₂O), BC: bladder capacity (ml), MV: micturition volume (ml), RV: residual volume (ml), MIP: mean inter-micturition pressure ((cm H₂O; the mean pressure over the entire inter-micturition interval minus the basal pressure on the same animal).
*Significantly different from sham-op; p < 0.05.
**Significantly different from control (obstructed but not treated); p < 0.05, One-Way ANOVA, with Newman Keuls post hoc pairwise comparisons.

TABLE 4
Summary of treatment effects on mean micturition parameters
in 2 week obstructed male rats and sham-operated controls.

| | Bcap | MV | RV | BP | TP | MP | IMP | SA | Bcom | BW |
|---|---|---|---|---|---|---|---|---|---|---|
| pVAX | 2.36 ± | 1.84 ± | 0.53 ± | 18.65 ± | 47.21 ± | 91.28 ± | 32.49 ± | 13.84 ± | 0.12 ± | 348.3 ± |
| (n = 8)^{b} | 0.48 | 0.31 | 0.21 | 5.38 | 8.61^{c} | 18.52^{c} | 7.5^{c} | 2.57^{c} | 0.04 | 105.3 |
| hSlo | 2.48 ± | 2.22 ± | 0.27 ± | 7.66 ± | 27.26 ± | 54.05 ± | 18.13 ± | 10.47 ± | 0.17 ± | 352.3 ± |
| (n = 16)^{b} | 0.30c | 0.26^{c} | 0.12 | 1.35^{d} | 3.7^{d} | 6.28^{d} | 2.8^{d} | 1.89^{c} | 0.03 | 42.99 |
| Sham | 1.35 ± | 1.32 ± | 0.03 ± | 10.6 ± | 18.47 ± | 46.58 ± | 13.96 ± | 3.39 ± | 0.18 ± | 274.4 ± |
| (n = 10)^{a} | 0.14 | 0.12 | 0.02 | 0.81 | 0.79 | 3.34 | 1.09 | 0.61 | 0.018 | 24.5 |

Bcap, bladder capacity (ml); MV, micturition volume (ml); RV, residual volume (ml); BP, basal pressure (cm H₂O); TP, threshold pressure (cm H₂O); MP, micturition pressure (cm H₂O); IMP, mean intermicturition pressure (cm H₂O; the mean pressure over the entire intermicturition interval minus the basal pressure on the same animal); SA, spontaneous activity (cm H₂O); Bcom, bladder compliance (ml/cm H₂O); BW, bladder weight (mg).
^{a}5 of these animals are 2-week sham controls, the other 5 are 1 month older (or 6-week sham controls) However, statistical analysis revealed that there were no significant differences in any of the micturition parameters, and thus, these 2 populations were considered to be homogeneous for the purposes of this analysis.
^{b}All treated rats were given 1000 μg pVAX alone or 100 μg hSlo/pVAX in 1 ml PBS with 20% sucrose. All data represent the mean ± S.E.M. and were analyzed using a one-way analysis of variance, with a post hoc Tukey's test for all pairwise (multiple) comparisons.
^{c}Significant difference from the corresponding sham control value.
^{d} Significant difference from the corresponding pVAX value.

TABLE 5
Summary of treatment effects on mean micturition parameters in 2 week obstructed female rats

| | | | | | | | MIP | | | |
|---|---|---|---|---|---|---|---|---|---|---|
| | MP | TP | BP | BC | MV | RV | (IP-BP) | MF | SA | BCOM |
| Control: pVAX | 68.1 ± | 34.2 ± | 9.1 ± | 2.3 ± | 2.2 ± | 1.1 ± | 24.0 ± | 4.6 ± | 14.9 ± | 0.1 ± |
| (n = 10) | 8.1 | 4.9 | 1.9 | 0.3 | 0.3 | 0.0 | 4.6 | 0.5 | 3.4 | 0.02 |
| ^{a}Obstructed: 10 | 65.3 ± | 30.3 ± | 7.2 ± | 2.5 ± | 2.4 ± | 0.2 ± | 20.0 ± | 4.4 ± | 12.8 ± | 0.1 ± |
| μg pVAX-hSlo | 10.5 | 3.6 | 1.0 | 0.3 | 0.3 | 0.1 | 3.5 | 0.5 | 3.0 | 0.02 |
| injected | | | | | | | | | | |
| (n = 7) | | | | | | | | | | |
| ^{b}Obstructed: 30 | 81.1 ± | 36.6 ± | 11.8 ± | 3.2 ± | 2.7 ± | 0.4 ± | 27.1 ± | 4.3 ± | 15.3 ± | 0.1 ± |
| μg pVAX-hSlo | 7.3 | 4.4 | 2.6 | 1.0 | 0.4 | 0.2 | 3.5 | 0.4 | 1.5 | 0.02 |
| injected | | | | | | | | | | |
| (n = 9) | | | | | | | | | | |
| ^{b}Obstructed: | 47.8 ± | 17.7*, ** ± | 6.3 ± | 2.3 ± | 2.2 ± | 0.3 ± | 10.3*, ** ± | 5.3 ± | 4.1*, ** ± | 0.2*, ** ± |
| 300 μg pVAX- | 3.7 | 1.6 | 1.1 | 0.4 | 0.3 | 0.2 | 1.2 | 0.6 | 0.4 | 0.02 |
| hSlo injected | | | | | | | | | | |
| (n = 10) | | | | | | | | | | |
| ^{b}Obstructed: | 57.2 ± | 21.4*, ** ± | 5.7 ± | 2.1 ± | 2.0 ± | 0.1 ± | 11.6*, ** ± | 5.2 ± | 5.9*, ** ± | 0.1*, ** ± |
| 1000 μg pVAX- | 6.2 | 1.8 | 1.1 | 0.1 | 0.1 | 0.04 | 1.3 | 0.3 | 0.5 | 0.01 |
| hSlo injected | | | | | | | | | | |
| (n = 12) | | | | | | | | | | |

^{a}10, 30, 300, 1000 μg pVAX-hSlo in 200 μl PBS-20% sucrose
^{b}Control: Obstructed age-matched control animals that received 1000 μg of pVAX only, WT: bladder weight (mg), MP: micturition pressure (cm H₂O).
TP: threshold pressure (cm H₂O), BP: basal pressure (cm H₂O), BC: bladder capacity (ml), MV: micturition volume (ml), RV: residual volume (ml).
MIP: mean inter-micturition pressure ((cm H₂O; the mean pressure over the entire inter-micturition interval minus the basal pressure on the same animal).
SA spontaneous activity (MIP-BP); BCOM Bladder compliance (bladder capacity/TP-BP)
*Significantly different from control; p < 0.05. All pairwise multiple comparison procedures (Holm-Sidak method).
Significantly different from control; p < 0.05, One-Way ANOVA.

### Rabbit Model Study

A rabbit study to evaluate the distribution of different volumes of gene transfer injected into the bladder wall was performed prior to initiation of the clinical trial in women with OAB using direct intravesicular injections (TABLE 6). Nine female Adult New Zealand white rabbits weighing an average of 6 pounds were used. The animals were anesthetized and pVAX-lacZ was to be injected into the detrusor in 0.05, 0.1, and 0.15 ml aliquots into 4, 8, and 10 sites in the bladder wall. An additional set of 3 animals was to be injected with carrier alone at only the highest volume of carrier (4, 8, or 10 sites×0.15 ml). The plasmids were in solution at a concentration of 4000 ug/ml. One week later the animals were euthanized and the bladders excised and weighed. Areas with blue color were prepared for histological examination and molecular analysis. Molecular analysis of hSlo expression tissue was done with RNA extraction and real time PCR. In addition, histopathology was performed on the various rabbit tissues.

Due to difficulty with direct bladder injections in this animal model, only one rabbit was given the 0.05 ml injection. Six rabbits had 0.1 ml at 4, 8, and 10 sites (3 from inside the bladder; 3 from outside the bladder). Three rabbits had 0.15 ml at 4, 8, and 10 sites. Results indicated that those rabbits with a greater number of injections (8-10 injections) had less expression than some animals with the smallest number of injections (4 injections). The overall conclusion was that the direct injection into the bladder wall resulted in expression of the gene; however, it seemed to work best with wider dispersion of the injections perhaps 1 cm apart. The gene was detected in the blood up until 30 minutes post treatment. There were granulomatous lesions observed due to the sutures (a common artifact in the rabbit model).

TABLE 6
Rabbit Intravesicular Injection Protocol

| N = 12 | 50-50 mixture of | | | |
|---|---|---|---|---|
| rabbits | p-VAX-hSlo (ml) | sites/rabbit | sites/rabbit | sites/rabbit |
| | 0.05 | 4 | 8 | 10 |
| | 0.1 | 4 | 8 | 10 |
| | 0.15 | 4 | 8 | 10 |

### Toxicology and Histopathology in Rat Model

For the OAB indication it was not technically possible to simulate the same transurethral route of intravesical administration of pVAX-hSlo in rats as used in the human trials. Therefore, in the toxicology and biodistribution studies evaluating intravesical injection of pVAX-hSlo, animals underwent surgical exposure of the bladder and study material was injected directly into the bladder using a needle

The effects of pVAX-hSlo on hematological and chemical parameters were assessed in fifteen 275-300 g normal female Sprague-Dawley rats. 1000 ug of either pVAX-hSlo (8 animals) or pVAX vector (7 animals) was injected directly into the lumen of the bladder following surgical exposure. Blood samples were collected via a heart stick immediately after the animals were euthanized by CO2 anesthesia at 4, 8, and 24 hours and at 1 week following injection of test material. Samples were analyzed for glucose, urea nitrogen, creatinine, total protein, total bilirubin, alkaline phosphatase, ALT, AST, cholesterol, sodium, potassium, chloride, A/G ratio, BUN/creatinine ratio, globulin, lipase, amylase, triglycerides, CPK, GTP, magnesium and osmolality. The laboratory parameters were similar between pVAX-hSlo and controls at the four time points.

The effect of pVAX-hSlo on the histopathology in female Sprague-Dawley rats (275 to 300 gr) was evaluated in two studies. In the first study, four rats underwent partial bladder obstruction surgery and 2 weeks later 100 ug pVAX-hSlo in 1,000 uL PBS-20% sucrose was administered directly into the lumen of the bladder with surgical exposure of the bladder. A single animal was euthanized at 1, 8, and 24 hours, and at one week after injection of pVAX-hSlo.

The tissues of 47 organs were immediately fixed in 10% formalin and processed for routine histopathological examination. Histopathological changes were noted only in the bladder and consisted of serositis, edema, hemorrhage, and fibrosis. These changes were consistent with those expected with partial urethral obstruction and were not considered related to injection of pVAX-hSlo.

Because of the histopathological changes in the bladder of rats with PUO administered pVAX-hSlo, the effect of pVAX-hSlo compared to vector (pVAX) and PBS-20% sucrose on histology of the bladder was evaluated in normal rats. Following surgical exposure, the following test material was injected directly into the bladder lumen: 1) 0.6 ml PBS-20% sucrose, 2) 1,000 ug pVAX in 0.6 ml PBS-20% sucrose, or 3) 1000 ug pVAX-hSlo in 0.6 ml PBS-20% sucrose. Animals were euthanized with CO₂ 72 hours after instillation and the bladders removed and immediately fixed in 10% formalin solution. The 72 hour time point was chosen to limit the mechanical effects of the needle puncture on the bladder wall and minimize any potential effects of inflammation that might be caused by the pVAX-hSlo, vector, or diluent.

There were no gross findings on examination of the bladder. Overall, there were no treatment-related differences between pVAX-hSlo and either the vehicle or pVAX. No treatment-related alterations in the urothelium were noted. The lesions seen on histological examination were consistent with trauma from the needle used for injection since they were focal rather than diffuse or multifocal in distribution.

### Biodistribution in Rat Model

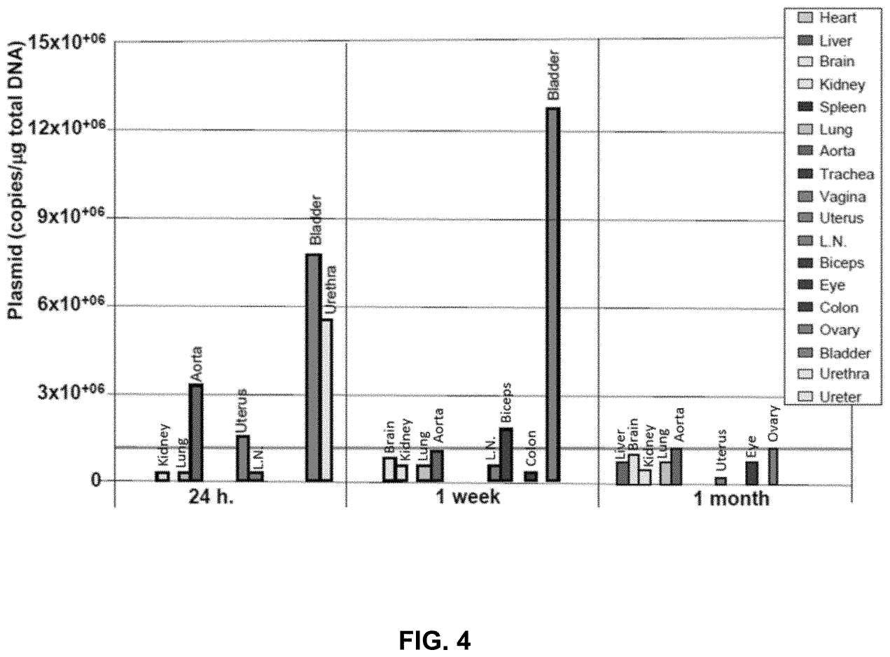

In the biodistribution study, test material was injected directly into the lumen of exposed bladders in 275-300 g normal female Sprague-Dawley rats. 1000 ug pVAX-hSlo in 0.6 ml of PBS-20% sucrose was administered to 12 animals and 0.6 ml PBS-20% sucrose administered to 5 animals (FIG. 4). Four animals each were sacrificed at 24 hours, 1 week, and 1 month following injection of test material. Tissue samples were collected in the specified order as follows: heart, liver, brain, kidney, spleen, lung, aorta, trachea, lymph node, eye, biceps, colon, vagina, and uterus.

Genomic DNA samples were analyzed for the kanamycin gene with a validated QPCR method. The results indicate that after injection of 1,000 ug pVAX-hSlo, the plasmid could be detected after 24 hours in the aorta, uterus, bladder, and urethra. At 1 week, approximately 13 million copies/ug total DNA were measured in the bladder and pVAX-hSlo could also be detected slightly in the biceps. The results are displayed in graphical format in FIG. 4.

Although these results differed from findings after intracavernous injection, the detection of 13 million copies/ug total DNA was still lower than the <30 copies plasmid/10⁵ host cells that persisted at the site of DNA vaccine injections after 60 days in clinical Investigational New Drug (IND) trials for these vaccines. These DNA vaccine studies demonstrated that intramuscular, subcutaneous, intradermal, or particle-mediated delivery did not result in long-term persistence of plasmid at ectopic sites. In addition, the procedure to inject pVAX-hSlo directly into the surgically exposed bladder in animals explained the ability to detect plasmid in tissue other than the bladder. In humans, hMaxi-K was instilled directly into the bladder using a transurethral catheter and the risk of plasmid distribution due to tissue damage or trauma was obviously markedly reduced.

### Example 2

### Human Clinical Trial with hMaxi-k Gene Transfer

### Trial Design

This was a Phase 1B, multicenter study evaluating the safety and potential activity of two escalating doses of hMaxi-K alpha subunit gene (hSlo) administered as a direct injection into the bladder wall in female patients with Idiopathic (Non-neurogenic) Overactive Bladder Syndrome (OAB) and Detrusor Overactivity (DO).

The study population consisted of women at least 18 years old of non-child bearing potential (e.g., hysterectomy, tubal ligation or postmenopausal defined as last menstrual cycle >12 months prior to study enrollment, or serum FSH >40 mIU/L) with overactive bladder (OAB) and detrusor overactivity who are otherwise in good health.

Inclusion criteria included clinical symptoms of overactive bladder of at least 6 months duration including at least one of the following:

1. Frequent micturition (at least 8/24 hrs)

2. Symptoms of urinary urgency (the complaint of sudden compelling desire to pass urine, which is difficult to defer) or nocturia (the complaint of waking at night two or more times to void)

3. Urge urinary incontinence (average of 5 per week—Urge urinary incontinence is defined as: the complaint of involuntary leakage accompanied by or immediately preceded by urgency)

Participants also had a bladder scan at screening demonstrating a residual volume of 200 ml or less and detrusor overactivity documented during baseline urodynamic testing of at least 1 uncontrolled contraction(s) of the detrusor of at least 5 cm/H₂O.

The primary objective of this study was to evaluate occurrence of adverse events and their relationship to a single treatment of approximately 20 to 30 bladder wall intramuscular injections of hMaxi-K compared to placebo (PBS-20% sucrose). This was a double blind, imbalanced placebo controlled sequential dose trial. Participants were healthy women of 18 years of age or older, of non-childbearing potential, with moderate OAB/DO of at least six months duration with at least one of the following: frequent micturition at least 8 times per day, symptoms of urinary urgency or nocturia (the complaint of waking at night two or more times to void), urge urinary incontinence (five or more incontinence episodes per week), and detrusor overactivity with at least 1 uncontrolled phasic contraction(s) of the detrusor of at least 5 cm/H₂O pressure documented on CMG. All of the participants had failed prior treatment with anticholinergics. Four had failed onabotulinum toxin A therapy.

Participants were randomly assigned to either hMaxi-K at one of two doses (16,000 ug, or 24,000 ug), or placebo. Treatment was administered as 20-30 IM injections into the bladder wall during cystoscopy. Participants were seen 8 times within a 24-week period with a study follow-up of 18 months. All reported adverse events occurring after study drug dosing were recorded. Complex CMG's were done at screening visit 1A (week −1) and at week 4 (visit 5) and week 24 (visit 8) post-injection. Post void residual volume (PVR) was measured at every visit with a BLADDERSCAN®.

The data to assess efficacy were evaluated using summary descriptive statistics by treatment group (combined placebo vs. 2 active treatment groups and combined placebo vs. combined treatment groups). Linear mixed effect models were used to estimate difference of changes from baseline between placebo and active treatment and to test whether there was dose-response for different outcomes. Generalized estimating equation (GEE) models were to be used to estimate effects for the binary endpoints.

There were 6 participants who received 16,000 ug, 3 participants who received 24,000 ug and 4 participants who received placebo. See TABLE 7.

TABLE 7
Final Dose-hMaxi-k

| | | | | |
|---|---|---|---|---|
| hMaxi-K Dose | 16,000 μg | PBS-20% sucrose | 24,000 μg | PBS-20% sucrose |

| | | |
|---|---|---|
| Volume | 4 mL | 6 mL |
| Number of Vials | 2 | 3 |
| Final Volume | 4 mL | 6 mL |
| Number of IM | 20 injections of 0.2 ml at specified | 30 injections of 0.2 ml at specified sites |
| injections | sites in bladder wall approx. 1 cm apart | in bladder wall approx. 1 cm apart |
| | (FIG. 5) | (FIG. 5) |

Note:
In each dose cohort 6 participants received hMaxi-K and 3 will receive PBS-20% sucrose (placebo).

TABLE 8 shows an overview of the treatment schedule and procedures performed by visit.

TABLE 8
Summary of Tests by Laboratory Visit

| | Phase | | | | | | | | | |
|---|---|---|---|---|---|---|---|---|---|---|
| | Screening Phase | | | Post-Treatment Follow up Visits | | | | | | |
| | Visit/Period | | | | | | | | | |
| | Visit | Visit | Visit | Telephone | Visit | Visit | Visit | Visit | Visit | Visit |
| | 1 | 1A ⁿ | 2 | Follow-up ^{j} | 3 | 4 | 5 | 6 | 7 | 8 |
| | Day | | | | | | | | | |
| | −14 | −14 to −8 | 0 (Baseline) | Day 1 & 3 | 8 | 15 | 29 | 57 | 85 | 169 (Final) |
| | Week | | | | | | | | | |
| | | | 0 | 0 | 1 | 2 | 4 | 8 | 12 | 24 |
| Visit Window (days) | −2 | +2 | +2 | Day 3 ± 1 | +2 | +2 | ±2 | ±3 | ±5 | ±5 |
| Signed Informed Consent | ▴ | | | | | | | | | |
| Evaluation of Inclusion/Exclusion | ▴ | ▴ | ▴ ^{f} | | | | | | | |
| Criteria | | | | | | | | | | |
| Demographics and Medical/Surgical | ▴ | | | | | | | | | |
| History | | | | | | | | | | |
| Physical Examination | ▴ | | ▴ ^{f} | | ▴ | ▴ | ▴ | ▴ | ▴ | ▴ |
| ECG | ▴ | | ▴ ^{a} | | ▴ | | ▴ | | | ▴ |
| Previous/Concomitant Medication | ▴ | ▴ | ▴ ^{f} | | ▴ | ▴ | ▴ | ▴ | ▴ | ▴ |
| Assessment | | | | | | | | | | |
| Vital Signs ^{h} | ▴ | | ▴ ^{f, l} | | ▴ | ▴ | ▴ | ▴ | ▴ | ▴ |
| Objective OAB/DO Evaluation | | ▴ ^{d} | | | | | ▴ | | | ▴ |
| (Cystometry) ^{b} | | | | | | | | | | |
| Bladder scan ^{c} | ▴ | | | | | ▴ | | ▴ | | ▴ |
| Dispense Daily Voiding Diary/Urgency | ▴ | ▴ | ▴ ^{f} | | ▴ | ▴ | ▴ | ▴ | ▴ | |
| questionnaire ⁱ | | | | | | | | | | |
| Pad Test ^{m} | | ▴ | ▴ | | ▴ | ▴ | ▴ | ▴ | ▴ | ▴ |
| QoL (King's Health Questionnaire) and | | | ▴ ^{f} | | | | ▴ | ▴ | ▴ | ▴ |
| SF-12 | | | | | | | | | | |
| Subjective Evaluation of Disease State ^{k} | | | ▴ ^{f} | | ▴ | ▴ | ▴ | ▴ | ▴ | ▴ |
| Subjective Evaluation of Response to | | | | | ▴ | ▴ | ▴ | ▴ | ▴ | ▴ |
| Treatment ^{k} | | | | | | | | | | |
| ICIQ-SF | | | ▴ ^{f} | | | | ▴ | ▴ | ▴ | ▴ |
| Urinalysis and Urine Cultures ^{d} | ▴ | ▴ | ▴ ^{f} | ▴ | ▴ | ▴ | ▴ | | ▴ | ▴ |
| Hematology Laboratory Tests ^{e} | ▴ | | ▴ | ▴ | ▴ | ▴ | ▴ | | ▴ | ▴ |
| Chemistry Laboratory Tests ^{e} | ▴ | | ▴ | ▴ | ▴ | ▴ | ▴ | | ▴ | ▴ |
| Pharmacokinetic Assessment (urine and | | | ▴ ^{f, g} | | ▴ | ▴ | ▴ | ▴ | ▴ | ▴ ^{g} |
| blood hSlo cDNA) | | | | | | | | | | |
| Adverse Event Assessment | | ▴ | ▴ ^{f} | ▴ ^{j} | ▴ | ▴ | ▴ | ▴ | ▴ | ▴ |
| Study Drug administered | | | ▴ | | | | | | | |

^{a} ECG was done prior to administration of study drug and at 2 hours post dosing.
^{b} Cystometry included: volume at first desire to void, detrusor pressure, abdominal pressure, detrusor pressure at beginning of voiding, detrusor pressure at maximum flow, maximum detrusor pressure, volume at strong urge to void, peak flow rate during voiding, voided volume, volume at DO, post-void residual volume, total bladder volume (voided volume + residual volume), number of detrusor contractions during procedure and duration of DO.
^{c} Inclusion criteria specify residual volume ≤200 ml. Bladder scans at V1 and V8 were done before catheterization.
^{d} Urinalysis with microscopic RBC and WBC, protein, glucose, nitrites, pH, and specific gravity at V 1, 3-5 and V7 and V8. At V1A and V2, urinalysis by Dipstick was done. Urine cultures at V1 (by catheterization with the urodynamic catheter), V3 (clean void); at V1A, V2, V5 and V8 prior to cystometry or cystoscopy (by catheterization with the urodynamic catheter) and before discharge by clean void (at V2 use first voided urine after drug administration). Visit 2 urinalysis by Dipstick was done prior to dosing and urine culture was performed both prior to study drug administration and prior to discharge.
^{e} Lab tests were done at V1, V2-5, V7 and V8 and included: Hematology- CBC with differential, platelet count, sedimentation rate, PTT, PT (no PT and PTT at V2 and V4), CRP, Antinuclear antibody; Chemistry- BUN, creatinine, Na⁺, K⁺, Mg⁺⁺, Ca⁺⁺, CO2, Cl⁻, albumin, alkaline phosphatase, ALT, AST, GGT, total bilirubin, total protein, CPK, LDH, glucose); Serum Pregnancy Test for beta-HCG was required for women of child bearing age who have not had hysterectomy at Screening V1 and on as need basis. In addition, FSH >40 IU/L if last menstrual cycle not >12 months prior to study enrollment. HbA1c was done at screening Visit 1 only. No chemistries were done at 2 (Week 0). At V4, chemistries included only BUN, creatinine, electrolytes (Na⁺, K⁺), CRP, glucose, and ANA. No lab tests were done at Visit 1A or V6. Lab tests were taken at the same time of day at all study visits.
^{f} Test or procedure was done prior to administration of study drug at Visit 2.
^{g} Pre-dosing at V2. If specimen was still positive at week 24, participant returned monthly until two successive specimens were negative for hSlo DNA.
^{h} Vital signs included height at V1 only; weight at V1 and V8; oral body temperature at all visits (except V1A). Same arm was used for all BP measurements and specified.
ⁱ Diaries were completed prior to V1A (to test for compliance and inclusion criteria), for 7 days prior to Visit 2 and 7 days prior to each visit, thereafter.
^{j} Participants were contacted by telephone on Study Day 1 and 3 (1 day and 3 days ±1, following drug administration at Visit 2) for assessment of adverse events.
^{k} Subjective assessments were based on the questions: “How bothersome do you consider your bladder problem?” and “Has the treatment been of benefit to you?”
^{l} BP was taken every 15 minutes for 2 hour post administration of study drug.
^{m} Participants brought in pads/diapers worn for 3 days prior to Visit 1A & 2 (if V1A after screening V1) and 3 days prior to all subsequent visits (Visit 3 to Visit 8); also brought in clean pad/diapers to use as baseline.
ⁿ Visit 1A occurred in some cases on same day as V1. In this case all V1A procedures not already to be done at V1 were completed. Cystoscopy was performed after all other V1 procedures and post cystoscopy urine culture obtained using clean void. If V1A coincided with V1, then since pad collection and diaries had not been completed prior to V1, these were checked for compliance at V2.
^{o} ECG was done prior to administration of study.

In both active treatment groups, the majority of adverse events (AEs) were mild in severity and all were considered unrelated to the study drug. Two women had mild unrelated UTIs post-treatment with hMaxi-K: one receiving 24,000 ug at month after dosing and the other receiving 16,000 ug at 6 months after dosing. There was one unrelated serious AE reported in the 16,000 ug group; exacerbation of pre-existing asthma due to the cold weather which required an ER visit and resolved after asthma treatment was given. No subject was discontinued due to an AE and all enrolled subjects completed the 6 month trial. In addition, during the 18 month long-term post study safety follow-up, no issues were reported in the subjects followed to date (9 of 13 completed 18 month follow-ups; 13 of 13 completed the 12 month follow-ups).

The average of diary data collected for 7 days prior to each visit revealed statistically significant (p<0.05) improvements vs. placebo and baseline with durable reduction in mean number of voids per day and mean number of urgency episodes per day over the 6 months of the trial. The changes displayed in TABLE 9 and TABLE 10 below were mean changes (+/−SE) from baseline compared to placebo.

TABLE 9
Mean Number of Voids/24 Hours and Reduction Over Time - Efficacy Population

| | | | | hMaxi-K | | | | | |
|---|---|---|---|---|---|---|---|---|---|
| Visit | | Placebo | | 16000 ug | | 24000 ug | | All Doses | |
| Visit 1A | n | 4 | | 6 | | 3 | | 9 | |
| (Screening) | Mean no. voids | 10.46 | (3.48) | 11.99 | (3.65) | 17.39 | (5.22) | 13.79 | (4.73) |
| | (SD) | | | | | | | | |
| Visit 2 | n | 4 | | 6 | | 3 | | 9 | |
| (Baseline) | Mean no. voids | 10.18 | (4.78) | 11.26 | (2.70) | 17.19 | (7.07) | 13.24 | (5.08) |
| | (SD) | | | | | | | | |
| Visit 3 | n | 4 | | 6 | | 3 | | 9 | |
| (Week 1) | Mean no. voids | 11.59 | (4.98) | 9.10 | (2.12) | 14.46 | (3.74) | 10.89 | (3.67) |
| | (SD) | | | | | | | | |
| | Mean change from | 1.41 | (0.78) | −2.16 | (1.80) | −2.73 | (7.29) | −2.35 | (3.92) |
| | baseline (SD) | | | | | | | | |
| | SEM | 0.39 | | 0.73 | | 4.21 | | 1.31 | |
| | P-value [1] | 0.251 | | 0.052 | | 0.074 | | 0.018 | |
| | P-value [2] | | | 0.044 | | 0.047 | | 0.027 | |
| | Difference of LS | | | −3.57 | | −4.14 | | −3.86 | |
| | Means vs. placebo | | | | | | | | |
| | 95% CI | | | −7.01, −0.13 | | −8.22, −0.07 | | −7.12, −0.59 | |
| Visit 4 | n | 4 | | 6 | | 3 | | 9 | |
| (Week 2) | Mean no. voids | 10.68 | (4.10) | 8.35 | (2.65) | 13.52 | (1.94) | 10.07 | (3.47) |
| | (SD) | | | | | | | | |
| | Mean change from | 0.51 | (1.22) | −2.92 | (2.04) | −3.67 | (6.48) | −3.17 | (3.64) |
| | baseline (SD) | | | | | | | | |
| | SEM | 0.61 | | 0.83 | | 3.74 | | 1.21 | |
| | P-value [1] | 0.667 | | 0.016 | | 0.026 | | 0.004 | |
| | P-value [2] | | | 0.051 | | 0.046 | | 0.029 | |
| | Difference of LS | | | −3.42 | | −4.17 | | −3.80 | |
| | Means vs. placebo | | | | | | | | |
| | 95% CI | | | −6.87, 0.02 | | −8.25, −0.10 | | −7.06, −0.53 | |
| Visit 5 | n | 4 | | 6 | | 3 | | 9 | |
| (Week 4) | Mean no. voids | 11.40 | (4.42) | 8.87 | (2.25) | 13.48 | (1.08) | 10.40 | (2.96) |
| | (SD) | | | | | | | | |
| | Mean change from | 1.22 | (0.69) | −2.40 | (2.11) | −3.71 | (7.27) | −2.84 | (4.05) |
| | baseline (SD) | | | | | | | | |
| | SEM | 0.35 | | 0.86 | | 4.20 | | 1.35 | |
| | P-value [1] | 0.315 | | 0.035 | | 0.025 | | 0.006 | |
| | P-value [2] | | | 0.042 | | 0.024 | | 0.017 | |
| | Difference of LS | | | −3.62 | | −4.93 | | −4.28 | |
| | Means vs. placebo | | | | | | | | |
| | 95% CI | | | −7.06, −0.17 | | −9.01, −0.86 | | −7.54, −1.01 | |
| Visit 6 | N | 4 | | 6 | | 3 | | 9 | |
| (Week 8) | Mean no. voids | 10.17 | (3.89) | 9.48 | (2.73) | 13.52 | (2.19) | 10.83 | (3.15) |
| | (SD) | | | | | | | | |
| | Mean change from | −0.01 | (1.20) | −1.79 | (2.15) | −3.67 | (7.75) | −2.41 | (4.33) |
| | baseline (SD) | | | | | | | | |
| | SEM | 0.60 | | 0.88 | | 4.47 | | 1.44 | |
| | P-value [1] | 0.996 | | 0.094 | | 0.026 | | 0.011 | |
| | P-value [2] | | | 0.261 | | 0.071 | | 0.090 | |
| | Difference of LS | | | −1.78 | | −3.66 | | −2.72 | |
| | Means vs. placebo | | | | | | | | |
| | 95% CI | | | −5.22, 1.66 | | −7.74, 0.41 | | −5.99, 0.55 | |
| Visit 7 | N | 4 | | 6 | | 3 | | 9 | |
| (Week 12) | Mean no. voids | 10.96 | (4.30) | 10.21 | (4.11) | 12.90 | (2.35) | 11.11 | (3.71) |
| | (SD) | | | | | | | | |
| | Mean change from | 0.79 | (1.67) | −1.05 | (2.90) | −4.29 | (6.97) | −2.13 | (4.47) |
| | baseline (SD) | | | | | | | | |
| | SEM | 0.84 | | 1.18 | | 4.02 | | 1.49 | |
| | P-value [1] | 0.509 | | 0.293 | | 0.013 | | 0.012 | |
| | P-value [2] | | | 0.248 | | 0.022 | | 0.041 | |
| | Difference of LS | | | −1.83 | | −5.07 | | −3.45 | |
| | Means vs. placebo | | | | | | | | |
| | 95% CI | | | −5.28, 1.61 | | −9.15, −1.00 | | −6.72, −0.19 | |
| Visit 8 | N | 4 | | 6 | | 3 | | 9 | |
| (ExitVisit - | Mean no. voids | 11.14 | (4.81) | 9.74 | (3.04) | 13.86 | (3.02) | 11.11 | (3.51) |
| Week 24) | (SD) | | | | | | | | |
| | Mean change from | 0.96 | (0.99) | −1.52 | (2.55) | −3.33 | (7.06) | −2.13 | (4.16) |
| | baseline (SD) | | | | | | | | |

| SEM | 0.50 | 1.04 | 4.08 | 1.39 |
|---|---|---|---|---|
| P-value [1] | 0.421 | 0.142 | 0.038 | 0.019 |
| P-value [2] | | 0.131 | 0.041 | 0.044 |
| Difference of LS | | −2.49 | −4.30 | −3.39 |
| Means vs. placebo | | | | |
| 95% CI | | −5.93, 0.96 | −8.37, −0.22 | −6.66, −0.13 |

[1]: p-value to test whether there was a statistically significant difference between values measured at certain time point vs. baseline measurement for certain treatment.
[2]: p-value for test whether there was a statistically significant difference between changes from baseline comparing to placebo.
All the p-values and estimates were derived from a linear mixed effect model with number of voids as dependent variables, treatments (placebo, 16000 ug, 24000 ug and total hMaxi-K), time point and interaction of time and treatment. All doses = all hMaxi-K doses.
SD = standard deviation; SEM = standard error of the mean.

TABLE 10
Mean Number of Urgency Episodes/24 Hours and Reduction Over Time - Efficacy Population

| | | | | hMaxi-K | | | | | |
|---|---|---|---|---|---|---|---|---|---|
| Visit | | Placebo | | 16000 ug | | 24000 ug | | All Doses | |
| Visit 1A | N | 4 | | 6 | | 3 | | 9 | |
| (Screening) | Mean no urgency | 10.04 | (3.80) | 11.12 | (4.08) | 17.27 | (5.33) | 13.17 | (5.19) |
| | episodes (SD) | | | | | | | | |
| Visit 2 | N | 4 | | 6 | | 3 | | 9 | |
| (Baseline) | Mean no urgency | 9.82 | (5.17) | 10.21 | (3.55) | 17.19 | (7.07) | 12.53 | (5.71) |
| | episodes (SD) | | | | | | | | |
| Visit 3 | N | 4 | | 6 | | 3 | | 9 | |
| (Week 1) | Mean no urgency | 11.27 | (5.25) | 7.89 | (3.11) | 14.46 | (3.74) | 10.08 | (4.51) |
| | episodes (SD) | | | | | | | | |
| | Mean change from | 1.45 | (0.83) | −2.31 | (2.17) | −2.73 | (7.29) | −2.45 | (4.03) |
| | baseline (SD) | | | | | | | | |
| | SEM | 0.42 | | 0.88 | | 4.21 | | 1.34 | |
| | P-value [1] | 0.240 | | 0.040 | | 0.074 | | 0.016 | |
| | P-value [2] | | | 0.036 | | 0.046 | | 0.024 | |
| | Difference of LS | | | −3.76 | | −4.18 | | −3.97 | |
| | Means vs. placebo | | | | | | | | |
| | 95% CI | | | −7.20, −0.32 | | −8.25, −0.11 | | −7.23, −0.71 | |
| Visit 4 | N | 4 | | 6 | | 3 | | 9 | |
| (Week 2) | Mean no urgency | 10.22 | (4.49) | 7.17 | (3.35) | 13.52 | (1.94) | 9.29 | (4.25) |
| | episodes (SD) | | | | | | | | |
| | Mean change from | 0.40 | (1.03) | −3.04 | (2.07) | −3.67 | (6.48) | −3.25 | (3.64) |
| | baseline (SD) | | | | | | | | |
| | SEM | 0.51 | | 0.85 | | 3.74 | | 1.21 | |
| | P-value [1] | 0.734 | | 0.013 | | 0.026 | | 0.004 | |
| | P-value [2] | | | 0.050 | | 0.050 | | 0.030 | |
| | Difference of LS | | | −3.43 | | −4.07 | | −3.75 | |
| | Means vs. placebo | | | | | | | | |
| | 95% CI | | | −6.87, 0.01 | | −8.14, 0.00 | | −7.01, −0.49 | |
| Visit 5 | N | 4 | | 6 | | 3 | | 9 | |
| (Week 4) | Mean no urgency | 11.04 | (4.75) | 7.87 | (3.92) | 13.48 | (1.08) | 9.74 | (4.22) |
| | episodes (SD) | | | | | | | | |
| | Mean change from | 1.22 | (0.69) | −2.34 | (2.07) | −3.71 | (7.27) | −2.80 | (4.04) |
| | baseline (SD) | | | | | | | | |
| | SEM | 0.35 | | 0.84 | | 4.20 | | 1.35 | |
| | P-value [1] | 0.315 | | 0.038 | | 0.025 | | 0.007 | |
| | P-value [2] | | | 0.044 | | 0.024 | | 0.018 | |
| | Difference of LS | | | −3.56 | | −4.93 | | −4.25 | |
| | Means vs. placebo | | | | | | | | |
| | 95% CI | | | −7.00, −0.12 | | −9.00, −0.86 | | −7.51, −0.98 | |
| Visit 6 | N | 4 | | 6 | | 3 | | 9 | |
| (Week 8) | Mean no urgency | 9.60 | (4.45) | 8.32 | (4.40) | 13.52 | (2.19) | 10.05 | (4.48) |
| | episodes (SD) | | | | | | | | |
| | Mean change from | −0.22 | (0.89) | −1.89 | (2.07) | −3.67 | (7.75) | −2.48 | (4.30) |
| | baseline (SD) | | | | | | | | |
| | SEM | 0.45 | | 0.85 | | 4.47 | | 1.43 | |
| | P-value [1] | 0.851 | | 0.079 | | 0.026 | | 0.010 | |
| | P-value [2] | | | 0.289 | | 0.085 | | 0.106 | |
| | Difference of LS | | | −1.67 | | −3.45 | | −2.56 | |
| | Means vs. placebo | | | | | | | | |
| | 95% CI | | | −5.11, 1.77 | | −7.52, 0.62 | | −5.82, 0.71 | |
| Visit 7 | N | 4 | | 6 | | 3 | | 9 | |
| (Week 12) | Mean no urgency | 10.86 | (4.35) | 10.00 | (4.31) | 12.86 | (2.38) | 10.95 | (3.88) |
| | episodes (SD) | | | | | | | | |
| | Mean change from | 1.04 | (2.15) | −0.21 | (2.41) | −4.33 | (7.05) | −1.58 | (4.51) |
| | baseline (SD) | | | | | | | | |
| | SEM | 1.07 | | 0.99 | | 4.07 | | 1.50 | |
| | P-value [1] | 0.389 | | 0.829 | | 0.013 | | 0.025 | |
| | P-value [2] | | | 0.421 | | 0.017 | | 0.048 | |
| | Difference of LS | | | −1.24 | | −5.37 | | −3.31 | |
| | Means vs. placebo | | | | | | | | |
| | 95% CI | | | −4.68, 2.20 | | −9.44, −1.30 | | −6.57, −0.04 | |
| Visit 8 | N | 4 | | 6 | | 3 | | 9 | |
| (Exit Visit) | Mean no urgency | 10.89 | (4.99) | 9.29 | (3.53) | 13.86 | (3.02) | 10.81 | (3.91) |
| (Week 24) | episodes (SD) | | | | | | | | |
| | Mean change from | 1.07 | (1.18) | −0.92 | (2.27) | −3.33 | (7.06) | −1.72 | (4.14) |
| | baseline (SD) | | | | | | | | |

| SEM | 0.59 | 0.92 | 4.08 | 1.38 |
|---|---|---|---|---|
| P-value [1] | 0.373 | 0.350 | 0.037 | 0.032 |
| P-value [2] | | 0.213 | 0.038 | 0.054 |
| Difference of LS | | −1.99 | −4.40 | −3.20 |
| Means vs. placebo | | | | |
| 95% CI | | −5.43, 1.45 | −8.47, −0.33 | −6.46, 0.06 |

[1]: p-value to test whether there was a statistically significant difference between values measured at certain time point vs. baseline measurement for certain treatment.
[2]: p-value for test whether there was a statistically significant difference between changes from baseline comparing to placebo.
All the p-values and estimates were derived from a linear mixed effect model with number of voids as dependent variables, treatments (placebo, 16000 ug, 24000 ug and total hMaxi-K), time point and interaction of time and treatment.
All doses = all hMaxi-K doses.
SD = standard deviation; SEM = standard error of the mean.

Quality of life parameters (King Health Questionnaire) showed statistically significant sustained mean changes for the individual active treatments and for the combined active treatment groups (all doses) vs. placebo and vs. baseline in the domains of Impact on Life, Role Limitations, Physical Limitations, Social Limitations and Sleep Energy.

Results from this phase 1B clinical trial showed a significant reduction of the number of voiding and urgency episodes after a single administration of hMaxi-K lasted for the 6 month duration of the trial. Those results were observed in the absence of a change in PVR and treatment-related serious adverse events. The results of this novel clinical trial showed for the first time that a single intradetrusor administration of human Maxi-K gene was safe.

Despite the small population enrolled, overall findings from the participant diaries showed significant reductions (p<0.05) for the mean number of voids and mean number of urgency episodes vs. placebo and vs. baseline for all active treatments and of urge incontinence episodes vs. baseline for all doses of study drug. Participant response to treatment showed some positive p values for all active doses vs. placebo at Visits 3 and 5 (see TABLE 11).

TABLE 11
Number of Urge Incontinence episodes and Reduction Over Time - Efficacy Population

| | | | | hMaxi-K | | | | | |
|---|---|---|---|---|---|---|---|---|---|
| Visit | | Placebo | | 16000 ug | | 24000 ug | | All Doses | |
| Visit 1A | n | 4 | | 6 | | 3 | | 9 | |
| (Screening) | Mean no. urge | 1.88 | (1.25) | 2.08 | (0.57) | 8.69 | (12.02) | 4.29 | (6.87) |
| | incontinence | | | | | | | | |
| | episodes/24 hrs (SD) | | | | | | | | |
| Visit 2 | n | 4 | | 6 | | 3 | | 9 | |
| (Baseline) | Mean no. urge | 1.82 | (1.52) | 1.91 | (0.83) | 3.81 | (3.30) | 2.54 | (2.01) |
| | incontinence | | | | | | | | |
| | episodes/24 hrs (SD) | | | | | | | | |
| Visit 3 | n | 4 | | 6 | | 3 | | 9 | |
| (Week 1) | Mean no. urge | 1.43 | (1.32) | 1.29 | (1.08) | 2.74 | (0.25) | 1.77 | (1.13) |
| | incontinence | | | | | | | | |
| | episodes/24 hrs (SD) | | | | | | | | |
| | Mean change from | −0.39 | (0.22) | −0.63 | (0.74) | −1.07 | (3.15) | −0.78 | (1.69) |
| | baseline (SD) | | | | | | | | |
| | SEM | 0.11 | | 0.30 | | 1.82 | | 0.56 | |
| | P-value [1] | 0.460 | | 0.164 | | 0.103 | | 0.045 | |
| | P-value [2] | | | 0.718 | | 0.395 | | 0.470 | |
| | Difference of LS Means | | | −0.24 | | −0.68 | | −0.46 | |
| | vs. placebo | | | | | | | | |
| | 95% CI | | | −1.75, 1.27 | | −2.47, 1.10 | | −1.89,0.97 | |
| Visit 4 | n | 4 | | 6 | | 3 | | 9 | |
| (Week 2) | Mean no. urge | 1.23 | (1.27) | 0.86 | (1.09) | 2.95 | (1.35) | 1.56 | (1.51) |
| | incontinence | | | | | | | | |
| | episodes/24 hrs (SD) | | | | | | | | |
| | Mean change from | −0.58 | (0.81) | −1.05 | (1.39) | −0.86 | (2.60) | −0.99 | (1.70) |
| | baseline (SD) | | | | | | | | |
| | SEM | 0.40 | | 0.57 | | 1.50 | | 0.57 | |
| | P-value [1] | 0.277 | | 0.035 | | 0.177 | | 0.029 | |
| | P-value [2] | | | 0.487 | | 0.728 | | 0.559 | |
| | Difference of LS Means | | | −0.47 | | −0.27 | | −0.37 | |
| | vs. placebo | | | | | | | | |
| | 95% CI | | | −1.98, 1.04 | | −2.06, 1.51 | | −1.80, 1.06 | |
| Visit 5 | n | 4 | | 6 | | 3 | | 9 | |
| (Week 4) | Mean no. urge | 1.14 | (0.95) | 0.66 | (0.81) | 3.10 | (2.08) | 1.47 | (1.72) |
| | incontinence | | | | | | | | |
| | episodes/24 hrs (SD) | | | | | | | | |
| | Mean change from | −0.67 | (0.98) | −1.25 | (1.16) | −0.71 | (1.76) | −1.07 | (1.30) |
| | baseline (SD) | | | | | | | | |
| | SEM | 0.49 | | 0.48 | | 1.01 | | 0.43 | |
| | P-value [1] | 0.216 | | 0.017 | | 0.251 | | 0.026 | |
| | P-value [2] | | | 0.393 | | 0.958 | | 0.623 | |
| | Difference of LS | | | −0.58 | | −0.04 | | −0.31 | |
| | Means vs. placebo | | | | | | | | |
| | 95% CI | | | −2.09, 0.93 | | −1.83, 1.74 | | −1.74, 1.12 | |
| Visit 6 | n | 4 | | 6 | | 3 | | 9 | |
| (Week 8) | Mean no. urge | 1.02 | (1.15) | 0.50 | (0.92) | 2.57 | (2.13) | 1.19 | (1.66) |
| | incontinence | | | | | | | | |
| | episodes/24 hrs (SD) | | | | | | | | |
| | Mean change from | −0.79 | (0.49) | −1.41 | (1.21) | −1.24 | (1.67) | −1.35 | (1.27) |
| | baseline (SD) | | | | | | | | |
| | SEM | 0.25 | | 0.49 | | 0.97 | | 0.42 | |
| | P-value [1] | 0.153 | | 0.010 | | 0.067 | | 0.007 | |
| | P-value [2] | | | 0.363 | | 0.573 | | 0.407 | |
| | Difference of LS | | | −0.62 | | −0.45 | | −0.53 | |
| | Means vs. placebo | | | | | | | | |
| | 95% CI | | | −2.13, 0.89 | | −2.23, 1.34 | | −1.97, 0.90 | |
| Visit 7 | n | 4 | | 6 | | 3 | | 9 | |
| (Week 12) | Mean no. urge | 1.25 | (1.09) | 0.64 | (0.75) | 3.29 | (2.27) | 1.52 | (1.84) |
| | incontinence | | | | | | | | |
| | episodes/24 hrs (SD) | | | | | | | | |
| | Mean change from | −0.57 | (0.71) | −1.27 | (1.17) | −0.52 | (1.57) | −1.02 | (1.27) |
| | baseline (SD) | | | | | | | | |
| | SEM | 0.35 | | 0.48 | | 0.90 | | 0.42 | |
| | P-value [1] | 0.290 | | 0.016 | | 0.389 | | 0.037 | |
| | P-value [2] | | | 0.306 | | 0.958 | | 0.601 | |
| | Difference of LS | | | −0.70 | | 0.04 | | −0.33 | |
| | Means vs. placebo | | | | | | | | |
| | 95% CI | | | −2.21, 0.81 | | −1.74, 1.83 | | −1.76, 1.10 | |
| Visit 8 | n | 4 | | 6 | | 3 | | 9 | |
| (Exit Visit) | Mean no. urge | 0.86 | (0.76) | 0.62 | (0.84) | 1.52 | (1.39) | 0.92 | (1.06) |
| (Week 24) | incontinence | | | | | | | | |
| | episodes/24 hrs (SD) | | | | | | | | |
| | Mean change from | −0.96 | (0.94) | −1.29 | (1.10) | −2.29 | (2.72) | −1.62 | (1.69) |
| | baseline (SD) | | | | | | | | |

| SEM | 0.47 | 0.45 | 1.57 | 0.56 |
|---|---|---|---|---|
| P-value [1] | 0.094 | 0.015 | 0.005 | 0.001 |
| P-value [2] | | 0.616 | 0.122 | 0.212 |
| Difference of LS Means | | −0.34 | −1.33 | −0.83 |
| vs. placebo | | | | |
| 95% CI | | −1.84, 1.17 | −3.11, 0.46 | −2.26, 0.60 |

[1]: P-value to test whether there was a statistically significant difference between values measured at certain time point vs. baseline measurement for certain treatment.
[2]: P-value for test whether there was a statistically significant difference between changes from baseline comparing to placebo.
All the P-values and estimates were derived from a linear mixed effect model with number of voids as dependent variables, treatments (placebo, 16000 ug, 24000 ug and total hMaxi-K), time point and interaction of time and treatment.
All doses = all hMaxi-K doses
SD = standard deviation;
SEM = standard error of the mean

For the reduction in number of voids and urgency episodes, these significant changes vs. placebo and vs. baseline were seen at all visits out to final Visit 8 (24 weeks). There were no significant differences seen between the two active treatments (16,000 ug and 24,000 ug) possibly due to the small number of participants enrolled in the 24,000 ug group (N=3).

Quality of life parameters (King Health Questionnaire) showed statistically significant mean improvement for the individual active treatments and for the combined active treatment groups (all doses) vs. placebo and vs. baseline in many of the domains. This included the following:

Domain 2: Impact on Life
P=0.014 for all active doses and p=0.007 for 24000 ug at Visit 5 vs. baseline,
P=0.016 for 24000 ug at Visit 5 vs. placebo;
P=0.016 for the 24000 ug group vs. 16000 ug group at Visit 5
P=0.043 for all active doses vs. baseline at Visit 6
P=0.010 for 16000 ug and p=0.005 for all active doses vs. baseline at Visit 7
P=0.026 for all active doses vs. baseline at Visit 8

Domain 3: Role Limitations
P=0.004, P=0.015, P<0.001 for 16000 ug, 24000 ug and all active doses, respectively, vs. baseline at Visit 5
P=0.030, P=0.035 and P=0.015 for 16000 ug, 24000 ug and all active doses, respectively, vs. placebo at Visit 5
P=0.023, P=0.014 and P=0.001 for 16000 ug, 24000 ug and all active doses, respectively, vs. baseline at Visit 6
P=0.047, P=0.020 and P=0.014 for 16000 ug, 24000 ug and all active doses, respectively, vs. placebo at Visit 6
P=0.012, P=0.014 and P<0.001 for 16000 ug, 24000 ug and all active doses, respectively, vs. placebo at Visit 7
P=0.032 and P=0.021 for 24000 ug and all active doses, respectively, vs. placebo at Visit 7
P=0.014 and P=0.005 for 24000 ug and all active doses, respectively, vs. baseline at Visit 8
P=0.047. P=0.007 and P=0.007 for 16000 ug, 24000 ug and all active doses, respectively, vs. placebo at Visit 8

Domain 4 Physical Limitations
P=0.018 and P=0.005 for 24000 ug and all active doses, respectively, vs. baseline at Visit 6
P=0.012, P=0.018 and P=0.001 for 16000 ug, 24000 ug and all active doses, respectively, vs. baseline at Visit 7
P=0.012, P=0.047 and P=0.003 for 16000 ug, 24000 ug and all active doses, respectively, vs. baseline at Visit 8

Domain 5: Social Limitations
P=0.032 and P=0.22, tor 24000 ug vs. baseline and placebo, respectively, at Visit 6
P=0.002 and P=0.004 for 24000 ug and all active doses, respectively, vs. baseline at Visit 7
P=0.008 and P=0.043 for 24000 ug and all active doses, respectively, vs. placebo at Visit 7
P=0.002 and P=0.014 for 24000 ug and all active doses, respectively, vs. baseline at Visit 8
P=0.006 for 24000 ug vs. placebo at Visit 8

Domain 8: Sleep Energy
P=0.047. P=0.007 and P=0.001 for 1 6000 ug, 24000 ug and all active doses, respectively, vs. baseline at Visit 5
P=0.020 and P=0.015 for 24000 ug and all active doses, respectively, vs. placebo at Visit 5
P=0.005 and P=0.006 for 24000 ug and all active doses, respectively, vs. baseline at Visit 6
P=0.001 and P=0.006 for 24000 ug and all active doses, respectively, vs. baseline at Visit 7
P=0.012 for 24000 ug vs. placebo at Visit 7

The 72 hour Pad Test (TABLE 12) showed statistically significant changes at Visit 3-6 and Visit 8 for hMaxi-K active doses vs. baseline, however, there were also statistically significant changes for placebo at Visits 3-5 and Visit 8. Overall the placebo group appeared to have less severe disease than the active treatment groups with baseline (V2) pad weights for active treatment being almost 2 times greater than that of the placebo group. In addition, the VIA mean pad weight for placebo was only 29 grams whereas the weight at V2 for this group was 259 grams (almost 9 times greater than VIA). This was due to the fact that participant 002-001 had thrown out her pads prior to VIA (so she was not included in the VIA means) and she appears to have had more severe disease than the other 3 placebo participants (her 3-day average pad weight at V2 was 295 grams vs. 3.3 to 36 grams for the other 3 participants).

TABLE 12
Participant Perception of Response to Treatment - Efficacy Population

| | | Placebo, n (%) | hMaxi-K, n (%) | | |
|---|---|---|---|---|---|
| | | Placebo | 16000 ug | 24000 ug | All Doses |
| V3 (N = 13) | No benefit | 3 (75.00) | 1 (16.67) | 0 | 1 (11.11) |
| | Yes, a little benefit | 1 (25.00) | 1 (16.67) | 3 (100.0) | 4 (44.44) |
| | Yes, very | 0 | 4 (66.67) | 0 | 4 (44.44) |
| | much benefit | | | | |
| | P-value | | 0.1429 | 0.1429 | 0.0190 |
| V4 (N = 13) | No benefit | 3 (75.00) | 1 (16.67) | 0 | 1 (11.11) |
| | Yes, a little benefit | 1 (25.00) | 1 (16.67) | 2 (66.67) | 3 (33.33) |
| | Yes, very | 0 | 4 (66.67) | 1 (33.33) | 5 (55.56) |
| | much benefit | | | | |
| | P-value | | 0.1429 | 0.2286 | 0.1202 |
| V5 (N = 13) | No benefit | 3 (75.00) | 1 (16.67) | 0 | 1 (11.11) |
| | Yes, a little benefit | 1 (25.00) | 0 | 2 (66.67) | 2 (22.22) |
| | Yes, very | 0 | 5 (83.33) | 1 (33.33) | 6 (66.67) |
| | much benefit | | | | |
| | P-value | | 0.0238 | 0.2286 | 0.0126 |
| V6 (N = 13) | No benefit | 3 (75.00) | 1 (16.67) | 0 | 1 (11.11) |
| | Yes, a little benefit | 1 (25.00) | 2 (33.33) | 2 (66.67) | 4 (44.44) |
| | Yes, very | 0 | 3 (50.00) | 1 (33.33) | 4 (44.44) |
| | much benefit | | | | |
| | P-value | | 0.2286 | 0.2286 | 0.2727 |
| V7 (N = 13) | No benefit | 3 (75.00) | 2 (33.33) | 0 | 2 (22.22) |
| | Yes, a little benefit | 1 (25.00) | 1 (16.67) | 2 (66.67) | 3 (33.33) |
| | Yes, very | 0 | 3 (50.00) | 1 (33.33) | 4 (44.44) |
| | much benefit | | | | |
| | P-value | | 0.2857 | 0.2286 | 0.2727 |
| V8 (N = 13) | No benefit | 3 (75.00) | 2 (33.33) | 0 | 2 (22.22) |
| | Yes, a little benefit | 1 (25.00) | 1 (16.67) | 2 (66.67) | 3 (33.33) |
| | Yes, very | 0 | 3 (50.00) | 1 (33.33) | 4 (44.44) |
| | much benefit | | | | |
| | P-value | | 0.2857 | 0.2286 | 0.2727 |

Note:
p-values were nominal and for chi-square test to see whether perception of response to treatment were different for patients received treatment and those received placebo.
All doses = all hMaxi-K doses

TABLE 13
Change in the Mean Number of Urge Incontinence Episode per 24 Hours - Efficacy Population

| | | | hMaxi-K | | |
|---|---|---|---|---|---|
| Visit | | Placebo | 16000 ug | 24000 ug | All Doses |
| Urge incontinence | N | 4 | 6 | 3 | 9 |
| episode per 24 | | | | | |
| hours | | | | | |
| | N | 4 | 6 | 3 | 9 |
| Visit 1A | Mean (SD) | 1.88 (1.25) | 2.08 (0.57) | 8.69 (12.02) | 4.29 (6.87) |
| | N | 4 | 6 | 3 | 9 |
| Visit 2 | Mean (SD) | 1.82 (1.52) | 1.91 (0.83) | 3.81 (3.30) | 2.54 (2.01) |
| | N | 4 | 6 | 3 | 9 |
| Visit 3 | Mean (SD) | 1.43 (1.32) | 1.29 (1.08) | 2.74 (0.25) | 1.77 (1.13) |
| | N | 4 | 6 | 3 | 9 |
| Visit 4 | Mean (SD) | 1.23 (1.27) | 0.86 (1.09) | 2.95 (1.35) | 1.56( 1.51) |
| | N | 4 | 6 | 3 | 9 |
| Visit 5 | Mean (SD) | 1.14 (0.95) | 0.66 (0.81) | 3.10 (2.08) | 1.47 (1.72) |
| | N | 4 | 6 | 3 | 9 |
| Visit 6 | Mean (SD) | 1.02 (1.15) | 0.50 (0.92) | 2.57 (2.13) | 1.19 (1.66) |
| | N | 4 | 6 | 3 | 9 |
| Visit 7 | Mean (SD) | 1.25 (1.09) | 0.64 (0.75) | 3.29 (2.27) | 1.52 (1.84) |
| | N | 4 | 6 | 3 | 9 |
| Visit 8 (Exit Visit) | Mean (SD) | 0.86 (0.76) | 0.62 (0.84) | 1.52 (1.39) | 0.92 (1.06) |
| Change from | | | | | |
| Baseline V2 | | | | | |
| Visit 3 | N | 4 | 6 | 3 | 9 |
| | Mean (SD) | −0.39 (0.22) | −0.63 (0.74) | −1.07 (3.15) | −0.78 (1.69) |
| | p-value [1] | 0.460 | 0.164 | 0.103 | 0.045 |
| | p-value [2] | | 0.718 | 0.395 | 0.470 |
| | Difference of LS | | −0.24 | −0.68 | −0.46 |
| | Means vs. placebo | | | | |
| | 95% CI | | −1.75, 1.27 | −2.47, 1.10 | −1.89, 0.97 |
| | p-value [3] | | | 0.545 | |
| | Difference of LS | | | −0.44 | |
| | Means 24000 ug vs. | | | | |
| | 16000 ug | | | | |
| | 95% CI | | | −2.10, 1.21 | |
| Visit 4 | N | 4 | 6 | 3 | 9 |
| | Mean (SD) | −0.58 (0.81) | −1.05 (1.39) | −0.86 (2.60) | −0.99 (1.70) |
| | p-value [1] | 0.277 | 0.035 | 0.177 | 0.029 |
| | p-value [2] | | 0.487 | 0.728 | 0.559 |
| | Difference of LS | | −0.47 | −0.27 | −0.37 |
| | Means vs. placebo | | | | |
| | 95% CI | | −1.98, 1.04 | −2.06, 1.51 | −1.80, 1.06 |
| | p-value [3] | | | 0.789 | |
| | Difference of LS | | | 0.19 | |
| | Means 24000 ug vs. | | | | |
| | 16000 ug | | | | |
| | 95% CI | | | −1.46, 1.85 | |
| Visit 5 | N | 4 | 6 | 3 | 9 |
| | Mean (SD) | −0.67 (0.98) | −1.25 (1.16) | −0.71 (1.76) | −1.07 (1.30) |
| | p-value [1] | 0.216 | 0.017 | 0.251 | 0.026 |
| | p-value [2] | | 0.393 | 0.958 | 0.623 |
| | Difference of LS | | −0.58 | −0.04 | −0.31 |
| | Means vs. placebo | | | | |
| | 95% CI | | −2.09, 0.93 | −1.83, 1.74 | −1.74, 1.12 |
| | p-value [3] | | | 0.465 | |
| | Difference of LS | | | 0.54 | |
| | Means 24000 ug vs. | | | | |
| | 16000 ug | | | | |
| | 95% CI | | | −1.11, 2.19 | |
| Visit 6 | N | 4 | 6 | 3 | 9 |
| | Mean (SD) | −0.79 (0.49) | −1.41 (1.21) | −1.24 (1.67) | −1.35 (1.27) |
| | p-value [1] | 0.153 | 0.010 | 0.067 | 0.007 |
| | p-value [2] | | 0.363 | 0.573 | 0.407 |
| | Difference of LS | | −0.62 | −0.45 | −0.53 |
| | Means vs. placebo | | | | |
| | 95% CI | | −2.13, 0.89 | −2.23, 1.34 | −1.97, 0.90 |
| | p-value [3] | | | 0.810 | |
| | Difference of LS | | | 0.17 | |
| | Means 24000 ug vs. | | | | |
| | 16000 ug | | | | |
| | 95% CI | | | −1.48, 1.83 | |
| Visit 7 | N | 4 | 6 | 3 | 9 |
| | Mean (SD) | −0.57 (0.71) | −1.27 (1.17) | −0.52 (1.57) | −1.02 (1.27) |
| | p-value [1] | 0.290 | 0.016 | 0.389 | 0.037 |
| | p-value [2] | | 0.306 | 0.958 | 0.601 |
| | Difference of LS | | −0.70 | 0.04 | −0.33 |
| | Means vs. placebo | | | | |
| | 95% CI | | −2.21, 0.81 | −1.74, 1.83 | −1.76, 1.10 |
| | p-value [3] | | | 0.321 | |
| | Difference of LS | | | 0.75 | |
| | Means 24000 ug vs. | | | | |
| | 16000 ug | | | | |
| | 95% CI | | | −0.91, 2.40 | |
| Visit 8 | N | 4 | 6 | 3 | 9 |
| (Exit Visit) | Mean (SD) | −0.96 (0.94) | −1.29 (1.10) | −2.29 (2.72) | −1.62 (1.69) |
| | p-value [1] | 0.094 | 0.015 | 0.005 | 0.001 |
| | p-value [2] | | 0.616 | 0.122 | 0.212 |
| | Difference of LS | | −0.34 | −1.33 | −0.83 |
| | Means vs. placebo | | | | |
| | 95% CI | | −1.84, 1.17 | −3.11, 0.46 | −2.26, 0.60 |
| | p-value [3] | | | 0.199 | |
| | Difference of LS | | | −0.99 | |
| | Means 24000 ug vs. | | | | |
| | 16000 ug | | | | |
| | 95% CI | | | −2.65, 0.66 | |

[1]: p-value to test whether there was a statistically significant difference between values measured at certain time point vs. baseline measurement for certain treatment.
[2]: p-value for test whether there was a statistically significant difference between changes from baseline comparing to placebo.
[3]: p-value for test whether there was difference between 24000 ug group vs. 16000 ug group.
Ss All the p-values and estimates were derived from a linear mixed effect model with number of urge incontinence episode per 24 hours as dependent variables, treatments (placebo, 16000 ug, 24000 ug and total hMaxi-K), time point and interaction of time and treatment.

TABLE 14
Change in the Weight (gm) of 72 Hour Pad Test - Safety Population

| | | | | hMaxi-K | | | | | |
|---|---|---|---|---|---|---|---|---|---|
| Visit | | Placebo | | 16000 ug | | 24000 ug | | All Doses | |
| Visit 1A | n | 3 | | 6 | | 3 | | 9 | |
| Screening | Mean (SD) weight of | 29.33 | (20.03) | 345.00 | (726.50) | 611.67 | (703.53) | 433.89 | (686.58) |
| | 72 hr. pad test | | | | | | | | |
| Visit 2 | n | 4 | | 6 | | 3 | | 9 | |
| Baseline | Mean (SD) weight of | 259.25 | (417.95) | 314.00 | (663.23) | 677.33 | (643.96) | 435.11 | (641.56) |
| | 72 hr. pad test | | | | | | | | |
| Visit 3 | n | 4 | | 6 | | 3 | | 9 | |
| (Week 1) | Mean (SD) weight of | 133.50 | (206.99) | 241.67 | (541.39) | 518.03 | (499.37) | 333.79 | (514.42) |
| | 72 hr. pad test | | | | | | | | |
| | Mean (SD) change | −125.75 | (211.14) | −72.33 | (123.08) | −159.30 | (144.90) | −101.32 | (128.87) |
| | from baseline in pad | | | | | | | | |
| | weight | | | | | | | | |
| | P-value [1] | 0.044 | | 0.127 | | 0.024 | | 0.013 | |
| | P-value [2] | | | 0.446 | | 0.598 | | 0.937 | |
| | Difference of LS | | | 53.42 | | −43.14 | | 5.14 | |
| | Means vs. placebo | | | | | | | | |
| | 95% CI | | | −102.87, 209.70 | | −228.08, 141.80 | | −143.13, 153.41 | |
| Visit 4 | n | 4 | | 6 | | 3 | | 9 | |
| (Week 2) | Mean (SD) weight of | 119.00 | (177.72) | 231.83 | (509.77) | 528.00 | (501.86) | 330.56 | (497.30) |
| | 72 hr. pad test | | | | | | | | |
| | Mean (SD) change | −140.25 | (242.66) | −82.17 | (155.66) | −149.33 | (142.12) | −104.56 | (146.02) |
| | from baseline in pad | | | | | | | | |
| | weight | | | | | | | | |
| | P-value [1] | 0.029 | | 0.090 | | 0.031 | | 0.013 | |
| | P-value [2] | | | 0.409 | | 0.818 | | 0.762 | |
| | Difference of LS | | | 58.08 | | −18.67 | | 19.71 | |
| | Means vs. placebo | | | | | | | | |
| | 95% CI | | | −98.20, 214.37 | | −203.61, 166.27 | | −128.57, 167.98 | |
| Visit 5 | n | 4 | | 6 | | 3 | | 9 | |
| (Week 4) | Mean (SD) weight of | 100.75 | (84.24) | 212.00 | (485.13) | 494.67 | (508.22) | 306.22 | (481.29) |
| | 72 hr. pad test | | | | | | | | |
| | Mean (SD) change | −158.50 | (345.31) | −102.00 | (179.22) | −182.67 | (153.16) | −128.89 | (166.03) |
| | from baseline in pad | | | | | | | | |
| | weight | | | | | | | | |
| | P-value [1] | 0.017 | | 0.045 | | 0.014 | | 0.005 | |
| | P-value [2] | | | 0.421 | | 0.679 | | 0.861 | |
| | Difference of LS | | | 56.50 | | −33.76 | | 11.37 | |
| | Means vs. placebo | | | | | | | | |
| | 95% CI | | | −99.79, 212.79 | | −218.69, 151.18 | | −136.90, 159.64 | |
| Visit 6 [3] | n | 4 | | 6 | | 3 | | 9 | |
| Week 8) | Mean (SD) weight of | 164.00 | (272.19) | 186.33 | (427.25) | 489.33 | (425.48) | 287.33 | (426.96) |
| | 72 hr. pad test | | | | | | | | |
| | Mean (SD) change | −95.25 | (145.96) | −127.67 | (236.90) | −188.00 | (361.87) | −147.78 | (262.15) |
| | from baseline in pad | | | | | | | | |
| | weight | | | | | | | | |
| | P-value [1] | 0.105 | | 0.018 | | 0.012 | | 0.003 | |
| | P-value [2] | | | 0.639 | | 0.232 | | 0.318 | |
| | Difference of LS | | | −32.42 | | −102.34 | | −67.38 | |
| | Means vs. placebo | | | | | | | | |
| | 95% CI | | | −188.70, 123.87 | | −287.28, 82.60 | | −215.65, 80.89 | |
| Visit 7 [3] | n | 4 | | 6 | | 3 | | 9 | |
| (Week 12) | Mean (SD) weight of | 177.50 | (307.75) | 307.50 | (709.54) | 545.3 | (621.50) | 386.78 | (652.19) |
| | 72 hr. pad test | | | | | | | | |
| | Mean (SD) change | −81.75 | (110.34) | −6.50 | (52.31) | −191.00 | (159.81) | −52.63 | (113.57) |
| | from baseline in pad | | | | | | | | |
| | weight | | | | | | | | |
| | P-value [1] | 0.154 | | 0.881 | | 0.224 | | 0.256 | |
| | P-value [2] | | | 0.292 | | 0.860 | | 0.671 | |
| | Difference of LS | | | 75.25 | | −16.46 | | 29.40 | |
| | Means vs. placebo | | | | | | | | |
| | 95% CI | | | −81.04, 231.54 | | −228.54, 195.63 | | −127.70, 186.49 | |
| Visit 8 [3] | n | 4 | | 6 | | 3 | | 9 | |
| (Week 24) | Mean (SD) weight of | 85.00 | (126.10) | 225.00 | (520.04) | 596.67 | (528.52) | 348.89 | (522.87) |
| | 72 hr. pad test | | | | | | | | |
| | Mean (SD) change | −174.25 | (293.32) | −89.00 | (145.01) | −80.67 | (189.03) | −86.22 | (148.64) |
| | from baseline in pad | | | | | | | | |
| | weight | | | | | | | | |

| P-value [1] | 0.011 | 0.071 | 0.171 | 0.042 |
|---|---|---|---|---|
| P-value [2] | | 0.238 | 0.318 | 0.219 |
| Difference of LS | | 85.25 | 83.99 | 84.62 |
| Means vs. placebo | | | | |
| 95% CI | | −71.04, 241.54 | −100.94, 268.93 | −63.65, 232.89 |

[1]: P-value to test whether there was a statistically significant difference between values measured at certain time point vs. baseline measurement for certain treatment.
[2]: P-value for test whether there was a statistically significant difference between changes from baseline comparing to placebo.
[3]: Results included a value of 0 for subject 002019 whose results were incorrectly entered into the database. Results verified by site and CRA.
All the P-values and estimates were derived from a linear mixed effect model with weight of 72-hour pad test as dependent variables, treatments (placebo, 16000 ug, 24000 ug and total hMaxi-K), time point and interaction of time and treatment.
All doses = all hMaxi-K doses
SD = standard deviation

### Example 3

### General Methods

Animal Model of Bladder Overactivity: Although there is no animal model that completely recapitulates all aspects of the corresponding human condition, the partial urethral obstruction (PUO) model to cause detrusor overactivity (DO) in the rat (the same animal model proposed herein) has been generally accepted in the peer reviewed literature and by the NIH. Furthermore this animal model was used by ICI to support their successful IND application for Maxi-K treatment for the OAB indication by the FDA. (Melman et al. Isr. Med. Assoc. J. 2007; 9: 143-146; Andersson J. Urol. 2013; 189: 1622-1623; Chang et al. Am. J. Physiol Renal Physiol 2010; 298: F1416-F1423; Christ et al. BJU, 2006, pp 1076-1083; Jin et al. Am. J. Physiol Regul. Integr. Comp Physiol 2011; 301: R896-R904; Melman et al. Urology 2005; 66: 1127-1133; Melman et al. BJU. Int. 2009; 104: 1292-1300).

Female Sprague-Dawley (250 g) rats were used in this study. PUO will be induced as previously described (Thorneloe et al. Am. J. Physiol Renal Physiol 2005; 289: F604-F610). Briefly, the urethra was isolated, a sterile metal bar with a diameter of 0.91 mm was placed on the urethral surface, and a 3-0 silk suture tied around both the urethra and the bar. When the suture was secured, the bar was removed, leaving the urethra partially obstructed. The abdominal muscle layer and skin were then closed. Controls (sham) underwent the same surgical procedure, except for tying of the suture around the urethra.

Suprapubic Bladder Catheterization: A second surgical procedure was conducted on all rats 2 weeks after the PUO procedure. A lower abdominal and perineal midline incision was made, the bladder was exposed, the obstructing urethral silk suture was removed, a small incision was made in the bladder dome and a cuffed polyethylene cannula was inserted into the bladder and secured with a purse string suture. The cannula was then tunneled through the subcutaneous space and exited through an incision on the back of the animal's neck, closed and secured with sutures. To prevent infections, all rats received an injection of sulfadoxin (24 mg/kg) and trimethoprim (4.8 mg/kg) subcutaneously.

Cystometry: Cystometric studies were performed in unrestrained rats 48 hours after bladder catheterization and removal of urethral obstruction (baseline measurements), and 48 hours after intravesical treatment with nanoparticles. Cystometry was performed as previously described (Suadicani et al. BJU Int 2009; 103: 1686-93; Christ et al. BJU, 2006, pp 1076-1083; Melman et al. BJU. Int. 2009; 104: 1292-1300). Briefly, the animals were placed in a metabolic chamber and the indwelling bladder catheter was connected to a two-way valve and attached to a pressure transducer and an infusion pump. The pressure transducer was connected via a transducer amplifier (ETH 400 CB Sciences) to a data-acquisition board (MacLab/8e, ADI Instruments). Real-time display and recording of pressure measurements were done on a Macintosh computer (MacLab software, version 3.4, ADI Instruments). The pressure transducers were calibrated (in cmH₂O) before each experiment. The rate of bladder infusion was set at 1.5 mL/min using a programmable Harvard infusion pump (model PHD 2000). Cystometric activity was continuously recorded after the first micturition and subsequently for at least ten additional reproducible micturition cycles; as micturitions occur approximately 20 min apart, at least 1.5 h of data were recorded from each animal. Relevant urodynamic parameters were then quantified offline from each cystometrogram (see details below) as previously described (Suadicani et al. BJU Int 2009; 103: 1686-93; Christ et al. BJU, 2006, pp 1076-1083; Melman et al. BJU. Int. 2009; 104: 1292-1300).

Intravesical Administration of Naked Plasmid and Nanoparticle Encapsulating Plasmid: One hour after cystometric evaluation (acquisition of baseline measurements) the animals were anesthetized with isoflurane, the bladder emptied by massaging the pelvic region, and the naked plasmid or the nanoparticle encapsulating plasmid were injected in the bladder lumen through the bladder indwelling catheter. The plasmid and nanoparticles were reconstituted in sterile 0.9% saline and 200 uL of the desired concentration were injected, followed by 100 μL of saline only to account for the 50 μL catheter “deadspace.”

Evaluation of Bladder Function: Bladder function was evaluated based on the following urodynamic parameters: 1) bladder capacity, the volume of infused saline at micturition; 2) basal pressure, the lowest bladder pressure recorded during cystometry between voiding; 3) threshold pressure, the bladder pressure immediately before micturition; 4) micturition pressure, the peak bladder pressure during micturition; 5) micturition volume, the volume of urine discharged during micturition; 6) residual volume, the volume of infused saline minus the micturition volume for each void; and 7) spontaneous activity (SA)=mean intermicturition pressure (IMP) minus mean basal pressure (BP), an approximate index of spontaneous bladder contraction between micturitions. The IMP is the average pressure recorded between micturitions. The mean value of BP was subtracted from the mean IMP to obtain a single SA of 6 to 8 voids during a study. As such, the SA served as an index of the fluctuations in bladder pressure, if any, between the recorded micturition reflexes, a measure of DO, and a presumptive clinical correlate of urinary urgency and a measure of response to gene transfer (Babaoglu et al. Int Urol. Nephrol. 2013; 45:1001-1008; Andersson J. Urol. 2013; 189: 1622-1623)

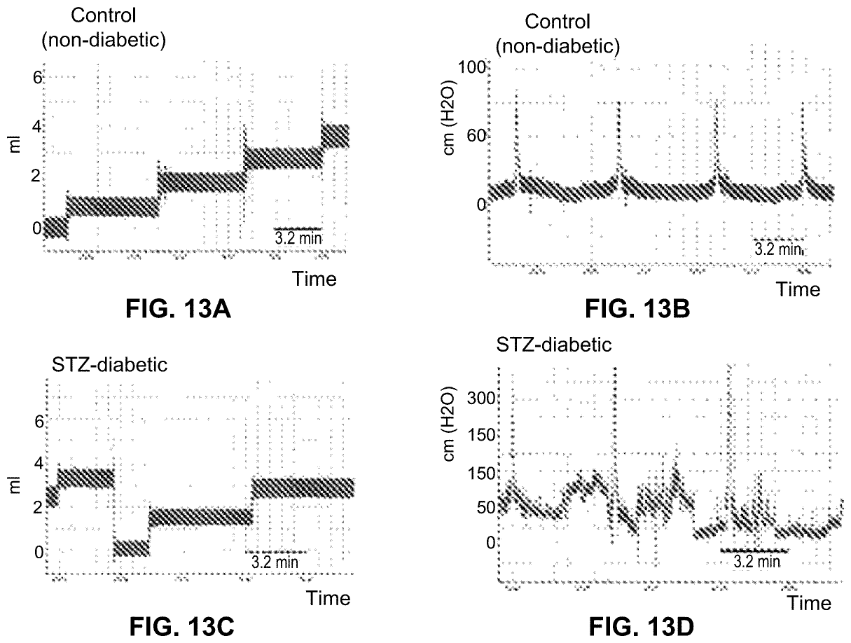

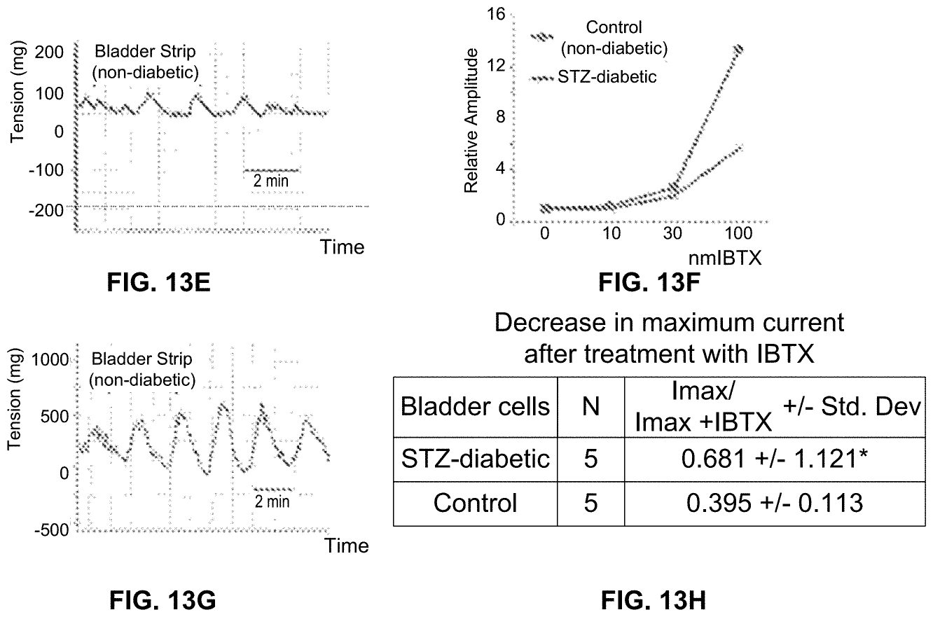

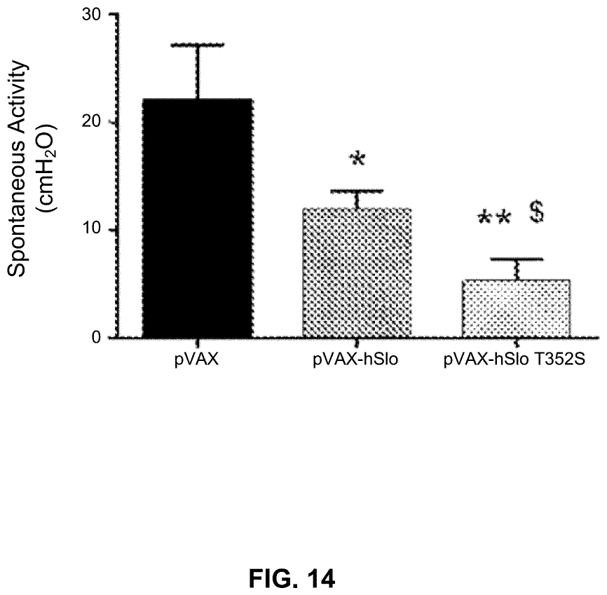

Ex Vivo Evaluation of Changes in Detrusor Function Induced by Treatment with hSlo and hSlo T352S: Effects on detrusor contractility and excitability were determined by organ bath and electrophysiology (patch clamping) in a similar manner as described for preliminary data (see FIG. 13H). In order to perform these evaluations, after cystometry bladders were harvested and cut in half, from the dome to the neck. One half was further cut into strips that were used in the organ bath studies, while the other half was used to isolate detrusor smooth muscle cells for electrophysiological studies.

Organ bath: Bladder strips were mounted in organ baths at 1.0 g resting tension and spontaneous phasic contractions were recorded with a force transducer as previously described (Wang et al. Int J Urol 2014; 21:1059-1064). See FIG. 13E and FIG. 13F.

Experiments were performed in the absence and presence of iberiotoxin (IBTX; 300 nM), a Maxi-K channel blocker, to evaluate the relative contribution of Maxi-K channel activity to development of detrusor spontaneous activity.

Electrophysiology: detrusor smooth muscle cells (SMCs) were isolated and single cell patch-clamping recordings will be performed, as previously described (Davies et al. Eur. Urol. 2007; 52: 1229-1237; Wang et al. Am J Physiol Cell Physiol 2001; 281: C75-88; Wang et al. Int J Impot Res 2000; 12: 9-18), in the absence and presence of IBTX to determine the overall contribution of Maxi-K to changes in detrusor excitability.

### Example 4

### Generation of the T352S Human BKA Construct (PVAX-hSlo-T352S)

Modifications of the hSlo gene can be used to effectively treat human disease that is caused, for example, by alterations of the BK channel by age and disease. The human BKα channel (hslo) cDNA was subcloned into the pVAX to generate pVAX-hSlo. The T352S human BKα construct (pVAX-hSlo-T352S) was prepared from pVAX-hSlo by using the QuickChange II site-directed mutagenesis kit (Agilent Technologies, Inc.) according to the manufacturer's instructions. The primers used for T352S mutation were as follows: 5′-ATGGTCACAATGTCCTCCGTTGGTTATGGGGAT-3′ (SEQ ID NO: 12) and 5′-ATCCCCATAACCAACGGAGGACATTGTGACCAT-3′ (SEQ ID NO: 13). The T352S mutation was verified by DNA sequencing. Transient transfection of HEK293 cells was performed with FuGENE® 6 (Roche) according to the manufacturer's instructions. The HEK cells were studied with electrophysiological patch clamp analysis under the following conditions: Currents were recorded with whole-cell patch-clamp at room temperature. Borosilicate glass electrodes had 4 to 20 MS2 tip resistances when filled with internal solution. The extracellular solution was composed of 137 mM NaCl, 5.4 mM KCl, 1 mM MgCl₂, 1 mM CaCl₂, 2.3 mM NaOH, 5 mM HEPES and 10 mM dextrose (pH 7.4 with NaOH). Internal solution contained 120 mM K-aspartate, 3 mM Na2ATP, 5 mM HEPES, and 5 mM EGTA (pH 7.2 with KOH). Currents were elicited with a holding potential of −80 mV with 200 ms duration testing pulses from −60 mV to +110 mV in 10 mV increments.

CLAMPFIT™ (Molecular Devices, Sunnyvale, Calif., USA) and GRAPHPAD™ PRISM™ (GraphPad Software, San Diego, Calif, USA) were used for data analysis. Data are presented as mean±SEM. P<0.05 by two-way ANOVA (for comparison among groups) or Student's t-test (for comparison of individual voltage steps) was considered to indicate statistical significance.

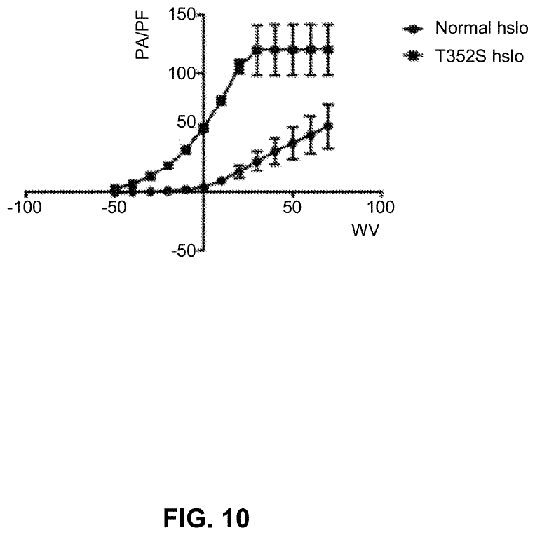

The result of the T352S site-directed mutagenesis demonstrates a leftward shift in the voltage-dependent activation curve, as shown in FIG. 10.

To test the effects of double point mutations on the electrical properties of the hSlo T352S channel, six separate double mutations were created. Each double point mutation was generated with the expectation that the double mutation would both inhibit the negative effect of peroxynitrite of the BK channel and increase the current state measured at low calcium. The double mutations were cytosine for adenine (C for A) and methionine for leucine (M for L) substitutions in the following constructs; pVAX-hSloT352S-C977A (C1), pVAX-hSloT352S-C496A (C2), pVAX-hSloT352S-C681A (C3), pVAX-hSloT352S-M602L (M1), pVAX-hSloT352S-M778L (M2) and pVAX-hSloT352S-M805L (M3).

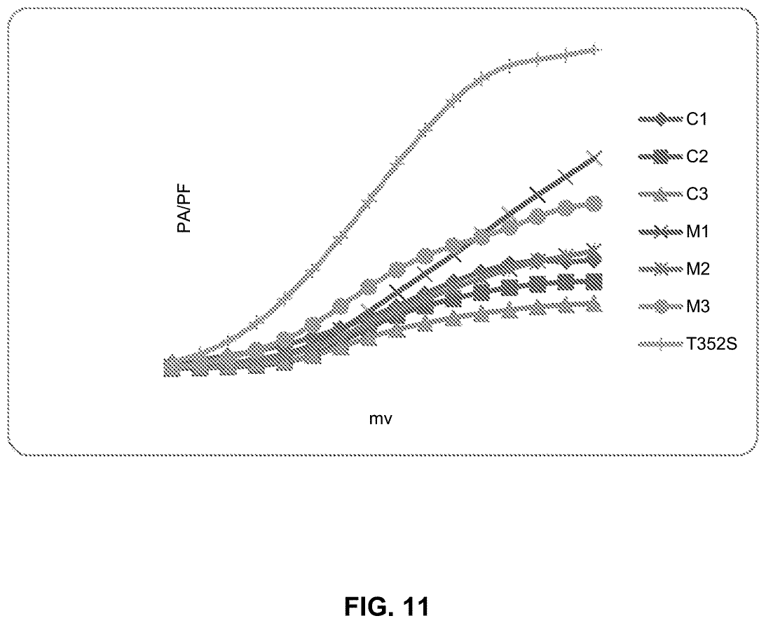

Electrophysiological patch clamp analysis of these substitution constructs was performed after transfection into HEK cells for 24-48 h in a high glucose (22.5 mM) environment. Although the T352S single point mutation is resistant to oxidative stress, the double point mutations (C1, C2, C3, M1, M2, and M3) appear to compromise the effect of the T352S single point mutation in a high glucose environment. The results of those patch clamp experiments are shown in FIG. 11.

### Example 5

### Evaluation of Vectors Expressing hSlo Gene T352S

Previous studies by our group in rats with bladder overactivity created by PUO have shown that the transfection of plasmid expressing Maxi-K (pVAX-hSlo) can ameliorate and, in some cases, virtually normalize many characteristics of detrusor overactivity in this animal model (Chang et al. Am. J. Physiol Renal Physiol 2010; 298: F1416-F1423). Those studies were extended to a human trial in 20 women with OAB and the results at the doses studied showed safety and some potential efficacy to treat OAB, although with more restricted efficacy than observed in our preclinical studies in the rat PUO model. In this Arm we used the PUO rat model to determine whether the beneficial effects of intravesical treatment of DO with pVAX-hSlo could be improved by using a vector expressing a hSlo mutant (T352S) that encodes a Maxi-K channel with higher sensitivity to calcium (pVAX-hSlo T352S) (FIG. 10 and Gordon et al. J Pharmacol Exp Ther 2010; 334: 402-9).

The study was designed to test activity of the gene at the half log dose concentration (0, 10, 30, and 100 m) to allow the determination of the lowest effective dose. Vectors expressing genes from the CMV (pVAX) and the smooth muscle alpha actin (pSMAA) promoters were tested. An estimated total of 172 rats were used, as indicated in the TABLE 15.

The effects of intravesical treatment of PUO rats with control empty vectors, and with hSlo and hSlo T352S driven by the CMV and SMAA promoters were evaluated by cystometry (see General Methods, above) and compared among groups (see TABLE 15). At conclusion of cystometric evaluations the animals were euthanized and the bladders harvested to be used in the organ bath and electrophysiology studies (see General Methods, above) that determined the effect of each treatment on overall detrusor contractility and SMC excitability.

Rationale and preliminary data: Isolated bladder strips from patients with OAB and from animal models of DO showed increased spontaneous phasic contractions (Kinder & Mundy Br J Urol 1987; 60: 509-15; Mills et al. J Urol 2000; 163: 646-51; Banks et al. BJU Int 2006; 97: 372-8; Milicic et al., Eur J Pharmacol 2006; 532: 107-14; Oger et al. BJU Int 2011; 108: 604-11). Potassium channels appeared to play a role in the development and regulation of these phasic contractions, with decreased activity of the Maxi-K channel being implicated in greater spontaneous activity (Oger et al. BJU Int 2011; 108: 604-11; Petkov, Nat Rev Urol 2012; 9: 30-40; Karicheti & Christ Curr Drug Targets 2001; 2: 1-20; Hypolite et al. Am J Physiol Renal Physiol 2013; 304: F451-62). Previous studies using the streptozotocin (STZ) Type 1 diabetic model of bladder overactivity further supported the involvement of Maxi-K in this phenomenon.

Cystometric studies of STZ rats indicated the characteristically higher voiding frequencies and hyperactive bladder pressures (FIGS. 13A, 13B, 13C, and 13D, and Davies et al. Eur. Urol. 2007; 52: 1229-1237) and organ bath studies demonstrated that bladder strips isolated from the same animal presented increased phasic activity.

FIG. 13E, FIG. 13F shows that treatment with the Maxi-K inhibitor, iberiotoxin (IBTX) a specific inhibitor of Maxi-K channels increased the amplitude of these phasic contractions. See, Vahabi et al. BJU Int 2011; 107: 1480-7; Stevens et al. Auton Autacoid Pharmacol 2006; 26: 303-9; Tammela et al. Br J Pharmacol 1994; 113: 195-203.However, this effect was lower in strips isolated from the diabetic animal, presumably because of lower activity of the Maxi-K activity in the diabetic bladder. This prediction was supported by electrophysiological studies using a standard single whole cell patch technique to look for the functional expression of these channels (FIG. 13H). See, Davies et al. Eur. Urol. 2007; 52: 1229-1237; Wang et al. Am J Physiol Cell Physiol 2001; 281: C75-88; Wang et al. Int J Impot Res 2000; 12: 9-18.

Stepwise application of voltage across the cell membrane resulted in opening of channels and outward current flow. Recordings were made from detrusor cells isolated from 5 animals in triplicate. There was no significant difference between the outward current and applied voltage between cells isolated from STZ-diabetic animals with bladder hyperactivity and control rats. However, after addition of IBTX there was a greater decrease (>50%) in the response to the applied voltage in control compared with diabetic detrusor cells (FIG. 13H) supporting a reduction in the activity of the Maxi-K channels in the bladder detrusor muscle of diabetic animals.

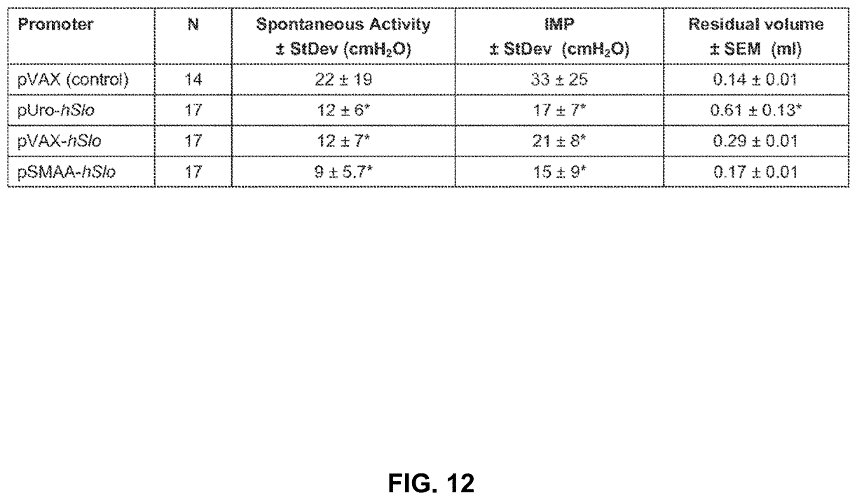

In our previous studies we observed that cystometric evaluation of PUO rats (similar to STZ rats) demonstrated a higher level of bladder spontaneous activity, a correlate for DO. Treatment with pVAX-hSlo and pSMAA-hSlo significantly ameliorated DO in these animals (see FIG. 12). Our initial cystometry studies with PUO rats treated with 30 μg of pVAX-hSlo T352S indicated that when compared to our previous data (FIG. 12) this hSlo mutant more efficiently reduced DO than the wild type gene. Based on this preliminary finding and the characteristic properties of the mutated Maxi-K channel (see FIG. 10), we expected that the mutant hSlo gene would provide a more efficient and attractive product to treat OAB.

Direct effects of hSlo and hSlo T352S expression in PUO detrusor contractility and excitability have been determined. In accordance with our preliminary cystometric findings of reduced bladder spontaneous activity in hSlo treated animals, and from our studies with the STZ model of DO demonstrating the close association of bladder overactivity with decreased Maxi-K expression, spontaneous phasic contractions of isolated bladder strips from PUO treated rats were significantly lower compared to bladder strips isolated from untreated PUO animals, and more sensitive to IBTX blockade, reflecting the increased Maxi-K expression (i.e. rescue of expression) in PUO detrusor.

Statistics: Distributions of all continuous variables were examined for normality. Those not normally distributed were transformed using a log scale and by experience the transformations were found to be reasonably normal. One-way analyses of variance were performed to determine the overall significance of differences among groups, and a Duncan's multiple comparison procedure was used to assess the significance of pair wise differences among groups. The overall level of significance was set a priori at α=0.05.

TABLE 15
Number of animals per experimental group and doses for
intravesical treatment with empty vectors (pVAX and pSMAA)
and vectors expressing hSlo and hSlo T352S (pVAX-hSlo,
pVAK-hSlo T352S, pSMAA-hSlo and pSMAA-hSlo T352S).

Dose (μg)

| | | | |
|---|---|---|---|
| 0 | 10 | 30 | 100 |

| | |
|---|---|
| Experimental groups | Number of animals |

| | | | | |
|---|---|---|---|---|
| pVAX (control) | 10 | | | |
| PVAX-hSlo | | 27 | 27 | 27 |
| PVAX-hSlo T352S | | 27 | 27 | 27 |
| pSMAA (control) | 10 | | | |
| pSMAA-hSlo | | 27 | 27 | 27 |
| pSMAA-hSlo T352S | | 27 | 27 | 27 |

### Example 6

### Generation of Nanoparticles Carrying hSlo Expression Vectors

Basic Protocol for Preparation of Hydrogel/Glass Composites:

Tetramethoxysilane (TMOS, 5 mL) was mixed with an HCl solution (560 μl of 0.2 mM HCl added to 600 μl of deionized water) and then immediately sonicated for 45 minutes in a cool water bath after which the mixture is placed on ice. D-glucose was then added to the solutions at 40 mg glucose/mL of buffered sodium nitrite solution. After the glucose had dissolved, polyethylene glycol (PEG) 400 was then added at a ratio of 1 mL PEG/20 mL of buffered solution. Chitosan [5 mg of chitosan/mL acidified distilled water (with 1 M HCl) pH 4.5] was then added at a ratio of 1 mL chitosan solution/20 mL of buffered solution. After the buffered solution was well stirred, the previously sonicated TMOS was slowly introduced at a ratio of 2 mL TMOS/20 mL buffer. The combined mixture was then stirred immediately and set aside. The resulting mixture gelled within 1-2 hours. These monolith (block) sol-gels samples were then taken out of their containers and crudely dried by blotting with paper towels prior to either heating or lyophilization. Several control samples were made with the same overall protocol, but with some lacking a specific individual component such as nitrite, glucose, chitosan and PEG. For example, an NO-free “empty gel” was made by withholding nitrite, i.e. incorporating only glucose, chitosan, and PEG.

Preparation of Heat Treated Hydrogel/Glass Composites: The sample was heated in a closed convection oven at 70° C. until the gel became a hard, white, glassy material (24-48 hours). Excessive heating resulted in a brown discoloration indicative of caramelization of the sugar. Caramelization was never observed when the sample was heated at temperature at or below 70° C. Discolored materials were discarded. The material was then placed in a planetary ball mill (Fritsch, “Pulverisette 6”) for 60 minutes at a speed of 140 rpm.

Preparation of Lyophilized Hydrogel/Glass Composites: The hydrogel monoliths generated using the above described protocols were placed into lyophilization flasks and lyophilized for 24 hours. The resulting material was a mix of coarse and fine white particulate matter. This mixture was then ground with a mortar and pestle resulting in a fine white powder.

Preparative Protocols for Nanoparticles Containing the hSlo Vectors: These protocols yielded a fine powder comprised of a relatively uniform distribution of nano-sized or nano-scale particles that were capable of sustained release of pVAX-hSlo when exposed to an aqueous environment. Hydrogel monoliths of varying thicknesses were air dried, crushed, and then heated as described above. The resulting powder was further ground using a ball mill for varying time periods.

Resultant powders and methods of making these powders can vary according to the following parameters, including, but not limited to, monolith thickness, initial drying time, heating temperature, duration of heating and duration of ball milling. Hydrogel monoliths of varying thicknesses can be air dried then lyophilized. The lyophilized material can be ground using either a mortar and pestle or ball mill. The resulting powder can then evaluated with and without a subsequent heating cycle at 50° C. for 45 minutes.

The newly formed hydrogel monoliths can be finely ground and then mixed with an equal volume of high molecular weight PEGs (oligomers or polymers of ethylene oxide, including, but not limited to, PEG3K or PEG5K) in the presence of a slight excess of buffer. The mixture can be vigorously stirred for several hours before drying and then be subjected to lyophilization. Coating the surface of hydrogel particles with large PEG molecules can enhance the dispersive properties of the resulting particles subsequent to lyophilization. Under some circumstances, PEG molecules irreversibly bind to the surface of TMOS derived hydrogels.

Tetramethoxysilane (TMOS) can be used as a foundation for hydrogel formation as described above. The following non-limiting combinations of components are contemplated:
TMOS+pVAX-hSlo;
TMOS+pVAX-hSlo+chitosan;
TMOS+pVAX-hSlo+PEG;
TMOS+pVAX-hSlo+PEG+chitosan;
TMOS contacted with monosubstituted organosilanes (e.g. alkyltrimethoxysilanes with the alkyl group being either methyl, ethyl or N-propyl)+pVAX-hSlo;
TMOS contacted with monosubstituted organosilanes (e.g. alkyltrimethoxysilanes with the alkyl group being either methyl, ethyl or N-propyl)+pVAX-hSlo+chitosan;
TMOS contacted with monosubstituted organosilanes (e.g. alkyltrimethoxysilanes with the alkyl group being either methyl, ethyl or N-propyl)+pVAX-hSlo+PEG;
TMOS contacted with monosubstituted organosilanes (e.g. alkyltrimethoxysilanes with the alkyl group being either methyl, ethyl or N-propyl)+pVAX-hSlo+glucose;
TMOS contacted with monosubstituted organosilanes (e.g. alkyltrimethoxysilanes with the alkyl group being either methyl, ethyl or N-propyl)+pVAX-hSlo+chitosan+PEG;
TMOS contacted with monosubstituted organosilanes (e.g. alkyltrimethoxysilanes with the alkyl group being either methyl, ethyl or N-propyl)+pVAX-hSlo+chitosan+glucose; and
TMOS contacted with monosubstituted organosilanes (e.g. alkyltrimethoxysilanes with the alkyl group being either methyl, ethyl or N-propyl)+pVAX-hSlo+PEG+glucose.

The strategy for this protocol was to tune the hydrophobicity of the interior of the particles by using small amounts of added alkylsubstituted silanes as a hydrophobic dopant in the sol-gel matrix (i.e. contacting an amount of alkylsubstituted silanes to a sol-gel matrix). This use of alkyl-substituted methoxysilanes generated sol-gels capable of enhancing the reactivity of encapsulated enzymes. These encapsulated enzymes had hydrophobic surfaces and lost activity and stability in pure TMOS derived sol-gel matrices. Increasing the hydrophobicity of the interior of the particles resulted in a slower release of pVAX-hSlo, thereby allowing for a sustained or more sustained delivery. Tuning the hydrophobicity of the particles was desirable if non-aqueous delivery vehicles were used for the powders.

### Example 7

### In Vitro Characterization of Nanoparticles Containing pVAX-hSlo Plasmid

pVAX-hSlo plasmid is a nucleic acid with an absorbance peak at 260 nm. Therefore, release kinetics from the nanoparticles can be determined by change in absorbance. Freshly prepared nanoparticles containing the hSlo vectors were incubated in aqueous solution for varying amounts of time (e.g. between 0 and 24 hours). Subsequently, the nanoparticles were centrifuged and the release of nucleic acids into the supernatant was determined through absorbance. Quantitative-RT-PCR, using vector-specific primers, was performed for a further characterization of the release kinetics of the nucleic acid from the nanoparticle. Stability was tested by retaining nanoparticles containing the hSlo vectors for various periods of time (ranging from, for example, 1 day to three months (or 90 days)) and determining the release kinetics of the retained nanoparticles by the same method used for freshly prepared nanoparticles. Integrity of the released plasmids was determined by agarose gel electrophoresis followed by nucleic acid staining. The results of this analysis indicated the physical form of the nucleic acid released from the nanoparticles, e.g. circular, nicked or supercoiled. Furthermore, the released nucleic acid was subjected to restriction enzyme analysis.

### Example 8

### Topical Administration of Nanoparticle Delivery System

Nanoparticles of the disclosure were used to encapsulate the Maxi-K for the present study. Data from this study demonstrated that the nanoparticles are capable of crossing the dermis. Rat models of ED showed demonstrable functional improvement following treatment.

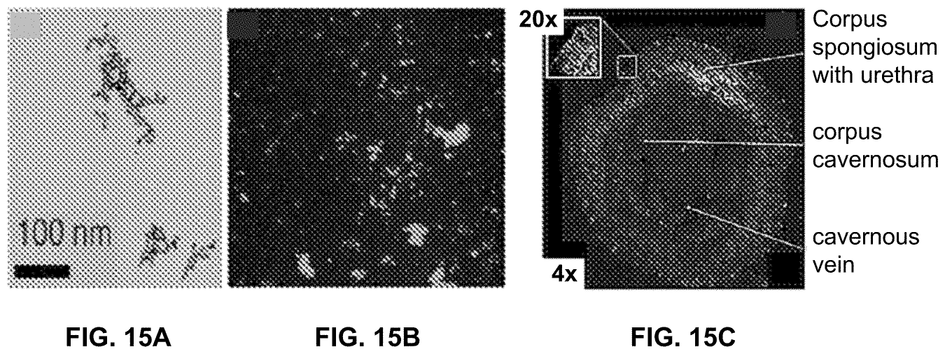

Fluorescently-labeled nanoparticles were applied to the penis of rats under anesthesia. After one hour the rat was euthanized and the entire penis washed in phosphate buffered saline and fixed in 5% paraformaldehyde for 24 hours. Cross sections were taken at various points along the shaft of the penis. A typical result is shown in FIG. 15C. Control animals (not treated with the nanoparticles) did not show any red spots. In all sections, spots could be observed at the dermis of the penis. The data indicated that these nanoparticles penetrated the dermis of the skin because washing and fixing of the penis would have removed external nanoparticles. Moreover, patches of red fluorescence could be seen in the corpora spongiosum and in the corpora vein.

Nanoparticles encapsulating erectogenic agents (NO or Sialorphin) facilitated erections in aging rats. The corpus cavernosum crus of nine month-old Sprague-Dawley rats was exposed and the intracorporeal pressure (ICP) was measured using a 23-gauge needle inserted therein. After determining a steady baseline, a viscous solution of NO- or sialorphin-containing nanoparticles was applied to the shaft of the penis. Of note, the skin of the penis remained intact and at a different location to the site of measurement of ICP). Control animals were treated with “empty” nanoparticles, containing only phosphate buffer.

A total of 7 experimental animals were used in this initial study. In 5 of the 7 animals, there was a pronounced positive effect on the intracorporeal pressure (ICP), resulting in a visible erection (tissue was prepared for histological analysis). Following histological analysis, there was no evidence of inflammation or congestion in these samples. Overall, the tissue appeared normal. These preliminary data demonstrated the ability of the engineered nanoparticles containing large molecules to cross “skin” barriers safely (without presentation of toxic effects).

### Example 9

### Biosafety/Biodistribution Profiles of Nanoparticles

There were two components to the nanoparticles: the nanoparticle and the hSlo vector. The biodistribution and pharmacokinetics of each of the components was determined. Pathology and histopathology analyses were performed to determine whether other organs were affected, and if so, which organs.

Pathology Determinations: During the physiological studies to determine the effects of the nanoparticle encapsulated hSlo vectors on bladder function the animals were monitored for potential systemic side-effects. Animals treated with the product and with nanoparticles encapsulating the empty vector (control) were monitored for several physiological parameters related to vascular well-being, such as basal heart rate, systolic pressure, diastolic pressure and mean arterial pressure. A tail cuff system was used (the CODA™2 mouse/rat tail cuff system from Kent Scientific Corp., Torrington, Conn.) which allowed non-invasive measurement of vascular physiological parameters. Following the physiological measurements animals were euthanized and gross pathology was performed. Sections of the bladder were prepared for histology and examination. In particular, signs of vascular pathology or inflammation were looked for.

Biodistribution: Nanoparticles containing the hSlo vector were instilled in the bladder lumen of healthy anesthetized rats through the indwelling bladder catheter used for cystometry. Animals were then be euthanized at different time points (from 1 hour to 1 week) and tissues removed for determination of the presence/amount of the hSlo vector or nanoparticle. The main tissues to be investigated were the bladder, blood, heart, liver, kidney, brain, spleen, testis, lung, eye, prostate, axillary lymph node, epididymis, biceps, penis and colon. The amount (dose) of product administered to perform the biodistribution studies was the same that has been shown in the studies of bladder function to induce the most significant physiological effect in reducing DO in PUO rats.

a) Nanoparticle detection: The nanoparticles used in the biodistribution experiments were labeled either by conjugation with a fluorophore (FITC or DsRed) (as in FIG. 15B) or biotinylated (to allow detection by antibodies). The organs cited above were isolated and histological sections and tissue extracts were prepared. For detection of biotinylated nanoparticles, immunohistochemistry and Western blot analysis of tissues was performed using an antibody against the biotinylated nanoparticles, which allowed for quantification of nanoparticles in individual tissues by densitometric analysis of the images. For fluorescent nanoparticles, tissue sections were examined by epifluorescence or confocal microscopy.

b) hSlo vector detection: Extensive biodistribution studies of pVAX-hSlo following its intracorporeal injection in rats were conducted. In these studies qRT-PCR was used to perform a temporal study of the plasmid distribution using primers for the kanamycin resistance gene of the pVAX vector. These studies were performed at various time points over the course of a week (4, 8, 24 hours and 1 week), which included the time points at which the physiological effect was determined. In the studies where the hSlo-nanoparticles were injected in the corpora, the plasmid could be detected in several tissues 4 hours after administration, though after one week its expression was restricted to the corpora.

A similar time course study was used to determine the biodistribution of the hSlo vectors after intravesical administration. Accordingly, the same procedure was followed to detect the hSlo vectors in the bladder tissue of PUO-treated rats. Bladders were harvested after functional cystometric assessment, the urothelial and detrusor tissues were separated under a dissecting microscope and tissues prepared for qRT-PCR analysis.

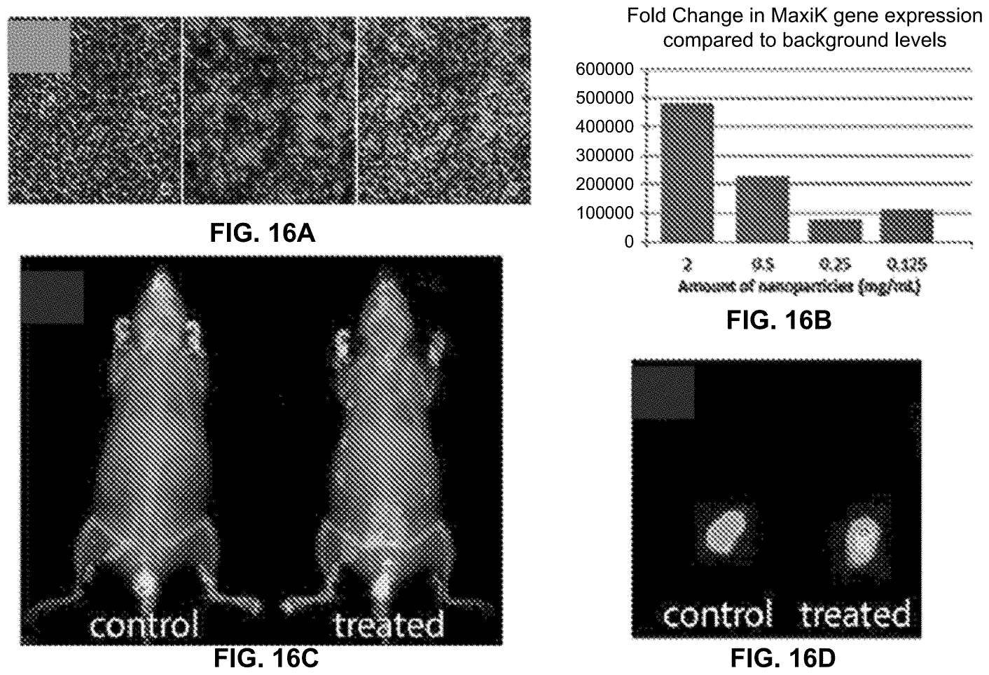

Monitoring Transfection Efficiency and hSlo Gene Expression in the Bladder: Two components determined the efficiency of transfection of cells targeted with the nanoparticles: uptake of the nanoparticles by cells and then expression of the encapsulated vector within transfected cells. Nanoparticle uptake was monitored as describe above, using biotinylated or fluorescent-tagged nanoparticles, while cargo (vector) intracellular release was determined by qRT-PCR targeting expression of the vectors' resistance genes. A similar approach, however, could not be used to detect and monitor hSlo gene expression, given that it is already endogenously expressed in the bladder. To ascertain, therefore, that upon uptake of the product the cells were actually efficiently expressing the hSlo gene, we tagged the gene with the mCherry fluorescent reporter (red) and encapsulated the product with FITC-labeled nanoparticles (see FIG. 15B). This allowed to simultaneously monitor the uptake and persistence of nanoparticles in the bladder (green fluorescence) and the hSlo expression (red fluorescence). The advantage of this approach was that it allowed for both in vivo, ex vivo and in vitro monitoring (see FIGS. 16A, 16B, 16C and 16D). Primers and antibodies for mCherry and FITC are commercially available.

Preliminary Data: In vitro studies performed with HeLa cells demonstrated that the efficiency of nanoparticle cellular uptake and expression of plasmids upon release from nanoparticles could be monitored using a fluorescent reporter gene. As show in FIG. 16A, shortly after addition of nanoparticles encapsulating a vector expressing mCherry, a high rate of transfection, approaching 95%, was observed in HeLa cells culture. Very high expression levels of the Maxi-K gene were also shown in HEK293 transfected with nanoparticles encapsulating pMaxi-K. HEK293 cells usually express very low levels of Maxi-K (FIG. 16B). Even at the lowest amount of Maxi-K-nanoparticle there was a 100,000-fold increase in gene expression after 20 h. The suitability of mCherry as a reporter for in vivo gene expression was shown in experiments in which we injected the bladder detrusor with pmCherry-N1. As shown in FIG. 16C, mCherry fluorescence can be clearly detected in the pelvic region of the treated animal, and after removal of the bladders a heat map was used to quantitate the expression (FIG. 16D).

Sample Size Considerations and Numbers of Animals: For each biodistribution study 8 animals were used for each of the five (5) time points. This number of animals was based on previous minimally acceptable numbers for biodistribution of pVAX-hSlo (accepted for safety studies for clinical trials by FDA) and also reasonable work level for analyzing 16 tissues from 8 animals in the second half period of the grant. A total of 40 female Sprague Dawley rats was used in these experiments.

### Example 10

### Determination of Nanoparticles for Intravesical Delivery

The efficacy of intravesical therapy is potentially limited by the very low permeability of the urothelium and by drug dilution with urine and washout with micturition. Chemical and physical methods have been used to enhance drug absorption by temporarily disrupting the urothelial barrier. Use of these methods, however, can cause side-effects and tissue damage. The goal in these experiments was to determine whether the use of nanoparticles as a platform for intravesical delivery of the hSlo product yielded better therapeutic results than using the hSlo alone to correct DO in PUO rats.

The plasmid construct that induced the most significant improvement in DO and the nanoparticle with the best plasmid cargo loading capability, best tissue penetration and cargo release profile, was used to manufacture the new product in sufficient quantities to be tested in the PUO model. The nanoparticle preparation was generated so that it contained the same quantity of naked vector to allow comparison between naked vector and the nanoparticle encapsulated vector.

The effects of the new product on bladder function of PUO rats was evaluated based on cystometric parameters. Cystometric data was compared to that obtained from animals treated with the naked vector. Statistical analysis was performed. The experimental groups and number of animals to be used in this Example are shown in TABLE 16.

After cystometric evaluation, the bladders from control treated and from nanoparticle+plasmid vector treated PUO rats were harvested and used for ex vivo evaluation of changes in detrusor function by organ bath and path-clamping studies.

TABLE 16
Number of animals per experimental group and doses for intravesical
treatment with control nanoparticles encapsulating the empty vector
and nanoparticles encapsulating the vector with the plasmic.

| | Dose (μg) | | |
|---|---|---|---|
| | 10 | 30 | 100 |
| Experimental groups | Number of animals | | |
| Nanoparticle + empty vector (control) | 27 | 27 | 27 |
| Nanoparticle + plasmid vector | 27 | 27 | 27 |

### Example 11

### Construction of PSMAA-hSlo Vector

The SMP8-BP-4 chimeric gene was constructed by fusing a 3.6-kb fragment of the mouse SM-α-actin to the rIGFBP-4 cDNA followed by the SV40 early polyadenylation signal fragment. SMP8 contains 21074 bp of the 59-flanking region, 63 bp of 59-UT, and the 2.5-kb first intron of SM-α-actin. A 3.6-kb SMP8 fragment, released from pSMP8 by digestion with BamHI and filled in by Klenow, was partially digested with HindIII and cloned into pRBP-4-SV at the HindIII and EcoRv sites, so that ratIGFBP-4 fused to SV40 early polyadenylation signal is driven by SMP8.

pSMAA-EYFP: A 3.7 kb fragment of pSMP8 (containing the SMAA promoter) was excised using BspluIIi/BamHI and cloned into pEYFP-N1 (Clontech) cut with the same enzymes.

pSMAA-hSlo (SEQ ID NO: 48): pVAX-hSlo was cut with BamHI to remove the hSlo gene that was ligated into pSMAA/EYFP cut with BamH1 and treated with calf intestinal alkaline phosphatase (CIP).

### Example 12

### Safety and Activity of hMaxik Gene Transfer by Intravesicular Instillation or Direct Injection

The safety and potential activity of hMaxi-K gene transfer by intravesical instillation or direct injection into the bladder wall was evaluated in female participants with idiopathic (non-neurogenic) overactive bladder syndrome (OAB) and detrusor overactivity (DO) in two double-blind, imbalanced, placebo-controlled randomized phase 1 trials. Two phase 1 trials were performed in healthy women with the OAB syndrome and urodynamically demonstrated DO, with the aim to demonstrate the safety and potential efficacy of a gene therapy plasmid vector expressing the human big potassium channel a subunit (URO-902).

ION-02 (intravesical instillation) and ION-03 (direct injection) were double-blind, placebo-controlled, multicenter studies. Active doses were administered and evaluated sequentially (lowest dose first) for safety. ION-02 participants received either 5000 μg or 10000 μg URO-902, or placebo. ION-03 participants received either 16000 μg or 24000 μg URO-902, or placebo, injected directly into the bladder wall using cystoscopy. Primary outcome variables were safety parameters occurring subsequent to URO-902 administration; secondary efficacy variables also were evaluated. Among the safety outcomes, there were no dose-limiting toxicities or significant adverse events (AEs) preventing dose escalation during either trial, and no participants withdrew due to AEs. For efficacy, in ION-02 (N=21), involuntary detrusor contractions on urodynamics were reduced at 24 weeks in patients receiving URO-902 (P<0.0508 vs. placebo), and mean urge incontinence episodes were reduced from baseline in the 5000 μg group (P=0.0812 vs. placebo). In ION-03 (N=13), significant reduction vs. placebo in urgency episodes (16000 μg, P=0.036; 24000 μg, P=0.046) and number of voids (16000 μg, 2.16, P=0.044; 24000 μg, 2.73, P=0.047) were observed 1 week after injection.

Introduction: OAB is a syndrome defined as urinary urgency, with or without incontinence, with increased daytime frequency and nocturia, in the absence of infection or other obvious pathological features. Abrams et al., Neurourol. Urodyn. 2002; 21(2):167-178. OAB is a common and significant problem that affects millions of men and women in the United States (Andersson et al., Nat. Clin. Pract. Urol. 2004; 1(2):103-108; Hashim & Abrams, Drugs. 2006; 66(5):591-606; Subak et al., Obstet. Gynecol. 2006; 107(4):908-916) with a major negative impact on quality of life (QOL). Stewart et al., World J Urol. 2003; 20(6):327-336.

Estimates for total cost of care for symptoms of OAB is upwards of $36.5 billion in the United States alone. Reynolds et al., Curr. Bladder Dysfunct. Rep. 2016; 11(1):8-13. OAB is a symptom diagnosis, which may or may not be associated with the urodynamic finding of detrusor overactivity (DO). Digesu et al., Neurourol. Urodyn. 2003; 22(2):105-108.

Primary pharmacologic therapy for OAB consists of oral antimuscarinics or adrenergic beta-3 receptor agonists. Lightner et al., J Urol. 2019:101097JU0000000000000309; Maman et al., Eur Urol. 2014; 65(4):755-765; Warren et al., Ther Adv Drug Saf. 2016; 7(5):204-216. However, these drugs lack bladder selectivity and are not effective in all patients. In addition, significant side effects such as dry mouth, constipation, and cognitive defects limit use of many antimuscarinic agents. Yamada et al., Pharmacol Ther. 2018; 189:130-148; Coupland et al., JAMA Intern Med. 2019; 179(8):1084-1093.

Lack of efficacy and side effects have resulted in low long-term treatment persistence (ranging from 5% to 47%). Chancellor et al., Clin Ther. 2013; 35(11):1744-1751; Yeowell et al., BMJ Open. 2018; 8(11):e021889.

Chemodenervation agents for treatment of OAB and DO, such as botulinum toxin (e.g., onabotulinumtoxinA) are limited by side effects, including incomplete bladder emptying/urinary retention requiring catheterization and urinary tract infections. Moga et al., Toxins (Basel). 2018; 10(4):169. Thus, more effective and/or tolerable alternative treatments would be welcomed.

The large-conductance Ca²⁺-activated K⁺ (also known as big potassium [BK], MaxiK⁺, BK_{Ca}, K_{Ca}1.1) channel is highly expressed on urinary bladder smooth muscle cells and is undeniably an important and physiologically relevant K⁺ channel that regulates bladder detrusor muscle function. Petkov, American journal of physiology. 2014; 307(6):R571-R584; Latorre et al., Physiol Rev. 2017; 97(1):39-87. BK channels are activated by changes in both voltage and cytoplasmic Ca²⁺ and control cellular excitability and, thus, degree of smooth muscle contraction. Petkov, American journal of physiology. 2014; 307(6):R571-R584; Latorre et al., Physiol Rev. 2017; 97(1):39-87. Activation of the BK channel reduces smooth muscle cell excitability and may be a potential therapeutic option for treatment of OAB. Hristov et al., Am J Physiol Cell Physiol. 2012; 302(11):C1632-1641. Gene therapy using a plasmid vector has demonstrated that overexpression of the human BK channel a subunit (pore forming unit) alters tissue/organ function in both animal and human applications. Christ et al., Eur Urol. 2009; 56(6):1055-1066; Christ et al., Urology. 2001; 57(6 Suppl 1):111; Melman et al., Isr. Med. Assoc. J. 2007; 9(3):143-146.

Data from two phase 1 trials demonstrating safety and potential efficacy of URO-902, comprising a gene therapy plasmid vector expressing the human BK channel a subunit, are presented below. In these studies, URO-902 was delivered either by a single intravesical instillation or by direct injections into bladder detrusor muscle.

### I. MATERIALS AND METHODS

URO-902 is a non-viral, double-stranded, naked plasmid DNA molecule (6880 bp) derived from a pVAX (Invitrogen) backbone and hSlo cDNA. Expression of hSlo is driven by the cytomegalovirus promoter, and transcript maturation is supported with the bovine growth hormone poly(A) site. The construct also contains the kanamycin resistance gene and the pUC origin of replication. Melman et al., Hum Gene Ther. 2006; 17(12): 1165-1176.

### Study Design

Both the intravesical instillation (ION-02, NCT00495053) and direct injection

(ION-03, NCT01870037) studies were double-blind, placebo-controlled, multicenter, sequential active-dose, phase 1 studies in healthy female of ≥18 years and non-childbearing potential, with moderate OAB of ≥6 months' duration with associated DO and at least one of the following: micturitions ≥8 times per day, symptoms of urinary urgency (sudden compelling desire to urinate) or nocturia (waking at night ≥2 times to void), urgency incontinence (≥5 incontinence episodes per week), and DO with ≥1 uncontrolled phasic contraction(s) of at least 5 cm/H₂O pressure documented on cystometrogram. Additional inclusion criteria were residual volume of ≤200 mL, non-response and/or poor tolerance to previous OAB treatments (e.g., antimuscarinic/anticholinergic agents, beta-3 agonists, or onabotulinum toxin A), and did not wish to continue these treatments. Exclusion criteria included a positive serum (HCG) pregnancy test or lactating, history of 3 or more urinary tract infections/year, and any significant genitourinary disorder, except incontinence.

In both studies, active doses were administered and evaluated sequentially (lowest dose first) for safety. Enrollment of the first 4 participants in each cohort was managed by the study sites with a 2-day waiting period following each participant's dosing. The next participant was enrolled only after the site had contacted the previously dosed participant on day 3 following transfer to determine if a clinically significant adverse event (AE) had occurred. If a clinically significant AE was reported, the medical monitor was to contact all the sites, and no further enrollment was to be done until the medical monitor or sponsor gave permission.

Participants in the intravesical instillation (ION-02) study received a single administration of either 5000 μg or 10000 μg URO-902, or placebo in PBS-20% sucrose solution (each dose was 90 mL total volume). Up to 13 female participants were to be enrolled per dose level (10 on active treatment, 3 on placebo). Patients in the direct injection study (ION-03) received a single administration of URO-902 in PBS-20% sucrose of either 16000 μg (4 mL total as 20 distributed 0.2 mL injections) or 24000 m (6 mL total as 30 distributed 0.2 mL injections), or placebo (either 20 or 30 distributed injections) directly into the bladder wall using cystoscopy. Up to 9 female participants were to be enrolled per dose level (6 on active treatment, 3 placebo).

Study periods for both ION-02 and ION-03 were 6 months following treatment with URO-902. Post-treatment visits occurred at weeks 1, 2, 4, 8, 16, and 24. At pre-specified intervals, physical examinations, electrocardiogram (including, chemistry, hematology and urine laboratory samples, cystometry, daily voiding diary information, pad test results, and bladder scans were performed and reviewed. Urine samples for detection of hSlo DNA were collected at each visit in both studies. Blood samples for detection of hSlo DNA were collected at two hours post-injection. All participants who received the study drug were surveyed post study to monitor for delayed AEs at 6, 12, and 18 months after completing the initial 6-month study period.

### Intravesical Instillation (ION-02) and Direct Injection (ION-03) Procedures

ION-02, intravesical instillation procedure: Each 90 mL dose was instilled through a small diameter catheter into the lumen of the bladder. Participants were requested to retain the solution for at least 2 hours (dwell time).

ION-03, direct injection procedure: Treatments were administered without general or regional anesthesia through a rigid cystoscope 10 to 20 minutes after 40 mL of 2% lidocaine was instilled into the bladder and 10 cc of 2% xylocaine gel was instilled into the urethra. URO-902 was injected with a BONES needle into the detrusor muscle, avoiding the trigone. The needle was inserted approximately 2 mm into the detrusor and 20 injections of either 0.2 mL (16000 μg dose) or 30 injections of 0.2 mL (24000 μg dose) each were spaced approximately 1 cm apart.

### Safety and Efficacy Assessment

The primary outcome variables for both ION-02 and ION-03 included all safety parameters occurring subsequent to administration of URO-902 compared with placebo, including all AEs, change from baseline for all clinical laboratory tests, measurements for the presence of hSlo in urine and/or blood, electrocardiograms (rate, rhythm, PR, QT, QT_{c}F, QT_{c}B, QRS), and physical examinations. Urinary tract infection was defined as a positive urine culture (≥1000 colonies/mL) of a urinary pathogen from a catheterized urine. Urinary retention was defined as ≥400 mL of urine measured by bladder scan. Only treatment emergent adverse events (TEAEs) were evaluated.

Secondary outcome variables were measured to determine efficacy and potential activity of URO-902 in participants with OAB/DO. The secondary efficacy variables were changes in mean scores from baseline to weeks 1, 2, 4, 8, 12, and 24 after the single administration of URO-902 and included diary variables, such as the number of daily micturitions, urgency incontinence episodes, and urgency episodes (daily volume voided per micturition also was recorded in the ION-03 study). Also included were the change in the mean rating from baseline of QOL scores from the King's Health Questionnaire (KHQ). Urodynamics were performed at baseline and at weeks 4 and 24. Urodynamic variables included cystometric capacity and assessment of involuntary detrusor contractions. The urodynamics were interpreted by a blinded central reader.

### Data Analysis

Both safety and efficacy data were summarized using summary descriptive statistics by treatment group (combined placebo vs. 2 active treatment groups and combined placebo vs. combined treatment groups) and the total study population. Linear mixed effect models were used to estimate difference of changes from baseline between placebo and active treatment and to test whether there was dose-response for different outcomes. Generalized estimating equation model was used to estimate effects for the binary endpoints.

For exploratory analysis, analysis of variance or analysis of covariance with baseline measure as covariate was applied to test for treatment difference at each separate week. Chi-square was used to test for difference in treatment vs. placebo in participants' perception of response to treatment. Given the small sample size and exploratory nature of the efficacy data, no adjustment was made for multiple comparisons. All the P-values presented were nominal P-values.

### II. RESULTS

### Patient Demographics

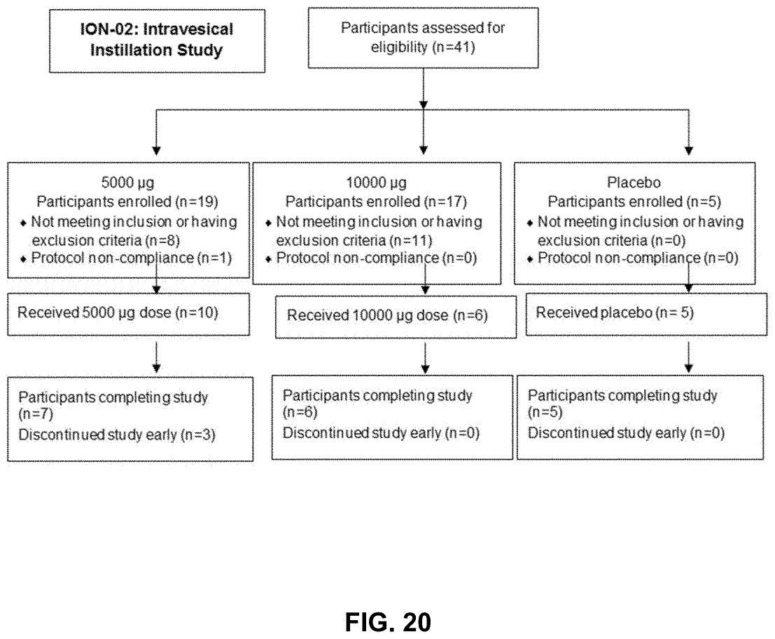

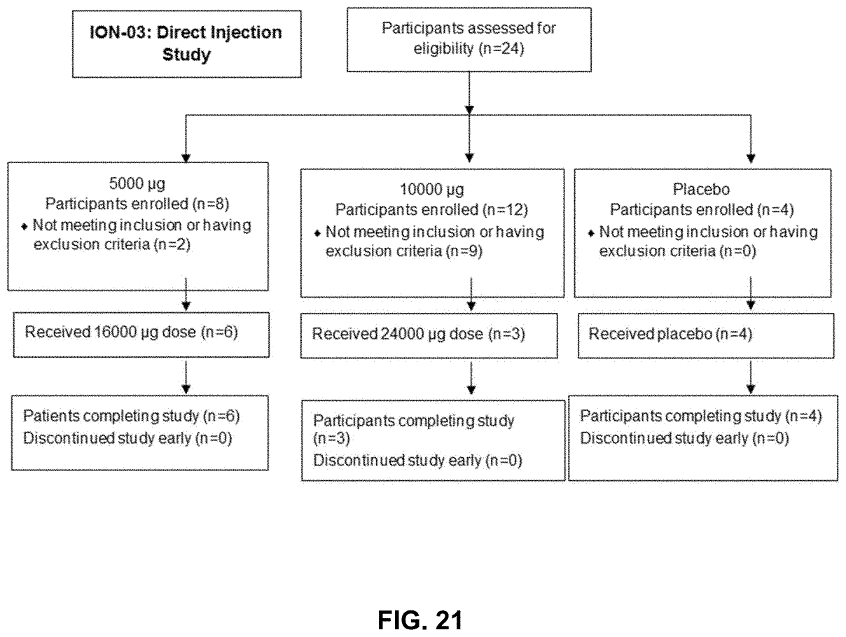

Forty-one participants were screened for ION-02 (intravesical instillation); 20 were excluded because they did not meet inclusion/exclusion criteria. In ION-3, 24 patients were assessed, and 9 were excluded. The full CONSORT diagrams for both studies can be seen in FIG. 20 and FIG. 21. All the participants in both studies had unsuccessful prior treatment with anticholinergics, and 4 had issues with a botulinum toxin A therapy in ION-03. Patient demographics and baseline characteristics were generally comparable between treatment groups in both studies (TABLES 17 and 18).

TABLE 17
Patient demographics from ION-02 intravesical instillation study

| | | URO-902 | URO-902 | |
|---|---|---|---|---|
| | | 5000 μg | 10000 μg | Placebo |
| N | | 10 | 6 | 5 |
| Age (years) | Mean (SD) | 62.6 (15.2) | 65.8 (14.4) | 69.8 (9.8) |
| | Min, Max | 45, 93 | 47, 80 | 56, 83 |
| Race | White | 9 | 6 | 4 |
| | Black/African American | 1 | 0 | 0 |
| | Latino/Hispanic | 0 | 0 | 1 |
| Baseline mean | Mean (SD) | 11.5 (3.2) | 11.2 (4.7) | 10.1 (3.2) |
| number of urgency | | | | |
| episodes (24 hrs) | | | | |
| Baseline micturition | Mean (SD) | 11.5 (3.4) | 11.2 (4.7) | 10.1 (3.2) |
| frequency (24 hrs) | | | | |
| Baseline mean | Mean (SD) | 2.7 (2.3) | 2.2 (2.2) | 5.3 (3.6) |
| number of urgency | | | | |
| incontinence | | | | |
| episodes (24 hrs) | | | | |

BMI, body mass index; max, maximum; min, minimum; SD, standard deviation.

TABLE 18
Patient demographics from ION-03 direct injection study

| | | URO-902 | | URO-902 | | | |
|---|---|---|---|---|---|---|---|
| | | 16000 μg | | 24000 μg | | Placebo | |
| N | | 6 | | 3 | | 4 | |
| Age (years) | Mean (SD) | 55.8 | (4.6) | 65.1 | (9.2) | 57.0 | (6.8) |
| | Min, Max | 50.2, 62.9 | | 57.8, 75.5 | | 51.0, 66.7 | |
| Race | White | 2 | | 2 | | 4 | |
| | Black/African | 4 | | 1 | | 0 | |
| | American | | | | | | |
| Ethnicity | Latino/Hispanic | 0 | | 1 | | 0 | |
| | Not | 6 | | 2 | | 4 | |
| | Latino/Hispanic | | | | | | |
| Height (cm) | Mean (SD) | 25.3 | (0.9) | 24.5 | (0.8) | 26.0 | (0.9) |
| | Min, Max | 24.4, 26.6 | | 23.6, 25.2 | | 24.8, 26.8 | |
| Weight (kg) | Mean (SD) | 86.4 | (29.8) | 62.6 | (14.7) | 78.6 | (23.4) |
| | Min, Max | 49.5, 120.0 | | 52.7, 79.5 | | 57.3, 109.1 | |
| BMI (kg/m2) | Mean (SD) | 32.7 | (12.6) | 24.9 | (5.6) | 27.7 | (7.0) |
| | Min, Max | 19.6, 48.3 | | 19.9, 31.0 | | 21.9, 36.5 | |
| Baseline mean | Mean (SD) | 10.21 | (3.55) | 17.19 | (7.07) | 9.82 | (5.17) |
| number of | | | | | | | |
| urgency episodes | | | | | | | |
| (24 hrs) | | | | | | | |
| Baseline | Mean (SD) | 11.26 | (2.70) | 17.19 | (7.07) | 10.18 | (4.78) |
| micturition | | | | | | | |
| frequency (24 | | | | | | | |
| hrs) | | | | | | | |
| Baseline mean | Mean (SD) | 1.91 | (0.83) | 3.81 | (3.30) | 1.82 | (1.52) |
| number of | | | | | | | |
| urgency | | | | | | | |
| incontinence | | | | | | | |
| episodes (24 hrs) | | | | | | | |

BMI, body mass index; max, maximum; min, minimum; SD, standard deviation.

Safety Results

There was no detectable evidence of URO-902 in the urine of any participant during ION-02. In ION-03, one participant had URO-902 detected in the blood, and 4 participants had URO-902 detected in the urine immediately after dosing (subsequent assays were negative). No dose-limiting toxicities or significant AEs occurred to prevent escalation to the next higher dose during either trial. Only one serious AE, unrelated to study drug, was reported in ION-03, in a woman with pre-existing asthma who had an exacerbation of her condition due to cold weather that required treatment.

Three participants in ION-02 had TEAEs considered related or possibly related to study treatment, all in the 5000 μg URO-902 dose group. One was a Mobitz type 11 second degree AV block at 170 days post treatment that resolved in one day. She had a first degree AV block predosing from week 0 to 1 week post dosing.

No participants withdrew from either study due to adverse events. No deaths occurred during the studies. The majority of AEs reported were mild in severity and unrelated to treatment. No medical problems were reported during the post study 18-month long-term follow-up. Urinary retention was not seen in any participants on active treatment. In addition, there were no participants on active treatment with worsening of symptoms of OAB as measured by diary, KHQ, or deterioration on urodynamics.

### Efficacy in ION-02

Although these were escalating-dose safety studies, secondary efficacy endpoints were evaluated. In ION-02 there were some positive findings to suggest that this gene therapy treatment could be efficacious. There was a near significant trend in the overall mean difference of the number of decreased detrusor contractions from baseline at 24 weeks after transfer, as measured by urodynamic evaluation (P<0.0508). At week 8, there was also a trend in the 5000 μg dose group with an observed >40% mean decrease in urgency incontinence episodes from baseline (P=0.0812).

### Efficacy in ION-03

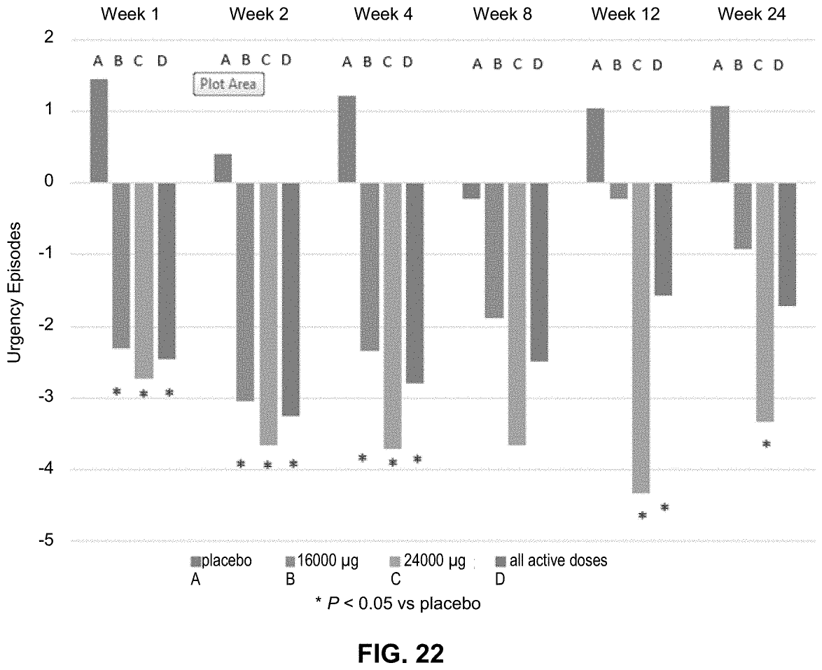

The utility of URO-902 as a viable treatment for OAB was more apparent when the plasmid was injected directly into the detrusor. Despite the small population that was enrolled, the ION-03 study demonstrated rapid and sustained improvements in multiple secondary efficacy endpoints in participants with OAB. Significant improvements also were observed in the mean reduction in number of voids/24 hours, comparing placebo with 1 week after injection of URO-902 (placebo, mean at 1 week: 11.27, mean change from baseline: +1.45; 16000 μg, mean at 1 week: 7.89,mean change from baseline: −2.31,P=0.036; 24000 μg, mean at 1 week: 14.46, mean change from baseline: −2.73, P=0.046) (FIG. 22). This improvement was generally maintained throughout the 24-week study with significant improvements in at least one dose group at weeks 2, 4, 12, and 24 after administration.

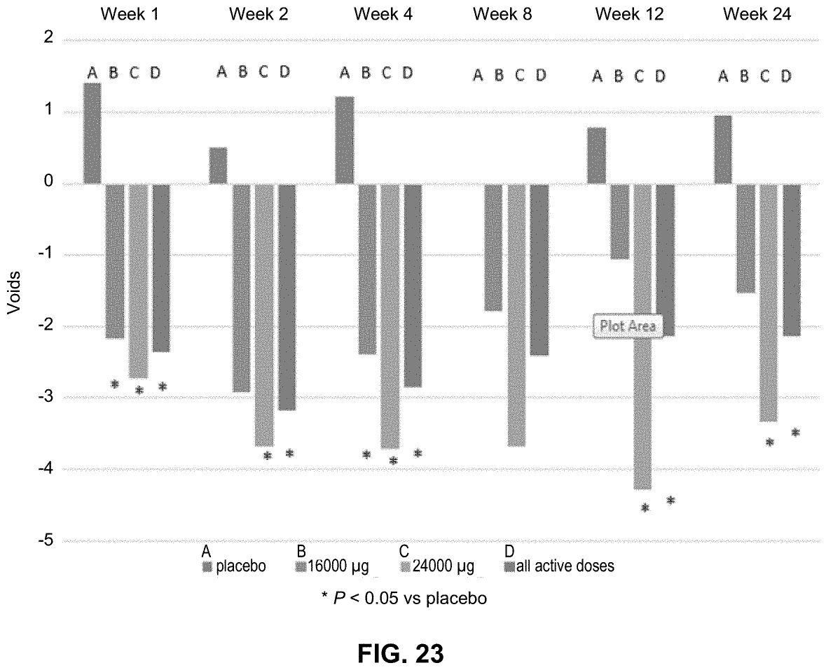

Significant improvements also were observed in the mean number of voids/24 hours compared with placebo 1 week after injection for both active doses (placebo, mean: 11.59 at 1 week, mean change from baseline: +1.41; 16000 μg,mean: 9.10 at 1 week, mean change from baseline: −2.16,P=0.044; 24000 μg,mean:14.46 at 1 week, mean change from baseline: −2.73, P=0.047) (FIG. 23). These improvements were generally maintained up to 24 weeks post injection with significant improvements observed in all testing weeks except for week 8. For both urgency episodes and voids, there were no significant differences between the 2 active treatments of URO-902 (16000 μg and 24000 μg), likely because of the small number of participants. However, there was a trend toward a longer duration of effect in the 24000 μg dose group (FIG. 22 and FIG. 23).

Significant reductions in the number of urgency incontinence episodes in the active treatment groups relative to placebo were not observed. However, significant reductions from baseline were seen at weeks 2, 4, 8, and 12, in at least one of the active treatment doses (16000 m or 24000 m), and at week 24 both active doses had significant reductions from baseline in urgency incontinence episodes (16000 82 g, −1.29, P=0.015; 24000 μg, −2.29, P=0.005). In the placebo group, no significant reductions from baseline in urgency incontinence episodes were observed at any timepoint.

Participant perception of response to treatment also was improved significantly in the combined active treatment dose group vs. placebo at weeks 1 (P=0.019) and 4 (P=0.0126) post treatment. At week 1, roughly 44% of the participants administered URO-902 reported a little benefit, and another 44% reported very much benefit. Only 25% of the participants administered placebo at week 1 reported a little benefit, and none reported very much benefit.

QOL parameters as assessed with KHQ showed statistically significant mean improvements for the individual active treatments and for the combined active treatment groups vs. baseline and vs. placebo in many of the domains (including Domain 2: Impact on Life, Domain 3: Role Limitations, Domain 4: Physical Limitations, Domain 5: Social Limitations, and Domain 8: Sleep Energy). Consistent and durable improvement throughout the study was especially observed in Domain 3 of the KHQ with both active doses. Significant improvements in Role Limitations scores from baseline and significant improvements relative to placebo were observed at all of the assessed timepoints (weeks 4, 8, 12, and 24).

### III. DISCUSSION

Current therapeutic options for OAB are limited, thus new approaches to treatment of this widespread condition are needed. The BK channel is an important regulator of detrusor muscle cell excitability, and modulation of this channel's activity using gene therapy is one such novel approach. Although mechanistically attractive, attempts at pharmacological activation of potassium channels has not been clinically successful in the treatment of OAB. Chapple et al., Eur Urol. 2006; 49(5):879-886.

URO-902 represents a localized gene therapy approach to treating a benign bladder condition of OAB/urgency incontinence. Instillation of vectors designed to overexpress the BK channel significantly decreases hypercontractility of the bladder of rat models and pre-clinical studies have shown that the tissue over expression lasts for up to 6 months. Christ et al., Urology. 2001; 57(6 Suppl 1):111. Modulating the expression levels of BK channels with URO-902 may possibly treat OAB/DO by reducing the excitability of the detrusor smooth muscle. This makes hSlo gene transfer using URO-902 a potentially attractive gene therapy option for OAB.

Regarding the safety outcomes in these studies, systemic exposure to URO-902 as measured by serial urine, blood, and EKG studies was minimal, supporting a local organ effect with little risk of systemic implications. Moreover, there were no organ specific safety signals such as urinary retention with URO-902. Urinary retention and the need for subsequent urinary catheterization can limit the application of other therapies, such as chemodenervation, in the treatment of OAB.

For the secondary efficacy outcomes, in ION-03, statistically significant reductions in the number of voids and urgency episodes were clearly observed when URO-902 was injected directly into the detrusor. Lesser efficacy was noted with the lower dose intravesical instillation (ION-02). This difference may be dose related or because of the relative difficulty in crossing the urothelial barrier with intravesical instillation compared with direct injection.

Direct injection into the bladder wall, relative to bladder instillation, appears to be a more definitive way to deliver the gene transfer product for optimal effect.

Overall, no significant difference between the 16,000 μg and 24,000 μg doses were observed, possibly due to the small number of participants in the 24,000 μg group. Nevertheless, the duration of the effect appeared to be longer for the 24,000 μg group than for the 16,000 μg group.

The efficacy results from the diary variables were mirrored when participants were asked for their opinion of their response to treatment using the KHQ, where multiple post dose visits throughout the study reported statistically significant improvements in many of the domains assessing QOL parameters (Impact on Life, Role Limitations, Physical Limitations, Social Limitations, and Sleep).

Although levels of the BK channel gene expression resulting from gene transfer of the plasmid were not determined, data from this and other studies indicated that enough gene was expressed to modulate smooth muscle tone and that it lasts for up to six months. Melman et al., Isr. Med. Assoc. J. 2007; 9(3):143-146; Melman et al., Hum. Gene Ther. 2006; 17(12):1165-1176; Christ et al., Am. J. Physiol. 1998; 275(2):H600-H608; Melman et al., J. Urol. 2003; 170(1):285-290.

Gap junctions (connexin 43) connecting urinary bladder smooth muscle cells create a syncytium throughout the detrusor that allows for the rapid passage of ions and second messenger signals along the entire structure, and thus, could enable functional effects even with relatively small changes in BK expression levels. As such, even limited uptake of URO-902 into a fraction of bladder cells is expected to have a robust effect on overall bladder function.

### IV. CONCLUSION

The safety and efficacy demonstrated in these two preliminary phase 1 studies suggested that modulation of BK channel expression levels using gene transfer can be used as therapy to treat OAB and other smooth muscle dysfunction-related diseases or conditions. Intravesical gene therapy is a minimally invasive, organ-specific approach with little risk of untoward collateral effects elsewhere in the body, haven the potential for a long duration of activity.

### Example 13

### Phase 2A Study Evaluating the Efficacy and Safety of Uro-902 in Subjects with Overactive Bladder and Urge Urinary Incontinence

### I. BACKGROUND

URO-902 (pVAX-hSlo) is a GMP manufactured double-stranded deoxyribonucleic acid (DNA)-plasmid vector based gene therapy product for the treatment of OAB. URO-902 is a GMP manufactured DNA-plasmid (pVAX vector) containing a cDNA insert encoding the pore-forming a subunit of the human smooth muscle Maxi-K channel, hSlo. The Maxi-K channel is a prominent and well-studied K channel subtype involved in smooth muscle relaxation. Because heightened smooth muscle tone can be a causative factor of OAB with DO, increased numbers of Maxi-K channels in the bladder detrusor smooth muscle cells associated with effective URO-902 treatment can improve this condition.

Treatment with URO-902 increases the number of Maxi-K channels in the cell membrane, resulting in a greater efflux of K⁺ from the cell after cell activation by a normal stimulus. The free intracellular calcium concentration is an important determinant of smooth muscle cell tone. An increase in the intracellular calcium level is associated with increased smooth muscle tone (contraction), and a decrease in intracellular calcium levels is associated with decreased smooth muscle tone (relaxation).

In smooth muscle, the outward movement of K⁺ causes a net movement of positive charge out of the cell, making the cell interior more negative with respect to the outside. This has two major effects. First, the increased membrane potential ensures that the calcium channel spends more time closed than open. Second, because the calcium channel is more likely to be closed, there is a decreased net flux of Ca²⁺ into the cell and a corresponding reduction in the intracellular calcium levels. The reduced intracellular calcium leads to smooth muscle relaxation. Having more Maxi-K channels in the cell membrane leads to greater smooth muscle cell relaxation. Detailed information on the Maxi-K channels and their role in OAB syndrome is also provided in EXAMPLE 14.

An extensive series of in vitro and in vivo nonclinical studies evaluating the activity and safety of URO-902 have been conducted. Data from completed URO-902 nonclinical studies are summarized in EXAMPLE 14 and the examples above. These studies included both OAB as well as erectile dysfunction (ED) animal models. The ability of pcDNA/hSlo to transfect cells, express hSlo, and localize the Maxi-K channel to the cell membrane was demonstrated in in vitro experiments using the 293 human embryonic kidney cell (HEK293) and Xenopus oocytes. In in vivo pharmacology studies, single administration by transperitoneal instillation into the rat bladder of 0.1, 0.3, and 1 mg URO 902 resulted in a nearly complete ablation of DO compared with controls in the partial urethral outlet (PUO) obstruction rat model.

In ED animal models (rats and monkeys), increases in erectile response were observed with hSlo compared with controls. A single administration of 0.01 mg, 0.1 mg, or 1 mg pcDNA/hSlo via intracorporal injection in rats was well tolerated and was associated with no histopathological changes in major organ tissues at any dose. Repeat administration of 0.1 mg pcDNA/hSlo intracorporally did not increase the intracorporal pressure/blood pressure (ICP/BP) ratio more than a single 0.1 mg dose and was not associated with detectable adverse effect on clinical cardiovascular parameters.

Extensive biodistribution studies at the 10 copy pVAX-hSlo level were conducted in rats administered doses ranging from 0.01 to 1 mg of intracavernous URO-902 and 0.1 to 1 mg URO-902 by transperitoneal intravesical administration. Major organs were examined at 1, 4, 8, and 24 hours and 1, 2, and 4 weeks after transfer. In the transperitoneal intravesical study, approximately 13 million copies of plasmid were detected at 1 week in the bladder per microgram of total DNA. No signal of gene transfer was detected at any time point in either cardiac tissue or testes tissue after administration of URO-902.

In another study, supercoiled pVAX-hSlo became nicked open circular plasmid DNA within 30 minutes in whole blood. Thus, active gene expression would be limited should URO-902 enter the systemic circulation.

To date, 4 clinical studies have been completed by the prior sponsor in a total of 80 subjects (34 women with OAB and 46 men with ED). Two Phase 1 studies evaluating single administrations of URO-902 have been completed in female subjects with OAB: Study ION-02 evaluated intravesical instillation and Study ION-03 evaluated intradetrusor injection (via cystoscopy).

Single administrations of URO-902 at 5 mg/90 mL and 10 mg/90 mL via intravesical instillation (Study ION-02) and single administrations of URO-902 at 16 mg and 24 mg via intradetrusor injections into the bladder were well-tolerated in female subjects with moderate OAB and DO. The majority of treatment-emergent adverse events (TEAEs) were unrelated to study treatment. No serious adverse events (SAE) were reported in Study ION-02 and the one SAE reported in Study ION-03 was considered unrelated to treatment by the investigator. No treatment-related deaths were reported and there were no study discontinuations due to TEAEs. Preliminary efficacy results from both studies indicated positive efficacy findings despite the small number of subjects in each study.

In Study ION-03, a Phase 1, multicenter, double-blind, placebo-controlled design study evaluating 2 escalating doses of URO-902 (16 mg and 24 mg) administered by direct injections into the bladder wall/detrusor muscle, statistically significant changes were observed vs. placebo and baseline at doses of 16 and 24 mg for 2 of the subject diary variables: number of voids and urgency episodes per 24 hours. In addition, the urgency incontinence episodes showed significant changes compared to baseline, although not placebo. These changes occurred over multiple visits out to the final Week 24 posttreatment follow-up visit.

In addition, Phase 1 (Study ION-301) and Phase 2 (Study ION-04 ED) studies evaluating single intracavernous injections of URO-902 have been completed in male subjects with ED. Single intracavernous injections of URO-902 at doses ranging from 0.5 mg to 16 mg were well tolerated in male subjects with ED (Studies ION-301 and ION-04 ED). The majority of adverse events reported were mild to moderate in severity and not treatment-related. Only 2 SAEs were reported in each study and all were unrelated to study treatment. No deaths occurred during either of the studies. Data from completed URO-902 clinical studies are summarized in Example 14.

### II. OBJECTIVES AND ENDPOINTS

The objectives of this study are (1) to evaluate the efficacy of a single dose of

URO-902 24 mg and 48 mg (administered via intradetrusor injection), compared with placebo, in subjects with OAB and UUI up to 48 weeks post-dose, and (2) to evaluate the safety and tolerability of a single dose of URO-902 24 mg and 48 mg (administered via intradetrusor injection), compared with placebo, in subjects with OAB and UUI up to 48 weeks post-dose. This study has no formal statistical primary endpoint hypothesis.

Study endpoints include efficacy endpoint, safety endpoints (e.g., adverse events), and other endpoints (e.g., hSlo cDNA concentrations in blood or urine). Efficacy endpoint include, e.g., change from baseline at Week 12 in average daily number of UUI episodes; change from baseline at Week 12 in average daily number of micturitions; change from baseline at Week 12 in average daily number of urinary incontinence (UI) episodes; change from baseline at Week 12 in average daily number of urgency episodes; proportion of subjects achieving ≥50%, ≥75%, and 100% reduction from baseline at Week 12 in UUI episodes per day; change from baseline at Week 12 in average volume voided per micturition; health outcomes parameters (e.g., change from baseline at Week 12 in total summary score from the Urinary Incontinence-Specific Quality-of-Life Instrument (I-QOL), change from baseline at Week 12 in OAB Questionnaire (OAB-q) scores, or overall change of bladder symptoms based on the Patient Global Impression of Change (PGI-C) scale score at Week 12), urodynamic parameters (e.g., cystometric volume at 1^{st} sensation to void (CV1^{st}sen), maximum cystometric capacity (MCC), maximum detrusor pressure during the storage phase (P_{detmax}), presence/absence of the first involuntary detrusor contraction (IDC) and, if present (i) volume at first IDC (V_{PmaxIDC}), (ii) maximum detrusor pressure during the first IDC (P_{maxIDC}),

### III. OVERALL STUDY DESIGN

Study Treatment Groups: URO-902 (24 mg or 48 mg) will be administered as intradetrusor injections via cystoscopy. A single treatment of URO-902 24 mg will be administered to subjects in Cohort 1. An independent Data and Safety Monitoring Board (DSMB) will make recommendations regarding dose escalation only after unblinded review of safety data from all subjects in Cohort 1 up to Week 6. Study treatment at the higher dose (URO-902 48 mg) will begin only after the DSMB has recommended it is safe to proceed to Cohort 2.

Controls: Matching placebo (phosphate buffered saline with 20% sucrose [PBS-20%]) in Cohort 1 and Cohort 2.

Dosage/Dose Regimen: For each subject in Cohort 1 or Cohort 2, a single treatment will be administered on Day 1 after fulfillment of the “day of treatment criteria.”

Randomization/Stratification: An estimated total of 78 subjects will be enrolled into 2 cohorts, with approximately 39 subjects randomized into each cohort. In both cohorts, subjects will be randomized in a 2:1 ratio to receive either URO-902 (24 mg or 48 mg) or placebo. Each cohort will be randomized separately, and enrollment will be sequential, starting with Cohort 1 (URO-902 24 mg [n=26] and placebo [n=13]) and followed by Cohort 2 (URO-902 48 mg [n=26] and placebo [n=13]). At the Randomization Visit, subjects in both Cohort 1 and Cohort 2 will be randomized centrally to receive either a single treatment of URO-902 or matching placebo. Randomization will be stratified by baseline UUI episodes per day and presence or absence of DO.

Visit Schedule: Study visits will be identical for Cohorts 1 and 2. Subjects will be evaluated during a 2-week screening period for eligibility (Days −35 to −21). Eligible subjects will be randomized to treatment at the Randomization Visit (Day −14 to Day −7) within each cohort; however, subjects will be administered the study treatment via cystoscopy on Day 1. All subjects will be evaluated at scheduled post-treatment clinic visits at Weeks 2, 6, 12, 18, and 24, or until the subject exits the study. Afterwards, 2 follow-up telephone visits will be performed at Week 36 and Week 48.

Additional OAB treatment: Starting at Week 24, subjects can request and be prescribed additional OAB treatment(s) at the clinical discretion of the investigator. Subjects who receive additional OAB treatment(s) at Week 24 or after will only be followed to assess adverse events at any future telephone visits (Week 36 and/or Week 48). No efficacy assessments will be performed once a subject is prescribed an additional OAB treatment.

Number of Subjects: Approximately 78 adult female subjects will be randomized into the 2 cohorts, with approximately 39 subjects randomized into each cohort.

Statistical Methods: The following analysis populations will be evaluated: safety, intent-to-treat exposed (ITT-E) and ITT-E (modified). The safety population will consist of all subjects who received the study medication and will be used to assess treatment-emergent adverse events and other safety evaluations based on actual treatment received. ITT-E will be used for demographics, baseline characteristics, and efficacy analyses up to Week 24.

The ITT-E population will consist of all subjects randomized and treated subjects from Cohorts 1 and 2. ITT-E (modified), which will consist of subjects in the ITT-E who did not receive additional OAB treatment(s) after Week 24, will be used to evaluate efficacy after Week 24. Interim analyses may be conducted when ≥50% of subjects in Cohort 1 and/or when ≥50% of subjects in Cohort 2 have completed at least 12 weeks of follow-up post-randomization (or prematurely exited the study prior to Week 12) for future planning purposes.

A planned interim analysis will be performed to evaluate the objectives of the protocol at Week 12, after all subjects in Cohorts 1 and 2 have completed the Week 12 Visit (or prematurely exited the study prior to Week 12). The final analysis will be performed after all subjects have completed the study. Details of the interim analyses and final analysis will be described in the Statistical Analysis Plan.

The study has no formal statistical primary endpoint hypothesis. Descriptive statistics will be used to evaluate the efficacy and safety endpoints. For continuous efficacy endpoints, estimates of least squares means, standard error, and 95% confidence intervals (CI) will be presented for each treatment group. Nominal p-values from comparisons to placebo may be provided for descriptive purposes. The point estimate of the treatment difference and 95% confidence interval for the change from baseline at each visit for each continuous efficacy variable relative to placebo will be analyzed using a mixed effect model for repeated measures (MMRM) method.

The analysis model will include terms for baseline value as a covariate, in addition to the terms for treatment, visit, and treatment by visit interaction. For the urodynamic variables evaluated, only the independent central reviewer's interpretation will be analyzed. The proportion of subjects who achieve ≥50% reduction from baseline UUI episodes at Week 12 will be calculated for each treatment group. In addition, responder analyses will also be calculated for subjects who achieve ≥75% and 100% decrease in episodes of UUI at Week 12 relative to baseline.

The Cochran-Mantel-Haenszel (CMH) method will be utilized to compare the proportion of responders between the 2 treatment groups by adjusting for the stratification factors. Data for all visits will also be presented. For safety variables, data from all subjects in the 2 cohorts who received study medication will be included. The incidence of adverse events will be summarized. The change from baseline in PVR urine volume will be analyzed.

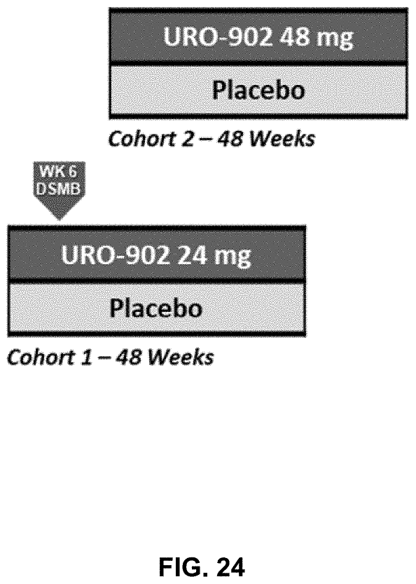

A schematic representation of the study is provided in FIG. 24.

### IV. DETAILED STUDY DESIGN

This is a multicenter, randomized, double-blind, placebo-controlled, single-treatment, 2 cohort, dose-escalation study evaluating the efficacy and safety of URO-902 (24 mg or 48 mg) in the treatment of OAB and UUI in female subjects aged 40 to 76 years old. Subjects must complete all screening procedures and must meet all eligibility requirements to qualify for enrollment and randomization. The total duration of the study is 53 weeks including a 2-week screening period (Days −35 to 21), randomization (Days 14 to 7), treatment on Day 1, and a 48-week double blind post-treatment/follow-up period. Study visits will be identical for Cohorts 1 and 2. Subjects will be evaluated during the screening period for eligibility.

Eligible subjects will be randomized to treatment within each cohort at the Randomization Visit; however, subjects will be administered study treatment via cystoscopy on Day 1. All subjects will be evaluated at scheduled post-treatment clinic visits at Weeks 2, 6, 12, 18, and 24, or until the subject exits the study. Afterwards, 2 follow-up telephone visits for assessment of safety will be performed at Week 36 and Week 48. An estimated total of 78 subjects will be enrolled into 2 cohorts, with approximately 39 subjects randomized into each cohort. In both cohorts, subjects will be randomized in a 2:1 ratio to receive either URO-902 (24 mg or 48 mg) or placebo.

Each cohort will be randomized separately, and enrollment will be sequential, starting with Cohort 1 (URO 902 24 mg [n=26] and placebo [n=13]) and followed by Cohort 2 (URO-902 48 mg [n=26] and placebo [n=13]). Subjects in both Cohort 1 and Cohort 2 will be randomized centrally (Days 14 to 7) to receive either a single treatment of URO-902 or matching placebo. Randomization will be stratified by baseline UUI episodes per day and presence or absence of DO.

In Cohort 1, an unblinded review of safety data by the DSMB will be performed after all subjects reach Week 6. Study treatment at the higher dose of URO-902 48 mg will begin only after the DSMB has recommended it is safe to proceed to Cohort 2. Details on tasks and responsibilities and assessments of safety parameters will be provided in the DSMB Charter. The independent DSMB will review the safety data throughout the entire study.

For each subject in Cohort 1 or Cohort 2, a single treatment will be administered on Day 1 after fulfillment of the “day of treatment criteria.” Subjects will receive a single treatment of URO-902 or placebo administered by intradetrusor injections via cystoscopy.

Subjects will be instructed to contact the study site to report any adverse events that occur within 48 hours following administration of study treatment. A 3-day bladder diary will be used to collect information to assess exploratory efficacy endpoints related to the number of UUI, micturition, urgency, and UI episodes per day as well as one 24-hr volume voided of urine.

Justification of Dose: An extensive series of in vitro and in vivo nonclinical studies evaluating the activity and safety of URO-902 have been conducted in OAB and ED animal models at doses up to 1 mg. The results of the nonclinical evaluations supported the initiation of URO-902 clinical studies. No toxicity was observed at any dose level in any of the preclinical studies at any dose in studies conducted to date, including multiple dosing in the ED rat model.

Extensive data in the ED and OAB animal models has shown neither histopathological abnormality at any time point in any of 40 organs evaluated or expression of the gene in any other organ other than the organ that underwent transfer more than 1 week after that transfer, as well as in ED studies conducted up to 1 month after transfer. The bladder preclinical studies evaluated single doses of 0.01, 0.03, 0.1, 0.3, and 1 mg based on an obstructed bladder surface area of 12.56 cm² and an average approximate bladder volume of 4 mL. The formula for surface area is 4πr². Hence, human bladder surface area (average bladder volume of 400 mL) is 263.5 cm². Therefore, the approximate dose relationship of human to rat bladder is 20:1.

In the completed OAB clinical studies, doses up to 25 mg by direct injection into the bladder wall/detrusor muscle were well-tolerated. Data from completed URO-902 nonclinical and clinical studies are summarized in the Example 14. In the Phase 1 proof of concept OAB study (ION-03) the equivalent doses in rat were 0.222 to 0.480 mg for the highest human dose of 24 mg and 0.148 to 0.240 mg for the lower human dose of 16 mg which were given as a single administration by multiple direct bladder injections into the detrusor muscle. No clinically meaningful safety signals were identified at either the 16 mg or 24 mg dose in Study ION-03.

A starting dose of 24 mg will be initially tested in the planned Phase 2a clinical study URO-902-2001 to evaluate the safety and efficacy of URO-902 in subjects with OAB and UUI. Study URO-902-2001 has a dose-escalation design. Based on the unblinded review of observed safety data from all subjects in Cohort 1 (URO-902 24 mg) up to Week 6, the DSMB will make the recommendation to proceed with Cohort 2.

Study treatment at the higher dose cohort (URO-902 48 mg) will begin only after the DSMB has recommended it is safe to proceed to Cohort 2. The dose equivalent to the 24 mg human dose and the 48 mg human dose in the rat is no more than 0.480 mg and 0.960 mg, respectively. As described above, the obstructed bladder preclinical studies in the rat evaluated single doses of up to 1 mg based on surface area of the bladder. In the rat ED model, doses up to 1 mg were administered by intracavernous injection. Thus, the starting dose concentration of URO-902 at 24 mg as well as the highest dose to be evaluated in the planned clinical study (48 mg) are well within the range of doses investigated in the preclinical studies.

End of Study Definition: The end of the study is defined as the date of the last visit or last scheduled procedure (Week 48) shown in the schedule of activities for the last subject in the study. A subject is considered to have completed the study if she was treated, has not been discontinued for any reason, attends the scheduled exit visit of the cohort she is enrolled in, and is properly discharged from the study.

Study population: The study is being conducted in female subjects with OAB and UUI. Specific inclusion and exclusion criteria are specified below. Prospective approval of protocol deviations to recruitment and enrollment criteria, also known as protocol waivers or exemptions, is not permitted.

Inclusion Criteria: Subjects must meet all of the following inclusion criteria to be eligible for participation in this study.
1. Capable of giving written informed consent, which includes compliance with the requirements and restriction listed in the consent form.
2. Subject is female, aged 40 to 76 years old, at screening.
3. Subject has symptoms of OAB (frequency and urgency) with UUI for a period of at least 6 months prior to screening, determined by documented subject history.
4. Subject experiences ≥1 episode of UUI per day (i.e., a total of ≥3 UUI episodes over the 3-day subject bladder diary completed during the screening period).
5. Subject experiences urinary frequency, defined as an average of ≥8 micturitions (toilet voids) per day (i.e., a total of ≥24 micturitions over the 3-day subject bladder diary completed during the screening period).
6. Subject has not been adequately managed with ≥1 oral or transdermal pharmacologic therapies for the treatment of their OAB symptoms (e.g., anticholinergics, beta-3 agonist, etc), in the opinion of the investigator. Not adequately managed is defined as meeting one of the following:
   an inadequate response after at least a 4-week period of pharmacologic therapy on Food and Drug Administration (FDA)-approved dose(s) (i.e., subject was still incontinent despite pharmacologic therapy), or
   limiting side effects after at least a 2-week period of pharmacologic therapy on FDA-approved dose(s)
7. Subject is willing to use clean intermittent catheterization (CIC) to empty the bladder at any time after receiving study treatment if it is determined to be necessary by the investigator.
8. Subject is of non-childbearing potential.
9. In the opinion of the investigator, subject is able to: complete study requirements, including using the toilet without assistance; collect urine volume voided per micturition measurements over a 24-hour period; complete bladder diaries and questionnaires; and attend all study visits.

Exclusion Criteria: Subjects will be excluded from participating in the study for any one of the following criteria assessed during the screening period and at the Randomization Visit:
1. Subject has symptoms of OAB due to any known neurological reason (e.g., spinal cord injury, multiple sclerosis, cerebrovascular accident, Alzheimer's disease, Parkinson's disease, etc).
2. Subject has a predominance of stress incontinence in the opinion of the investigator, determined by subject history.
3. Subject currently uses or plans to use medications or therapies to treat symptoms of OAB, including nocturia. Subjects previously receiving these medications must have discontinued their use prior to the start of the Screening Visit as follows:
   for desmopressin, at least one day prior
   for anticholinergic therapy, at least 14 days prior
   for intravesical anticholinergic therapy, at least 4 weeks prior
   for β₃ agonists, at least 14 days prior
4. Subjects who have previously been treated with onabotulinumtoxinA (or any other toxin) for urological indications. Subjects who have been treated with onabotulinumtoxinA (or other toxins) for non-urological indications are eligible.
5. Subject uses CIC or indwelling catheter to manage their urinary incontinence.
6. Subject has been treated with any intravesical pharmacologic agent (e.g., capsaicin, resiniferatoxin, onabotulinumtoxinA or other toxins) within 12 months of randomization.
7. Subject has history or evidence of any pelvic or urological abnormalities, bladder surgery or disease, other than OAB, that may affect bladder function including but not limited to:
   Bladder stones and/or bladder stone surgery at the time of screening or within 6 months prior to screening.
   Surgery (including minimally invasive surgery) within 1 year of screening for: stress incontinence, uterine prolapse, rectocele, or cystocele.
   Current or planned use of an implanted electrostimulation/neuromodulation device for treatment of urinary incontinence for the duration of the study)
   use of other non-implantable electrostimulatory devices for the duration of the study.
8. Subject has a history of interstitial cystitis/painful bladder syndrome, in the opinion of the investigator.
9. Subject has an active genital infection, other than genital warts, either concurrently or within 4 weeks prior to screening.
10. Subject has uterine prolapse of grade 3 or higher (i.e., cervix descends outside of the introitus)
11. Subject has a history or current diagnosis of bladder cancer or other urothelial malignancy, and/or has un-investigated suspicious urine cytology results. Suspicious urine cytology abnormalities require that urothelial malignancy is ruled out to the satisfaction of the investigator according to local site practice.
12. Subject has evidence of bladder outlet obstruction, in the opinion of the investigator at screening or randomization.
13. Subject has evidence of urethral outlet obstruction or urethral injury or stricture, in the opinion of the investigator at screening or randomization.
14. Subject has a PVR urine volume of >100 mL at screening. The PVR measurement can be repeated once on the same day; the subject is to be excluded if the repeated measure is above 100 mL.
15. Subject has had urinary retention or an elevated PVR urine volume that has been treated with an intervention (such as catheterization), within 6 months of screening. Note: voiding difficulties as a result of surgical procedures that resolved within 24 hours are not exclusionary.
16. Subject has a 24-hour total volume of urine voided >3000 mL, collected over 24 consecutive hours during the 3-day bladder diary collection period prior to randomization.
17. Subject has a history of 3 or more UTIs within 6 months of screening or is taking prophylactic antibiotics to prevent chronic UTIs. Subjects with a current acute UTI during screening can be treated appropriately and are eligible.
18. Subject has a serum creatinine level >2 times the upper limit of normal at screening.
19. Subject has current or previous uninvestigated hematuria. Subjects with investigated hematuria may enter the study if urological/renal pathology has been ruled out to the satisfaction of the investigator.
20. Subject has a known allergy or sensitivity to URO-902, anesthetics, or antibiotics to be used during the study.
21. Subject needs a walking aid on a permanent basis.
22. Subject is currently participating in or has previously participated in another therapeutic study within 30 days of screening (or longer if local requirements specify).
   Subject has a history or current evidence of any condition, therapy, laboratory abnormality or other circumstances that might, in the opinion of the investigator, confound the results of the study, interfere with the subject's ability to comply with the study procedure, or make participation in the study not in the subject's best interest.

Study Drugs Administered: All eligible subjects enrolled into the study will receive a single double-blind treatment of either URO-902 or placebo based on the cohort they are enrolled in. URO-902 (24 mg or 48 mg) or matching placebo will be administered as intradetrusor injections via cystoscopy. For Cohort 1, a single treatment of URO-902 24 mg or placebo will be administered. Based on the unblinded review of observed safety data from all subjects in Cohort 1 up to Week 6, the DSMB will make the recommendation to proceed with Cohort 2. Study treatment at the higher dose (URO-902 48 mg) will begin only after the DSMB has recommended it is safe to proceed to Cohort 2. Cohort 1: URO-902 24 mg or matching placebo (phosphate buffered saline with 20% sucrose [PBS-20%]). Cohort 2: URO-902 48 mg or matching placebo (PBS-20%). TABLE 19 provides a summary on study drugs.

TABLE 19
Summary of Study Drugs

| Study Drug Name | URO-902 | Matched Placebo |
|---|---|---|
| Identity of | URO-902 Drug Product | Phosphate Buffered Saline |
| Formulation | | with 20% Sucrose (PBS-20%) |
| Dosage | URO-902 is clear and colorless | PBS-20% is a clear and |
| Formulation | sterilized drug product solution | colorless sterilized solution |
| | supplied for intradetrusor | provided in the same matching |
| | injections. URO-902 plasmid is | container system as the URO- |
| | dissolved in PBS-20%. The | 902 product. Each vial |
| | solution is filtered and filled into a | contains 2 mL. |
| | sterilized vial and capped with a | |
| | sterilized gray stopper. Each vial | |
| | contains 2 mL at the concentration | |
| | of 4 mg/mL, which equate to 8 mg | |
| | of URO-902 per vial. | |
| Dose | 24 mg or 48 mg | Placebo |
| Route of | Intradetrusor injection via | Intradetrusor injection via |
| Administration | cystoscopy | cystoscopy |
| Dosing | Single treatment administered by | Single treatment administered |
| Instructions | the investigators or study site | by the investigators or study |
| | personnel qualified to perform | site personnel qualified to |
| | cystoscopy. | perform cystoscopy. |
| Packaging and | URO-902 will be provided in vials | Placebo will be provided |
| Labeling | in identical packaging to placebo. | in vials in identical packaging |
| | Each vial will contain 2 mL of | to URO-902. Each vial will |
| | study drug solution and will be | contain 2 mL of placebo |
| | labeled as required per regulatory | solution and will be labeled as |
| | requirement. | required per regulatory |
| | | requirement. |

Day of Treatment Criteria: For each subject in Cohort 1 or Cohort 2, a single treatment will be administered on Day 1 after fulfillment of the following “day of treatment criteria”:

(a) Negative urine dipstick reagent strip test (for nitrates and leukocyte esterase),

(b) if evaluated, negative urinalysis/urine culture/sensitivity results for a possible UTI have been reviewed,

(c) Subject is asymptomatic for a UTI, in the opinion of the investigator,

(d) No presence of bladder stones prior to or at cystoscopy,

(e) Investigator continues to deem that no condition or situation exists which, in the investigator's opinion, puts the subject at significant risk from receiving URO-902.

Treatment Administration: If a subject is taking any anticoagulants or anti-platelet drugs, consult with the subject's primary care physician (or internist, cardiologist, etc), as deemed clinically necessary by the investigator, if the subject can discontinue these drugs for 2-3 days prior to the intradetrusor injections treatment and on the day of treatment. Subjects on an anticoagulant and/or anti-platelet therapy must be managed appropriately to decrease the risk of bleeding, per the clinical judgment of the investigator.

All subjects must receive prophylactic antibiotics on Day 1 prior to treatment administration and for 1 additional day post-treatment. Prior to administration of study treatment, subjects will be instructed to void their bladder and then assume a supine position. Use of anesthesia during treatment administration will be determined by the investigator. All study procedures are to be conducted using the appropriate antiseptic technique per local site practice for a cystoscopy. After disinfection of the urethral meatus, Lubricating gel, with or without local anesthetic, to facilitate insertion of the sterile, single use transurethral catheter per local site practice is permitted.

For all subjects, local anesthesia instillation in the bladder will proceed as follows:

(1) instillation into the bladder of 1% to 4% lidocaine (or similar acting local anesthetic) prior to the procedure,

(2) instillation solution should remain in the bladder for at least 15 minutes to achieve sufficient anesthesia;

afterwards, the bladder will be drained of lidocaine, rinsed with saline, and drained again.

A flexible or rigid cystoscope will be used for administration of study treatment. Per local site practice lubricating gel will be used to insert the cystoscope. The bladder will be instilled with a sufficient amount of saline to visualize the study injections. One 12-mL syringe prefilled with 12 mL of study medication and one 1-mL syringe prefilled with PBS-20% will be prepared and ready for treatment administration. The injection needle will be primed with approximately 0.5 mL of study drug. The 12 mL of study drug will be administered as 20 injections, each approximately 0.6 mL. Under direct visualization via cystoscopy, injections will be distributed evenly across the detrusor wall and spaced approximately 1 cm apart, avoiding the bladder dome and trigone.

To administer study medication (from the 12-mL syringe), the needle should be inserted approximately 2 mm into the detrusor for each injection. For the final injection site, a sufficient amount of PBS-20% (from the 1-mL syringe) will be pushed through the injection needle to ensure delivery of the remaining amount of study medication.

After injections are administered, the saline used for visualization must not be drained from the bladder to allow subjects to demonstrate the ability to void prior to leaving the clinic. Subjects must remain in the clinic for at least 30 minutes for observation, and until a spontaneous void has occurred.

Subjects will be instructed to contact the study site to report any adverse events that occur within 48 hours following administration of study treatment.

Preparation/Handling/Storage: When URO-902 and placebo are shipped to the clinical site, the site must store both products at −20° C. The day prior to administrations, the product is to be thawed and stored in the refrigerator at 2 to 8° C. overnight. The product shall not be re-frozen after thawing. Study medication (URO-902 or placebo) can remain in the refrigerator (2 to 8° C., in the original vial) for up to 14 days.

Measures to Minimize Bias (Randomization and Blinding): In both cohorts, subjects will be randomized in a 2:1 ratio to receive either URO-902 (24 mg or 48 mg) or placebo. Each cohort will be randomized separately, and enrollment will be sequential, starting with Cohort 1 and followed by Cohort 2. At the Randomization Visit, subjects in both Cohort 1 and Cohort 2 will be randomized centrally to receive either a single treatment of URO-902 or matching placebo. Randomization will be stratified by baseline UUI episodes per day and presence or absence of DO. Subjects will be centrally assigned to randomized study drug using an interactive web response system (IWRS) and the randomization schedule generated by the sponsor or designee.

Urodynamic Parameters: Urodynamic assessments will only be performed at baseline after confirmation of subject eligibility during the Randomization Visit or at Day 1 (prior to treatment administration). A historical urodynamic study, performed no more than 90 days prior to the first day of screening, may serve as the baseline urodynamic assessment if the criteria detailed below are satisfied. At Week 12, all subjects will undergo a second urodynamic assessment.

Historical urodynamic study may be substituted for the baseline urodynamic assessment, if the following 3 criteria are met: (1) historical urodynamic study was obtained no more than 90 days prior to the first day of screening, (2) historical urodynamic results are available for evaluation by the central reader, and (3) subject was not being treated with any OAB medication or had discontinued OAB treatment.

The following urodynamic parameters are to be measured: (a) Cystometric volume at 1^{st} sensation to void (CV1^{st}sen), (b)Maximum cystometric capacity (MCC), (c) Maximum detrusor pressure during the storage phase (P_{detmax}), (d)Presence/absence of the first involuntary detrusor contraction (IDC) and, if present: Volume at first IDC (V_{PmaxIDC}) and Maximum detrusor pressure during the first IDC (P_{maxIDC}). Additional related instructions will be provided in the study manual.

Pharmacokinetic Assessments: Urine and blood samples for hSlo cDNA assessment will be collected pre-treatment from subjects on Day 1 (treatment administration), Week 6 follow-up clinic visit and Week 24 follow-up clinic/exit visit.

Efficacy, Health Outcome, and Urodynamics Endpoints: For the purposes of this study, the number of UUI episodes will be defined as the number of times a subject has marked “urge” as the main reason for the leakage as indicated on the Bladder Diary; regardless of whether more than one reason for leakage in addition to “urge” is checked. Average daily number of UUI episodes is calculated using the daily entries in the Bladder Diary, which is completed prior to each study visit. Average daily number of UUI episodes will be calculated as the total number of UUI episodes that occur on a Diary Day divided by the number of Diary Days in the Bladder Diary. A micturition is defined as “Urinated in Toilet.” Average daily micturitions at each study visit will be calculated in the same manner as described above for UUI episodes. Urinary incontinence is defined as having any reason for leakage or “Accidental Urine Leakage.” An urgency episode is defined as the “Need to Urinate Immediately.”

Statistical Methods for Efficacy Analysis: Baseline will be defined as the diary assessments collected during the screening period for all diary related efficacy endpoints and results of the questionnaires collected at the Day 1 Visit for all health outcome endpoints. For the analysis of continuous change from baseline endpoints (e.g., change from baseline in average daily number of UUI episodes, change from baseline in average daily number of micturitions, change from baseline in average daily number of UI episodes, change from baseline in average daily number of urgency episodes, change from baseline in average volume voided per micturition, change from baseline in average I-QOL total summary score, change from baseline in OAB-q score, and change from baseline in PGI-C score), a mixed model for repeated measures (MMRM) with restricted maximum likelihood estimation will be used. This model corrects for dropout and accounts for the fact that measurements taken on the same subject over time tend to be correlated, by using all available information on subjects within the same covariate set to derive an estimate of the treatment effect for a drop-out free population.

The proportion of subjects who has ≥50% reduction from baseline in UUI episodes at Week 12 will be calculated for each treatment group. In addition, responder analyses will also be calculated for subjects who achieve ≥75% and 100% decrease in episodes of UUI at Week 12 relative to baseline. The Cochran-Mantel-Haenszel (CMH) method will be utilized to compare the proportion of responders between the active and placebo groups.

### Example 14

### URO-902 Drug Product

Physical and chemical properties: URO-902 (also known as pVAX-hSlo) drug substance is a double stranded naked plasmid DNA molecule carrying the human cDNA encoding the alpha, or pore forming subunit of the human smooth muscle channel hSlo. hSlo is under control of the CMV promoter positioned upstream of the transgene and the construct also contains the bovine growth hormone poly A site, kanamycin resistance gene and pUC origin of replication. See FIG. 8.

The URO-902 drug substance was tested for plasmid weight, restriction enzyme, purity (% supercoiled), residual ribonucleic acid (RNA), isopropanol, ethanol, residual kanamycin, appearance, concentration, endotoxin, and bioburden. The general physical and chemical properties of URO-902 drug substance were determined to be stable at release.

Formulation: URO-902 is a clear and colorless sterilized drug product solution and is supplied for intravesical injection. URO-902 is dissolved in phosphate buffered saline (PBS) containing 20% sucrose (PBS-20%). The solution is then filtered and filled into a sterilized vial and capped with a sterilized gray stopper. Each vial contains 2 mL at a concentration of 4 mg/mL, which equate to 8 mg 9of URO-902 per vial. The URO-920 drug product is tested for plasmid weight, restriction enzyme, purity (% supercoiled), residual RNA, appearance, concentration, endotoxin, sterility, particulate matter, and bioactivity. The product is stable at release and on stability. The matching placebo contained PBS-20% in 2 mL/vial.

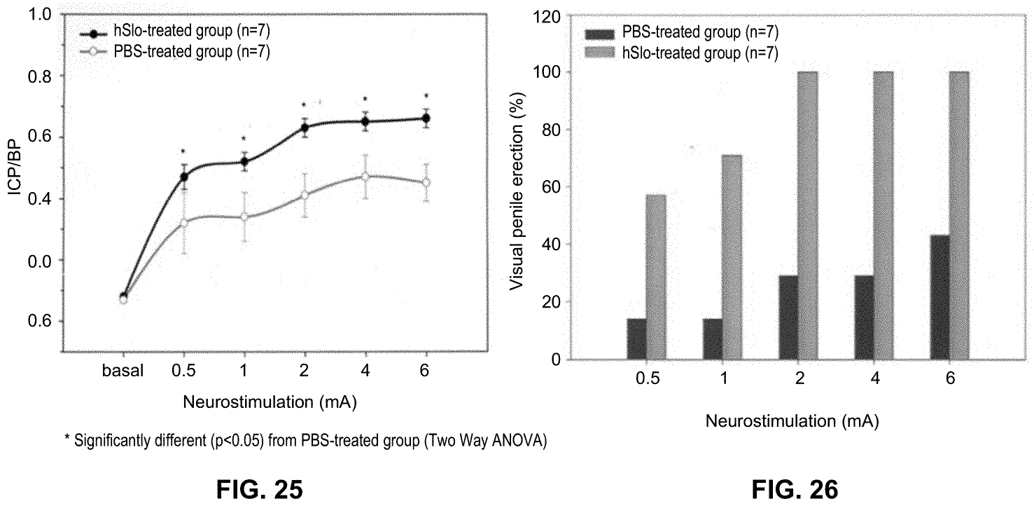

Biological Activity of the URO-902 Plasmid Construct: Historically, bioactivity of the URO-902 plasmid construct was evaluated using an in vivo erectile function bioassay in retired breeder Sprague-Dawley rats that have age-related erectile dysfunction. The assay has been previously described (see Christ, 1998; Melman, 2003). URO-902 product is injected intracorporally. One-week post-injection, rats are anesthetized and subjected to surgical procedures to allow direct cavernous nerve stimulation. Cavernous nerve stimulation is performed at the 4.0 mA level and an increased intracavernous (intracorporal) pressure to blood pressure ratio (i.e, ICP/BP) is used to show improvement in erectile dysfunction. URO-902 treated animals produce ICP/BP ratios of 0.6-0.8, and these are associated with visible erectile responses. The historical specification for URO-902 bioactivity required that the animals treated with the URO-902 plasmid attain an average ICP/BP ratio of 0.6 to 0.8 and the control animals have an ICP/BP ratio of <0.6 when stimulated at the 4 mA level. FIG. 25 and FIG. 26 shows the assay's ability to indicate bioactivity of the URO-902 plasmid.

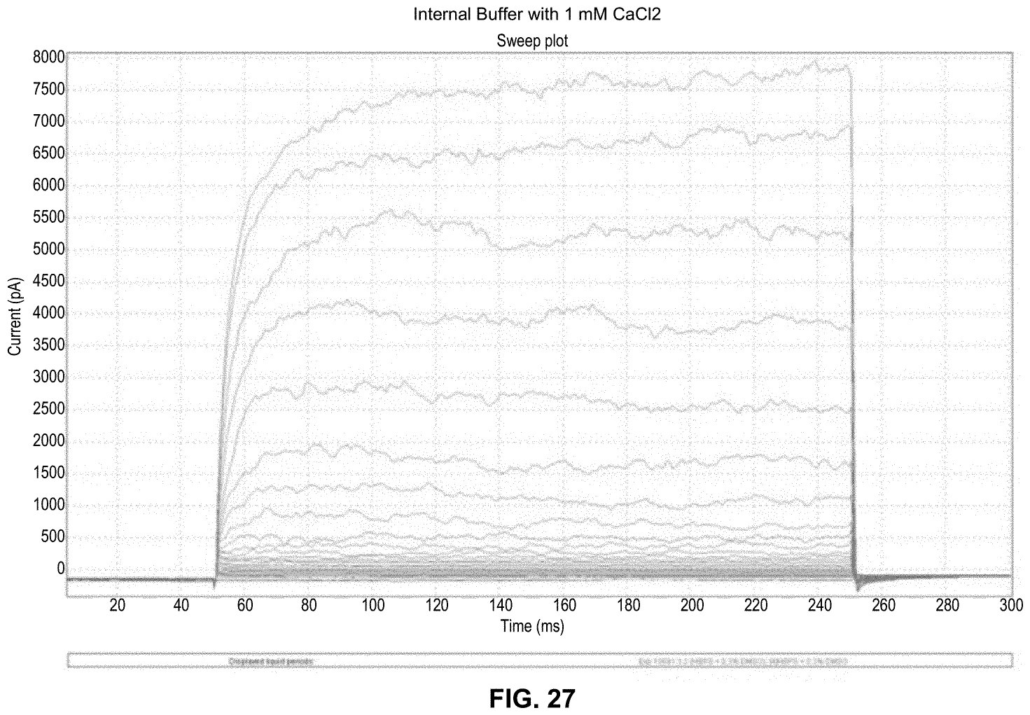

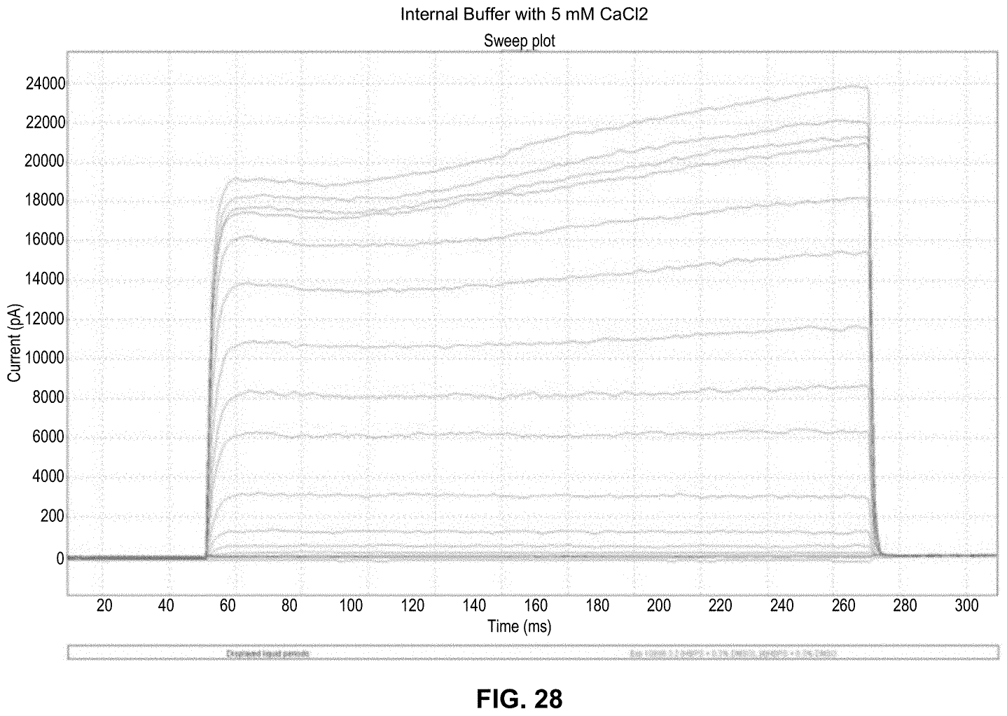

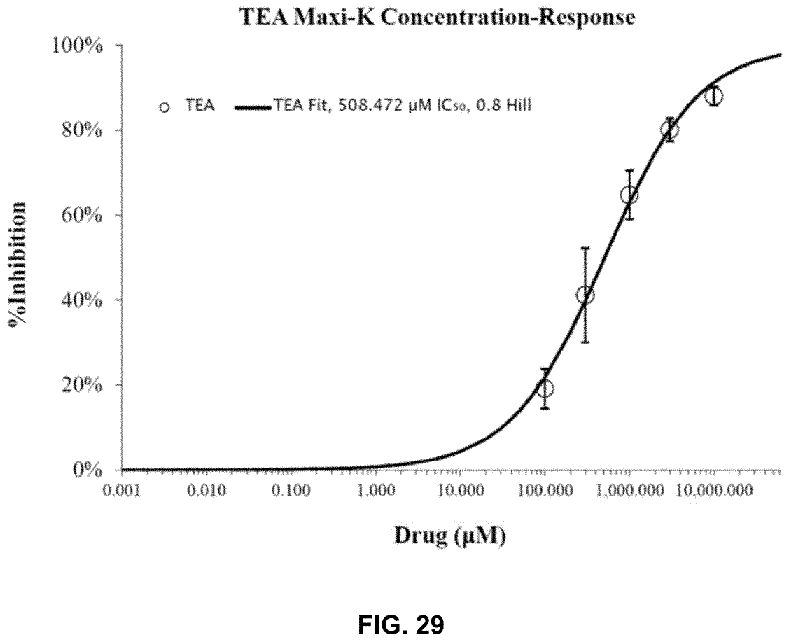

In Vitro Cell-Based Patch Clamp Model: Biological activity of the URO-902 plasmid can alternatively be evaluated in a cell-based assay showing URO-902-mediated ion channel current. In this method, the URO-902 plasmid is transiently transfected into Human Embryonic Kidney (HEK) cells. Effective transfection of the plasmid, transcription of the hSlo cDNA, translation of the hSlo protein, and insertion of the hSlo protein into the HEK cell membrane is reflected by measurable potassium (K+)-ion efflux using patch clamp technology. Ion flow specific to hSlo is confirmed using the potassium (K+) channel blocker, tetraethylammonium chloride (TEACl). Data from the in vitro patch clamp assay demonstrates URO-902 channel activity. FIGS. 27, 28, and 29 show URO-902 associated ion current, at a series of different applied voltages and internal Ca++ concentrations, that is sensitive to TEACl inhibition.

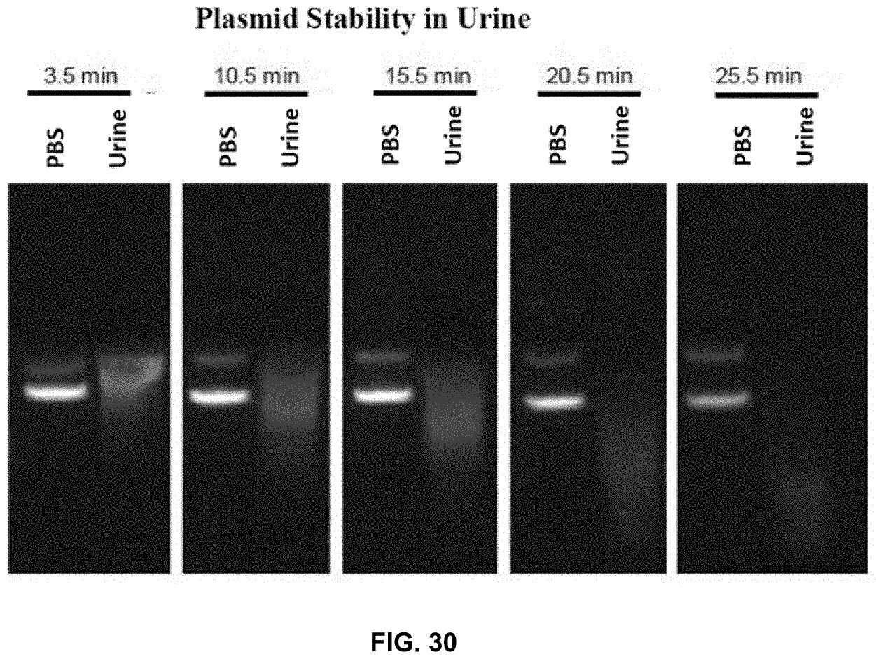

Plasmid Half-Life in Urine: The half-life of the plasmid in human urine has been determined by incubating 20 ng/μL URO-902 in 1 mL urine from a male and a female subject or in PBS. The half-life of the plasmid in the urine run at body temperature was determined to be approximately 3.5 minutes (see FIG. 30), as compared with approximately a 30-minute half-life of the plasmid in blood. Similar results were found for both the male and female urine sample. Note that in the results presented in FIG. 30 20 ng/mL URO-902 was incubated at 37° C. in either human urine or PBS. At the times indicated, samples were run on a 0.6% agarose gel and DNA was visualized with ethidium bromide. The DNA was rapidly degraded, with a half-life of approximately 3.5 minutes.

Determination of URO-902 Concentration in Tissues: URO-902 plasmid levels in tissues were determined using PCR, with primers recognizing the bacterial kanamycin resistance sequence. In each experiment, a known amount of URO-902 (10⁻¹⁶ to 10⁻⁹ g, representing approximately 12-12×10⁷ copies) was plotted against the crossover threshold determined by real-time PCR to create a standard curve. Over this concentration range, the relationship between crossover threshold and URO-902 concentration is linear. The sensitivity of the assay (using 500 ng total DNA per assay) therefore would be at least 6 copies/μg genomic DNA. The standard curve was used to derive the concentration of URO-902 in tissues by comparison of the crossover threshold obtained from tissue. These values were averaged for 4 tissues, except in gender-specific tissues, where the values of 2 tissues were averaged.

Monitoring the Structure of the Added DNA and Type of DNA (Integrated or Extrachromosomal): The amounts of plasmid that can be re-isolated in vivo after injection are insufficient for a direct analysis of the URO-902 plasmid; therefore, RT-PCR of the kanamycin gene was used to detect the presence of plasmid. In related in vitro studies, where the plasmid was incubated with rat blood, it was possible to re-isolate sufficient plasmid to perform agarose gel electrophoresis and to determine levels of intact, supercoiled, and nicked circular plasmid DNA. These experiments have demonstrated that in blood, naked supercoiled plasmid DNA degrades with a half-life of 2 hours and that the conversion of supercoiled to nicked circular DNA occurs with a half-life of less than 0.5 hours. Approximately 13 million copies plasmid/μg total DNA were detected at 1 week in the bladder.

Number of Copies Present per Cell and Stability of the Added DNA: After intracorporal injection of URO-902, plasmid could not be detected in the corpora after 1 week at the 1 copy/μg total DNA level. Bladder biodistribution studies demonstrate that 13 million copies plasmid/μg of total DNA are detectable at 1 week. The transcript can be measured up to 6 months after injection in the rat corporal smooth muscle.

Effects in Humans: URO-902 is currently being developed as a treatment for OAB. To date, 4 clinical studies have been completed in a total of 80 subjects (34 women and 46 men). Two Phase 1 studies evaluating single administrations of URO-902 have been completed in subjects with OAB; Study ION-02 evaluated intravesical instillation and Study ION-03 evaluated intradetrusor injection (via cystoscopy). Single administrations of URO-902 at 5 mg/90 mL and 10 mg/90 mL via intravesical instillation (Study ION-02) and single administrations of URO-902 at 16 mg and 24 mg via direct intradetrusor injections into the bladder (Study ION-03) were well-tolerated in female subjects with moderate OAB and DO. The majority of TEAEs were unrelated to study treatment. No SAEs were reported in Study ION-02 and the 1 SAE reported in Study ION-03 was considered unrelated to treatment by the investigator. No treatment-related deaths were reported and there were no study discontinuations due to TEAEs. Efficacy results from both studies indicated positive efficacy findings, as reflected in clinical improvements in OAB symptoms and measures of health outcomes.

In addition, Phase 1 (Study ION-301) and Phase 2 (Study ION-04-ED) studies evaluating single intracavernous injections of URO-902 have been completed in male subjects with ED. Single intracavernous injections of URO-902 at doses ranging from 0.5 mg to 16 mg were also well tolerated in male subjects with ED. The majority of adverse events reported were mild to moderate in severity and not treatment-related. Two SAEs were reported in each study and all were deemed unrelated to study treatment. No deaths occurred during either of the studies.

All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A method to treat a smooth muscle dysfunction in a subject in need thereof comprising administering at least one dose of a composition comprising an isolated nucleic acid encoding a Maxi-K potassium channel polypeptide to the subject, wherein the expression of the Maxi-K potassium channel polypeptide in smooth muscle cells of the subject modulates smooth muscle contractility, wherein the Maxi-K potassium channel polypeptide comprises
(i) a polypeptide encoding a Maxi-K alpha subunit (Slo) or a fragment, variant, mutant, or derivative thereof;
(ii) a polypeptide encoding a Maxi-K beta subunit or a fragment, variant, mutant, or derivative thereof, wherein the Maxi-K beta subunit is a beta1 subunit, a beta2 subunit, a beta3 subunit, a beta4 subunit, or a combination thereof; or,
(iii) a combination thereof,
wherein the smooth muscle dysfunction is non-neurogenic,
and wherein the administration of the composition results in the amelioration of at least one symptom of the smooth muscle dysfunction.

2-15. (canceled)

16. The method of claim 1, wherein the smooth muscle dysfunction is selected from the group consisting of overactive bladder (OAB); erectile dysfunction (ED); asthma; benign prostatic hyperplasia (BPH); coronary artery disease; genitourinary dysfunctions of the bladder, endopelvic fascia, prostate gland, ureter, urethra, urinary tract, and vas deferens; irritable bowel syndrome; migraine headaches; premature labor; Raynaud's syndrome; detrusor overactivity; glaucoma; ocular hypertension; and thromboanginitis obliterans or a symptom or sequel thereof.

17. The method of claim 1, wherein the smooth muscle dysfunction is idiopathic.

18-19. (canceled)

20. The method of claim 1, wherein the isolated nucleic acid is a DNA or an RNA.

21-27. (canceled)

28. The method of claim 1, wherein the isolated nucleic acid is a vector.

29-39. (canceled)

40. The method according to claim 28, wherein the vector is administered via parenteral administration by injection.

41. The method of claim 40, wherein the injection is intramuscular injection.

42. The method of claim 41, wherein the intramuscular injection is administered at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more injection sites.

43. (canceled)

44. The method of claim 42, wherein the injections sites are on the bladder wall.

45. The method of claim 42, wherein the injection sites are in the bladder detrusor muscle.

46. The method of claim 42, wherein the injection sites are in the bladder trigone.

47. (canceled)

48. The method of claim 40, wherein the volume of each injection is about 0.5 ml, about 1 ml, about 1.5 ml, or about 2 ml.

49. The method of claim 42, wherein the injection sites are about 0.5 cm, about 1 cm, about 1.5 cm, or about 2 cm apart.

50. The method of claim 40, wherein each injection is administered at a depth of injection of about 2 mm, 2.5 mm, 3 mm, 3.5 mm or 4 mm.

51. (canceled)

52. The method of claim 1, wherein the dose is a single unit dose.

53. The method of claim 1, wherein the dose is between 5,000 and 100,000 mcg.

54. The method of claim 1, wherein the dose is at least 10,000 mcg.

55. The method of claim 1, wherein the dose is 16,000 mcg, 24,000 mcg, or 48,000 mcg.

56. (canceled)

57. A composition for the treatment of overactive bladder (OAB) in a subject in need thereof comprising an isolated nucleic acid encoding a Maxi-K potassium channel polypeptide, wherein the Maxi-K potassium channel polypeptide comprises a polypeptide encoding a Maxi-K alpha subunit (Slo) or a fragment, variant, mutant, or derivative thereof.

58. A method to manufacture a composition for the treatment of overactive bladder (OAB) in a subject in need thereof, the method comprising inserting a nucleic acid encoding a Maxi-K potassium channel polypeptide in an expression vector, wherein the Maxi-K potassium channel polypeptide comprises
(i) a polypeptide encoding a Maxi-K alpha subunit (Slo) or a fragment, variant, mutant, or derivative thereof; or,
(ii) a polypeptide encoding a Maxi-K beta subunit or a fragment, variant, mutant, or derivative thereof, wherein the Maxi-K beta subunit is a beta1 subunit, a beta2 subunit, a beta3 subunit, a beta4 subunit, or a combination thereof.
